(19) United States
(12) Patent Application Publication
(10) Pub. No.: US 2004/0106635 A1
(43) Pub. Date: Jun. 3, 2004

(54) Spiroisoquinoline compound, a method for preparing the same and an intermediate thereof

(76) Inventors: Iwao Takamuro, Saitama-ken (JP); Koichi Homma, Osaka-fu (JP); Akihiko Ishida, Saitama-ken (JP); Hiroyuki Taniguchi, Saitama-ken (JP); Yuichi Onoda, Saitama-ken (JP)

Correspondence Address: BIRCH STEWART KOLASCH & BIRCH, PO BOX 747, FALLS CHURCH, VA, 22040-0747, US

(21) Appl. No.: 10/473,064

(22) PCT Filed: Mar. 28, 2002
(86) PCT No.: PCT/JP02/03051

(30) Foreign Application Priority Data
Mar. 29, 2001 (JP) 2001-094710
Jun. 22, 2001 (JP) 2001-189010
Oct. 24, 2001 (JP) 2001-326866

(51) Int. Cl. A61K 31/4747; C07D 487/10

(52) U.S. Cl. 514/278; 546/18

(57) ABSTRACT

The present invention provides a novel spiroistiquinoline derivative of the formula [I]: which has a small-conductance potassium channel (SK) blocking activity and is useful as a medicament, a method for preparing the same and an intermediate thereof.

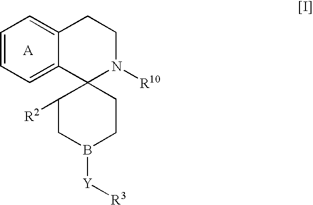

### TECHNICAL FIELD

The present invention relates to a novel spiroisoquinoline compound, which has a small-conductance potassium channel (SK) blocking activity and is useful as a medicament, a method for preparing the same and an intermediate thereof.

### BACKGROUND OF THE INVENTION

Ca²⁺-activated potassium (K) channels consist of at least three subtypes: Big-(BK), Intermediate-(IK) and Small-conductance K channel. These channels are activated by increase in intracellular Ca²⁺ level. Although BK and IK channels are sensitive to changes in membrane voltage and increase in intracellular Ca²⁺ level, SK channels are not significantly sensitive to the change in membrane voltage. Besides, SK channels are characterized in that the channels have a low conductance of 6 to 20 pS to single channel and a higher sensitivity to apamine. SK channels are present not only in excitable cells such as nerve cells and muscle cells but also in other kinds of cells such as liver cells or blood cells, and may be responsible for various cell functions including chemokine release, muscle contraction and secretion.

Apamine is a well-known selective SK channel blocker, and it has been reported that this agent activates gastrointestinal peristaltic function (S. A. Waterman and M. Costa, J. Physiology 477, 459-468, 1994; N. Spencer et al., J. Physiology 517, 889-898, 1999), facilitates learning and memory disorder (S. Ikonen et al., Eur. J. Pharmacol. 347, 13-21, 1998; C. Ghelardini et al., Br. J. Pharmacol. 123, 1079-1084, 1998) and decreases immobility time in mouse forced swimming test (N. Galeotti et al., Br. J. Pharmacol. 126, 1653-1659, 1999). Moreover, it is reported that a specific receptor for apamine exists in skeletal muscle cells and administration of this agent alleviates the symptoms in patients with myotonic muscle dystrophy (J. F. Renaud et al., Nature 319, 678-680, 1986; M. I. Behrens et al., Muscle & Nerve 17, 1264-1270, 1994). Furthermore, it was reported that mice showed abnormal respiratory responses to hypoxia under conditional overexpression of SK subtype (SK3)(C. T. Bond et al., Science 289, 1942-1946, 2000).

As compounds having a SK channel-blocking activity, bis(benzimidazol) compounds such as 1,1′-(α,α′-p-xylene)-3,3′-(α, α′-m-xylene)-bis(benzimidazolium), cyclophan compounds such as 7,18-diaza-3,4(1,4)-dibenzena-1,6(1,4)-diquinolin-acyclo-octadecaphan3trifluoroacetate hydrate, and cross-linked bisquinoline compounds such 1,4-bis-(2-methyl-quinolin-4-yl)-[1,4]-diazepane are disclosed in International Patent Publication WO00/01676, W097/48705 and the U.S. Pat. No.5,866,562, respectively. However, these publications disclose no compounds including spiroisoquinoline moiety.

### SUMMARY OF THE INVENTION

The object of the present invention is to provide a novel spiroisoquinoline compound as a medicament having an excellent SK channel blocking activity, a method for preparing the same and an intermediate thereof. Moreover, the present invention provides a medicament for prophylaxis or treatment of constipation which comprises a compound having SK channel blocking activity as an active ingredient, and a medicament for prophylaxis or treatment of constipation or central nervous system disorders which comprises a compound having a SK channel blocking activity and acetylcholine esterase inhibitory activity as an active ingredient.

The present invention relates to a spiroisoquinoline compound of the formula [I]:

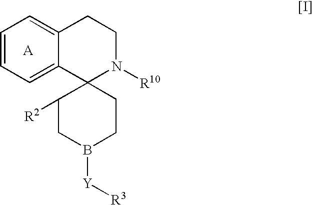

wherein ring A is an optionally substituted benzene ring,

R¹⁰ is a hydrogen atom or a group of the formula: —Z—R¹,

wherein R¹ is a hydrogen atom, an optionally substituted lower alkyl group, or an optionally substituted lower alkenyl group,

Z is a group of the formula: —CH₂— or —CO—,

R² is a hydrogen atom or an optionally substituted heterocyclic group,

B is a group of the formula:

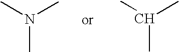

R³ is an optionally substituted amino group or an optionally substituted nitrogen-containing aliphatic heterocyclic group, and

Y is a group of the formula: —CH₂— or —CO—,

or a pharmaceutically acceptable salt thereof.

### DETAILED DESCRIPTION OF THE PREFERRED EMBODIMENTS

Examples of the substituent in the Ring A of the compound [I] include a lower alkyl group, a lower alkoxy group, an optionally protected hydroxyl group, a halogen atom, an amino group or a lower alkylenedioxy group.

The Ring A may be substituted by the same or different one to two groups selected from the group consisting of a lower alkoxy group, an optionally protected hydroxyl group, a halogen atom and an amino group, or substituted by one or two lower alkylenedioxy groups.

In case that the group R¹ in the compound [I] is an optionally substituted lower alkyl group, examples of the substituent include:

(i) a halogen atom,

(ii) an optionally protected hydroxyl group,

(iii) an amino group which may be substituted by a group(s) selected from a lower alkyl group; a lower cycloalkyl group; an aryl-lower alkyl group; a lower alkoxycarbonyl group; an acyl group; 1-amino-2-nitrovinyl group; 1-(mono- or di-)lower alkyl amino-2-nitrovinyl group; 1-amino-2,2-dicyanovinyl group; 1-(mono- or di-)lower alkylamino-2,2-dicyanovinyl group; 3-aminocyclobut-3-en-1,2-dion-4-yl group; 3-(mono- or di-)lower alkylaminocyclobut-3-en-1,2-dion-4-yl group; and a group which can be removed by an enzymatic or chemical metabolic process in vivo,

(iv) a guanidino group which may be substituted by a group(s) selected from a lower alkyl group, a lower cycloalkyl group and a cyano group,

(v) an ureido group which may be substituted by a group(s) selected from a lower alkyl group and a lower cycloalkyl group, and

(vi) a thioureido group which may be substituted by a group(s) selected from a lower alkyl group and a lower cycloalkyl group.

The groups in R¹ which can be removed by an enzymatic or chemical metabolic process in vivo mean any groups removable by hydrolysis, oxidation or reduction in vivo. Concrete examples include groups of the formula:

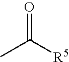

wherein R⁵ is a group of the formula:

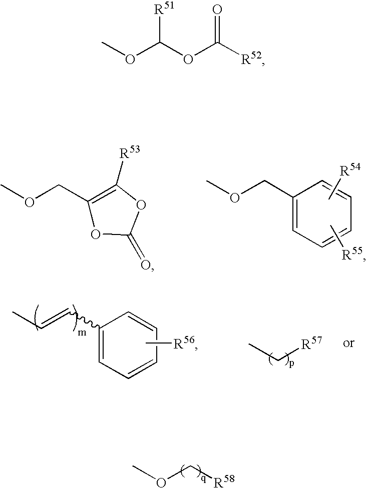

wherein R⁵¹ is a hydrogen atom or a lower alkyl group,

R⁵² is a lower alkyl group (said lower alkyl group being optionally substituted by a carboxyl group), a lower cycloalkyl group, a lower alkoxy group, a lower cycloalkoxy group or an aryl group,

R⁵³ is a lower alkyl group or an aryl group,

R⁵⁴ and R⁵⁵ are the same or different and each a hydrogen atom, a lower alkanoyloxy group, an arylcarbonyloxy group, a lower alkoxycarbonyloxy group, a lower alkanoyloxy-methyloxy group, a halogen atom or a lower alkyl group,

R⁵⁶ is a hydrogen atom, a lower alkanoyloxy-lower alkyl group or an arylcarbonyloxy-lower alkyl group,

m is an integer of 0 or 1,

R⁵⁷ is an optionally protected amino group, a lower alkoxy group, a carbamoyloxy group, a (mono- or di-)lower alkylcarbamoyloxy group, or an acyl group,

P is an integer of 1 or 2,

R⁵⁸ is a lower alkoxy group, an acyl group, a carbamoyloxy group, or a (mono- or di-)lower alkylcarbamoyloxy group, and

q is an integer of 1 or 2.

The lower alkyl group in R¹ may be substituted by the same or different one to three groups selected from the substituents mentioned above.

In case that the group R² is an optionally substituted heterocyclic group, examples of the subsituent include:

(i) a lower alkyl group,

(ii) a lower alkoxy group,

(iii) an optionally protected hydroxyl group,

(iv) a halogen atom,

(v) a lower alkylenedioxy group, and

(vi) an acyl group.

The heterocyclic group in R² may be substituted by the same or different one to four substituents mentioned above.

Examples of the heterocyclic group in R² include a mono- or bi-cyclic nitrogen-containing heterocyclic group such as a 1,2,3,4-tetrahydroisoquinolyl group, 3,4-dihydroisoquinolyl group or isoquinolyl group.

Concrete examples of R² include a group of the formula:

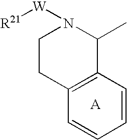

wherein R²¹ is a hydrogen atom or a lower alkyl group,

W is a group of the formula: —CH₂— or —CO— and the other symbol is the same as defined above.

In case that the group R³ in the compound [I] is an optionally substituted amino group, examples of the substituent in the amino group include the following groups:

(i) a lower alkyl group which may be substituted by a group(s) selected from an oxo group, an optionally protected amino group, a (mono- or di-)lower alkylamino group, an aryl-lower alkylimidazolylthio group, and a pyridylamino group (the pyridyl moiety of said pyridylamino group being optionally substituted by a lower alkyl group(s)),

(ii) an acyl group,

(iii) an amino group which may be substituted by a group(s) selected from a nitrogen-containing heterocyclic group which may be substituted by a lower alkyl group(s), and a lower alkyl group, and

(iv) a nitrogen-containing heterocyclic group which may be substituted by a group(s) selected from a lower alkyl group, a lower alkoxy group, an aryl-lower alkyl group, an optionally protected hydroxyl group, and an amino group.

The amino group in R³ may be substituted by the same or different one or two groups mentioned above.

In case that the R³ in the compound [I] is an optionally substituted nitrogen-containing aliphatic heterocyclic group, examples of the substituent for the aliphatic heterocyclic group include:

(i) a nitroso group,

(ii) an optionally protected amino group,

(iii) a nitrogen-containing heterocyclic group or its onium salt on nitrogen atom which may be substituted by a group(s) selected from an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, mono- or di(lower alkyl)amino group, a lower alkoxy-lower alkyl group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom and a lower alkoxy group), a furyl-lower alkyl group (the furyl moiety of said furyl-lower alkyl group being optionally substituted by a group selected from a lower alkyl group and a mono- or di-lower alkylamino-lower alkyl group), an imidazolyl-lower alkyl group, a thiazolyl-lower alkyl group (the thiazolyl moiety of said thiazolyl-lower alkyl group being optionally substituted by a group(s) selected from a lower alkyl group, a hydroxyl group, a mono- or di-lower alkylamino group and a lower alkoxy-lower alkyl group), a pyrazolyl-lower alkyl group, a pyrimidinyl-lower alkyl group (the pyrimidinyl moiety of said pyrimidinyl-lower alkyl group being optionally substituted by a lower alkyl group), a pyridazinyl-lower alkyl group, a pyridyl-lower alkyl group (the pyridyl moiety of said pyridyl-lower alkyl group being optionally substituted by a group selected from a lower alkyl group, a halogen atom, a lower alkoxy group, a lower alkoxy-lower alkyl group, a mono- or di-lower alkylamino group, a lower alkoxycarbonyl group, a mono- or di-lower alkyl-carbamoyl group, a mono- or di-lower alkylamino-lower alkyl group, a hydroxy-lower alkyl group, an oxo group and an oxide group), a carboxyl group, a lower alkoxycarbonyl group, a halogen atom, a hydroxy-lower alkyl group, a carboxyl-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group), and

(iv) a lower alkyl group which may be substituted by a group(s) selected from an oxo group, a pyridyl group, an amino group, a pyrazolyl group (said pyrazolyl group being optionally substituted by a group(s) selected from a lower alkyl group and a benzyl group), a carbamoyl group (said carbamoyl group being optionally substituted by a group(s) selected from a pyridyl group and a lower alkyl group), a thiocarbamoyl group (said thiocarbamoyl group being optionally substituted by a group(s) selected from a pyridyl group and a lower alkyl group), an amino group (said amino group being optionally substituted by a group(s) selected from an N-lower alkyl-N-pyridylcarbamoyl group, a lower alkylcarbamoyl group, a pyridylcarbamoyl group, a lower alkyl group, an amino-protecting group, a pyridylcarbonyl group, a pyridylthiocarbonyl group, a pyridyl group, and a 1-cyanoimino-1-pyridylmethyl group)

The nitrogen-containing aliphatic heterocyclic group in R³ may be substituted by the same or different one to four substituents mentioned above.

Example of the nitrogen-containing aliphatic heterocyclic group in R³ includes a 4- to 8-membered nitrogen-containing aliphatic heteromonocyclic groups such as an azetidinyl group, a pyrrolidinyl group, an imidazolidinyl group, a pyrazolidinyl group, a piperidyl group, a piperazinyl group, an azepinyl group, a diazepinyl group, an azeocinyl group, or a diazeocinyl group.

Examples of the nitrogen-containing heterocyclic group in R³ include a nitrogen-containing hetero(mono-, bi- or tri-)cyclic group such as a pyrrolyl group, an oxazolyl group, an isoxazolyl group, a thiazolyl group, an imidazolyl group, an imidazolinyl group, a pyrazolyl group, a pyridyl group, a dihydropyridyl group, a pyridazinyl group, a pyrimidinyl group, a tetrahydropyrimidinyl group, a pyrazinyl group, a pyrrolidinyl group, an imidazolidinyl group, a pyrazolidinyl group, a piperidyl group, a piperazinyl group, a triazinyl group, a morpholinyl group, an indolyl group, a quinolyl group, an isoquinolyl group, a purinyl group, a 1H-indazolyl group, a quinazolinyl group, a cinnolinyl group, a quinoxalinyl group, a phthalazinyl group, a pteridinyl group, a pyrazolopyrimidinyl group, a triazolopyrimidinyl group, an imidazopyrimidinyl group, a pyrazolopyridyl group, a triazolopyridyl group and an imidazopyridyl group.

Examples of the aryl group in R¹ or R³ include a phenyl group, a naphthyl group, an anthryl group and a phenanthryl group.

In case that the above-mentioned compound [I] of the present invention has an optionally protected amino group, examples of the protecting group include an optionally substituted lower alkoxycarbonyl group or an acyl group such as an ethoxycarbonyl group, a methoxycarbonyl group, a benzyloxycarbonyl group, a 4-methoxybenzyloxycarbonyl group, an aryloxycarbonyl group, a 9-fluorenylmethoxycarbonyl group, a tert-butoxycarbonyl group, a 2,2,2-trichloroethyloxycarbonyl group, a formyl group, an acetyl group, a propionyl group and a butyryl group. Other examples of the protecting group include a benzyl group, a 4-methoxybenzyl group and an aryl group. Among the protecting groups, preferred examples include an optionally substituted lower allkoxycarbonyl group such as a benzyloxycarbonyl group and a tert-butoxycarbonyl group.

Besides, in case that the above-mentioned compound [I] of the present invention has an optionally protected hydroxyl group, examples of the protecting group include an optionally substituted aryl-lower alkyl group, an acyl group and a tri-alkylsilyl group. Among the protecting groups, preferred example is an unsubstituted aryl-lower alkyl group such as a benzyl group or a phenethyl group, an acyl group such as a formyl group, an acetyl group, a propionyl group, a malonyl group, an acryloyl group, a benzoyl group, a methoxycarbonyl group or an ethoxycarbonyl group, a trialkylsilyl group such as a trimethylsilyl group, a triethylsilyl group or a tert-butyldimethylsilyl group, a triphenylmethyl group, and a 2-cyanomethyl group.

Furthermore, in case that the above-mentioned compound [I] of the present invention has an optionally protected carboxyl group, examples of the protecting group include any groups which can be removed by hydrolysis or hydrogenolysis, such as a lower alkyl group or a benzyl group being optionally substituted by one to two groups selected from a halogen atom, a lower alkyl group and a lower alkoxy group such as a methyl group, an ethyl group, a propyl group, a tert-butyl group, a benzyl group, a 4-chlorobenzyl group, a 4-fluorobenzyl group, a 4-methylbenzyl group or a 4-methoxya benzyl group. Among the protecting groups, preferred examples are a methyl group, an ethyl group and a benzyl group.

Among the objective compounds of the present invention, preferred examples are those wherein R¹ is a group of the formula:

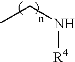

wherein R¹ is a hydrogen atom or a lower alkyl group, and n is an integer from 1 to 6, or a prodrug thereof.

Among the objective compounds [I] of the present invention, more preferred examples include compounds wherein, in the structure of the compound [I], R¹ is a group of the formula:

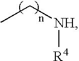

and the nitrogen atom binding to R⁴ is further substituted by a group which is removable by an enzymatic or chemical metabolic process in vivo such as a group removable in vivo by hydrolysis, oxidation or reduction.

More concrete examples are compounds wherein, in the structure of the compound [I], R¹ is a group of the formula:

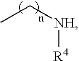

and the nitrogen atom binding to R⁴ is further substituted by a group of the formula:

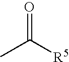

wherein R⁵ is a group of the formula:

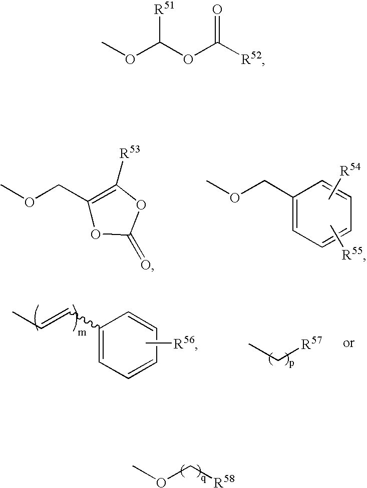

wherein R⁵¹ is a hydrogen atom or a lower alkyl group,

R⁵² is a lower alkyl group (said lower alkyl group being optionally substituted by a carboxyl group), a lower cycloalkyl group, a lower alkoxy group, a lower cycloalkoxy group or an aryl group,

R⁵³ is a lower alkyl group or an aryl group,

R⁵⁴ and R⁵⁵ are the same or different and each a hydrogen atom, a lower alkanoyloxy group, an arylcarbonyloxy group, a lower alkoxycarbonyloxy group, a lower alkanoyloxymethyloxy group, a halogen atom or a lower alkyl group,

R⁵⁶ is a hydrogen atom, a lower alkanoyloxy-lower alkyl group or an arylcarbonyloxy-lower alkyl group,

m is an integer of 0 or 1,

R⁵⁷ is an optionally protected amino group, a lower alkoxy group, a carbamoyloxy group, a (mono- or di-)lower alkylcarbamoyloxy group, or an acyl group,

P is an integer of 1 or 2,

R⁵⁸ is a lower alkoxy group, an acyl group, a carbamoyloxy group, or a (mono- or di-)lower alkylcarbamoyloxy group, and

q is an integer of 1 or 2.

Among the compounds mentioned above, particularly preferred compounds are those wherein R⁵ is a group of the formula:

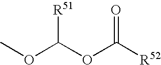

in which R⁵¹ and R⁵² are the same as defined above.

Among the objective compounds [I] of the present invention, pharmaceutically preferred examples (Group A) include compounds [I] wherein ring A is a benzene ring optionally substituted by the same or different two groups selected from a lower alkoxy group and an optionally protected hydroxyl group,

R¹⁰ is a group of the formula: —Z—R¹,

wherein R¹ is a hydrogen atom or a lower alkyl group and

Z is a group of the formula: —CH₂—,

R² is a 1,2,3,4-tetrahydroisoquinolyl group optionally substituted by a group(s) selected from a lower alkyl group, an acyl group, a lower alkoxy group and an optionally protected hydroxyl group,

R³ is

(1) a lower alkylamino group substituted by a (mono- or di-)lower alkylamino group, or

(2) a piperazinyl group which may be substituted by a group(s) selected from the group consisting of:

(i) a nitrogen-containing heteromonocyclic group or its onium salt on nitrogen atom which may be substituted by a group selected from a lower alkyl group, a carboxyl group, a lower alkoxycarbonyl group, a halogen atom, an oxo group, an oxide group and a hydroxy-lower alkyl group, and

(ii) a lower alkyl group which may be substituted by a group selected from an N-pyridyl-N-lower alkylcarbamoyl group, an oxo group, an amino group, an amino group and a pyridyl group,

Y is a group of the formula: —CO—.

Other pharmaceutically preferred compounds [I] (Group B₁) are those in which ring A is a benzene ring optionally substituted by the same or different two groups selected from a lower alkoxy group and an optionally protected hydroxyl group,

R¹⁰ is a group of the formula: —Z—R¹,

wherein R¹ is a lower alkyl group substituted by a (mono- or di-)lower alkylamino group, and

Z is a group of the formula: —CH₂— or —CO—,

R² is

(1) a hydrogen atom, or

(2) a 1,2,3,4-tetrahydroisoquinolyl group which may be substituted by a group(s) selected from a lower alkyl group, an acyl group, a lower alkoxy group and an optionally protected hydroxyl group,

R³ is a piperazinyl group substituted by a nitrogen-containing hetero(mono- or bi-)cyclic group which may be substituted by a group(s) selected from the group consisting of an amino group, a lower alkyl group, a carboxyl-lower alkyl group, a lower alkoxycarbonyl-lower alkyl group, a hydroxy-lower alkyl group, an N-pyridyl-N-lower alkylcarbamoyl group, an aryl-lower alkyl group (the aryl moiety of said aryl-lower alkyl group being optionally substituted by a halogen atom or a lower alkyl group), a pyridyl-lower alkyl group (the pyridyl moiety of said pyridyl-lower alkyl group being optionally substituted by an oxide group), a thienyl-lower alkyl group, a lower alkylamino group, a halogenobenzyloxy-lower alkyl group, a lower alkenyl group, a lower cycloalkyl group, and an aryl group (said aryl group being optionally substituted by a group(s) selected from a trifluoromethyl group, a lower alkoxy group, and a nitro group),

Y is a group of the formula: —CO—.

Another pharmaceutically preferred compounds [I] (Group B₂) are those in which ring A is a ring of the formula:

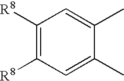

and R⁸ is a lower alkoxy group,

R¹⁰ is a group of the formula: —Z—R¹,

Wherein R¹ is a lower alkyl group substituted by a (mono- or di-)lower alkylamino group and

Z is a group of the formula: —CO—,

R² is a group of the formula:

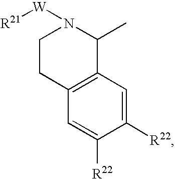

wherein R²¹ is a hydrogen atom or a lower alkyl group,

W is a group of the formula: —CH₂— or —CO—, and

R²² is a lower alkoxy group,

R³ is a piperazinyl group substituted by a group(s) selected from the group consisting of:

(1) a pyrazolopyrimidinyl group substituted by a group(s) selected from a lower alkyl group, a pyridyl-lower alkyl group and an aryl-lower alkyl group (the aryl moiety of said aryl-lower alkyl group optionally substituted by a halogen atom or a lower alkyl group),

(2) an imidazopyridyl group substituted by a group(s) selected from a lower alkyl group or an aryl-lower alkyl group (the aryl moiety of said aryl-lower alkyl group being optionally substituted by a halogen atom or a lower alkyl group), and

(3) a triazolopyrimidinyl group substituted by a lower alkyl group or an aryl-lower alkyl group (the aryl moiety of said aryl-lower alkyl group being optionally substituted by a halogen atom or a lower alkyl group), and

Y is a group of the formula: —CO—.

Some other pharmaceutically preferred compounds [I] (Group C) are those in which ring A is a ring of the formula:

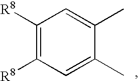

wherein R⁸ is a lower alkoxy group,

R¹⁰is a group of the formula: —Z—R¹,

wherein R¹ is a lower alkyl group substituted by an amino group optionally substituted by a 1-(mono- or di-)lower alkylamino-2-nitrovinyl group), and

Z is a group of the formula: —CH—,

R² is a hydrogen atom,

R³ is a piperazinyl group substituted by a pyrazolopyrimidinyl group substituted by a pyridyl-lower alkyl group or an aryl-lower alkyl group (the aryl moiety of said aryl-lower alkyl group being optionally substituted by a nitro group, a halogen atom or a lower alkyl group), and

Y is a group of the formula: —CO—.

Among the objective compounds [I] of the present invention, pharmaceutically more preferred examples (Group D₁) are compounds [I] in which ring A is a benzene ring optionally substituted by the same or different two groups selected from a lower alkoxy group and an optionally protected hydroxyl group,

R¹⁰ is a group of the formula: —Z—R¹,

wherein R¹ is an amino-substituted lower alkyl group (the amino group of said amino-substituted lower alkyl group may be substituted by a lower alkyl group or a group which is removable by an enzymatic or chemical metabolic process in vivo),

Z is a group of the formula: —CH₂— or —CO—,

R² is

(1) a hydrogen atom, or

(2) a 1,2,3,4-tetrahydroisoquinolyl group which may be substituted by a group(s) selected from a lower alkyl group, an acyl group, a lower alkoxy group and an optionally protected hydroxyl group,

R³ is a piperazinyl group substituted by a nitrogen-containing hetero(mono- or bi-)cyclic group which may be substituted by a group(s) selected from an amino group, a lower alkyl group, a carboxyl-lower alkyl group, a lower alkoxycarbonyl-lower alkyl group, a hydroxy-lower alkyl group, an N-pyridyl-N-lower alkylcarbamoyl group, an aryl-lower alkyl group (the aryl moiety of said aryl-lower alkyl group being optionally substituted by a halogen atom or a lower alkyl group), a pyridyl-lower alkyl group (the pyridyl moiety of said pyridyl-lower alkyl group being optionally substituted by an oxide group), a thienyl-lower alkyl group, a lower alkylamino group, a halogenobenzyloxy-lower alkyl group, a lower alkenyl group, a lower cycloalkyl group, and an aryl group (said aryl group being optionally substituted by a group(s) selected from a trifluoromethyl group, a lower alkoxy group, and a nitro group), and

Y is a group of the formula: —CO—.

Among the objective compounds [I] of the present invention, pharmaceutically more preferred examples (Group D₂) are compounds [I] wherein ring A is a group of the formula:

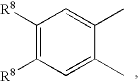

wherein R⁸ is a lower alkoxy group,

R¹⁰ is a group of the formula: —Z—R¹,

wherein R¹ is an amino-substituted lower alkyl group (the amino group of said amino-substituted lower alkyl group being optionally substituted by a lower alkyl group or a group which is removable by an enzymatic or chemical metabolic process in vivo), and

Z is a group of the formula: —CO—,

R² is a group of the formula:

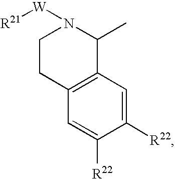

wherein R²¹ is a hydrogen atom or a lower alkyl group,

W is a group of the formula: —CH— or —CO—, and

R² is a lower alkoxy group,

R³ is a piperazinyl group substituted by a group selected from the group consisting of:

(1) a pyrazolopyrimidinyl group substituted by a lower alkyl group, a pyridyl-lower alkyl group or an aryl-lower alkyl group (the aryl moiety of said aryl-lower alkyl group being optionally substituted by a halogen atom or a lower alkyl group),

(2) an imidazopyridyl group substituted by a lower alkyl group or an aryl-lower alkyl group (the aryl moiety of said aryl-lower alkyl group being optionally substituted by a halogen atom or a lower alkyl group), or

(3) a triazolopyrimidinyl group substituted by a lower alkyl group or an aryl-lower alkyl group (the aryl moiety of said aryl-lower alkyl group being optionally substituted by a halogen atom or a lower alkyl group), and

Y is a group of the formula: —CO—.

Among the objective compounds [I] of the present invention, particularly pharmaceutically preferred examples are compounds [I] of the formula [I-h]:

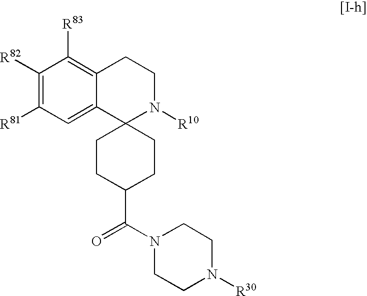

wherein R⁸¹, R⁸² and R⁸³ are the same or different groups selected from the group of a hydrogen atom, a lower alkoxy group, an optionally protected hydroxyl group and a halogen atom,

R¹⁰ is a hydrogen atom or a group of the formula: —Z—R¹,

wherein Z is a group of the formula: —CH₂— or —CO—,

R¹ is

(1) a hydrogen atom,

(2) a lower alkyl group which may be substituted by a group(s) selected from the group consisting of:

(i) a halogen atom,

(ii) an optionally protected hydroxyl group,

(iii) an amino group which may be substituted by a group(s) selected from a lower alkyl group; a lower cycloalkyl group; an aryl-lower alkyl group; a lower alkoxycarbonyl group; an acyl group; 1-amino-2-nitrovinyl group; 1-(mono-or di-)lower alkyl amino-2-nitrovinyl group; 1-amino-2,2-dicyanovinyl group; 1-(mono- or di-)lower alkylamino-2,2-dicyanovinyl group; 3-aminocyclobut-3-en-1,2-dion-4-yl group; 3-(mono- or di-)lower alkylaminocyclobut-3-en-1,2-dion-4-yl group; and a group which is removable by an enzymatic or chemical metabolic process in vivo,

(iv) a guanidino group which may be substituted by a group(s) selected from a lower alkyl group, a lower cycloalkyl group and a cyano group,

(v) an ureido group which may be substituted by a group(s) selected from a lower alkyl group and a lower cycloalkyl group, and

(vi) a thioureido group which may be substituted by a group(s) selected from a lower alkyl group and a lower cycloalkyl group, or

(3) a lower alkenyl group, and

R30 is a nitrogen-containing heterocyclic group or its onium salt on nitrogen atom which may be a group(s) selected from the group consisting of an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom and a lower alkoxy group), a furyl-lower alkyl group, an imidazolyl-lower alkyl group, a thiazolyl-lower alkyl group (the thiazolyl moiety of said thiazolyl-lower alkyl group being optionally substituted by a lower alkyl group), a pyrazolyl-lower alkyl group, a pyrimidinyl-lower alkyl group, a pyridazinyl-lower alkyl group, a pyridyl-lower alkyl group (the pyridyl moiety of said pyridyl-lower alkyl group being optionally substituted by a group selected from a lower alkyl group and an oxide group), a carboxyl group, a lower alkoxycarbonyl group, a halogen atom, a hydroxy-lower alkyl group, a carboxy-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group).

Among the objective compounds [I] of the present invention, other particularly pharmaceutically preferred examples are compounds [I-h] wherein R³⁰ is a group of the formula:

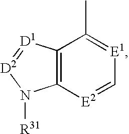

wherein D¹ and D² are the same or different and each a group of the formula: —N═ or —CH═,

one of E¹ and E² is a group of the formula: —N═ , and the other is a group of the formula: —NH═ or —CH═, and

R³¹ is a group selected from the group consisting of a hydrogen atom, an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom and a lower alkoxy group), a furyl-lower alkyl group, an imidazolyl-lower alkyl group, a thiazolyl-lower alkyl group (the thiazolyl moiety of said thiazolyl-lower alkyl group being optionally substituted by a lower alkyl group), a pyrazolyl-lower alkyl group, a pyrimidinyl-lower alkyl group, a pyridazinyl-lower alkyl group, a pyridyl-lower alkyl group (the pyridyl moiety of said pyridyl-lower alkyl group being optionally substituted by a group selected from a lower alkyl group and an oxide group), a carboxyl group, a lower alkoxycarbonyl group, a halogen atom, a hydroxy-lower alkyl group, a carboxy-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group).

Among the objective compounds [I] of the present invention, another particularly pharmaceutically preferred examples are compounds [I-h] wherein R³⁰ is a group of the formula:

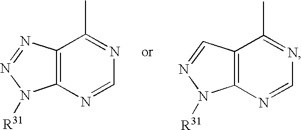

wherein R³¹ is a group selected from the group consisting of a lower alkyl group, a pyridyl-lower alkyl group (the pyridyl moiety of the pyridyl-lower alkyl group being optionally substituted by a lower alkyl group), a thiazolyl-lower alkyl group (the thiazolyl moiety of said thiazolyl-lower alkyl group being optionally substituted by a group(s selected from a lower alky group), or a pheriyl-lower alkyl group (the phenyl moiety of said phenyl-lower alkyl group may be substituted by a group(s) selected from a lower alkyl group).

Among the objective compounds [I] of the present invention, concrete examples of the preferred compound include the following compounds:

(1α,4β)-2′-methyl-3′,4′-dihydro-6′,7′-dimethoxy-4-[4-(3-methyl-3H-1,2,3-triazolo[4,5-d]pyrimidin-7-yl)piperazin-1-yl]carbonyl-spiro[cyclohexane-1,1′(2′H)-isoquinoline],

(1α,4β)-3′,4′-dihydro-6′,7′-diethoxy-4-[4-(1-(3-ethoxy-phenylmethyl)-1H-pyrazolo[3,4-d]pyrimidin-4-yl)piperazin-1-yl]carbonyl-spiro[cyclohexane-1,1′(2′H)-isoquinoline]

(1α,4β)-2′-methyl-3′,4′-dihydro-6′,7′-diethoxy-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-2′-dimethylaminoacetyl-3′,4′-dihydro-6′-methoxy-4-[4-(1-(6-methylpyridin-2-ylmethyl)-1H-pyrazolo[3,4-d]pyrimidin-4-yl) piperazin-1-yl]carbonyl-spiro[cyclohexane-1,1′(2′H)-isoquinoline],

(1α,4β)-3′,4′-dihydro-6′,7′-dimethoxy-4-[4-(1-(6-ethyl-pyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-diethoxy-4-[4-(1-(6-ethylpyridin-2-ylmethyl)-1H-pyrazolo[3,4-d]pyrimidin-4-yl)-piperazin-1-yl]carbonyl-spiro[cyclohexane-1,1′(2′H)-isoquinoline],

(1α,4β)-2′-methyl-3′,4-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-2′-methyl-3′,4′-dihydro-6′,7′-diethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-2′-ethyl-3′,4′-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-2′-dimethylaminoacetyl-3′,4′-dihydro-6′,7′-diethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-4-[4-(1-(2-ethylthiazo1-4-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-2′-dimethylaminoacetyl-3,4′-dihydro-6′-ethoxy-4-[4-(1-(6-methylpyridin-2-ylmethyl)-1H-pyrazolo[3,4-d]pyrimidin-4-yl)piperazin-1-yl)carbonyl-spiro[cyclohexane-1,1′(2′H)-isoquinoline],

(1α,4β)-2′-dimethylaminoacetyl-3′,4′-dihydro-6′,7′-dimethoxy-4-[4-(1-(2-methylthiazo1-4-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-2′-[3-(N,N-dimethylamino)propionyl]-4-[4-(1-(3-methylbenzyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-2′-[3-(N,N-dimethylamino)propionyl)-4-[4-(1-(3-methoxybenzyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-2′-[2-(N,N-dimethylamino)ethyl]-4-[4 -(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-diethoxy-2′-[2-(N,N-dimethylamino)ethyl]-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-diethoxy-2′-(N,N-dimethylaminoacetyl)-4-[4-(1-(3-methylbenzyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-4-[4-(1-(3-ethoxybenzyl)-1H-pyrazolo(3,4-d]pyrimidin-4-yl)piperazin-1-yl]carbonyl-spiro[cyclohexane-1,1′(2′H)-isoquinoline],

(1α,4β)-3′,4′-dihydro-6′-methoxy-4-[4-(1-(3-ethoxybenzyl)-1H-pyrazolo[3,4-d]pyrimidin-4-yl)piperazin-1-yl]carbonyl-spiro[cyclohexane-1,1′(2′H)-isoquinoline],

(1α,4β)-2′-(N-methylaminoacetyl)-3′,4′-dihydro-6′,7′-dimethoxy-4-[4-(1-( 3-ethoxybenzyl)-1H-pyrazolo[3,4-d]pyrimidin-4-yl)piperazin-1-yl]carbonyl-spiro[cyclohexane-1,1′(2′H)-isoquinoline],

(1α,4β)-2′-(N-methylaminoacetyl)-3′,4′-dihydro-6′,7′-dimethoxy-4-[4-(1-(3-trifluoromethoxybenzyl)-1H-pyrazolo[3,4-d]pyrimidin-4-yl)piperazin-1-yl]carbonyl-spiro[cyclohexane-1,1′(2′H)-isoquinoline],

(1α,4β)-2′-(N-methylaminoacetyl)-3′4′-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane -1,1′(2′H)-isoquinoline],

(1α,4β)-3′,4′-dihydro-6′,7′-dimethoxy-4-[4-(1-(6-n-propylpyridin-2-ylmethyl)-1H-pyrazolo[3,4-d]pyrimidin-4-yl)piperazin-1-yl]carbonyl-spiro[cyclohexane-1,1′(2′H) -isoquinoline],

(1α,4β)-2′-(N,N-dimethylaminoacetyl)-3′,4′-dihydro-6′-ethoxy-4-[4-(1-(6-ethoxypyridin-2-ylmethyl)-1H-pyrazolo[3,4-d]pyrimidin-4-yl)piperazin-1-yl]carbonyl-spiro[cyclohexane-1,1′(2′H)-isoquinoline],

(1α,4β)-3′,4′-dihydro-6′,7′-dimethoxy-2′-[3-(1-methyl-amino-2-nitrovinylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(1-amino-2-nitrovinylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(N-(n-butyl)ureido)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(N-ethylureido)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7-dimethoxy-2′-[3-(3-dimethylamino-3-cyclobuten-1,2-dion-4-yl)aminopropyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(3-methylamino-3-cyclobuten -1,2-dion-4-yl) aminopropyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(3-amino-3-cyclobuten-1,2-dion-4-yl)aminopropyl]-4-[4-[1-(2-nitro-benzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(1-amino-2,2-dicyanovinylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(1,3-dimethyl-2-cyanoguanidino)propyl]-4-[4-[1-(2-nitrobenzyl)-1H-pyrazolo[3,4-d]pyrimidin-4-yl]piperadin-1-yl ]carbonyl-spiro[cyclohexane-1,1′(2′H)-isoquinoline],

(1α,4β)-3′,4′-dihydro-6′,7′-dimethoxy-2′-[3-(N-isopropylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-(N,N-dimethylaminoacetyl)-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-ethyl-4-[4-[1-(3-methylbenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-methyl-4-[4-[1-(2-fluorobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(N-ethoxy-carbonylamino)propyl]-4-[4-[1-(2-bromobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′4′-dihydro-6′,7′-dimethoxy-2′-[3-(N-ethoxy-carbonylamino)propyl]-4-[4-[1-(2-chlorobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-dimethoxy-2′-[3-(N-ethoxy-carbonylamino)propyl]-4-[4-[1-(2-cyanobenzyl)-1H-pyrazolo[3,4-d]pyrimidin-4-yl]piperadin-1-yl]carbonyl-spiro[cyclohexane-1,1′(2′H)-isoquinoline],

(1α,4β)-3′,4′-dihydro-6′,7′-diethoxy-2′-methyl-4-[4-[1-(3-nitrobenzyl)-1H-pyrazolo[3,4-d]pyrimidin-4-yl]piperadin-1-yl]carbonyl-spiro[cyclohexane-1,1′(2′H)-isoquinoline], and

(1α,4β)-3′,4′-dihydro-6′,7′-diethoxy-2′-methyl-4-[4-[1-(3-chlorobenzyl)-1H-pyrazolo[3,4-d]pyrimidin-4-yl]piperadin-1-yl]carbonyl-spiro[cyclohexane-1,1′(2′H)-isoquinoline],

or a pharmaceutically acceptable salt thereof.

Among the objective compounds[I] of the present invention, other concrete examples of the preferred compound include the following compounds:

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(3-pyridylmethyl)-1H-pyrazolo[3,4-d]pyrimidin-4-yl]-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(4-butyl-4H-imidazo[4,5-b]pyridin-7-yl)-1-piperazinyl]carbonyl-spiro[cyclohexane-1,1′(2′H)-isoquinoline],

2′-[3-(methylamino)propionyl)-3′,4′-dihydro-6′,7′-dimethoxy-2-(2-ethyl-1,2,3,4-tetrahydro-6,7-dimethoxy-1-isoquinolyl)-4-[4-(3-methyl-3H-1,2,3-triazolo (4,5-d]pyrimidin -7-yl)-1-piperazinyl]carbonyl-spiro[cyclohexane-1,1′(2′H)-isoquinoline],

2′-[3-(methylamino)propiony]-3′,4′-dihydro-6′,7′-dimethoxy-2-(2-ethyl-1,2,3,4-tetrahydro-6,7-dimethoxy-1-isoquinolyl)-4-[4-(1-propyl-1H-pyrazolo[3,4-d]pyrimidin-4-yl )-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(2-chrolophenylmethyl)-1H-pyrazolo[3,4-d]pyrimidin-4-yl)-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(3-methylphenylmethyl)-1H-pyrazolo[3,4-d]pyrimidin-4-yl]-1-piperazinyl]carbonyl-spiro[cyclohexane-1,1′(2′H)-isoquinoline],

2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(1-n-butyl-1H-pyrazolo[3,4-d]pyrimidin-4-yl )-1-piperazinyl]carbonyl-spiro[cyclohexane-1,1′(2′H)-isoquinoline],

2′-(3-aminopropyl)-3′,4′-dihydro-6′,7′-dimethoxy-4-[4-[1-(2-pyridylmet hyl)-1H-pyrazolo[3,4-d]pyrimidin-4-yl]-1-piperazinyl]carbonyl-spiro[cyclohexane-1,1′(2′H) -isoquinoline],

2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline],

2′-[3-(2-cyano-3, 3-dimethylguanidino)propyl]-3′4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline],

2′-[3-(1-dimethylamino-2-nitrovinylamino)propyl]-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline],

2′-(3-aminopropyl)-3′,4′-dihydro-6′,7′-dimethoxy-4-[4-[1-(2-bromophenylmethyl)-1H-pyrazolo[3,4-d]pyrimidin-4-yl]-1-piperazinyl]carbonyl-spiro[cyclohexane-1,1′(2′H)-isoquinoline],

2′-[3-(N-(propionyloxymethyloxycarbonyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H) -isoquinoline],

2′-[3-[N-(pivaloyloxymethyloxycarbonyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],

2′-[3-[N-(pivaloyloxymethyloxycarbonyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-(3-methyl-3H-1,2,3-triazolo[4,5-d]pyrimidin-7-yl)-1-piperazinyl]carbonyl-spiro[cyclohexane-1,1′(2′H)-isoquinoline],

2′-[3-[N-(cyclopropylcarbonyloxymethyloxycarbonyl)-N-methyl-amino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],

2′-[3-(pivaloyloxymethyloxycarbonylamino)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline],

(1α,4β)-3′,4′-dihydro-6′,7′-dimethoxy-4-[4-[1-(3-nitrobenzyl)-1H-pyrazolo[3,4-d]pyrimidin-4-yl]-1-piperazinyl]carbonyl-spiro[cyclohexane-1,1′(2′H)-isoquinoline ],

(1α,4β)-2′-dimethylaminoacetyl-3′,4′-dihydro-6′,7′-dimethoxy-4-[4-[1-(3-methylbenzyl)-1H-pyrazolo[3,4-d]pyrimidin-4-yl]-1-piperazinyl]carbonyl-spiro[cyclohexane-1,1′(2′H)-isoquinoline],

(1α,4β)-2′-methyl-3′,4′-dihydro-6′,7′-dimethoxy-4-[4-(3-methyl-3H-1,2,3-triazolo[4,5-d]pyrimidin-7-yl)-1-piperazinyl]carbonyl-spiro[cyclohexane-1,1′(2′H)-isoquinoline],

(1α,4β)-2′-ethyl-3′,4′-dihydro-6′,7′-diethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline], and

or a pharmaceutically acceptable salt thereof.

Among those compounds, particularly preferred embodiments are

(1R*,2R*(S*),4R*)-2′-[3-(methylamino)propionyl]-3′,4′-dihydro-6′,7′-dimethoxy -2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],

(1R*,2R*(S*),4R*)-2′-[3-[N-(propionyloxymethyloxycarbonyl)-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline], and

(1α,4β)-2′-methyl-3′,4′-dihydro-6′,7′-dimethoxy-4-[4-(3-methyl-3H-1,2,3-triazolo[4,5-d]pyrimidin-7-yl)-1-piperazinyl]carbonyl-spiro[cyclohexane-1,1′(2′H)-isoquinoline],

or a pharmaceutically acceptable salt thereof.

Besides, a spiroisoquinoline derivative, being useful as an intermediate compound for preparing the compound [I], of the formula[II-A]:

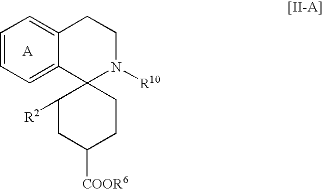

wherein ring A is an optionally substituted benzene ring,

R¹⁰ is a hydrogen atom or a group of the formula: —Z—R¹,

R¹ is a hydrogen atom, an optionally substituted lower alkyl group or an optionally substituted lower alkenyl group,

Z is a group of the formula: —CH₂— or —CO—,

R² is a hydrogen atom or an optionally substituted heterocyclic group, and R⁶ is a hydrogen atom, a lower alkyl group or a benzyl group,

or a salt thereof, is a novel compound.

Moreover, a spiroisoquinoline derivative, being useful as an intermediate compound for preparing the compound [I], of the formula:

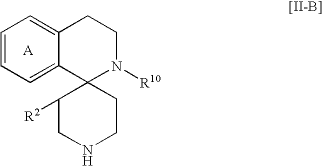

wherein ring A is an optionally substituted benzene ring,

R¹⁰ is a hydrogen atom or a group of the formula: —Z—R¹,

wherein R¹ is a hydrogen atom, an optionally substituted lower alkyl group or an optionally substituted lower alkenyl group, and

Z is a group of the formula: —CH₂— or —CO—, and

R² is a hydrogen atom or an optionally substituted heterocyclic group,

or a salt thereof is a novel compound.

When the compound [I] of the present invention has an asymmetric carbon atom(s) at the substituent in groups R¹, R² and/or R³ or the spiro-ring moiety, it may exist in the form of a stereo-isomer thereof (diastereoisomers, optical isomers) owing to said asymmetric carbon atom(s) thereof, and the present invention also includes these optical isomers and a mixture thereof.

A compound [I] of the present invention or a pharmaceutically acceptable salt thereof shows a significant antagonizing activity against apamine, which is known as a selective SK channel blocker, in a competitive binding assay. Therefore, the compound [I] or a pharmaceutically acceptable salt thereof is useful as a SK channel blocker which is applicable to treatment and/or prophylaxis of SK channel-related diseases such as gastrointestinal motility disorders (e.g., constipation, irritable bowel syndrome, gastroesophageal reflux disease or post-operative ileus), central nervous system disorders (e.g., memory and learning disorders including Alzheimer's disease), emotional disorders, myotonic muscular dystrophy or sleep apnea.

The compounds [I] of the present invention include compounds having both SK channel blocking activity and acetylcholinesterase (AChE)-inhibitory activity. These compounds may be also applicable to treatment and/or prophylaxis of gastrointestinal motility disorders (e.g., constipation, irritable bowel syndrome, gastroesophageal reflux disease or post-operative ileus), central nervous system disorders (e.g., memory and learning disorders including Alzheimer's disease), emotional disorders, myotonic muscular dystrophy or sleep apnea. Examples of the compounds having both SK channel blocking activity and AChE-inhibitory activity include compounds in Group B₁, B₂, D₁ or D₂ mentioned above.

The present invention also includes a novel medicament for prophylaxis or treatment of constipation, irritable bowel syndrome, gastroesophageal reflux disease or post-operative ileus, which comprises as an active ingredient a compound having SK-channel blocking activity.

Moreover, the compound [I] of the present invention shows a low toxicity and are safe as medicaments.

The compound [I] of the present invention can be clinically used either in the free form or in the form of a pharmaceutically acceptable salt thereof. The pharmaceutically acceptable salt of the compound [I] includes a salt with an inorganic acid such as hydrochloride, sulfate, phosphate or hydrobromide, or a salt with an organic acid such as acetate, fumarate, oxalate, citrate, methanesulfonate, benzenesulfonate, tosylate or maleate. Besides, when the compound [I] has a carboxyl group(s) in its molecule, examples of the pharmaceutically acceptable salt include salts with a base such as alkaline metal (e.g., sodium salt, potassium salt) or alkaline earth metal (e.g., calcium salt).

The compound [I], a salt thereof, or its intermediate or a salt of the present invention includes either intramolecular salt or an additive thereof, and solvates or hydrates thereof.

The present compound [I] or a pharmaceutically acceptable salt thereof can be either orally or parenterally, and can be formulated into a conventional pharmaceutical preparation such as tablets, granules, fine granules, capsules, powders, injections or inhalants.

The dose of the compound [I] of the present invention or a pharmaceutically acceptable salt thereof may vary in accordance with the administration routs, and the ages, weights and conditions of the patients. For example, when administered in an injection preparation, it is usually in the range of about 0.0001 to 1 mg/kg/day, preferably in the range of about 0.001 to 0.1 mg/kg/day. When administered in an oral preparation, it is usually in the range of about 0.001 to 100 mg/kg/day, preferably in the range of 0.01 to 10 mg/kg/day.

### BEST MODE FOR CARRYING OUT THE INVENTION

The compound [I] of the present invention may be prepared by the following Process A to G.

Process A:

Among the compound [I] of the present invention, the compound of the formula [I-a]:

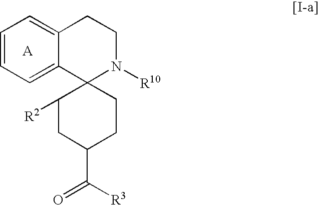

wherein the symbols are the same as defined above, can be prepared by reacting a compound of the formula[II-A]:

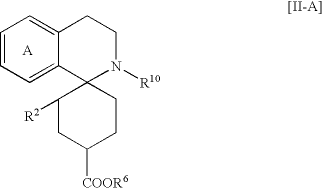

wherein the symbols are the same as defined above, or a salt thereof,

with a compound of the formula [16):

R³—H [16]

wherein R³ is the same as defined above.

When R⁶ is hydrogen atom, the above-mentioned reaction can be carried out in a solvent in the presence of a condensing agent, and in the presence or absence of an activating agent and a base. Examples of the solvent include any solvent which does not disturb the reaction, such as methylene chloride, chloroform, dimethylformamide, dimethylacetamide, tetrahydrofuran, dioxane, toluene, benzene, 1,2-dichloroethane, 1-methylpyrrolidinone or 1,2-dimethoxyethane.

The condensing agent includes, for example, dicyclo-hexylcarbodiimide (DCC), 1-ethyl-3-(3-dimethylamino-propyl]carbodiimide.hydrochloride (WSC.HCl), diphenyl-phosphorylazide (DPPA), carbonyldiimidazole (CDI), diethylcyano-phosphonate (DEPC), diisopropylcarbodiimide (DIPCI), benzotriazole-1-yloxy-tripirrolidinophosphonium hexafluoro-phosphate (PyBOP), and carbonylditriazole. Examples of the activating agent include 1-hydroxybenzotriazole (HOBt), hydroxysuccinimide (HOSu), dimethylaminopyridine (DMAP), 1-hydroxy-7-azabenzotriazole (HOAt), hydroxyphthalimide (HOPht) and pentafluorophenol (Pfp-OH). The base includes, for example, pyridine, triethylamine, diisopropylethylamine, 4-methyl-morpholine and 1,8-diazabicyclo[5,4.0]-7-undecene (DBU).

Concomitantly, when R⁶ is hydrogen atom, the reaction process A can be carried out by converting the compound [II-A] to a reactive derivative at the carboxyl group (e.g., an acid halide, a mixed acid anhydride) and reacting the reactive derivative with the compound [16] in the presence of the base mentioned above.

When R⁶ in the compound [II-A] is a lower alkyl group or benzyl group, the reaction process A can be carried out by removing the ester residue to convert the R⁶ to a free carboxyl group by a conventional hydrolysis or reduction and treating the thus-obtained product in the same manner as described above.

Besides, when R⁶ in the compound [II-A) is a lower alkyl group or benzyl group, the reaction process can be also carried out by directly reacting the compound [II-A] with the compound [16] in the presence of a base in a solvent or without solvent. The solvent includes any solvent which does not disturb the reaction, such as methylene chloride, chloroform, dimethylformamide, dimethylacetamide, tetrahydrofuran, dioxane, toluene, benzene, 1,2-dichloroethane, 1-methylpyrrolidine, methanol, ethanol and isopropyl alcohol. The base includes, for example, triethylamine, diisopropylethylamine, 4-methylmorpholine,1,8-diazabicyclo[5,4,0)-7-undecene (DBU) and dimethylaminopyridine (DMAP).

Process B:

Among the compound of the present invention, the compound of the formula[I-e]:

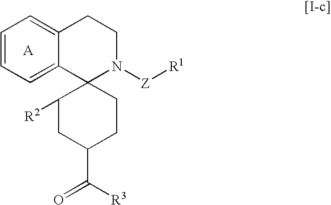

wherein the symbols are the same as defined above can be prepared by reacting the compound of the formula[II-c]:

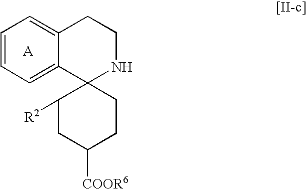

wherein the symbols are the same as defined above, or a salt thereof,

with a compound of the formula[9]:

X—Z—R¹ [9]

wherein X is a leaving group and the other symbols are the same as defined above, or a salt thereof,

to give a compound of the formula[II-b]:

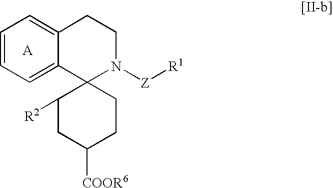

wherein the symbols are the same as defined above, and then reacting thus-obtained compound [II-b] with a compound of the formula[16]:

R³—H [16]

wherein R³ is the same as defined above, or a salt thereof.

The preparation of the compound [II-b) from the compound [II-c] can be conducted by the following manner.

When the group Z is a group of the formula: —CO—, the reaction can be carried out in the presence of a base in a solvent. The solvent include, for example, any solvent which does not disturb the reaction, such as methylene chloride, chloroform, dimethylformamide, dimethylacetamide, tetrahydrofuran, dioxane, toluene, benzene, 1,2-dichloroethane and 1-methylpyrrolidine. Examples of the base include pyridine, triethylamine, diisopropylethylamine, 4-methylmorpholine and 1,8-diazabicyclo[5,4,0]7-undecene (DBU).

When the group X is a hydroxyl group and the group Z is a group of the formula: —CO—, the present reaction can be also conducted by converting the compound [9] to a reactive derivative thereof at the carboxyl group (e.g., an acid halide, a mixed acid anhydride) and reacting the reactive derivative with the compound [II-c] in the presence of the base mentioned above.

When X is a hydroxyl group, the present reaction can be also carried out by the same manner as described for Process A.

When the group Z is a group of the formula: —CH₂—, the present reaction can be conducted in the presence of a base and in the presence or absence of additives in a solvent. The solvent include, for example, any solvent which does not disturb the reaction, such as methylene chloride, chloroform, dimethylformamide, dimethylacetamide, tetrahydrofuran, dioxane, toluene, benzene, 1,2-dichloroethane and 1-methylpyrrolidine. Examples of the base include pyridine, triethylamine, diisopropylethylamine, 4-methylmorpholine, 1,8-diazabicyclo-[5,4,0]-7-undecene (DBU), potassium carbonate and sodium carbonate. The additive includes, for example, potassium iodide. Among them, when X is a hydroxyl group, the compound [II-b] can be prepared by converting the hydroxyl group to a reactive residue (e.g., a halogen atom, p-toluenesulfonyloxy group) and reacting the reactive derivative with the compound [II-c].

The reaction process for preparing the compound [I-e] from the compound [II-b] can be carried out by the same as described for the process A.

Process C:

Among the compound [I] of the present invention, the compound [I-e] can be prepared by reacting a compound of the formula [I-f]:

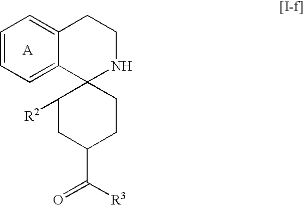

wherein the symbols are the same as defined above, or a salt thereof,

with the compound [9] or a salt thereof, in the same manner as described for the reaction process B to prepare the compound [II-b] from the compound [II-c].

Process D:

Among the compound [I] of the present invention, the compound [I-e] can be also produced by reacting the compound [II-c] or a salt thereof with the compound [16] or a salt thereof to obtain the compound [I-f] and then reacting the thus-obtained product with the compound [9] or a salt thereof.

The reaction process for preparing the compound [I-f] from the compound [II-c] can be conducted by the same manner as described for the process A mentioned above.

The reaction process for preparing the compound [I-e] from the compound [I-f] can be carried out by the same manner as described for the process C

Concomitantly, when the group of the formula: —Z—R¹ in [I-e] is a protecting group for an amino group (e.g., formyl group, acetyl group, propionyl group), the compound [I-f] can be prepared by removing the protecting group from the compound [I-e] by a conventional manner for deprotection of the amino group.

Process E:

Among the compound [I] of the present invention, the compound of the formula [I-b]:

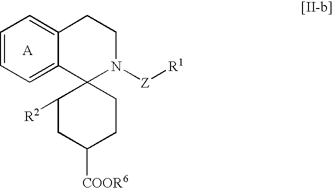

wherein the symbols are the same as defined above, can be obtained by reducing the compound [I-e] or salt thereof.

The present reaction process can be conducted in the presence of a reducing agent in a solvent. The solvent may be any solvent which does not disturb the reaction, for example, tetrahydrofuran, dioxane, toluene and benzene. Examples of the reducing agent include borane-tetrahydrofuran complex, borane-dimethylsulfide complex, lithium aluminum hydride and aluminum hydride.

Process F

Among the compound [I] of the present invention, the compound of the formula[I-d]:

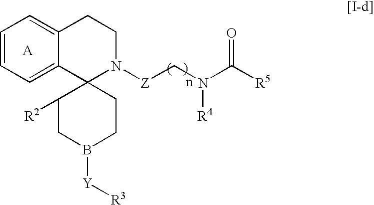

wherein the symbols are the same as defined above, can be prepared by the following reaction scheme.

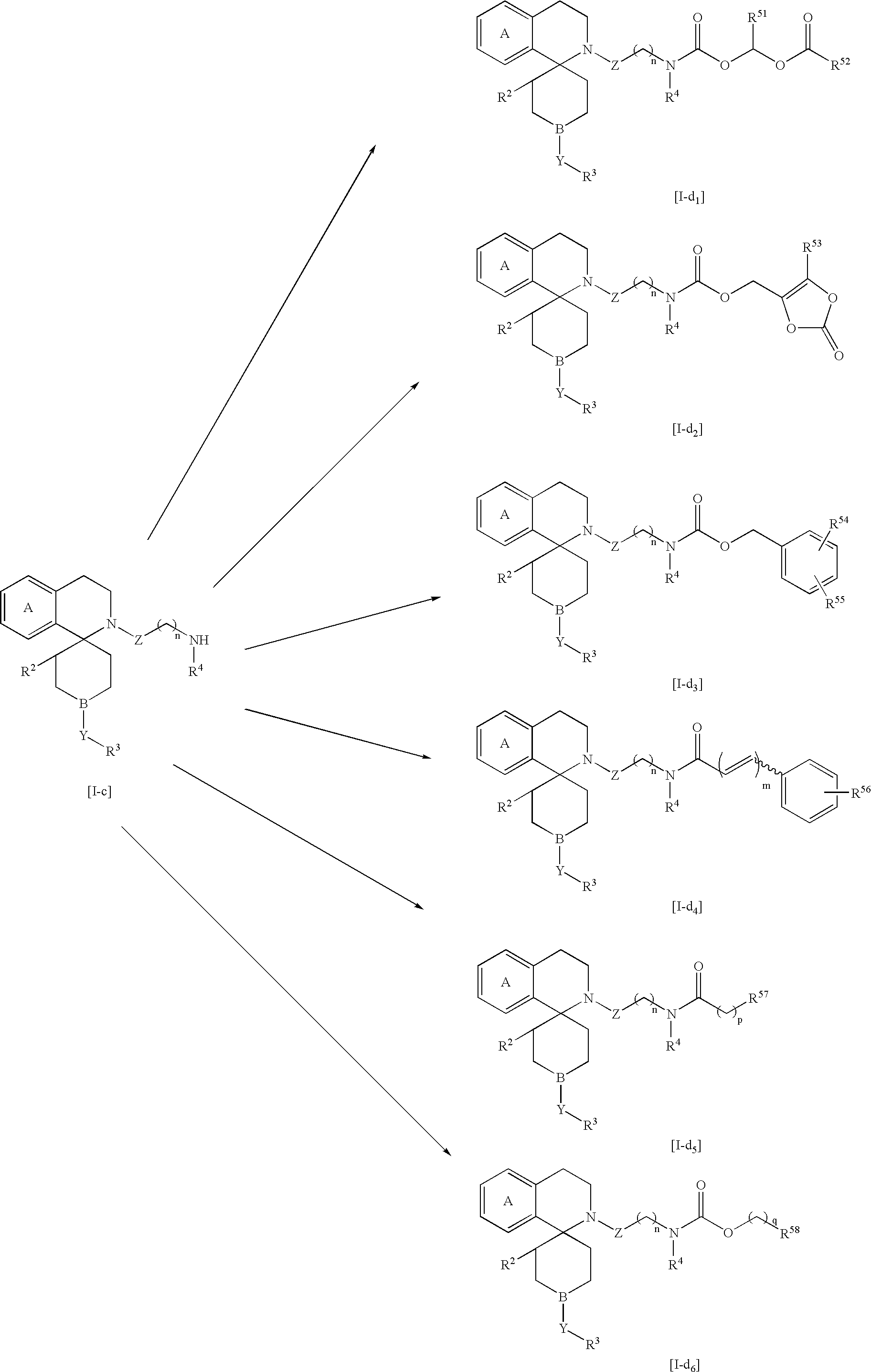

(The symbols in the scheme are the same as defined above.)

The reaction process for preparing the compound [I-d₁] from the compound [I-c] can be carried out in accordance with the following manner.

When R⁵² is a lower alkyl group being optionally substituted by a carboxyl group, a cyclo-lower alkyl group or an aryl group, the present reaction process can be conducted by reacting the compound [I-c] with a compound of the formula [17]:

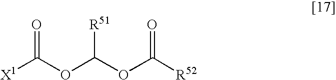

wherein X₁ is a leaving group and the other symbols are the same as defined above.

The solvent may be any solvent which does not disturb the reaction, for example, methylene chloride, chloroform, acetonitrile, tetrahydrofuran, 1,2-dimethoxyethane and dioxane. Examples of the base include triethylamine, diisopropylethylamine, cesium carbonate, potassium carbonate and sodium hydrogen bicarbonate. The leaving group X¹ includes, for example, a halogen atom such as chlorine atom or bromine atom, 2-pyridyloxy group, p-nitrophenoxy group and succinimidooxy group.

Alternatively, the compound [I-d₁] can be obtained by reacting the compound [I-c] with a compound of the formula [18]:

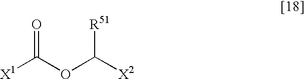

wherein X² is a halogen atom and the other symbols are the same defined above, or a salt thereof, to obtain a compound of the formula (I-d₇]:

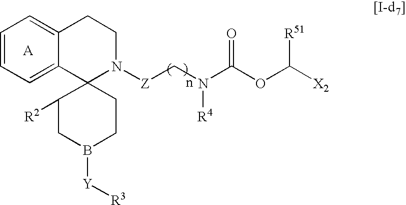

wherein the symbols are the same as defined above, and then reacting the thus-obtained compound [I-d₇] with a compound of the formula[19]:

R⁵²—COOH [19]

wherein the symbols are the same defined above.

The reaction process for preparing the compound [I-d₇] from the compound [I-c] and the compound [18] can be carried out in the presence of a base and in the presence or absence of an additive in a solvent. The solvent may be any solvent which does not disturb the reaction, for example, acetonitrile, dimethylformamide and dimethylacetamide. Examples of the base include cesium carbonate, potassium carbonate, sodium hydrogen bicarbonate, silver nitrate and mercury acetate. The additive includes, for example, molecular sieves.

The reaction process for preparing the compound [I-d₁] from the compound [I-d₇] and the compound [19] can be carried out in the same manner as described for the process of the compound [I-d₇] from the compound [I-c].

Concomitantly, the aforementioned reactions for preparing the compound [I-d₁] via the compound [I-d₇] from the compound [I-c] can be also carried out in a single vessel throughout these reactions.

When R⁵² is a lower alkoxy group or a cyclo-lower alkyloxy group, the present reaction mentioned above can be conducted in the presence of a base and the compound [18], in the presence or absence of an additive, in the presence of carbon dioxide gas and in the presence of a lower alcohol or a cyclo-lower alcohol, in a solvent. The solvent may be any solvent which does not disturb the reaction, for example, chloroform, methylene chloride, acetonitrile and dimethylformamide. The base includes, for example, triethylamine, diisopropylethylamine and cesium carbonate. Examples of the additive include tetrabutyl ammonium iodide and tetraethyl ammonium iodide.

The reaction process for preparing the compound [I-d₂], [I-d₃] or [I-d₆] from the compound [I-c] can be carried out by reacting the compound [I-c] with a compound of the formula [20]:

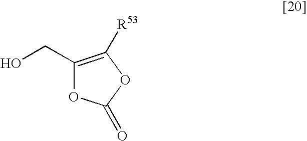

wherein the symbol is the same as defined above,

a compound of the formula [21]:

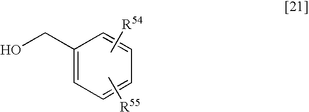

wherein the symbols are the same as defined above, or a compound of the formula [22]:

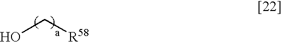

wherein the symbols are the same as defined above, in the presence of a base and a phosgene-equivalent in a solvent.

The solvent may be any solvent which does not disturb each reaction mentioned above, for example, chloroform and methylene chloride. The phosgene-equivalents include, for example, phosgene, diphosgene, triphosgene and carbonyldiimidazole (CDI). Examples of the base include triethylamine, diisopropylethylamine and pyridine.

The reaction process for preparing the compound [I-d₄] or [I-d₅] from the compound [I-c] can be carried out by reacting the compound [I-c] with a compound of the formula [23]:

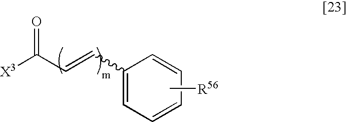

wherein X³is a leaving group and the other symbols are the same as defined above or a compound of the formula [24]:

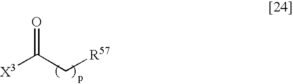

wherein the symbols are the same as defined above in the presence of a base in a solvent.

The solvent may be any solvent which does not disturb the reaction, for example, chloroform and methylene chloride. Examples of the base include triethylamine, diisopropylethylamine and pyridine.

When X³ is a hydroxyl group, the compound [I-d₄] or [I-d₅] can be also prepared by converting the compound [23] or [24] to a corresponding reactive derivative (e.g., an acid halide or a mixed acid anhydride) and then reacting the reactive derivative with the compound [I-c] in the presence of the above base. Besides, when X³ is a hydroxyl group, the present reaction process can be conducted by using a conventional condensing agent.

Among the compound [I] of the present invention, the compound of the formula [I-g]:

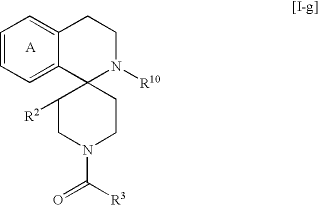

wherein the symbols are the same defined above, can be prepared by reacting a compound of the formula [II-B]:

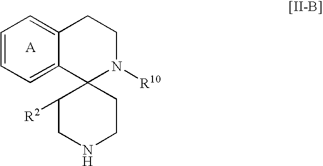

wherein the symbols are the same as defined above, or a salt thereof,

with a compound [16] or a salt thereof.

The present reaction can be conducted in the presence of a base and a phosgene-equivalent in a solvent. The solvent may be any solvent which does not disturb the reaction, for example, chloroform, methylene chloride, tetrahydrofuran, ethyl acetate and toluene. The phosgene-equivalents include, for example, phosgene, diphosgene, triphosgene and carbonyldiimidazole (CDI), phenyl chlorocarbonate and diethyl carbonate. Examples of the base include triethylamine, diisopropylethylamine and pyridine.

### PREPARATION of INTERMEDIATE COMPOUNDS

Among the intermediate compounds of the present invention, the compound [II-A] and [II-B] are novel compounds. These compounds can be prepared by the following methods.

Namely, among the intermediate compounds [II-A], a compound [II-a] or [II-e] can be obtained by the following manner.

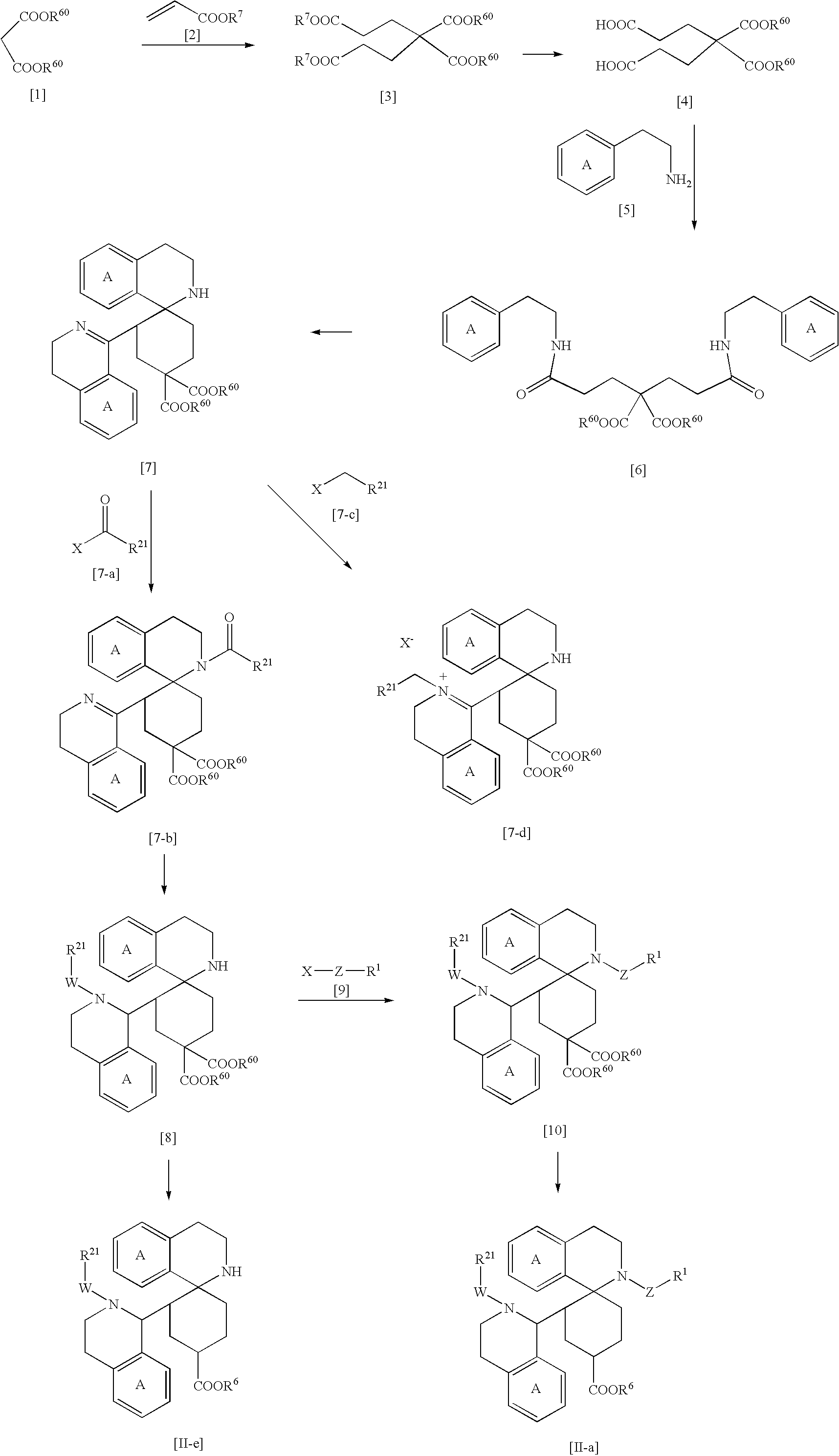

(In the above scheme, R⁶⁰ is a lower alkyl group, R⁷ is a protecting group for a carboxyl group, R²¹ is hydrogen atom or a lower alkyl group, W is a group of the formula: —CH₂— or —CO—, and other symbols are the same as defined above.)

The reaction process for preparing the compound [3] from the compound [1] and [3] can be carried out in the presence of a base in a solvent. The solvent may be any solvent which does not disturb the reaction, for example, tetrahydrofuran, tert-butylalcohol, dioxane, toluene, benezene and a mixture thereof. Examples of the base include potassium hydroxide, sodium hydroxide, potassium carbonate and triethylamine.

The reaction process for preparing the compound [4] from the compound [3] can be carried out in the presence of a suitable deprotecting agent in the presence or absence of a solvent. The solvent may be any solvent which does not disturb the reaction, for example, methylene chloride, chloroform, tetrahydrofuran, dioxane, 1,2-dimethoxyethane, methanol, ethanol, benzene, toluene, ethyl acetate, water and a mixture thereof. Examples of the deprotecting agents include potassium hydroxide, sodium hydroxide, formic acid, trifluoromethanesulfonic acid, p-toluenesulfonic acid, hydrochloric acid, sulfuric acid, palladium-carbon/hydrogen, palladium-carbon/formic acid and trifluoroacetic acid.

The reaction process for preparing the compound [6] from the compound [4] and [5] can be carried out by converting the compound [4] to a corresponding reactive derivative thereof (e.g., an acid halide, a mixed acid anhydride), and then reacting the reactive derivative with the compound [5] in the presence of a base in a solvent. The solvent may be any solvent which does not disturb the reaction, for example, dimethoxyethane, ethyl acetate, water, methylene chloride, chloroform, dimethylformamide, dimethylacetamide and a mixture thereof. Examples of the base include potassium carbonate, sodium carbonate and sodium bicarbonate. Besides, the present process can be also carried out by reacting directly the compound [4] with the compound [5] in the presence of a conventional condensing agent. The condensing agents include, for example, dicyclohexyl-carbodiimide (DCC), 1-ethyl-3-(3-dimethylarrminopropyl)carbodiimide.hydrochloride (WSC.HCl), diphenylphosphoryl azide (DPPA), carbonyldiimidazole (CDI), diethylcyanophosphate (DEPC), diisopropylcarbodiimide (DIPCI), benzotriazo1-1-yloxy-tripirrolidinophophonium-hexafluorophosphate (PyBOP) and carbonylditriazole.

The reaction process for preparing the compound [7] from the compound [6] can be carried out in the presence of a suitable dehydrating agent in a solvent. The solvent may be any solvent which does not disturb the reaction, for example, acetonitrile, benzene, toluene, chlorobenzene, methylene chloride, chloroform, nitromethane and a mixture thereof. Examples of the dehydrating agents include phosphorus oxychloride, polyphosphoric acid (PPA), polyphosphate ester (PPE) and phosphorus pentachloride.

The reaction process for preparing the compound [7-b] from the compound [7] and [7-a] can be carried out in the same manner as described for the Process C in which Z is a group of the formula: —CO—.

The reaction process for preparing the compound [8] from [7-b] can be carried out in the presence of a reducing agent in a solvent. The solvent may be any solvent which does not disturb the reaction, for example, ethanol, tetrahydrofuran, methanol, methyl cellosolve, dimethoxyethane, isopropanol, dioxane, methylene chloride, chloroform, acetic acid and a mixture thereof. Examples of the reducing agent include sodium borohydride, platinum oxide, palladium-carbon, lithium borohydride, calcium borohydride, zinc borohydride, borane-dimethylsulfide complex, borane-tetrahydrofuran complex, diisobutyl aluminum hydride and bis(2-methoxyethoxy)aluminum hydride (Red-Al). In the present reaction process, either the compound [8] in which W is a group of the formula: —CH₂— or the compound [8] in which W is a group of the formula: —CO— can be prepared by selecting the reducing agent and the reaction condition.

The reaction process for preparing the compound [7-d] from the compound [7] and [7-c] can be carried out in the same manner as described for the Process C (production of a compound in which Z is a group of the formula: —CH₂—)

The reaction process for preparing the compound (8] in which W is a group of the formula: —CH₂—from the compound [7-d] can be carried out in the same manner as described for the process to prepare the compound [8] from the compound [7-b].

The reaction process for preparing the compound [10] from the compound [8] and [9] can be conducted in the same manner as described for the Process A.

The process for preparing the compound [II-a] from the compound [10] can be carried out by hydrolysis of the compound [10] and subjecting the resultant product to decarboxylation reaction. The hydrolysis of the compound [10] can be conducted in the presence of a base or an acid in a solvent. The solvent may be any solvent which does not disturb the reaction, for example, ethanol, methanol, tetrahydrofuran, water, dioxane or a mixture thereof. The bases include sodium hydroxide, potassium hydroxide and lithium hydroxide. Examples of the acid include trifluoroacetic acid, formic acid and p-toluenesulfonic acid. The decarboxylation reaction can be conducted in the presence or absence of a base and a solvent under heating. The solvent may be any solvent which does not disturb the reaction, for example, dimethylformamide, tetrahydrofuran, dioxane, acetonitrile, benzene, acetic acid, toluene and pyridine. Examples of the base include pyridine and dimethylaminopyridine. The heating temperature is 40° C. to 200° C., preferably 50° C. to 150° C.

Concomitantly, the hydrolysis and decarboxylation can be also carried out in a single vessel throughout these reactions.

If necessary, the carboxylic acid compound obtained by the present reaction can be esterified in a conventional manner.

The compound [10] or [II-a] in which Z is a group of the formula: —CH₂—can be prepared by reducing the corresponding compound in which Z is a group of the formula: —CO—. The reaction can be conducted in the presence of a reducing agent in a solvent. The solvent may be any solvent which does not disturb the reaction, for example, tetrahydrofuran, dioxane, dimethoxyethane and diethylether. Examples of the reducing agent include borane-dimethylsulfide complex, lithium aluminum hydride and borane-tetrahydrofuran complex.

The reaction for preparing the [II-e] from the compound [8] can be carried out in the same manner as described for the process to prepare the compound [II-a] from the compound [10].

Besides, among the intermediate compounds [II-A], a compound of the formula [II-b], [II-c] or [II-d] can be prepared in the following manner.

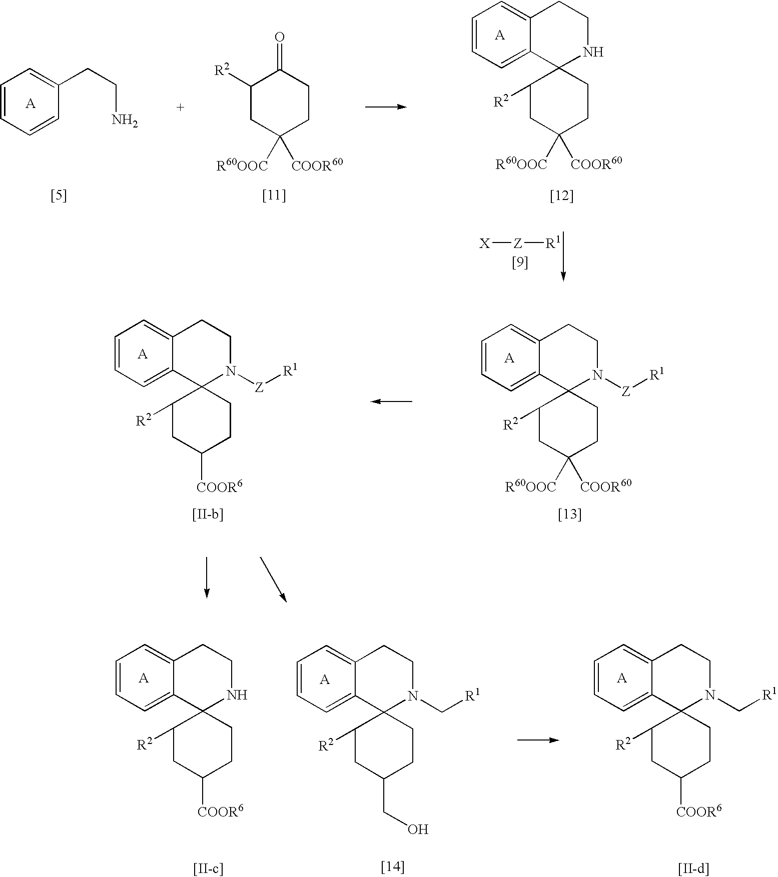

(In the above scheme, the symbols are the same as defined above.)

The reaction for preparing the compound [12] from the compound [5] and [11] can be carried out in the presence or absence of a dehydrating agent in the presence or absence of a solvent. The solvent may be any solvent which does not disturb the reaction, for example, ethanol, methanol, isopropanol, toluene, xylene, chlorobenzene, dimethylformamide. Examples of the dehydrating agents include polyphosphoric acid (PPA), polyphosphate ester (PPE), phosphorus pentaoxide and sillyl polyphosphate (PPSE).

The reaction for preparing the [13] from the compound [12] and [9] can be carried out in the same manner as described for the process (Process B) to prepare the compound [II-b] from the compound [II-c].

Besides, when a group of the formula: —Z—R¹ in the compound [13] is a protecting group for an amino group (e.g., formyl group, acetyl group, propionyl group), the compound [II-a] can be prepared by subjecting the compound [13] to a conventional deprotection reaction.

The reaction for preparing the compound [II-b] from the compound [13] can be carried out in the same manner as described for the process to prepare the compound [II-a] from the compound [10].

Moreover, when a group of the formula: —Z—R¹ in the compound [II-b] is a protecting group for an amino group (e.g., formyl group, acetyl group, propionyl group), the compound [II-c] can be prepared by subjecting the compound [II-b] to a conventional deprotection reaction.

Furthermore, the compound [14] can be prepared by reducing the compound [II-b]. The present reaction can be conducted in the presence of a reducing agent in a solvent. The solvent may be any solvent which does not disturb the reaction, for example, tetrahydrofuran, dioxane, dimethoxyethane and diethylether. Examples of the reducing agent include borane-dimethyl sulfide complex, lithium aluminum hydride and borane-tetrahydrofuran complex.

In addition, the compound [II-d] can be obtained by oxidizing the compound [14]. The present reaction can be carried out in the presence of an oxidizing agent in a solvent. The solvent may be any solvent which does not disturb the reaction, for example, methylene chloride, chloroform, water, tert-butylalcohol, acetonitrile and acetone. Examples of the oxidizing agent include chromic acid and pyridinium dichromate. In this process, the compound [II-d] can be also prepared by converting the compound [14] to an aldehyde compound of the formula [15]:

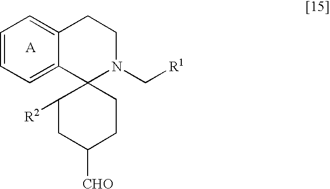

wherein the symbols are the same as defined above, and then oxidizing the aldehyde compound. The oxidizing agents for preparing the compound [15] from the compound [14] include oxalyl chloride/dimethylsulfoxide/triethylamine (Swan oxidizing agent), sulfatrioxide-pyridine complex and pyridinium dichromate (PDC). Examples of the oxidizing agent for producing the compound [II-b] from the compound [15] include sodium hydrochlorite, silver nitrate and sodium hydrochlorate. The-thus obtained carboxylic acid compound can be esterified in a conventional manner.

Among the intermediate compounds [II-B], the compound [II-f] or [II-g] can be prepared by the following manner.

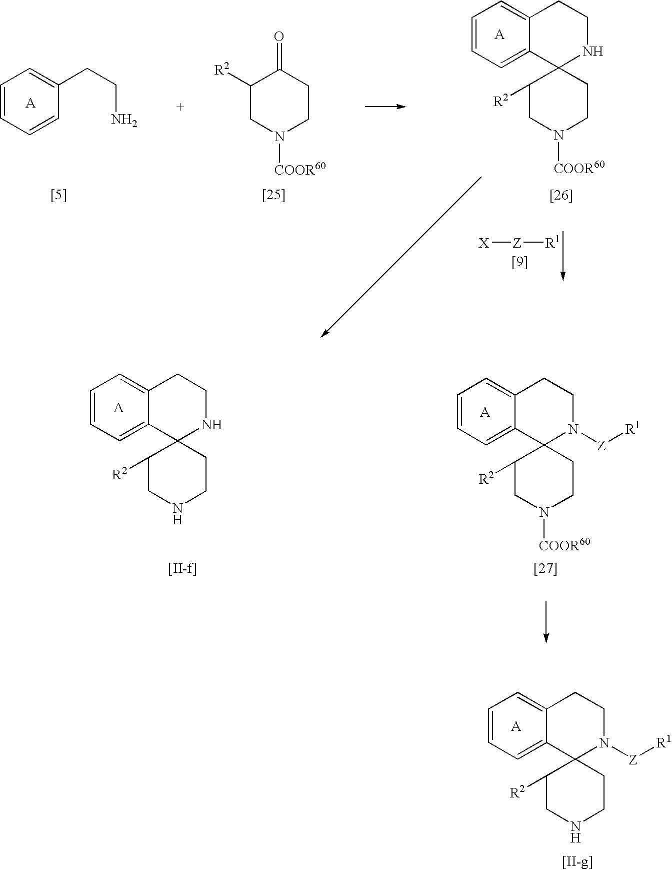

(The symbols in the above scheme are the same as defined above.)

The reaction process for preparing the compound [26] from the compounds [5] and [25] can be carried out by the same manner as described for the process for preparing the compound [12] from the compounds [5] and [11].

The reaction process for preparing the compound [II-f] from the compound [26] can be conducted in the presence of a base or an acid in a solvent. The solvent may be any solvent which does not disturb the reaction, for example, ethylene glycol, ethanol, methanol, water, methylene chloride and chloroform. Examples of the base include potassium hydroxide, sodium hydroxide and lithium hydroxide. The acids include, for example, formic acid, trifluoroacetic acid and hydrochloric acid. The base or acid suitable for R⁶⁰ in the compound [26] can be selected in the light of any methods known to the ordinary skilled in the art.

The reaction process for preparing the compound [27] from the compound [26] and [9] can be carried out by the same manner as described for the process C.

The reaction process for preparing the compound [II-g] from the compound [27] can be carried out by the same manner as described for the process for preparing the compound [II-f] from the compound [26].

Among the intermediate compound [16] in the present invention, the compound [III] of the formula:

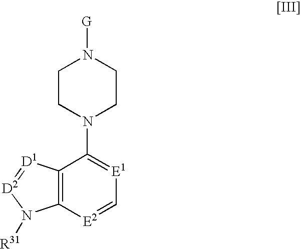

wherein G is a protecting group for an amino group and the other symbols are the same as defined above, can be prepared by reacting a compound of the formula[28]:

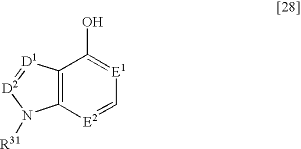

wherein the symbols are the same as defined above, or a salt thereof,

with a compound of the formula[29]:

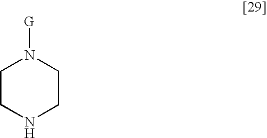

wherein the symbol is the same as defined above, or a salt thereof.

The reaction process for preparing the compound [III] from the compounds [28] and [29] can be conducted in the presence of an activating agent and an additive in the presence or absence of a solvent. This reaction can be carried out even without solvent, provided that a solvent is used in the reaction, the solvent may be any solvent which does not disturb the reaction, for example, hexamethyldisilazane, N,O-bistrimethyl-silylacetamide and chloromethylsilazane. The activating agents include, for example, ammonium sulfate, chlorotrimethylsilane, triethylamine hydrochloride, pyridine hydrochloride and triethylamine.

In particular, when hexamethyldisilazane as an activating agent and ammonium sulfate as an additive are used, the present reaction will provide the objective compound in a high yield with less by-products.

The compound [29] can be used in an amount of 1 to 4 moles, preferably 2 to 3 moles per mole of the compound [28]. The reaction can be suitably carried out at 100° C. to 200° C., particularly at 130° C. to 150° C.

The protecting group represented as G may be any conventional protecting group for an amino group, such as a benzyl group and a lower alkoxycarbonyl group.

An optically active isomer of the compound [II-A] can be prepared by treating an optical resolution agent with a racemic mixture of the compound [II-A] obtained above except for compounds in a meso-form. Namely, the optically active compound [II-A] can be prepared by treating the racemic mixture with an optical resolution agent to give a mixture of stereoisomers thereof and separating the mixture in a conventional manner such as column chromatography followed by removal of the optical resolution agent therefrom. The optical resolution agent may be, for example, a compound of the formula [30]:

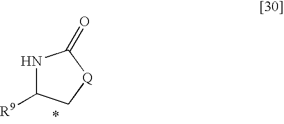

wherein R⁹ is a lower alkyl group, aryl-lower alkyl group, a cyclo-lower alkyl group or an aryl group, Q is oxygen atom or sulfur atom and an asterisk (*) means an asymmetric carbon atom.

Concretely, the optically active isomer of the compound [II-A] can be prepared by condensing a racemic mixture of the compound [II-A] with the compound (30] to give a mixture of diastereo isomers of a compound of the formula [II-A′]:

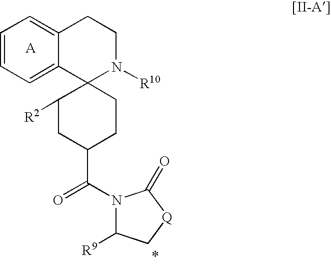

wherein the symbols are the same as defined above, separating the mixture by column chromatography, and removing the optical resolution agent from each separated isomer by a conventional hydrolysis reaction with using sodium hydroxide, potassium hydroxide, or mixture of lithium hydroxide and hydrogen peroxide.

The objective compounds [I] of the present invention, their intermediate compounds [II] and/or the starting materials therefore can be obtained by intramolecularly converting the substituent(s) in the Ring A, R¹, R² and/or R³ in these compounds prepared as described above to a desired substituent(s) within the scope of the present invention. The intramolecular conversion processes may be carried out in the following manner of method (a) to (t).

[Method (a): O-alkylation]

The compounds [I], [II] or their starting materials, in which the substituent(s) in Ring A is a lower alkoxy group, can be obtained by reacting the corresponding compounds thereof, in which the substituent(s) in the Ring A is a hydroxyl group, with a lower alkyl halide (e.g., methyl iodide, ethyl iodide, propyl iodide) in the presence of a base (e.g., sodium hydride, potassium carbonate).

[Method (b): Halogenation]

The compounds [I], [II] or the starting materials, in which the substituent(s) in Ring A is a halogen atom, can be obtained by reacting the corresponding compound having no substituent(s) at the corresponding position of Ring with a halogenating agent (e.g., sulfuryl chloride, N-chlorosuccinimide, N-bromo-succinimide).

[Method (c): Michael Reaction/Addition of Amino Group]

The compound [I] or [II] in which a group of the formula: —Z—R¹ is a group of the formula:

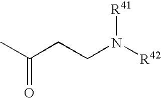

wherein R⁴¹ and R⁴² are the same or different group selected from hydrogen atom, a lower alkyl group, a cyclo-lower alkyl group and an aryl-lower alkyl group, can be prepared by reacting a corresponding compound [I] or [II] in which a group of the formula: —Z—R¹ is a group of the formula:

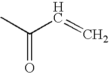

with a compound of the formula (31):

H—N(R⁴¹)(R⁴²) [31]

wherein the symbols are the same as defined above or a salt thereof in the presence or absence of a base (e.g., triethylamine, sodium hydroxide, potassium hydroxide).

[Method (d): Reduction of Double Bond]

The compound [I] or [II] in which R¹ is a lower alkyl group can be prepared by reducing a compound [I] or [II] in which R¹ is a lower alkenyl group in a conventional manner (in the presence of a reducing agent such as palladium-carbon/hydrogen).

[Method (e): Deprotection of Protected Amino Group]

The compound [I] or [II] in which R¹ is a lower alkylamino group can be prepared by subjecting a corresponding compound [I] or [II] in which R¹ is a phthalimido-lower alkyl group to a conventional deprotection reaction in the presence of hydrazine, sodium hydroxide, methylhydrazine.

[Method (f): Substitution Reaction]

The compound [I] or [II] in which R¹ and/or R² is an amino group or a lower alkylamino group (e.g., methylamino group) can be prepared by subjecting a corresponding compound [I] or [II] in which R¹ and/or R² is a protected amino group (said protecting group being benzyloxycarbonyl group, benzyl group, tert-butoxycarbonyl group) to a conventional deprotection reaction in the presence of trimethylsilyl iodide, palladium-carbon/hydrogen, palladium-carbon/formic acid, hydrobromic acid/acetic acid, trifluoroacetic acid, formic acid.

[Method (h): Reductive Amination]

The compound [I] or [II] in which R¹ and/or R² is an amino group substituted by a lower alkyl group, an aryl-lower alkyl group or a cyclo-lower alkyl group (e.g., dimethylamino group, diethylamino group, benzylamino group, cyclohexylamino group) can be prepared by reacting a corresponding compound [I] or [II] in which R¹ and/or R² is a group containing a primary or secondary amino group with a lower alkenyl, an aryl-lower alkenyl (e.g., formaldehyde, benzaldehyde) or a cyclo-lower alkanone (e.g., cyclohexanone) in the presence of a reducing agent (e.g., sodium borohydride, sodium triacetoxyborohydride, sodium cyanoborohydride).

[Method (i): Cyanoguanidination]

The compound [I] in which R¹ is a group of the formula:

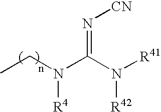

wherein the symbols are the same as defined above, can be obtained by reacting the compound [I-c] or a salt thereof with diphenylcyanocarbonimidate to give a compound in which R¹ is a group of the formula:

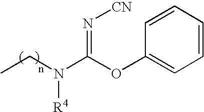

wherein the symbols are the same as defined above, and then reacting the resultant product with the compound [31].

[Method (j): Nitrovinylation]

The compound [I] in which R¹ is a group of the formula:

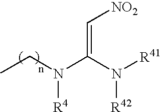

wherein the symbols are the same as defined above, can be obtained by reacting the compound [I-c] or a salt thereof with 1,1-bis(methylthio)-2-nitroethylene to give a compound in which R¹ is a group of the formula:

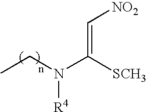

wherein the symbols are the same as defined above, and then reacting the resultant product with the compound [31].

[Method (k): Guanidination]

The compound [I] or [II] in which R¹ or R³ is a group containing a guanidine group can be prepared by reacting a corresponding compound [I] or [II], in which R¹ or R³ is a group containing a primary or secondary amino group, with 1H-pyrazo1-1-carboxamidine in the presence of a base (e.g., triethylamine, diisopropylethylamine). The 1H-pyrazo1-1-carboxamidine may be protected by a suitable protecting group (e.g., tert-butoxycarbonyl group, benzyloxycarbonyl group) which is removed after completion of the reaction.

[Method (1): N-biscyanovinylation]

The compound [I] in which R¹ is a group of the formula:

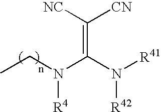

wherein the symbols are the same as defined above, can be obtained by reacting the compound [I-c] or a salt thereof with (bis(methylthio)methylene)propandinitrile to give a compound in which R¹ is a group of the formula:

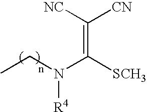

wherein the symbols are the same as defined above, and then reacting the resultant product with the compound [31].

[Method (m): N-cyclobutenylation]

The compound [I] in which R¹ is a group of the formula:

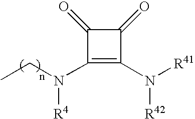

wherein the symbols are the same as defined above, can be obtained by reacting the compound [I-c] or a salt thereof with 3,4-diethoxy-3-cyclobuten-1,2-dione to give a compound in which R¹ is a group of the formula:

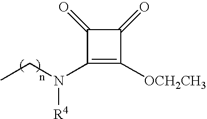

wherein the symbols are the same as defined above, and then reacting the resultant product with the compound [31].

[Method (n): N-carbamoylation]

The compound [I] in which R¹ is a group of the formula:

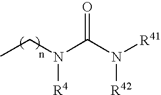

wherein the symbols are the same as defined above, can be obtained by reacting the compound [I-c] or a salt thereof with a compound of the formula [32]:

(R⁴¹)(R⁴²)N═N═O [32]

wherein the symbols are the same as defined above.

[Method (o): N-thiocarbamoylation]

The compound [I] in which R¹ is a group of the formula:

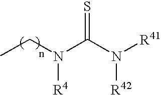

wherein the symbols are the same as defined above, can be obtained by reacting the compound [I-c] or a salt thereof with a compound of the formula [33]:

(R⁴¹)(R⁴²)N═C═S [33]

wherein the symbols are the same as defined above.

[Method (p): O-carbamoylation]

The compound [I] in which R¹ is group containing a carbamoyloxy group or a mono- or di-lower alkylcarbamoyl group can be prepared by reacting a corresponding compound [I] in which R¹ is a group containing hydroxyl group with the compound [31] in the presence of a phosgene-equivalent compound and a base. Alternatively, the corresponding compound [I] in which R¹ is a group containing hydroxyl group with a corresponding carbamoylhalide in the presence of a base.

[Method (q): C-alkenylation]

The compound [I] or [II] in which —Z—R¹ is a lower alkenoyl group (e.g., 4-pentenoyl group) can be prepared by reacting a corresponding compound [I] or [II] in which a group of the formula: —Z—R¹ is an acetyl group with an allyl halide (e.g., allyl bromide) in the presence of a base (e.g., lithium diisopropylamide, lithium hexamethyldisilazide).

[Method (r): Aldo1-Reduction]

The compound [I] or [II] in which a group of the. formula: —Z—R¹ is a butyryl group can be prepared by reacting a corresponding compound [I] or [II] in which a group of the formula: —Z—R¹ is an acetyl group with acetaldehyde in the presence of a base (e.g., lithium diisopropylamide, lithium hexamethyl-disilazide) to convert the acetyl group to 3-hydroxybutyryl group, converting the hydroxyl group in said 3-hydroxybutyryl group to a reactive residue (e.g., methanesulfonyloxy group) and reducing the resultant compound.

[Method (s): Reduction]

The compound [I] in which R³ is a group containing an amino group can be prepared by reducing a corresponding compound [I] in which R³ is a group containing a nitroso group in the presence of a conventional reducing agent (e.g., palladium-carbon/hydrogen, zinc/acetic acid).

[Method (t): Reduction of Hydroxyl Group]

The compound [I], [II] or the starting materials, in which the Ring A is an unsubstituted benzene ring, can be prepared by reacting a corresponding compound, in which the Ring A is a hydroxy-substituted benzene ring, with a trifluoromethane-sulfonyl compound (e.g., trifluoromethane-sulfonic acid anhydride) to convert the hydroxyl group to trifluoromethanesulfonyloxy group and then treating the resultant product with a reducing agent (e.g., palladium acetate/formic acid/triphenylphosphine/ triethylamine).

If necessary, the compounds [I] of the present invention obtained in the aforementioned Processes A to G or Method (a) to (t) can be converted to a pharmaceutically acceptable salt thereof by a known manner to the skilled in the art.

In the preparation of the compound [I], [II] and/or the starting material therefore, these intermediate compounds available for the production should not construed to be limited within the scope of the above description or reaction scheme, and any salts or reactive derivative thereof which does not disturb the illustrated reactions can be used in the present invention. Examples of the salt include, for example, salts with an alkaline metal or alkaline earth metal such as sodium, potassium, lithium, calcium or magnesium, an organic base such as pyridine, triethylamine or diisopropylethylamine, an inorganic acid such as hydrochloric acid, sulfuric acid, nitric acid, hydrobromic acid or phosphoric acid and an organic acid such as acetic acid, oxalic acid, citric acid, benzenesulfonic acid, benzoic acid, malonic acid, formic acid, fumaric acid, maleic acid, methanesulfonic acid, p-toluenesulfonic acid or trifluoroacetic acid.

Furthermore, in the preparation of the objective compound [I] of the present invention or the intermediate compounds, when the intermediate compounds have a functional group(s), if necessary, any appropriate protecting groups other than such groups as described hereinbefore may be also applicable to the present invention.

Throughout the present description and claims, an “alkyl group” means a straight- or branched-chain alkyl group having 1 to 16 carbon atoms, preferably 1 to 8 carbon atoms, an “lower alkyl group” or a “lower alkoxy group” means a straight- or branched-chain alkyl or alkoxy group having 1 to 6 carbon atoms, preferably 1 to 4 carbon atoms. A “lower alkanoyl group” means a straight- or branched-chain alkanoyl group having 2 to 7 carbon atoms, preferably 2 to 5 carbon atoms. A “cycloalkyl group” means a cycloalkyl having 3 to 20 carbon atoms, preferably 3 to 12 carbon atoms. A “cyclo-lower alkyl group” means a cycloalkyl having 3 to 8 carbon atoms, preferably 3 to 6 carbon atoms. A “alkenyl group” means a straight- or branched-chain alkenyl group having 2 to 16 carbon atoms, preferably 2 to 10 carbon atoms. A “lower alkenyl group” means a straight- or branched-chain alkenyl group having 2 to 8 carbon atoms, preferably 2 to 4 carbon atoms. A “alkylene group” means a straight- or branched-chain alkylene group having 1 to 16 carbon atoms, preferably 1 to 10 carbon atoms. A “lower alkylene group” means a straight- or branched-chain alkylene group having 1 to 6 carbon atoms, preferably 1 to 5 carbon atoms. Moreover, a “halogen atom” means fluorine, chlorine, bromine or iodine atom.

### EXPERIMENTS

### Experiment 1

(Assay of ¹²⁵I-Apamine-Binding Inhibition)

Animals Used:

Male Hartley guinea pigs (Age: 4 weeks or more)

Preparation of Test Drug Solution:

Each test drug was dissolved in a buffer (5 mM Tris-HCl, 5.4 mM KC1, 0.1% bovine serum albumin (BSA), pH 7.4; This buffer is hereinafter referred as buffer (3)). Hardly water-soluble test drugs were dissolved in dimethylsulfoxide followed by being diluted with buffer (3).

Methods:

The experiments were carried out according to the method described by Hugues et al (Life Sciences 31, 437-443, 1982) and Catterall et al (Journal of Biochemistry 254, 11379-11387, 1979).

Preparation of Membrane Fraction:

The colon was isolated from guinea pigs and mucosal cells were removed therefrom. The colon was homogenized together with a buffer solution (1)(40 mM Tris-HCl, 8% sucrose, pH 7.4) and the homogenate was centrifuged at 130,000 g for 60 minutes. The pellet was suspended in the buffer solution (1) and the suspension was layered on top of a discontinuous sucrose gradient composed of 33% sucrose and 40% sucrose. After centrifugation at 160,000 g for 90 minutes, the 8/33%-layer was collected and suspended in a buffer solution (2)(5 mM Tris-HCl, pH 7.4). The suspension was further centrifuged at 160,000 g for 60 minutes. The resultant pellet was suspended in buffer (3) at a final concentration of 500 μg of membrane protein per mL and used for binding assay (If necessary, this suspension was frozen and stored in a liquid nitrogen).

Binding Assay:

The membrane (10 μg/mL), ¹²⁵I-apamin (10 pM) and a test drug solution were added to the buffer (3) and the mixture was incubated at 4° C. for 60 minutes. After incubation, the mixture was filtered through cell-harvester and the radioactivity of ¹²⁵I-apamin retained by the filter was counted with a gamma-counter. The inhibitory activity (IC₅₀) of the test drug on specific binding of ¹²⁵I-apamine to the membrane was calculated. The binding level of ¹²⁵I-apamine in the presence of large excess(100 nM) of apamine was defined as non-specific binding of ¹²⁵I-apamin. The results of the biding assay are shown in the following table (Table.1).

TABLE 1

| | ¹²⁵I-apamine-binding |
|---|---|
| Test Drug | inhibitory activity |
| (No. of Example) | (IC₅₀; μM) |
| 165 (2) | 0.05 |
| 170 (2) | 0.05 |
| 172 (2) | 0.004 |
| 177 (2) | 0.009 |
| 234 (2) | 0.01 |
| 236 (2) | 0.06 |
| 241 (2) | 0.01 |
| 282 (2) | 0.02 |

### Experiment 2

(Inhibitory Activity on acetylcholinesterase (AChE))

Preparation of Test Drug Solution:

Each test drug was dissolved in distilled water containing 0.1% BSA (pH 7.4). Hardly water-soluble test drugs were dissolved in dimethylsulfoxide followed by being diluted with distilled water containing 0.1% BSA (pH 7.4).

Methods:

Measurements of AChE activity were carried out according to the method described by Ellman et al (Biochem Pharmacol 7, 88-95, 1961). A solution of 12 mM 5,5,-dithiobis-(2-nitrobenzoic acid) (50 μL; final concentration: 0.3 mM), a solution of AChE (2-4 U/mL; 50 μL) and a test drug solution (50 μL) were added, in this order, to 110 mM phosphate buffer (1.8 mL; final concentration: 100 mM; pH 7.4) and the mixture was incubated for a predetermined period (3 to 60 minutes) at 25° C. After incubation, a solution of 20 mM acetylthiocholin iodide (50 μL; final concentration: 0.5 mM) was added to the mixture. Immediately after stirring the mixture, absorbance at 412 nm of the reaction mixture was measured. In terms of change in the absorbance per unit time (z,900 E/min), the AChE-inhibitory activity of the test drug (IC50) was calculated. Concomitantly, the measurements of the present experiment were carried out in the presence of 1% BSA. The results of the biding assay are shown in the following table (Table.2).

TABLE 2

| | Acetylcholinesterase |
|---|---|
| Test Drug | inhibitory activity |
| (No. of Example) | (IC₅₀; μM) |
| 139 (2) | 0.06 |
| 165 (2) | 0.002 |
| 170 (2) | 0.005 |
| 181 (2) | 0.0003 |
| 185 (2) | 0.00008 |
| 295 (4) | 0.02 |

### Experiment 3

(Evaluation of Evacuation-Promoting Activity)

Animals Used:

Male Hartley guinea pigs (Age: 4 to 5 weeks; n=6 to 10/group)

Preparation of Test Drug Solution:

Each test drug was dissolved in distilled water. Hardly water-soluble test drugs were suspended in a 0.5% carboxymethyl cellulose solution. Dose of the test drug solution was 5 mL/kg.

Methods:

Guinea pigs (2 animals/cage) were encaged and acclimatized for a few days prior to the examinations. Fifteen minutes after the administration of clonidine (30 μg/kg, i.p.), each test compound solution (5 mL/kg) was administered intraperitoneally or orally to the animals. One hour after the administration of the test compound, number of evacuated fecal pellets in each cage was measured and recorded. Effective dose of the test compound was determined as a minimum dose in which the number of evacuated fecal pellets was grater than that in control group (group of guinea pigs to which clonidine and vehicle of the test compound were administered d).

The results are shown in the following tables (Table.3and 4).

TABLE 3

| | Evacuation-promoting activity in |
|---|---|
| Test Drug | guinea pig |
| (No. of Example) | (Effective dose: mg/kg i.p.) |
| 241 (2) | 0.03 |
| 165 (2) | 0.01 |

TABLE 4

| | Evacuation-promoting activity |
|---|---|
| Test Drug | in guine pig |
| (No. of Example) | (Effective dose: mg/kg p.o.) |
| 313 (1) | 0.1 |
| 313 (2) | 0.1 |
| 315 (2) | 0.3 |
| 330 (2) | 0.1 |
| 386 (2) | 1 |
| 396 (2) | 0.3 |
| 391 (2) | 0.3 |

The results of Experiment 1 show that the compounds of the present invention [I] exhibit SK channel-blocking activity.

The results of Experiment 2 show that, the compounds [I] of the present invention include compounds having both SK channel-blocking activity and AChE-inhibitory activity.

The results of Experiment 3show that the compounds [I] of the present invention are useful as a medicament for treating and/or prophylaxis of constipation.

Examples of the compound [I] of the present invention which can be prepared by the above exemplified methods are illustrated below, but the present invention should not be construed to be limited thereto.

Each compound represented by a chemical structure or a chemical name in the present specification is, unless such compound is referred as an optically active compound, in a mixture of enantiomers (racemic mixture) having a relative configuration determined by the chemical structure or the chemical name. On the other hand, when the compound is indicated as an optically active compound, the compound is either one of the enantiomers having a relative configuration determined by the chemical structure or the chemical name.

Concretely, when a compound is referred as “(1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-pyridyl)-1-piperazinyl]carbonyl-spiro[cyclohexane-1,1′(2′H)-isoquinoline]”, unless such compound is indicated as an optically active compound, this compound is in a racemic mixture comprising (1R, 2R(S), 4R)-2′-ethyl-3′,4′-dihydro-6′,7′-dimethoxy-2-( 2-ethyl-1,2,3,4-tetrahydro-6,7-dimethoxy-1-isoquinolyl)-4-[4-(2-pyridyl)-1-piperazinyl]carbonyl-spiro[cyclohexane-1,1′(2′H)-isoquinoline] and (1S, 2S(R), 4S)-2′-ethyl-3′,4′-dihydro-6′,7′-dimethoxy-2-(2-ethyl-1,2,3,4-tetrahydro-6,7-dimethoxy-1-isoquinolyl)-4-[4-(2-pyridyl)-1-piperazinyl]carbonyl-spiro[cyclohexane-1,1′(2′H)-isoguinoline]. On the other hand, when a compound is referred as an optically active compound, this compound is either one of enatiomomers. Concomitantly, in the indications for the configuration of the compounds, for example, “(S*)” in “2R*(S*)” for the indication of “(1R*,2R*(S*),4R*)”, represents the configuration of the substituent (i.e., 1-position of tetrahydro-isoquinoline ring) at 2-position of cyclohexane moiety.

In addition, when a compound is described as a compound of the formula [A]:

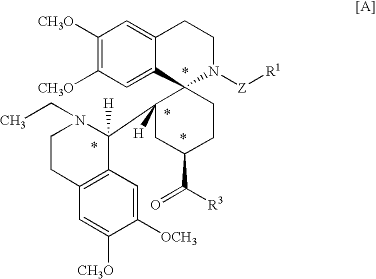

unless such compound is indicated as an optically active compound, the compound [A] is in a form of a racemic mixture of a compound of the formula [B]:

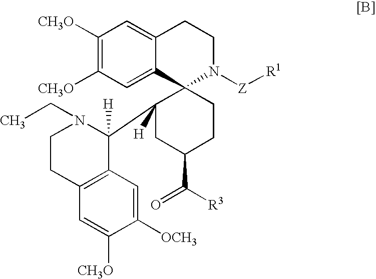

and a compound of the formula [C]:

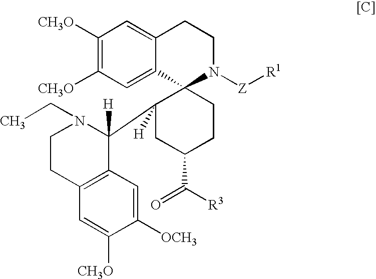

On the other hand, when the compound [A] is referred as an optically active compound, this compound is either one of enatiomomers [B] and [C].

### Example 1

1) To a solution of (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (Compound obtained in Reference example 2(5))(660 mg) in methylene chloride (25 mL) is added a oxalyl chloride (0.22 mL) and a drop of dimethylformamide under ice-cooling and the mixture is stirred at room temperature for 1 hour. The solvent and the excess of oxalyl chloride are removed in vacuo. The residue is dissolved in methylene chloride (25 mL), and triethylamine (1 mL) and a solution of 2-pyridylpiperazine (682 mg) in methylenechloride (10 mL) are added dropwise to the solution with ice-cooling. The mixture is stirred at room temperature for 30 minutes. To the mixture is added water and the reaction mixture is concentrated. To the residue is added ethyl acetate, the mixture is washed with water and saturated aqueous NaCl solution, successively, dried over sodium sulfate, and concentrated. The residue is purified by column chromatography on NH-silica gel (Solvent; ethyl acetate:n-hexane 1:1) and recrystallized from ethanol to give (1R*,2R*(S*),4R*)-2′-ethyl-3′,4′-dihydro-6′,7′-dimethoxy -2-(2-ethyl-1,2,3,4-tetrahydro-6,7-dimethoxy-1-isoquinolyl)-4-[4-(2-pyridyl)-1-piperazinyl]carbonyl-spiro[cyclohexane-1,1′(2′H)-isoquinoline](782 mg, 93%) as crystals.

M.p. 115-117° C., MS(FAB) m/z: 698, IR(Nujol)⁻¹:1640, 1590, 1510.

2) The compound obtained in the above step (1) is treated with a solution of HCl-ethanol to give 3hydrochloric acid salt thereof as crystals. M.p. 180-183° C.

### Example 2

1)(1R*,2R*(S)4R*)-2′-Ethyl-3′4′-dihydro-6′7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline ] (Compound obtained in Reference example 2(5)) and [N-(2-pyridyl)-N-methylcarbamoylmethyl]piperazine are treated in the same manner as described in Example 1(1) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-[N-(2-pyridyl)-N-methylcarbamoylmethyl]-1-piperazinyl]carbonyl-spiro[cyclohexane-1,1′(2′H)-isoquinoline]as an amorphous powder.

MS(FAB) m/z: 770(M+H), IR(Nijol)cm⁻¹ :1670, 1640

2) The compound obtained in the above step (1) and two equimolar amount of fumaric acid are dissolved in water and the mixture is lyophilized to give 2 fumaric acid salt thereof as an amorphous powder. MS(FAB) m/z: 770(M+H)

3) The compound obtained in the above step (1) and 1.5 molar amount of fumaric acid are dissolved in water and the mixture is recrystallized from ethanol-diisopropyl ether to give 1.5 fumaric acid salt thereof as crystals. M.p. 172-174° C.

### Examples 3 to 21

1)manner as described in Example 1(1) or Example 2(1) to give compounds as shown in the following tables (Table.5).

In the tables as described hereinafter, unit of MS value is “m/z”, and Me, Et, n-Pr, n-Bu and Ac mean methyl group, ethyl group, n-propyl group, n-butyl group and acetyl group, respectively.

TABLE 5
(No. 1)

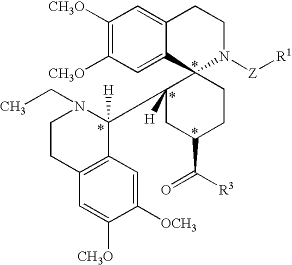

| Ex. | | | Physicochemical |
|---|---|---|---|
| No. | —Z—R¹ | R³ | properties etc. |
| 3(1) | 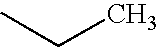 | 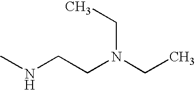 | Amorphous powder MS(FAB) 651(M + H) |
| 4(1) | 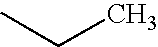 | 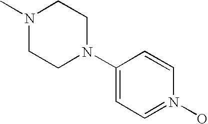 | Amorphous powder MS(APCI) 714(M + H) |
| 5(1) | 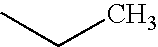 | 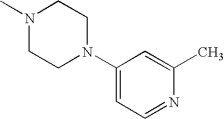 | Amorphous powder MS(APCI) 712(M + H) |
| 6(1) | 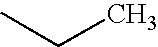 | 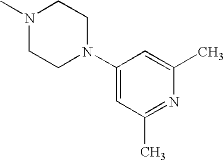 | Amorphous powder MS(APCI) 726(M + H) |
| 7(1) | 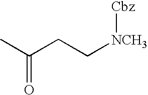 | 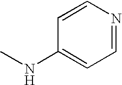 | Amorphous powder MS(APCI) 820(M + H) |
| 8(1) | 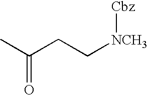 | 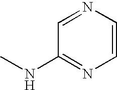 | Amorphous powder MS(APCI) 821.5(M + H) |

Cbz: Benzyloxycarbonyl group

TABLE 5
(No. 2)

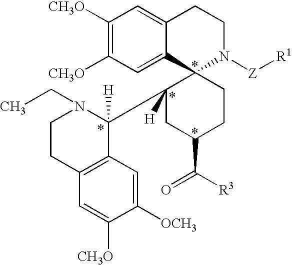

| Ex. | | | Physicochemical |
|---|---|---|---|
| No. | —Z—R¹ | R³ | Properties etc. |
| 9(1) | 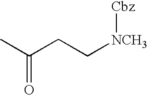 | 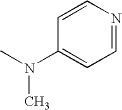 | Amorphous powder MS(APCI) 834(M + H) |
| 10(1) | 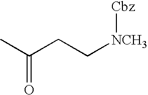 | 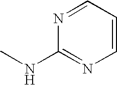 | Amorphous powder MS(APCI) 821(M + H) |
| 11(1) | 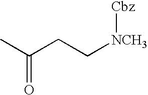 | 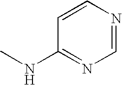 | Amorphous powder MS(APCI) 821.6(M + H) |
| 12(1) | 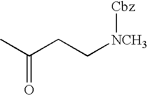 | 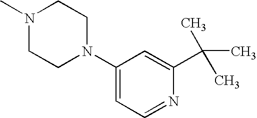 | Amorphous powder MS(APCI) 945(M + H) |
| 13(1) | 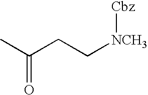 | 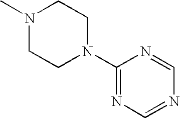 | Amorphous powder MS(APCI) 891(M + H) |
| 14(1) | 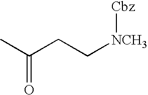 | 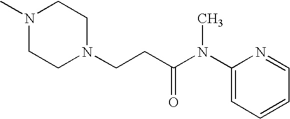 | Amorphous powder MS(APCI) 974(M + H) |

Cbz: Benzyloxycarbonyl group

TABLE 5
(No. 3)

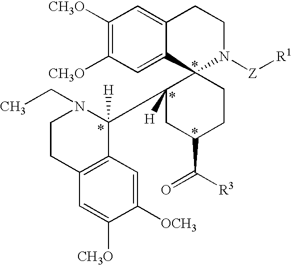

| Ex. | | | Physicochemical |
|---|---|---|---|
| No. | —Z—R¹ | R³ | Properties etc. |
| 15(1) | 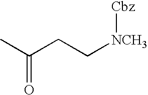 | 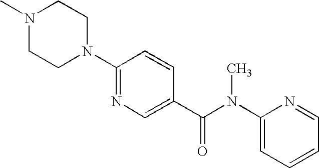 | Amorphous powder MS(APCI) 1023(M + H) |
| 16(1) | 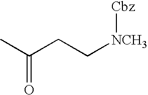 | 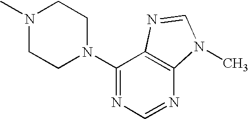 | Amorphous powder MS(APCI) 944(M + H) |
| 17(1) | 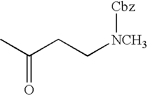 | 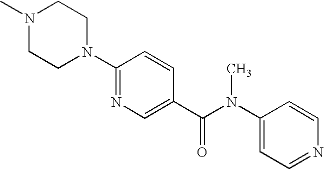 | Amorphous powder MS(APCI) 1023(M + H) |
| 18(1) | 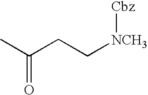 | 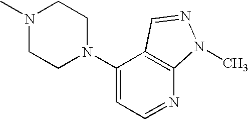 | Amorphous powder MS(APCI) 943(M + H) |

Cbz: Benzyloxycarbonyl group

TABLE 5
(No. 4)

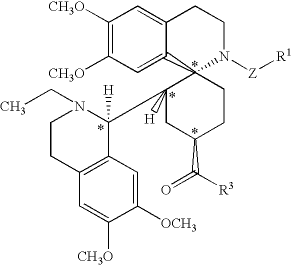

| Ex. | | | Physicochemical |
|---|---|---|---|
| No. | —Z—R¹ | R³ | properties etc. |
| 19(1) | 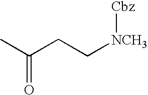 | 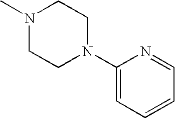 | Optically active isomer Amorphous powder MS(APCI) 889(M + H) [α]_{D} −45.7°(c0.6, CHCl₃) |
| 20(1) | 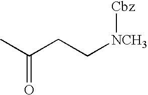 | 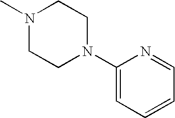 | Optically active isomer Amorphous powder MS(APCI) 889(M + H) [α]_{D} +48.7°(c0.6, CHCl₃) |
| 21(1) | 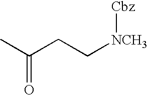 | 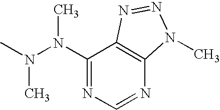 | Amorphous powder MS(APCI) 919.7(M + H) |

2) The compounds obtained in the above Examples 1(1) to 21(1) are treated in the same manner as described in Example 1(2), Example 2(2) or Example 2(3) to give the following compounds (salt).

Example 3(2): 3 HCl salt of the compound obtained in Example 3(1); M.p. 174-176° C. (Decomp.)

Example 4(2): 2 Fumaric acid salt of the compound obtained in Example 4(1); Lyophilized amorphous powder, MS(ESI)m/z: 714(M+H), IR(Nujol)cm⁻¹: 3230, 1694

Example 5(2): 2 Fumaric acid salt of the compound obtained in Example 5(1); Lyophilized amorphous powder, MS(APCI)m/z: 712(M+H), IR(Nujol)cm⁻¹: 1701, 1635

Example 6(2): 2 Fumaric acid salt of the compound obtained in Example 6(1)

### Example 22

1) A solution of (1R*,2R*(S*),4R*)-2′-ethyl-3′,4′-dihydro-6′,7′-dimethoxy -2-(2-ethyl-1,2,3,4-tetrahydro-6,7-dimethoxy-1-isoquinolyl)-4-carboxy-spiro[cyclohexane-1,1′(2′H)-isoquinoline] (Compound obtained in Reference example 2(5))(150 mg),2-[N-(2-pyridyl)-N-acetylamino]-ethylpiperazine (82 mg), diethylcyanophosphonate (0.065 mL), and triethyl amine (69 mg) in dimethylformamide (2 mL) are stirred at room temperature overnight. The reaction mixture is extracted with ethyl acetate after addition of a solution of sodium hydrogencarbonate. The organic layer is washed with saturated aqueous NaCl solution, and dried over sodium sulfate. The organic layer is concentrated and the residue is purified by column chromatography on silica gel (Solvent; chloroform:methanol:28% aqueous ammonia solution=100:10:1) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-[2-[N-(2-pyridyl)-N-acetylamino]ethylpiperrazin-yl]carbonyl-spiro[cyclohexane-1,1′(2′H)-isoquinoline] (120 mg, 56%) as an oil.

MS(APCI)m/z: 783(M+H), IR(neat)cm⁻¹: 1665, 1635

2) The compound obtained in the above step (1) is treated in the same manner as described in Example 2(2) to give 2 fumaric acid salt thereof as an amorphous powder.

MS (APCI)m/z: 783 (M+H).

### Examples 23 to 51

The corresponding materials are treated in the same manner as described in Example 22(1) to give compounds as shown in the following tables (Table.6).

TABLE 6
(No. 1)

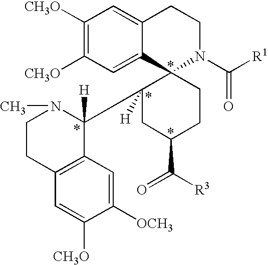

| Ex. | | Physicochemical |
|---|---|---|
| No. | R³ | properties etc. |
| 23(1) | 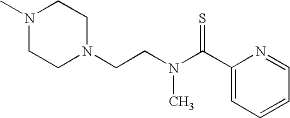 | Amorphous powder MS(APCI) 799(M + H) |

TABLE 6
(No. 2)

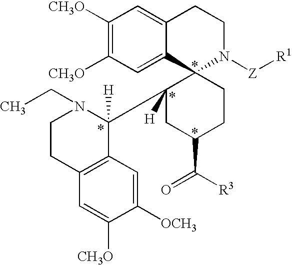

| Ex. | | | Physicochemical |
|---|---|---|---|
| No. | —Z—R¹ | R³ | properties etc. |
| 24(1) | 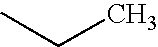 | 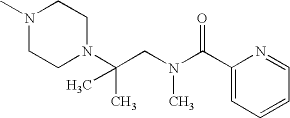 | Amorphous powder MS(APCI) 811(M + H) |
| 25(1) | 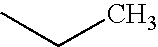 | 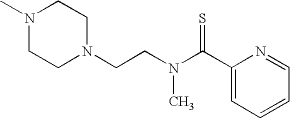 | M.p. 82.6-86.4° C. MS(ESI) 799(M + H) |
| 26(1) | 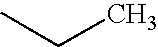 | 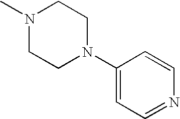 | M.p. 184-184.5° C. MS(ESI) 698(M + H) |
| 27(1) | 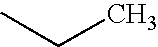 | 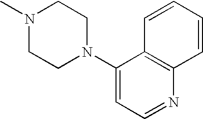 | Amorphous powder MS(APCI) 748(M + H) |
| 28(1) | 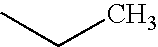 | 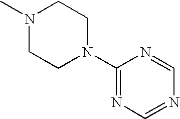 | Amorphous powder MS(APCI) 700(M + H) |
| 29(1) | 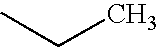 | 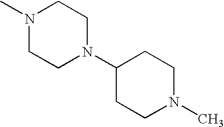 | Amorphous powder MS(APCI) 718.6(M + H) |

TABLE 6
(No. 3)

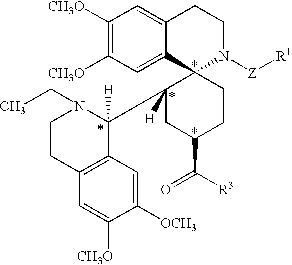

| Ex. | | | Physicochemical |
|---|---|---|---|
| No. | —Z—R¹ | R³ | properties etc. |
| 30(1) | 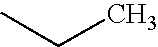 | 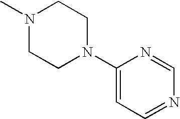 | Amorphous powder MS(APCI) 699.5(M + H) |
| 31(1) | 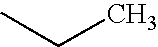 | 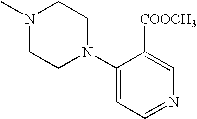 | Amorphous powder MS(APCI) 756.5(M + H) |
| 32(1) | 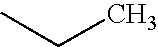 | 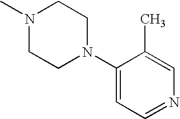 | Amorphous powder MS(APCI) 712.5(M + H) |

TABLE 6
(No. 4)

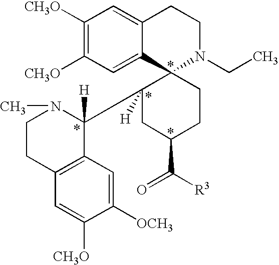

| Ex. | | Physicochemical |
|---|---|---|
| No. | R³ | properties etc. |
| 33(1) | 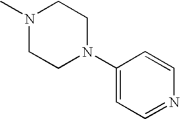 | Foam MS(APCI) 684(M + H) |

TABLE 6
(No. 5)

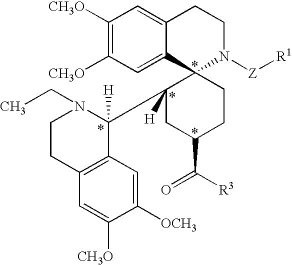

| Ex. | | | Physicochemical |
|---|---|---|---|
| No. | —Z—R¹ | R³ | properties etc. |
| 34(1) | 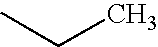 | 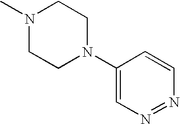 | Foam MS(APCI) 699.5(M + H) |
| 35(1) | 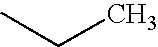 | 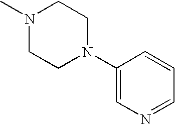 | Foam MS(APCI) 698.5(M + H) |
| 36(1) | 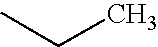 | 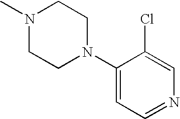 | Foam MS(APCI) 732.2(M + H) |
| 37(1) | 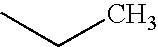 | 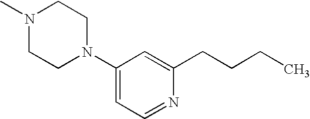 | Foam MS(APCI) 754(M + H) |
| 38(1) | —CH₃ | 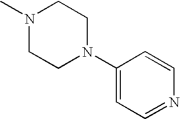 | Foam MS(APCI) 684(M + H) |

TABLE 6
(No. 6)

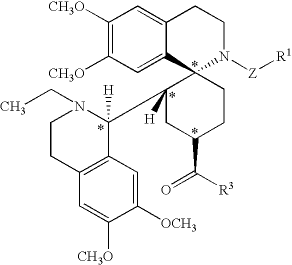

| Ex. | | | Physicochemical |
|---|---|---|---|
| No. | —Z—R¹ | R³ | properties etc. |
| 39(1) | 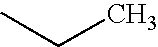 | 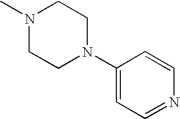 | Foam MS(APCI) 698(M + H) |

TABLE 6
(No. 7)

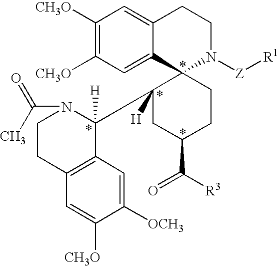

| Ex. | | | Physicochemical |
|---|---|---|---|
| No. | -Z-R¹ | R³ | properties etc. |
| 40 (1) | 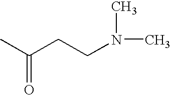 | 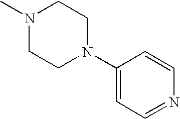 | Amorphous powder MS(APCI) 783 (M + H) |

TABLE 6
(No. 8)

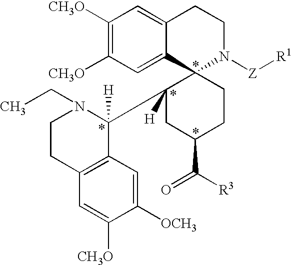

| Ex. | | | Physicochemical |
|---|---|---|---|
| No. | -Z-R¹ | R³ | properties etc. |
| 41 (1) | 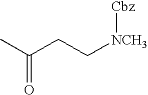 | 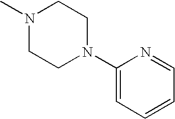 | Amorphous powder MS (APCI) 889 (M + H) |
| 42 (1) | 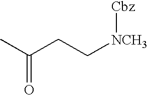 | 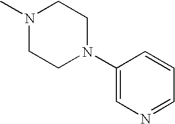 | Amorphous powder MS (APCI) 889 (M + H) |
| 43 (1) | 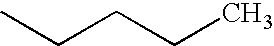 | 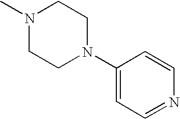 | Amorphous powder MS (APCI) 726 (M + H) |
| 44 (1) | 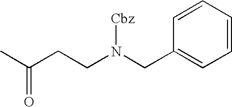 | 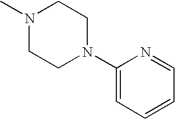 | Amorphous powder MS (APCI) 966 (M + H) |
| 45 (1) | 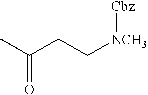 | 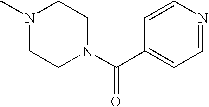 | Amorphous powder MS (APCI) 917 (M + H) |
| 46 (1) | 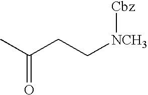 | 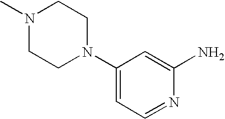 | Amorphous powder MS (APCI) 904 (M + H) |

Cbz: Benzyloxycarbonyl group

TABLE 6
(No. 9)

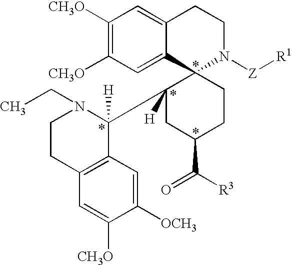

| Ex. | | | Physicochemical |
|---|---|---|---|
| No. | -Z-R¹ | R³ | properties etc. |
| 47 (1) | 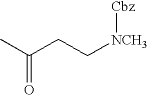 | 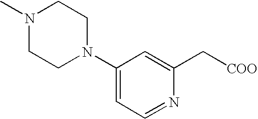 | Amorphous powder MS (APCI) 961 (M + H) |
| 48 (1) | 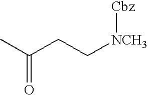 | 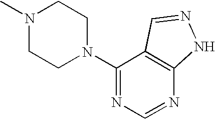 | Amorphous powder MS (APCI) 930 (M + H) |
| 49 (1) | 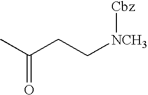 | 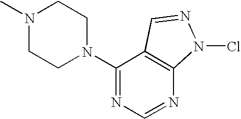 | Amorphous powder MS (APCI) 944 (M + H) |
| 50 (1) | 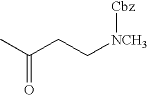 | 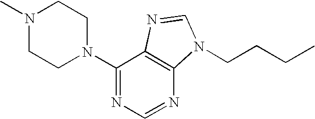 | Amorphous powder MS (APCI) 986 (N + H) |
| 51 (1) | 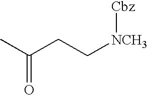 | 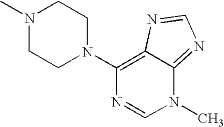 | Amorphous powder MS (APCI) 944 (M + H) |

Cbz: Benzyloxycarbonyl group

2) The compounds obtained in the above Examples 23(1) to 51(1) are treated in the same manner as described in Example 2(2) to give the following compounds (salt)

Example 23(2): 2 Fumaric acid salt of the compound obtained in Example 23(1); Lyophilized amorphous powder, MS(APCI)m/z: 799(M+H), IR(Nujol)⁻¹: 1705, 1635, 1612

Example 24(2): 2 Fumaric acid salt of the compound obtained in Example 24(1); Lyophilized amorphous powder, MS(APCI)m/z: 811(M+H), IR(Nujol)cm⁻¹: 1640

Example 25(2): 3 Fumaric acid salt of the compound obtained in Example 25(1); Lyophilized amorphous powder, MS(ESI)m/z: 799(M+H), IR(Nujol)⁻¹: 1641, 1460

Example 26(2): 2 Fumaric acid salt of the compound obtained in Example 26(1)

Example 27(2): 2 Fumaric acid salt of the compound obtained in Example 27(1)

Example 28(2): 3 Fumaric acid salt of the compound obtained in Example 28(1)

Example 29(2): 2.5 Fumaric acid salt of the compound obtained in Example 29(1)

Example 30(2): 3 Fumaric acid salt of the compound obtained in Example 30(1), Amorphous powder, IR(neat)cm⁻¹: 2605, 1711, 1640

Example 32(2): 2 Fumaric acid salt of the compound obtained in Example 32(1)

Example 33(2): 2 Fumaric acid salt of the compound obtained in Example 33(1)

Example 34(2): 2 Fumaric acid salt of the compound obtained in Example 34(1)

Example 35(2): 2 Fumaric acid salt of the compound obtained in Example 35(1)

Example 36(2): 2.5 Fumaric acid salt of the compound obtained in Example 36(1)

Example 37(2): 2 Fumaric acid salt of the compound obtained in Example 37(1)

Example 38(2): 2 Fumaric acid salt of the compound obtained in Example 38(1)

Example 39(2): 2 Fumaric acid salt of the compound obtained in Example 39(1)

Example 40(2): 2.5 Fumaric acid salt of the compound obtained in Example 40(1), Amorphous powder, IR(neat+chloroform)cm⁻¹: 1706, 1639

Example 43(2): 2 Fumaric acid salt of the compound obtained in Example 43(1)

Example 44(2): 2 Fumaric acid salt of the compound obtained in Example 44(1), Amorphous powder, MS(APCI)m/z: 741.5(M+H), IR(neat+chloroform)cm⁻¹: 1703

### Example 52

1) A mixture of (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H) -isoquinoline] (Compound obtained in Reference example 2(5))(200 mg),2-amino-4-piperazinylpyridine (77.4 mg),1-ethyl-3-(3-dimethylaminopropyl]carbodiimide hydrochloride (103.8 mg),1-hydroxybenzotriazole (70.6 mg) and dimethylformamide (4 mL) is stirred at room temperature overnight. To the reaction mixture are added a solution of sodium hydrogen carbonate and the mixture is extracted with ethyl acetate. The organic layer is washed with saturated aqueous NaCl solution and dried over sodium sulfate. The organic layer is concentrated and the residue is purified by column chromatography on silica gel (Solvent; chloroform:methanol=100:1) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-aminopyridin -4yl)-1-piperazinyl]carbonyl-spiro[cyclohexane-1,1′(2′H)-isoquinoline] (171 mg, 67%) as an amorphous powder.

MS(APCI) m/z: 713, IR(Nujol)⁻¹: 1599.

2) The compound obtained in the above step (1) is treated in the same manner as described in Example 2(2) to give 2 fumaric acid salt thereof as a Lyophilized amorphous powder. IR(neat+chloroform)cm⁻¹: 1638.

Examples 53 to 163

The corresponding materials are treated in the same manner as described in Example 52(1) to give the compounds as shown in the following tables (Table.7).

TABLE 7
(No. 1)

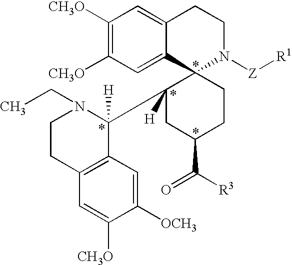

| | | | Physicochemical |
|---|---|---|---|
| Ex. No. | -Z-R¹ | R³ | properties etc. |
| 53 (1) | 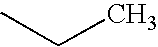 | 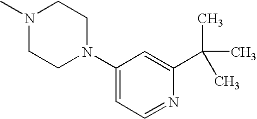 | Foam MS (APCI) 754 (M + H) |
| 54 (1) | 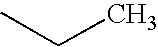 | 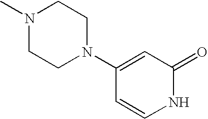 | Foam MS (APCI) 714 (M + H) |
| 55 (1) | 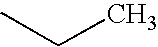 | 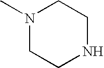 | Foam MS (APCI) 621 (M + H) |
| 56 (1) | 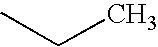 | 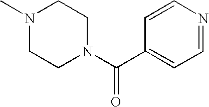 | Foam MS (APCI) 726 (M + H) |
| 57 (1) | 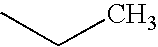 | 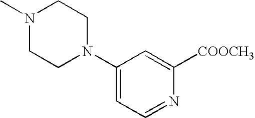 | Amorphous powder MS (APCI) 756 (M + H) |
| 58 (1) | 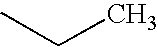 | 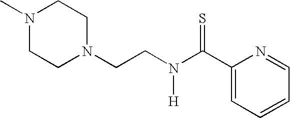 | Amorphous powder MS (APCI) 785 (M + H) |

TABLE 7
(No. 2)

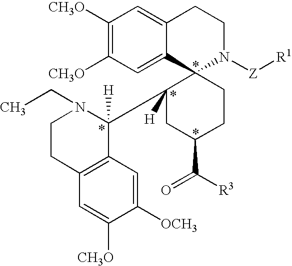

| Ex. | | | Physicochemical |
|---|---|---|---|
| No. | -Z-R¹ | R³ | properties etc. |
| 59 (1) | 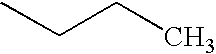 | 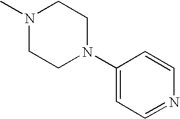 | Amorphous powder MS (APCI) 712 (M + H) |
| 60 (1) | 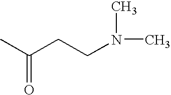 | 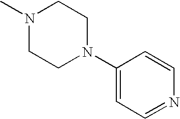 | Amorphous powder MS (APCI) 769 (M + H) |
| 61 (1) | 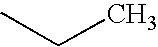 | 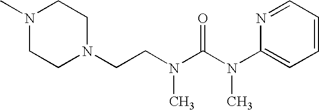 | Amorphous powder MS (APCI) (828 M + H) |
| 62 (1) | 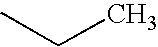 | 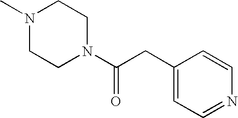 | Amorphous powder MS (APCI) 740 (M + H) |
| 63 (1) | 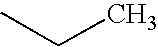 | 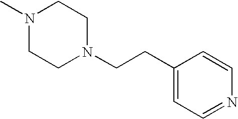 | Amorphous powder MS (APCI) 726 (M + H) |
| 64 (1) | 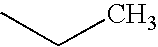 | 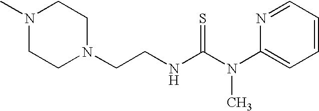 | Amorphous powder MS (APCI) 814 (M + H) |
| 65 (1) | 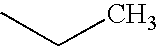 | 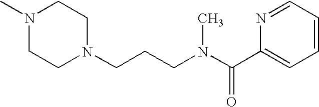 | Amorphous powder MS (APCI) 797 M + H) |

TABLE 7
(No. 3)

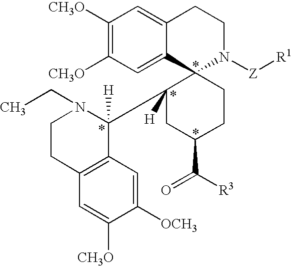

| Ex. | | | Physicochemical |
|---|---|---|---|
| No. | -Z-R¹ | R³ | properties etc. |
| 66 (1) | 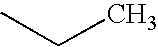 | 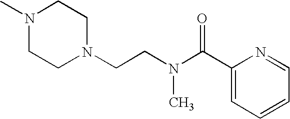 | Amorphous powder MS (APCI) 783.6 (M + H) |
| 67 (1) | 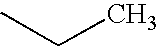 | 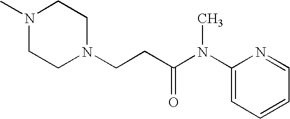 | Amorphous powder MS (APCI) 783 (M + H) |
| 68 (1) | 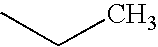 | 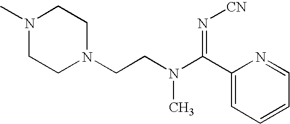 | Amorphous powder MS (APCI) 807 (M + H) |
| 69 (1) | 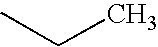 | 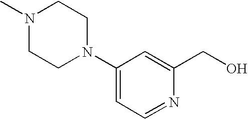 | Amorphous powder MS (APCI) 728 (M + H) |
| 70 (1) | 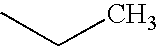 | 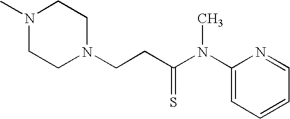 | Amorphous powder MS (APCI) 799 (M + H) |
| 71 (1) | 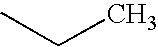 | 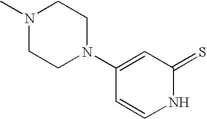 | Amorphous powder MS (APCI) 730 (M + H) |
| 72 (1) | 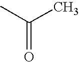 | 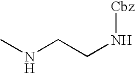 | Amorphous powder MS (APCI) 743 (M + H) |

Cbz: Benzyloxycarbonyl group

TABLE 7
(No. 4)

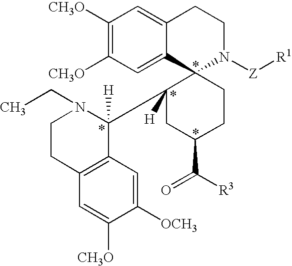

| Ex. | | | Physicochemical |
|---|---|---|---|
| No. | -Z-R¹ | R³ | properties etc. |
| 73 (1) | 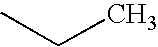 | 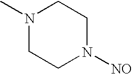 | Amorphous powder MS (APCI) 650 (M + H) |
| 74 (1) | 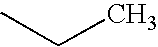 | 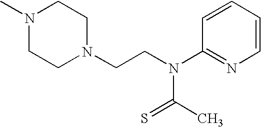 | Amorphous powder MS (APCI) 799 (M + H) |
| 75 (1) | 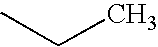 | 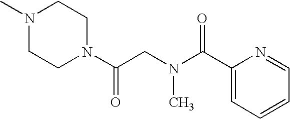 | Amorphous powder MS (APCI) 797 (M + H) |
| 76 (1) | 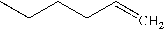 | 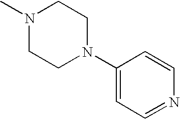 | Foam MS (ESI) 738 (M + H) |
| 77 (1) | 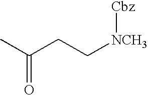 | 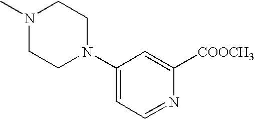 | Amorphous powder MS (APCI) 947 (M + H) |
| 78 (1) | 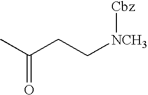 | 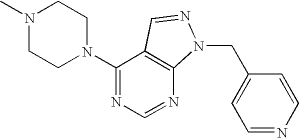 | M.p. 148-150° C. |
| 79 (1) | 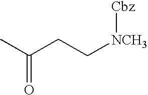 | 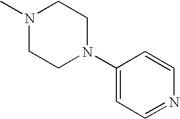 | Amorphous powder MS (APCI) 889 (M + H) |

Cbz: Benzyloxycarbonyl group

TABLE 7
(No. 5)

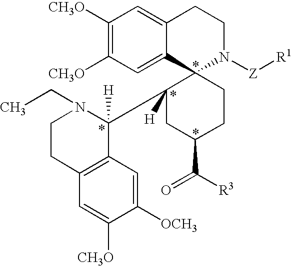

| Ex. | | | Physicochemical |
|---|---|---|---|
| No. | -Z-R¹ | R³ | properties etc. |
| 80 (1) | 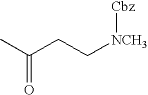 | 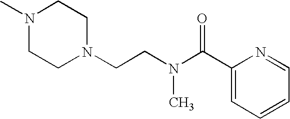 | Amorphous powder MS (ESI) 974 (M + H) |
| 81 (1) | 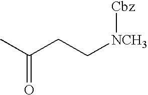 | 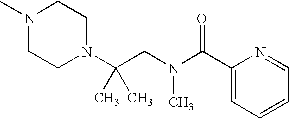 | Amorphous powder MS (APCI) 1002 (M + H) |
| 82 (1) | 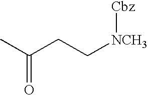 | 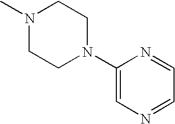 | Amorphous powder MS (APCI) 890.6 (M + H) |
| 83 (1) | 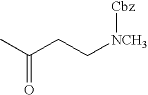 | 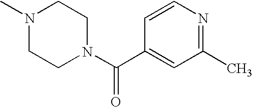 | Amorphous powder MS (ESI) 904 (M + H) |
| 84 (1) | 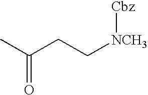 | 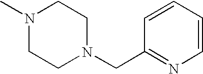 | Amorphous powder MS (APCI) 903 (M + H) |
| 85 (1) | 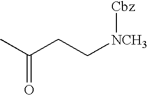 | 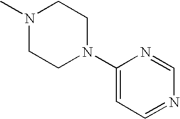 | Amorphous powder MS (APCI) 890 (M + H) |

Cbz: Benzyloxycarbonyl group

TABLE 7
(No. 6)

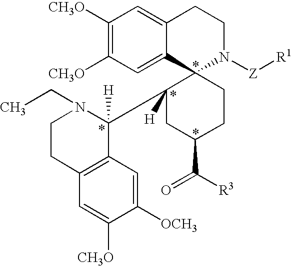

| Ex. | | | Physicochemical |
|---|---|---|---|
| No. | -Z-R¹ | R³ | properties etc. |
| 86 (1) | 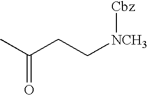 | 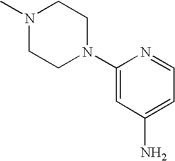 | Amorphous powder MS (APCI) 905 (M + H) |
| 87 (1) | 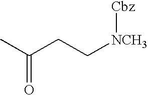 | 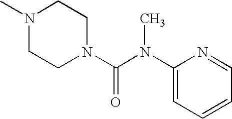 | Amorphous powder MS (APCI) 946 (M + H) |
| 88 (1) | 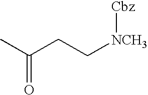 | 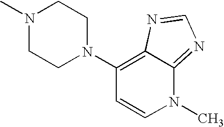 | Amorphous powder MS (APCI) 943 (M + H) |
| 89 (1) | 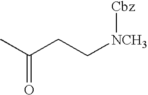 | 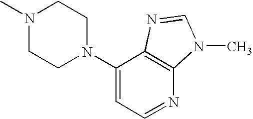 | Amorphous powder MS (APCI) 943.5 (M + H) |
| 90 (1) | 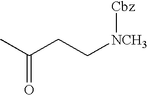 | 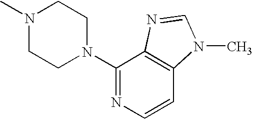 | Amorphous powder MS (APCI) 943 (M + H) |
| 91 (1) | 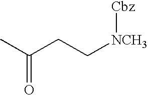 | 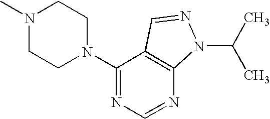 | Amorphous powder MS (APCI) 972 (M + H) |
| 92 (1) | 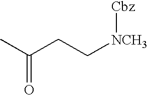 | 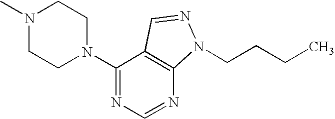 | Amorphous powder MS (APCI) 986 (M + H) |

Cbz: Benzyloxycarbonyl group

TABLE 7
(No. 7)

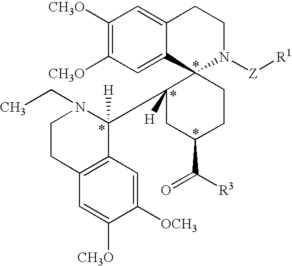

| Ex. | | | Physicochemical |
|---|---|---|---|
| No. | -Z-R¹ | R³ | properties etc. |
| 93 (1) | 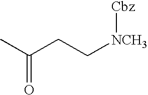 | 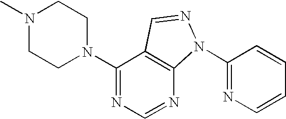 | Amorphous powder MS (APCI) 1007 (M + H) |
| 94 (1) | 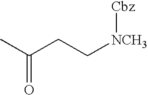 | 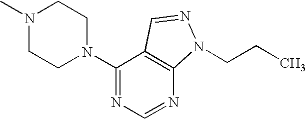 | Amorphous powder MS (APCI) 972 (M + H) |
| 95 (1) | 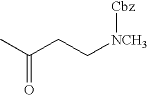 | 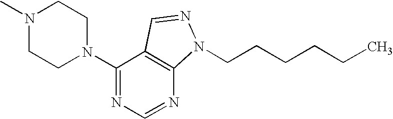 | Amorphous powder MS (APCI) 1014 (M + H) |
| 96 (1) | 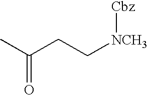 | 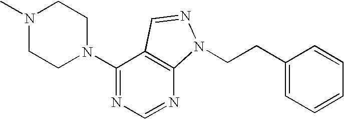 | Amorphous powder MS (APCI) 1034 (M + H) |
| 97 (1) | 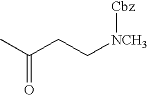 | 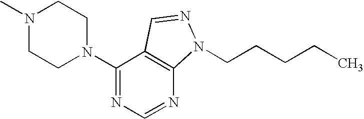 | Amorphous powder MS (APCI) 1000 (M + H) |
| 98 (1) | 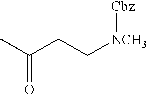 | 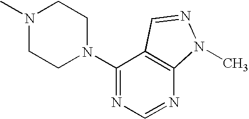 | Amorphous powder MS (APCI) 945 (M + H) |
| 99 (1) | 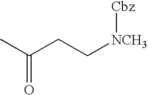 | 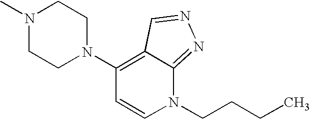 | Amorphous powder MS (APCI) 985 (M + H) |

Cbz: Benzyloxycarbonyl group

TABLE 7
(No. 8)

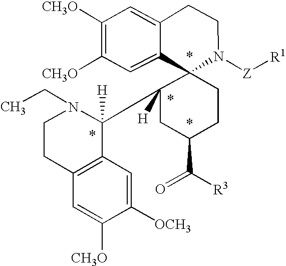

| Ex. | | | Physicochemical |
|---|---|---|---|
| No. | —Z—R¹ | R³ | properties etc. |
| 100(1) | 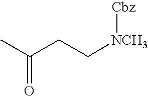 | 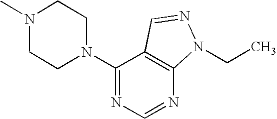 | Amorphous powder MS(APCI)958(M + H) |
| 101(1) | 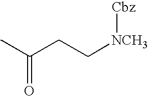 | 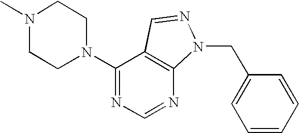 | Amorphous powder MS(APCI)1020(M + H) |
| 102(1) | 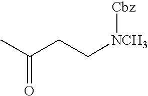 | 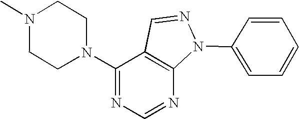 | Amorphous powder MS(APCI)1006(M + H) |
| 103(1) | 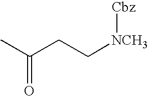 | 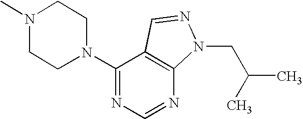 | Amorphous powder MS(APCI)986(M + H) |
| 104(1) | 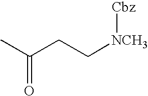 | 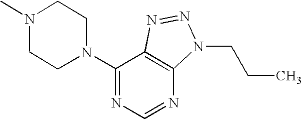 | Amorphous powder MS(APCI)973(M + H) |
| 105(1) | 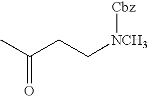 | 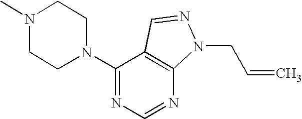 | Amorphous powder MS(APCI)970(M + H) |
| 106(1) | 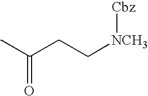 | 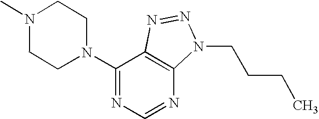 | Amorphous powder MS(APCI)987(M + H) |

Cbz: Benzyloxycarbonyl group

TABLE 7
(No. 9)

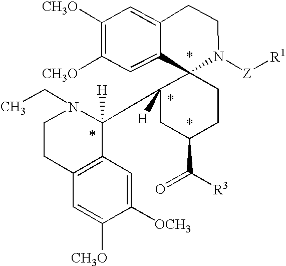

| Ex. | | | Physicochemical |
|---|---|---|---|
| No. | —Z—R¹ | R³ | properties etc. |
| 107(1) | 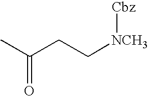 | 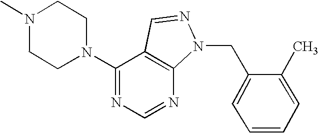 | Amorphous powder MS(APCI)1034(M + H) |
| 108(1) | 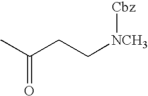 | 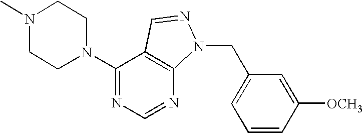 | Amorphous powder MS(APCI)1050(M + H) |
| 109(1) | 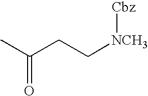 | 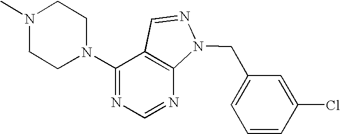 | Amorphous powder MS(APCI)1054(M + H) |
| 110(1) | 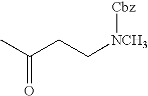 | 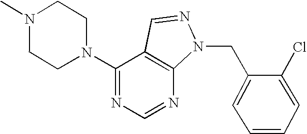 | Amorphous powder MS(APCI)1054(M + H) |
| 111(1) | 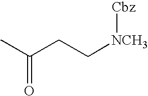 | 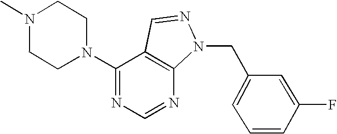 | Amorphous powder MS(APCI)1038(M + H) |
| 112(1) | 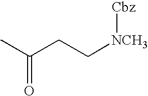 | 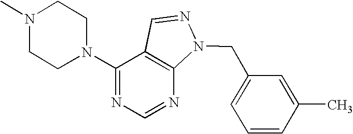 | Amorphous powder MS(APCI)1034(M + H) |
| 113(1) | 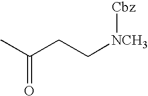 | 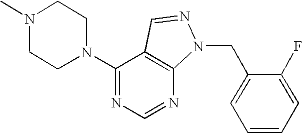 | Amorphous powder MS(APCI)1038(M + H) |

Cbz: Benzyloxycarbonyl group

TABLE 7
(No. 10)

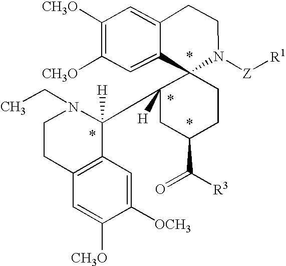

| Ex. | | | Physicochemical |
|---|---|---|---|
| No. | —Z—R¹ | R³ | properties etc. |
| 114(1) | 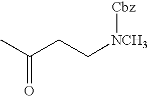 | 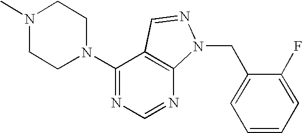 | Amorphous powder MS(APCI)1026(M + H) |
| 115(1) | 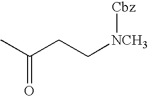 | 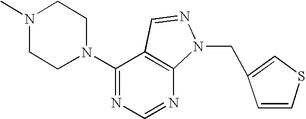 | Amorphous powder MS(APCI)1026(M + H) |
| 116(1) | 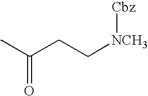 | 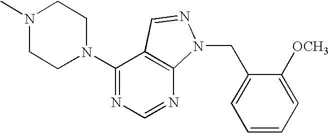 | Amorphous powder MS(APCI)1050(M + H) |
| 117(1) | 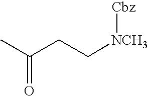 | 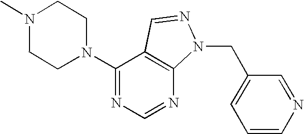 | Amorphous powder MS(APCI)1021(M + H) |
| 118(1) | 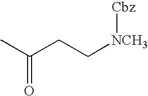 | 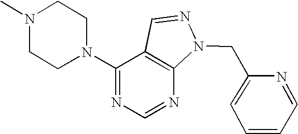 | Amorphous powder MS(APCI)1021(M + H) |
| 119(1) | 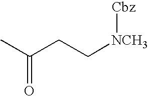 | 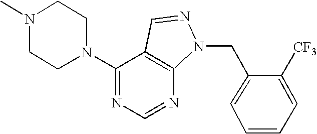 | Amorphous powder MS(APCI)1088(M + H) |
| 120(1) | 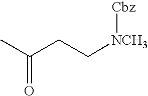 | 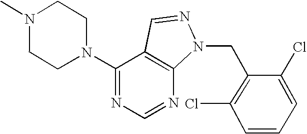 | Amorphous powder MS(APCI)1088(M + H) |

Cbz: Benzyloxycarbonyl group

TABLE 7
(No. 11)

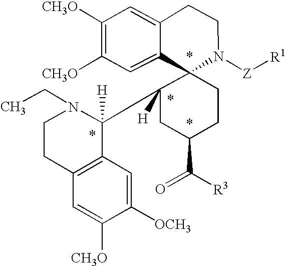

| Ex. | | | Physicochemical |
|---|---|---|---|
| No. | —Z—R¹ | R³ | properties etc. |
| 121(1) | 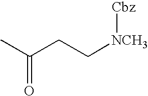 | 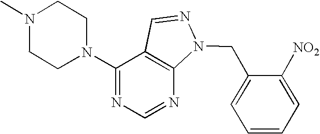 | Amorphous powder MS(APCI)1065(M + H) |
| 122(1) | 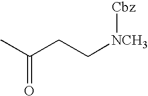 | 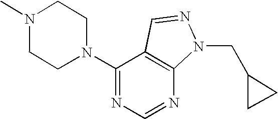 | Amoephous powder MS(APCI)984(M + H) |
| 123(1) | 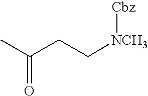 | 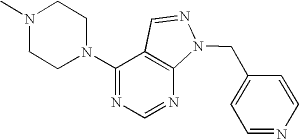 | Optically-active isomer (Amorphous powder) [α]_{D}-53.6°(c1.0, CHCl₃) |
| 124(1) | 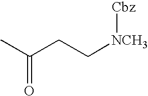 | 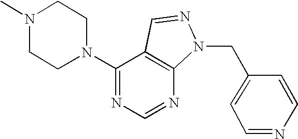 | Optically-active isomer (Amorphous powder) MS(APCI)1021(M + H) |
| 125(1) | 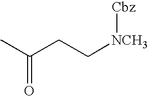 | 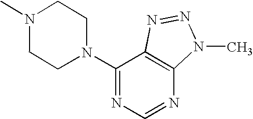 | Optically-active isomer (Amorphous powder) [α]_{D}-62.29°(c1.0, CHCl₃) |
| 126(1) | 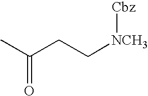 | 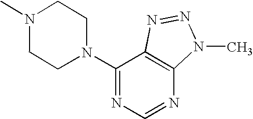 | Optically-active isomer (Amorphous powder) MS(APCI)945(M + H) |

Cbz: Benzyloxycarbonyl group

TABLE 7
(No. 12)

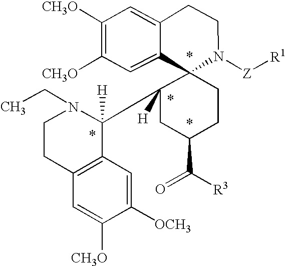

| Ex. | | | Physicochemical |
|---|---|---|---|
| No. | —Z—R¹ | R³ | properties etc. |
| 127(1) | 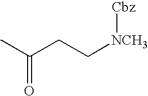 | 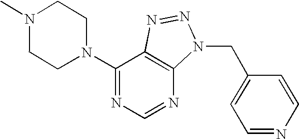 | Amorphous powder MS(APCI)1022.6(M + H) |
| 128(1) | 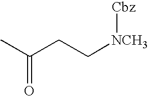 | 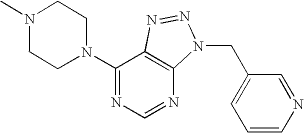 | Amorphous powder MS(APCI)1022.5(M + H) |
| 129(1) | 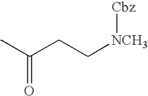 | 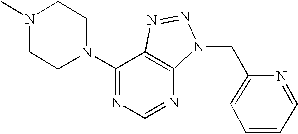 | Amorphous powder MS(APCI)1022.6(M + H) |
| 130(1) | 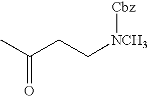 | 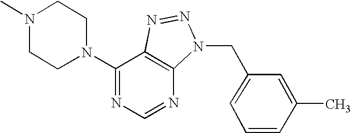 | Amorphous powder MS(APCI)1035.6(M + H) |
| 131(1) | 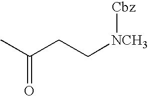 | 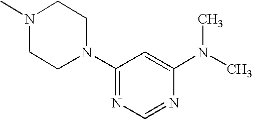 | Amorphous powder MS(APCI)933.5(M + H) |
| 132(1) | 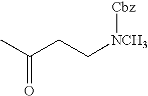 | 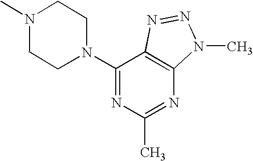 | Amorphous powder MS(APCI)959.7(M + H) |

Cbz: Benzyloxycarbonyl group

TABLE 7
(No. 13)

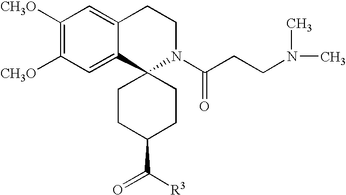

| | | Physicochemical |
|---|---|---|
| Ex. No. | R³ | properties etc. |
| 133(1) | 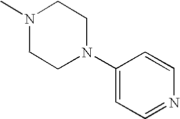 | Amorphous powder MS(APCI)550(M + H) |
| 134(1) | 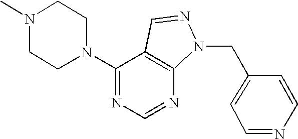 | Amorphous powder MS(APCI)682(M + H) |
| 135(1) | 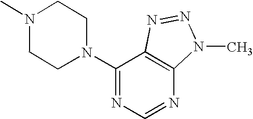 | Amorphous powder MS(APCI)606(M + H) |
| 136(1) | 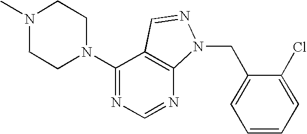 | Amorphous powder MS(APCI)715(M + H) |
| 137(1) | 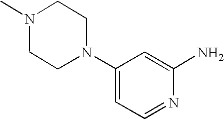 | Amorphous powder MS(APCI)565(M + H) |

TABLE 7
(No. 14)

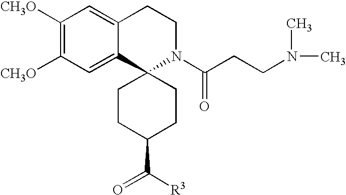

| | | Physicochemical |
|---|---|---|
| Ex. No. | R³ | properties etc. |
| 138(1) | 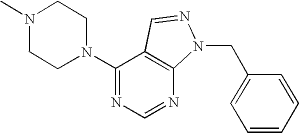 | Amorphous powder MS(APCI)681.6(M + H) |
| 139(1) | 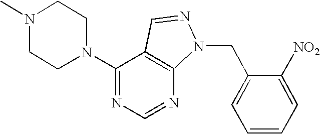 | Amorphous powder MS(APCI)726(M + H) |
| 140(1) | 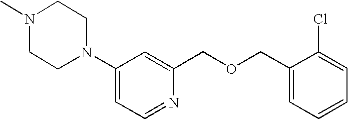 | Amorphous powder MS(APCI)704(M + H) |

TABLE 7
(No. 15)

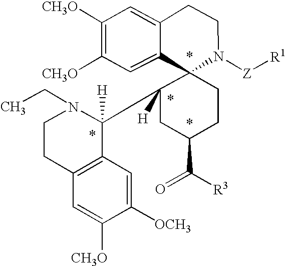

| Ex. | | | Physicochemical |
|---|---|---|---|
| No. | —Z—R¹ | R³ | properties etc. |
| 141(1) | 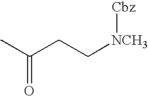 | 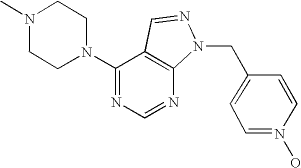 | Amorphous powder MS(APCI)1037(M + H) |
| 142(1) | 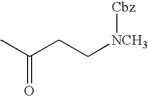 | 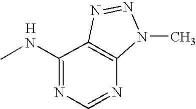 | Amorphous powder MS(APCI)876.6(M + H) |
| 143(1) | 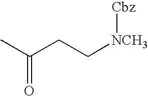 | 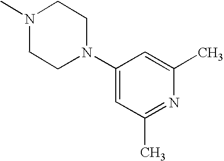 | Amorphous powder MS(APCI)917.6(M + H) |
| 144(1) | 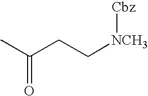 | 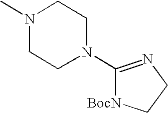 | Amorphous powder MS(APCI)980.6(M + H) |
| 145(1) | 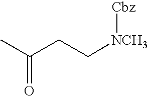 | 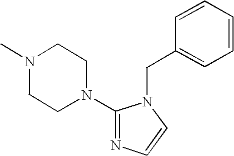 | Amorphous powder MS(APCI)968(M + H) |
| 146(1) | 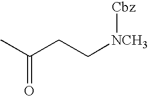 | 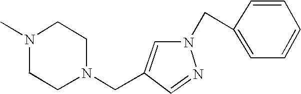 | Amorphous powder MS(APCI)982(M + H) |

Cbz: Benzyloxycarbonyl group, Boc: tert-butoxycarbonyl group

TABLE 7
(No. 16)

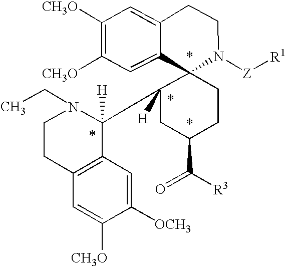

| Ex. | | | Physicochemical |
|---|---|---|---|
| No. | —Z—R¹ | R³ | properties etc. |
| 147(1) | 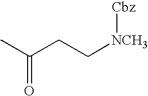 | 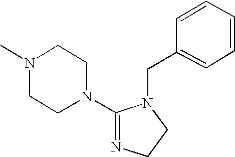 | Amorphous powder MS(APCI)971(M + H) |
| 148(1) | 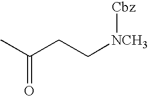 | 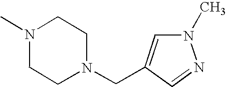 | Amorphous powder MS(APCI)906(M + H) |
| 149(1) | 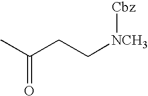 | 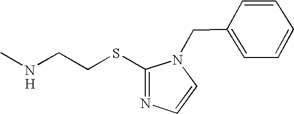 | Amorphous powder MS(APCI)959.6(M + H) |
| 150(1) | 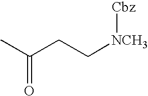 | 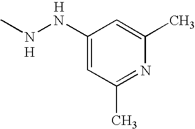 | Amorphous powder MS(APCI)863.6(M + H) |

Cbz: Benzyloxycarbonyl group

TABLE 7
(No. 17)

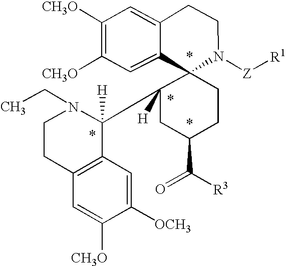

| Ex. | | | Physicochemical |
|---|---|---|---|
| No. | —Z—R¹ | R³ | properties etc. |
| 151(1) | 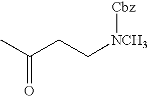 | 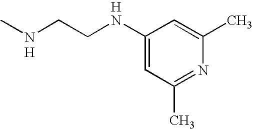 | Amorphous powder MS(APCI)891.6(M + H) |
| 152(1) | 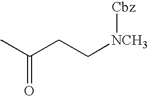 | 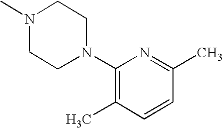 | Amorphous powder MS(APCI)917(M + H) |

Cbz: Benzyloxycarbonyl group

TABLE 7
(No. 18)

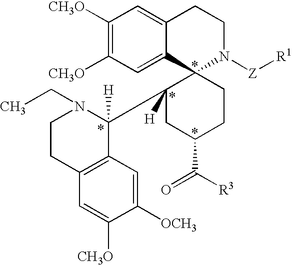

| Ex. No. | —Z—R¹ | R³ | Physicochemical properties etc. |
|---|---|---|---|
| 153(1) | 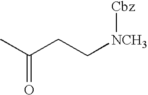 | 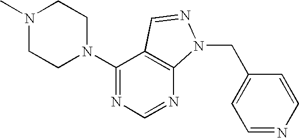 | Amorphous powder MS(APCI) 1021(M + H) |

Cbz: Benzyloxycarbonyl group

TABLE 7
(No. 19)

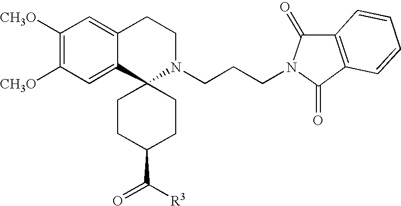

| Ex. No. | R³ | Physicochemical properties etc. |
|---|---|---|
| 154(1) | 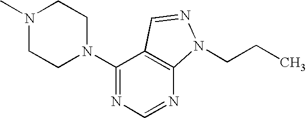 | Amorphous powder MS(APCI) 721(M + H) |
| 155(1) | 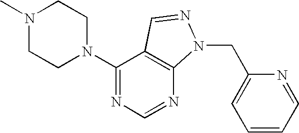 | Amorphous powder MS(APCI) 770(M + H) |
| 156(1) | 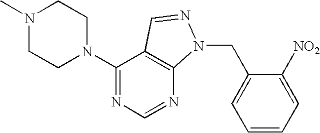 | Amorphous powder MS(APCI) 814(M + H) |
| 157(1) | 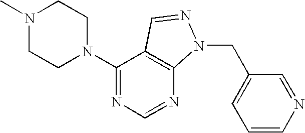 | Amorphous powder MS(APCI) 770(M + H) |
| 158(1) | 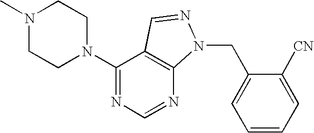 | Amorphous powder MS(APCI) 794(M + H) |

TABLE 7
(No. 20)

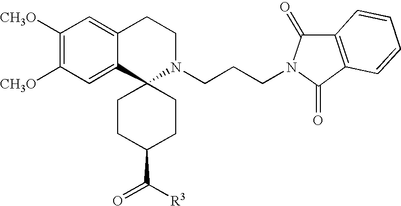

| Ex. No. | R³ | Physicochemical properties etc. |
|---|---|---|
| 159(1) | 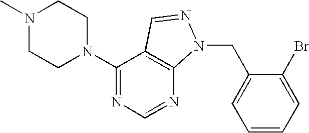 | Amorphous powder MS(APCI) 849(M + H) |
| 160(1) | 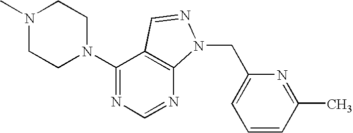 | Amorphous powder MS(APCI) 784(M + H) |

TABLE 7
(No. 21)

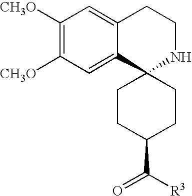

| Ex. No. | R³ | Physicochemical properties etc. |
|---|---|---|
| 161(1) | 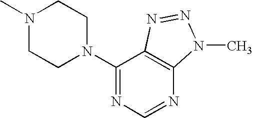 | M.p. 251-254° C. |
| 162(1) | 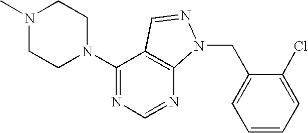 | M.p. 211-213° C. |

TABLE 7
(No. 22)

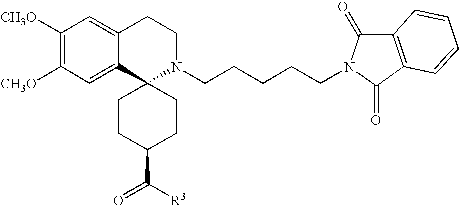

| Ex. No. | R³ | Physicochemical properties etc. |
|---|---|---|
| 163(1) | 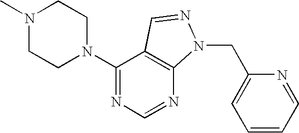 | Amorphous powder MS(APCI) 784.6(M + H) |

2) The compounds obtained in the above Examples 53(1) to 163(1) are treated in the same manner as described in Example 2(2) to give the following compounds (salt).

Example 53(2): 2 Fumaric acid salt of the compound obtained in Example 53(1)

Example 54(2): 2 Fumaric acid salt of the compound obtained in Example 54(1)

Example 56(2): 2 Fumaric acid salt of the compound obtained in Example 56(1); Amorphous powder, IR(neat+chloroform)cm⁻¹: 1710, 1637

Example 57(2): 2 Fumaric acid salt of the compound obtained in Example 57(.1); Amorphous powder, MS(APCI)m/z: 756(M+H), IR(neat+chloroform)cm⁻¹: 1707, 1635

Example 58(2): 2 Fumaric acid salt of the compound obtained in Example 58(1)

Example 59(2): 2 Fumaric acid salt of the compound obtained in Example 59(1)

Example 60(2): 2 Fumaric acid salt of the compound obtained in Example 60(1); Amorphous powder, MS(APCI)m/z: 769(M+H), IR(neat+chloroform)cm⁻¹: 1703, 1639

Example 61(2): 2 Fumaric acid salt of the compound obtained in Example 61(1)

Example 62(2): 2 Fumaric acid salt of the compound obtained in Example 62(1); Amorphous powder, MS(APCI)m/z: 740(M+H), IR(neat+chloroform)cm⁻¹: 1706, 1637

Example 63(2): 2 Fumaric acid salt of the compound obtained in Example 63(1); Amorphous powder, MS(APCI)m/z: 726(M+H), IR(neat+chloroform)cm⁻¹: 1705, 1612

Example 64(2): 2 Fumaric acid salt of the compound obtained in Example 64(1)

Example 65(2): 2 Fumaric acid salt of the compound obtained in Example 65(1)

Example 66(2): 2 Fumaric acid salt of the compound obtained in Example 66(1); Lyophilized amorphous powder, MS(APCI)m/z: 783.6(M+H), IR(Nujol)cm⁻¹: 1706, 1637

Example 67(2): 2 Fumaric acid salt of the compound obtained in Example 67(1); Lyophilized amorphous powder, MS(APCI)m/z: 783(M+H), IR(Nujol)cm⁻¹: 1705, 1641

Example 68(2): 2 Fumaric acid salt of the compound obtained in Example 68(1)

Example 69(2): 2 Fumaric acid salt of the compound obtained in Example 69(1)

Example 70(2): 2 Fumaric acid salt of the compound obtained in Example 70(1); Amorphous powder, MS(APCI)m/z: 799.8(M+H), IR(neat+chloroform)cm⁻¹: 1703, 1640

Example 71(2): 2 Fumaric acid salt of the compound obtained in Example 71(1)

Example 74(2): 2 Fumaric acid salt of the compound obtained in Example 74(1)

Example 75(2): 2 Fumaric acid salt of the compound obtained in Example 75(1)

Example 76(2): 2 Fumaric acid salt of the compound obtained in Example 76(1)

Example 135(2): 1 Fumaric acid salt of the compound obtained in Example 135(1); Lyophilized amorphous powder, MS(APCI)m/z: 606(M+H), IR(Nujol)cm⁻¹: 1702, 1635

Example 136(2): 1 Fumaric acid salt of the compound obtained in Example 136(1); Lyophilized amorphous powder, MS(APCI)m/z: 583(M+H), IR(Nujol)cm⁻¹: 1706, 1637

Example 137(2): 1 Fumaric acid sale of the compound obtained in Example 137(1); Lyophilized amorphous powder, MS(APCI)m/z: 565.6(M+H), IR(Nujol)cm⁻¹: 1631

Example 138(2): 1 Fumaric acid salt of the compound obtained in Example 138(1); Lyophilized amorphous powder, MS(APCI)m/z: 681.6(M+H), IR(Nujol)cm⁻¹: 1707, 1633

Example 139(2): 1 Fumaric acid salt of the compound obtained in Example 139(1); Lyophilized amorphous powder, MS(APCI)m/z: 726(M+H), IR(Nujol)cm⁻¹: 1634

### Example 164

1) To a solution of (1R*,2R*(S*),4R*)-2′-aceyl-3′,4′-dihydro-6′,7′-dimethoxy-2-(2-ethyl-1,2,3,4-tetrahydro-6,7-dimethoxy-1-isoquinolyl)-4-[2-(N-benzyloxycarbonylamino)ethyl]aminocarbonyl-spiro[cyclohexane-1,1′(2′H)-isoquinoline] (Compound obtained in Example 72(1)) (2.47 g) in tetrahydrofuran (30 mL) is added borane-tetrahydrofuran complex (1M solution in tetrahydrofuran; 35.55 mL) and the mixture is stirred at room temperature for 3 days. To the reaction mixture is added methanol and 10% hydrochloric acid and the mixture is stirred at room temperature for 3 hours. The reaction mixture is concentrated and ethyl acetate is added to the residue. The mixture is washed with saturated aqueous sodium hydrogencarbonate solution and dried over sodium sulfate. The mixture is concentrated and the residue is purified by column chromatography on silica gel (Solvent; chloroform:methanol:28% aqueous ammonia=10:1:0.1) to give (1R*,2R*(S),4R)-2′-ethyl-3′,4′-dihydro-6′,7′-dimethoxy-2-(2-ethyl-1,2,3,4-tetrahydro-6,7-dimethoxy-1-isoquinolyl)-4-[2-(N-benzyloxycarbonylamino)ethyl]-aminomethyl-spiro[cyclohexane-1,1′(2′H)-isoquinoline] (1.32 g, 52%) as an amorphous powder.

MS(APCI) m/z: 715(M+H), IR(neat)cm⁻¹: 3366, 2931, 1609.

2) To a solution of the compound obtained in the above step (1) (494 mg) and triethylamine (80 mg) in methylene chloride (10 mL) is added acetyl chloride (60 mg), and the mixture is stirred at room temperature for 3 hours. The reaction mixture is washed with saturated aqueous sodium hydrogencarbonate solution and dried over sodium sulfate. The mixture is concentrated and the residue is purified by column chromatography on silica gel (Solvent; chloroform:methanol:28% aqueous ammonia=20:1:0.05) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[2-(N-benzyloxycarbonylamino)ethyl]-N-acetylaminomethyl-spiro[cyclohexane-1,1′(2′H)-isoquinoline] (427 mg, 82%) as an amorphous powder.

MS(ESI)m/z: 757(M+H), IR(Nujol)cm⁻¹: 1718, 1633.

### Example 165

1) To a solution of (1R*,2R*(S*),4R*)-2′-[3-(N-benzyloxycarbonyl-N-methylamino)propionyl]-3 ′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 78(1)) (351.1 mg) in acetonitrile (5 mL) is added iodotrimethylsilane (0.25 mL) and the mixture is stirred at room temperature for 1 hour. To the reaction mixture is added 10% hydrochloric acid and the mixture is washed with diisopropylether. The aqueous layer is basified with an aquenus potasium carbonate solution and extracted with chloroform. The organic layer is washed with saturated aqueous NaCl solution and dried over sodium sulfate. The extract is concentrated and the residue is purified by column chromatography on silica gel (Solvent; chloroform:methanol:28% aqueous ammonia=200:10:1) to give (1R*,2R*(S),4R)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (209.6 mg, 69%) as crystals.

M.p. 178-180° C., MS(APCI)m/z: 887(M+H).

2) The compound obtained in the above step (1) are treated in the same manner as described in Example 2(2) to give 2 fumaric acid salt thereof as an amorphous powder.

MS(ESI)m/z: 887.5(M+H), IR(neat+chloroform)cm⁻¹: 1704, 1633.

### Examples 166 to 199

1) The corresponding materials are treated in the same manner as described in Example 165(1) to give the following compounds as shown in the following tables (Table. 8).

TABLE 8
(No. 1)

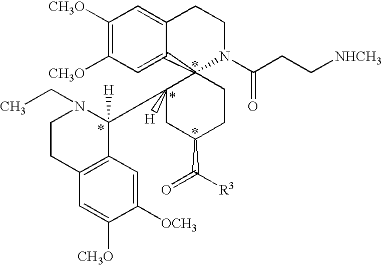

| Ex. No. | R³ | Physicochemical properties etc. |
|---|---|---|
| 166(1) | 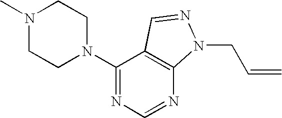 | Amorphous powder MS(APCI) 836(M + H) |
| 167(1) | 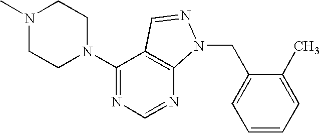 | Amorphous powder MS(APCI) 900(M + H) |
| 168(1) | 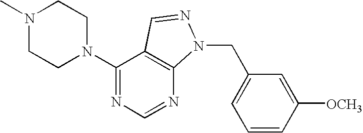 | Amorphous powder MS(APCI) 916(M + H) |
| 169(1) | 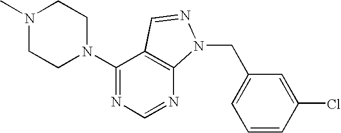 | Amorphous powder MS(APCI) 920(M + H) |
| 170(1) | 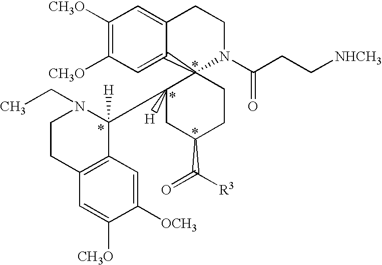 | Amorphous powder MS(APCI) 920(M + H) |

TABLE 8
(No. 2)

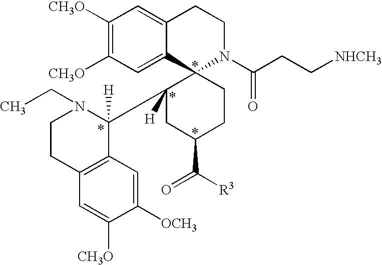

| Ex. No. | R³ | Physicochemical properties etc. |
|---|---|---|
| 171(1) | 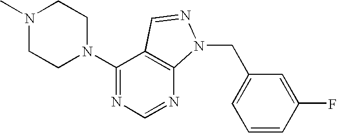 | Amorphous powder MS(APCI) 904(M + H) |
| 172(1) | 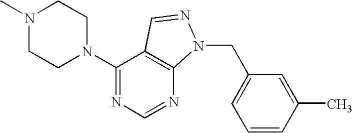 | Amorphous powder MS(APCI) 900(M + H) |
| 173(1) | 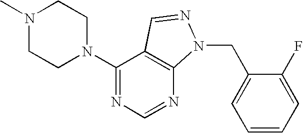 | Amorphous powder MS(APCI) 904(M + H) |
| 174(1) | 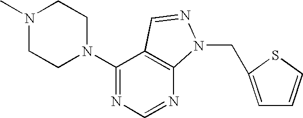 | Amorphous powder MS(APCI) 892(M + H) |
| 175(1) | 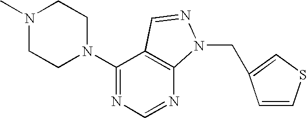 | Amorphous powder MS(APCI) 892(M + H) |
| 176(1) | 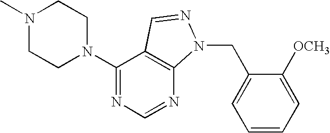 | Amorphous powder MS(APCI) 916(M + H) |

TABLE 8
(No. 3)

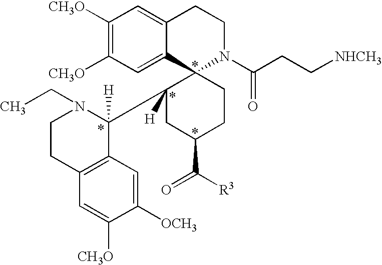

| Ex. No. | R³ | Physicochemical properties etc. |
|---|---|---|
| 177(1) | 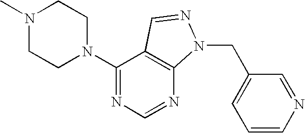 | Amorphous powder MS(APCI) 887(M + H) |
| 178(1) | 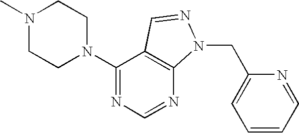 | Amorphous powder MS(APCI) 887(M + H) |
| 179(1) | 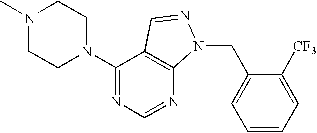 | M.p. 171-172.5° C. MS(APCI) 954(M + H) |
| 180(1) | 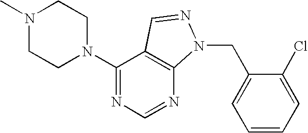 | Amorphous powder MS(APCI) 954(M + H) |
| 181(1) | 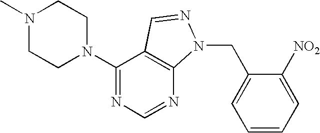 | Amorphous powder MS(APCI) 931(M + H) |
| 182(1) | 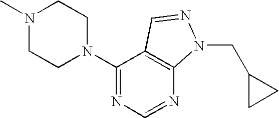 | Amorphous powder MS(APCI) 850(M + H) |

TABLE 8
(No. 4)

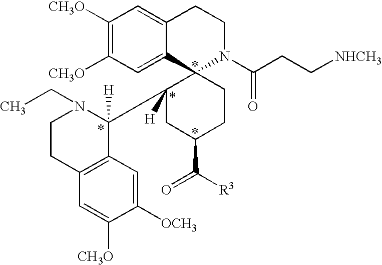

| Ex. No. | R³ | Physicochemical properties etc. |
|---|---|---|
| 183(1) | 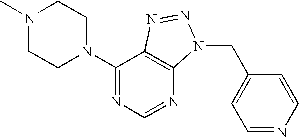 | Amorphous powder MS(APCI) 888.5(M + H) |
| 184(1) | 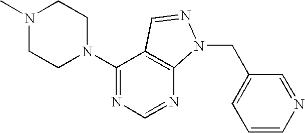 | Amorphous powder MS(APCI) 888.6(M + H) |
| 185(1) | 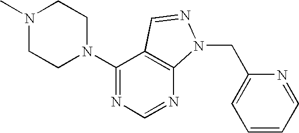 | Amorphous powder MS(APCI) 888.6(M + H) |
| 186(1) | 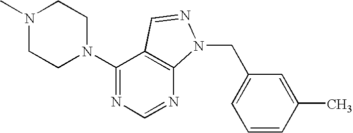 | Amorphous powder MS(APCI) 901.6(M + H) |
| 187(1) | 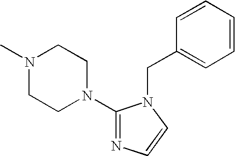 | Amorphous powder MS(APCI) 834(M + H) |
| 188(1) | 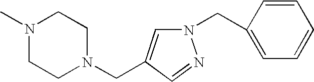 | Amorphous powder MS(APCI) 848(M + H) |

TABLE 8
(No. 5)

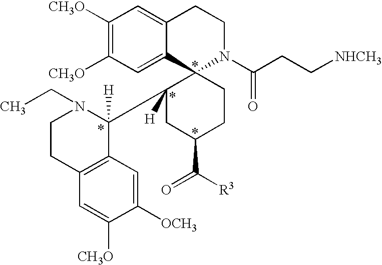

| Ex. No. | R³ | Physicochemical properties etc. |
|---|---|---|
| 189(1) | 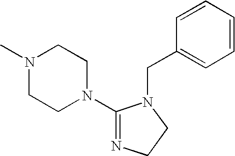 | Amorphous powder MS(APCI) 836(M + H) |
| 190(1) | 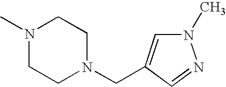 | Amorphous powder MS(APCI) 772(M + H) |
| 191(1) | 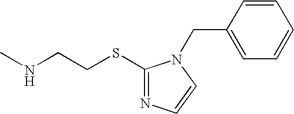 | Amorphous powder MS(APCI) 825(M + H) |
| 192(1) | 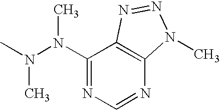 | Amorphous powder MS(APCI) 785(M + H) |
| 193(1) | 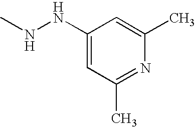 | Amorphous powder MS(APCI) 729.5(M + H) |
| 194(1) | 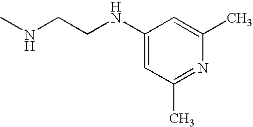 | Amorphous powder MS(APCI) 757.6(M + H) |

TABLE 8
(No. 6)

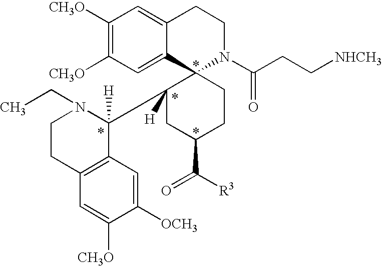

| Ex. No. | R³ | Physicochemical properties etc. |
|---|---|---|
| 195(1) | 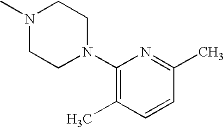 | Amorphous powder MS(APCI) 783(M + H) |
| 196(1) | 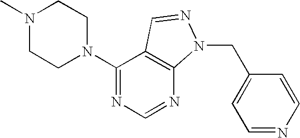 | Optically-active isomer Amorphous powder MS(APCI) 887(M + H) [α]_{D} −56.79° (c1.0, CHCl₃) |
| 197(1) | 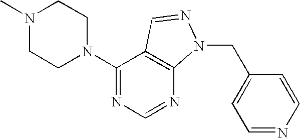 | Optically-active isomer Amorphous powder MS(APCI) 887(M + H) [α]_{D} +57.0° (c1.0, CHCl₃) |
| 198(1) | 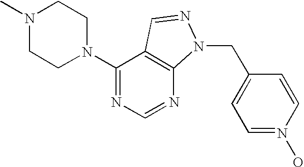 | Optically-active isomer Amorphous powder MS(APCI) 903(M + H) |

TABLE 8
(No. 7)

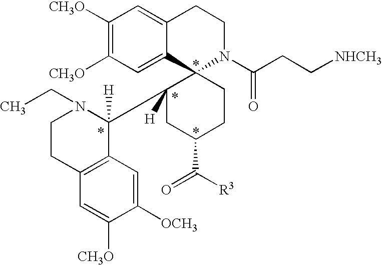

| Ex. No. | R³ | Physicochemical properties etc. |
|---|---|---|
| 199(1) | 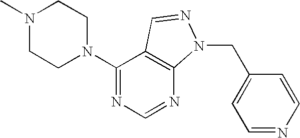 | Amorphous powder MS(APCI) 887(M + H) |

2) The compounds obtained in the above Examples 166(1) to 199(1) are treated in the same manner as described in Example 2(2) to give the following compounds (salt).

Example 166(2): 2 Fumaric acid salt of the compound obtained in Example 166(1)

Example 167(2): 2 Fumaric acid salt of the compound obtained in Example 167(1)

Example 168(2): 1 Fumaric acid salt of the compound obtained in Example 168(1)

Example 169(2): 1 Fumaric acid salt of the compound obtained in Example 169(1)

Example 170(2): 1 Fumaric acid salt of the compound obtained in Example 170(1)

Example 171(2): 1 Fumaric acid salt of the compound obtained in Example 171(1)

Example 172(2): 1 Fumaric acid salt of the compound obtained in Example 172(1)

Example 173(2): 1 Fumaric acid salt of the compound obtained in Example 173(1)

Example 174(2): 1 Fumaric acid salt of the compound obtained in Example 174(1)

Example 175(2): 1 Fumaric acid salt of the compound obtained in Example 175(1)

Example 176(2): 1 Fumaric acid salt of the compound obtained in Example 176(1)

Example 177(2): 1 Fumaric acid salt of the compound obtained in Example 177(1)

Example 178(2): 2 Fumaric acid salt of the compound obtained in Example 178(1); Amorphous powder, MS(APCI)m/z: 887(M+H), IR(neat+chloroform)cm⁻¹: 1705, 1698, 1633

Example 179(2): 2 Fumaric acid salt of the compound obtained in Example 179(1); Powder, MS(APCI)m/z: 954(M+H), IR(Nujol)cm⁻¹: 1703, 1634, 1573

Example 180(2): 1 Fumaric acid salt of the compound obtained in Example 180(1)

Example 181(2): 2 Fumaric acid salt of the compound obtained in Example 180(1); Powder, MS(APCI)m/z: 931(M+H), IR(Nujol)cm⁻¹: 1636, 1572

Example 182(2): 1 Fumaric acid salt of the compound obtained in Example 182(1)

Example 183(2): 1 Fumaric acid salt of the compound obtained in Example 183(1)

Example 184(2): 1 Fumaric acid salt of the compound obtained in Example 184(1)

Example 185(2): 1 Fumaric acid salt of the compound obtained in Example 185(1)

Example 186(2): 1 Fumaric acid salt of the compound obtained in Example 186(1)

Example 187(2): 1 Fumaric acid salt of the compound obtained in Example 187(1)

Example 188(2): 1 Fumaric acid salt of the compound obtained in Example 188(1)

Example 189(2): 1 Fumaric acid salt of the compound obtained in Example 189(1)

Example 190(2): 1 Fumaric acid salt of the compound obtained in Example 190(1)

Example 191(2): 1 Fumaric acid salt of the compound obtained in Example 191(1); Powder, MS(APCI)m/z: 825.6(M+H), IR(Nujol)cm⁻: 1653, 1637.

Example 192(2): 1 Fumaric acid salt of the compound obtained in Example 192(1); Lyophilized amorphous powder, MS(APCI)m/z: 785.6(M+H), IR(Nujol)cm⁻¹: 1653

Example 193(2): 1 Fumaric acid salt of the compound obtained in Example 193(1); Powder, MS(APCI)m/z: 729.5(M+H), IR(Nujol)cm⁻¹: 1652.

Example 194(2): 1 Fumaric acid salt of the compound obtained in Example 194(1); Powder, MS(APCI)m/z: 757.6(M+H), IR(Nujol)cm⁻¹: 3347, 1693, 1645

Example 195(2): 1 Fumaric acid salt of the compound obtained in Example 195(1)

Example 196(2): 1 Fumaric acid salt of the compound obtained in Example 196(1); Powder, MS(APCI)m/z: 887(M+H), [α]D −39.59° (c1.0, ethanol)

Example 197(2): 1 Fumaric acid salt of the compound obtained in Example 197(1); Powder, MS(APCI)m/z: 887(M+H), [α]D +40.0° (c1.0, ethanol)

Example 198(2): 1 Fumaric acid salt of the compound obtained in Example 198(1); Amorphous powder, MS(APCI)m/z: 903.7(M+H), IR(Nujol)cm⁻¹: 1639

Example 199(2): 1 Fumaric acid salt of the compound obtained in Example 199(1); Powder, MS(APCI)m/z: 887.9(M+H), IR(Nujol)cm⁻¹: 3406, 1634, 1573

### Example 200

1) A mixture of (1R*,2R*(S*),4R*)-2′-[3-(N-benzyloxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(4-pyridyl)piperazine-1-yl]carbonyl-spiro[cyclohexane-1,1′(2′H)-isoquinoline] (Compound obtained in Example 79(1)) (190 mg) and 10% palladium-carbon (380 mg) in ethanol (10 mL) is subjected to catalytic hydrogenation under atmospheric pressure at room temperature for 16 hours. The reaction mixture is filtered and the infiltrate is washed with ethanol. The combined filtrate is concentrated. The residue is purified by column chromatography on silica gel (Solvent; chloroform:methanol:28% aqueous ammonia=200:10:1) to give (1R,2R(S),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(4-pyridyl)piperazine-1-yl]carbonyl-spiro[cyclohexane-1,1′(2′H)-isoquinoline] (128 mg, 77%) as an amorphous powder.

NMR(CDCl3) δ: 0.78 (3H, t, J=7.1), 2.49 (3H, s), 3.41 (1H, d, J=9.7), 3.66 (3H, s), 3.86 (3H, s), 3.98 (3H, s), 6.42 (1H, s), 6.56 (1H, s), 6.60 (1H, s), 7.57 (1H, s).

2) The compound obtained in the above step (1) is treated in the same manner as described in Example 2(2) to give 2 fumaric acid salt thereof as an amorphous powder.

MS(APCI)m/z: 755.6(M+H), IR(neat+chloroform)cm⁻¹: 1704, 1639, 1515.

### Example 201 to 212

1) The corresponding materials are treated in the same manner as described in Example 200(1) to give the compounds as shown in the following tables (Table.9).

TABLE 9
(No. 1)

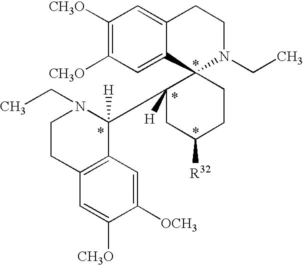

| Ex. No. | R³² | Physicochemical properties etc. |
|---|---|---|
| 201(1) | 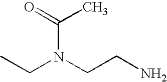 | Amorphous powder MS(APCI) 623(M + H) |

TABLE 9
(No. 2)

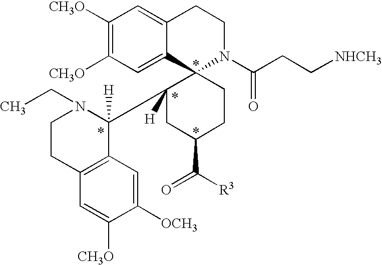

| Ex. No. | R³ | Physicochemical properties etc. |
|---|---|---|
| 202(1) | 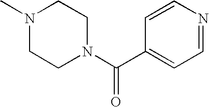 | Amorphous powder MS(APCI) 783(M + H) |
| 203(1) | 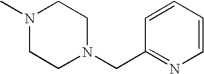 | Amorphous powder MS(APCI) 769(M + H) |
| 204(1) | 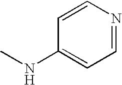 | Amorphous powder MS(APCI) 686(M + H) |
| 205(1) | 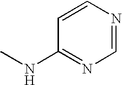 | Amorphous powder MS(APCI) 687(M + H) |
| 206(1) | 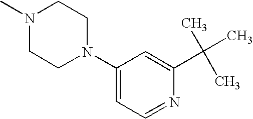 | Amorphous powder MS(APCI) 811(M + H) |
| 207(1) | 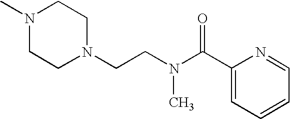 | Amorphous powder MS(APCI) 840(M + H) |
| 208(1) | 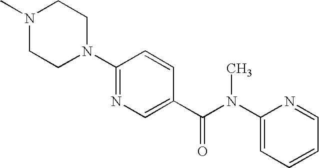 | Amorphous powder MS(APCI) 889(M + H) |

TABLE 9
(No. 3)

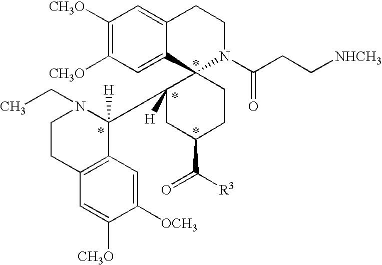

| Ex. No. | R³ | Physicochemical properties etc. |
|---|---|---|
| 209(1) | 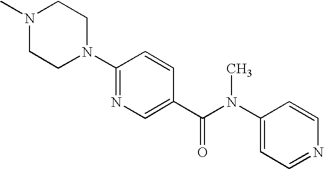 | Amorphous powder MS(APCI) 889(M + H) |
| 210(1) | 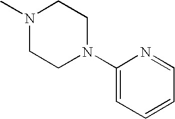 | Optically-active isomer (Amorphous powder) MS(APCI) 755(M + H) [α]_{D} −49.1° (c1.0, CHCl₃) |
| 211(1) | 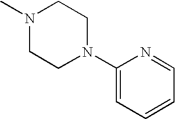 | Optically-active isomer (Amorphous powder) MS(APCI) 755(M + H) [a]_{D} +50.7° (c1.0, CHCl₃) |
| 212(1) | 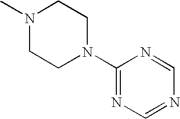 | Amorphous powder MS(APCI) 757(M + H) |

2) The compounds obtained in the above Examples 201(1) to 212(1) are treated in the same manner as described in Example 2(2) to give the following compounds (salt).

Example 201(2): 2 Fumaric acid salt of the compound obtained in Example 201(1)

Example 202(2): 2 Fumaric acid salt of the compound obtained in Example 202(1)

Example 203(2): 2 Fumaric acid salt of the compound obtained in Example 203(1)

Example 204(2): 2 Fumaric acid salt of the compound obtained in Example 204(1)

Example 205(2): 2 Fumaric acid salt of the compound obtained in Example 205(1)

Example 206(2): 2 Fumaric acid salt of the compound obtained in Example 206(1)

Example 207(2): 2 Fumaric acid salt of the compound obtained in Example 207(1)

Example 208(2): 2 Fumaric acid salt of the compound obtained in Example 208(1)

Example 209(2): 2 Fumaric acid salt of the compound obtained in Example 209(1)

Example 210(2): 2 Fumaric acid salt of the compound obtained in Example 210(1)

Example 211(2): 2 Fumaric acid salt of the compound obtained in Example 211(1)

Example 212(2): 2 Fumaric acid salt of the compound obtained in Example 212(1)

### Example 213

1) A mixture of (1R*,2R*(S*),4R*)-2′-[3-(N-benzyloxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-pyridyl)piperazine-1-yl]carbonyl-spiro[cyclohexane-1,1′(2′H)-isoquinoline] (Compound obtained in Example 41(1))(60 mg),10% palladium-carbon (60 mg), ammonium formate (85 mg) and methanol (6 mL) is refluxed for 1 hour. The reaction mixture is filtered and the infiltrate is washed with methanol. The combined filtrate is combined and concentrated. The residue is purified by column chromatography on silica gel (Solvent; chloroform:methanol:28% aqueous ammonia=200:10:1) to give (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-pyridyl)-piperazine-1-yl]carbonyl-spiro[cyclohexane-1,1′(2′H)-isoquinoline] (41 mg, 80%) as an amorphous powder.

NMR(CDCl3) δ: 0.79 (3H, t, J=7.1), 2.53 (3H, s), 3.70 (3H, s), 3.86 (3H, s), 3.88 (3H, s), 3.97 (3H, s), 6.45 (1H, s), 6.56 (1H, s), 6.58(1H, s), 7.55 (1H, s).

2) The compound obtained in the above step (1) is treated in the same manner as described in Example 2(2) to give 2 fumaric acid salt thereof as an amorphous powder.

MS(APCI)m/z: 755(M+H), IR(neat+chloroform)cm⁻¹: 1700, 1635, 1593.

### Example 214 to 253

1) The corresponding materials are treated in the same manner as described in Example 213(1) to give the compounds as shown in the following tables (Table.10).

TABLE 10
(No. 1)

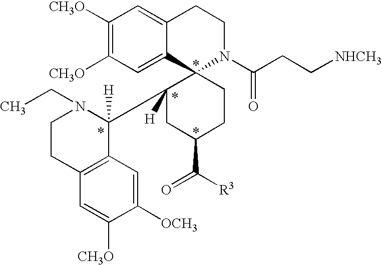

| | | Physicochemical |
|---|---|---|
| Ex. No. | R³ | properties etc. |
| 214(1) | 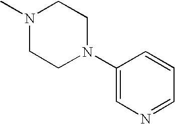 | Amorphous powder MS(APCI) 755(M + H) |
| 215(1) | 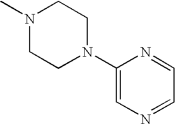 | Amorphous powder MS(APCI) 756.5(M + H) |
| 216(1) | 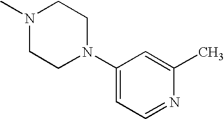 | Amorphous powder MS(APCI) 769(M + H) |
| 217(1) | 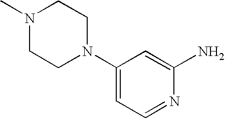 | Amorphous powder MS(APCI) 770(M + H) |
| 218(1) | 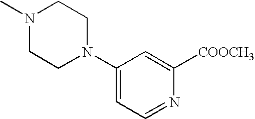 | Amorphous powder MS(APCI) 813(M + H) |

TABLE 10
(No. 2)

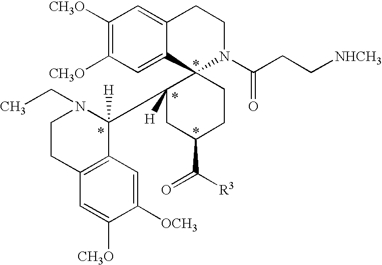

| Ex. No. | R³ | Physicochemical properties etc. |
|---|---|---|
| 220(1) | 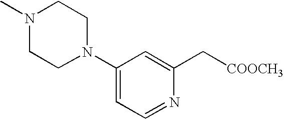 | Amorphous powder MS(APCI) 827(M + H) |
| 221(1) | 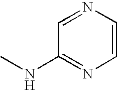 | Amorphous powder MS(APCI) 687.5(M + H) |
| 221(1a) | 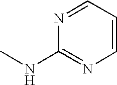 | Amorphous powder MS(APCI) 687(M + H) |
| 221(1b) | 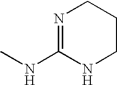 | Amorphous powder MS(APCI) 691(M + H) |
| 222(1) | 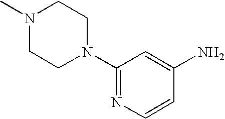 | Amorphous powder MS(APCI) 770(M + H) |
| 223(1) | 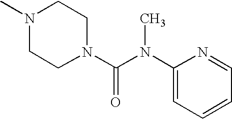 | Amorphous powder MS(APCI) 812.5(M + H) |
| 224(1) | 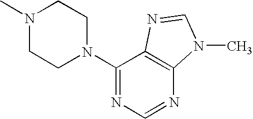 | Amorphous powder MS(APCI) 810(M + H) |

TABLE 10
(No. 3)

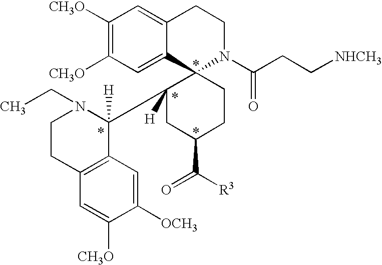

| Ex. No. | R³ | Physicochemical properties etc. |
|---|---|---|
| 225(1) | 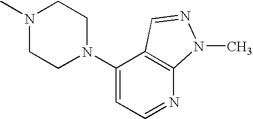 | Amorphous powder MS(APCI) 809(M + H) |
| 226(1) | 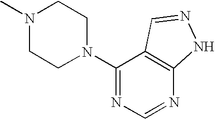 | Amorphous powder MS(APCI) 796(M + H) |
| 227(1) | 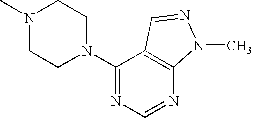 | Amorphous powder MS(APCI) 810(M + H) |
| 228(1) | 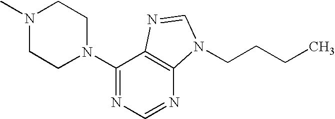 | Amorphous powder MS(APCI) 852(M + H) |
| 229(1) | 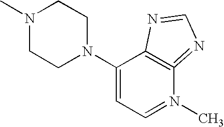 | Amorphous powder MS(APCI) 809(M + H) |
| 230(1) | 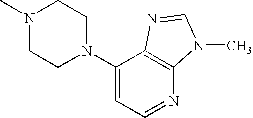 | Amorphous powder MS(APCI) 809(M + H) |
| 231(1) | 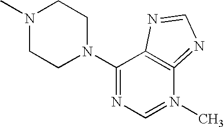 | Amorphous powder MS(APCI) 810(M + H) |

TABLE 10
(No. 4)

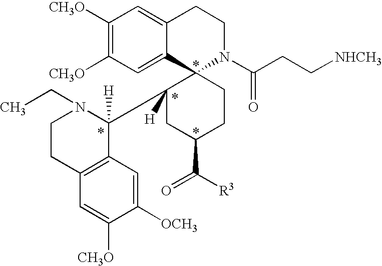

| Ex. No. | R³ | Physicochemical properties etc. |
|---|---|---|
| 232(1) | 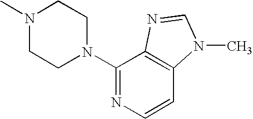 | Amorphous powder MS(APCI) 809(M + H) |
| 233(1) | 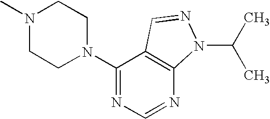 | Amorphous powder MS(APCI) 838(M + H) |
| 234(1) | 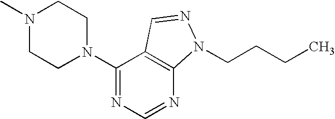 | Amorphous powder MS(APCI) 852(M + H) |
| 235(1) | 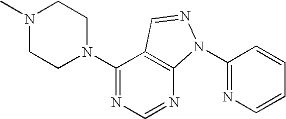 | Amorphous powder MS(APCI) 873(M + H) |
| 236(1) | 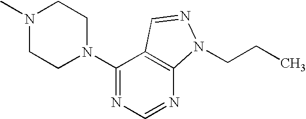 | Amorphous powder MS(APCI) 838(M + H) |
| 237(1) | 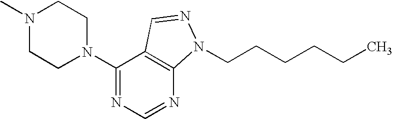 | Amorphous powder MS(APCI) 880(M + H) |
| 238(1) | 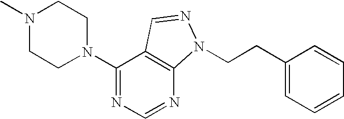 | Amorphous powder MS(APCI) 900(M + H) |

TABLE 10
(No. 5)

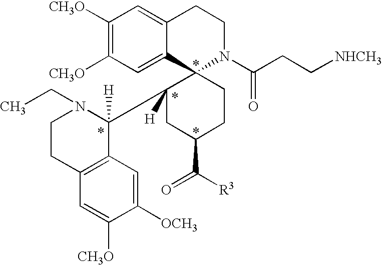

| Ex. No. | R³ | Physicochemical properties etc. |
|---|---|---|
| 239(1) | 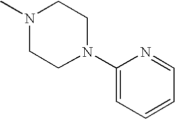 | Amorphous powder MS(APCI) 741.5(M + H) |

TABLE 10
(No. 6)

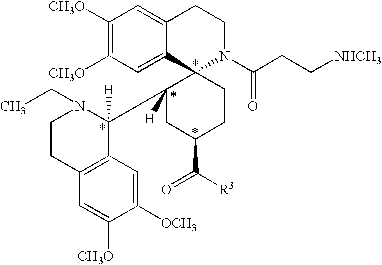

| Ex. No. | R³ | Physicochemical properties etc. |
|---|---|---|
| 240(1) | 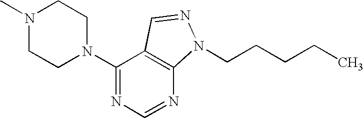 | Amorphous powder MS(APCI) 866(M + H) |
| 241(1) | 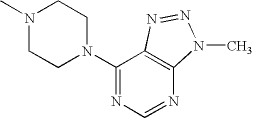 | M. p. 186-187° C. MS(APCI) 811(M + H) |
| 242(1) | 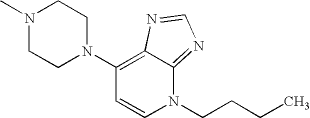 | Amorphous powder MS(APCI) 851(M + H) |
| 243(1) | 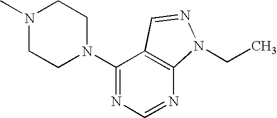 | Amorphous powder MS(APCI) 824(M + H) |
| 244(1) | 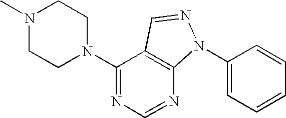 | Amorphous powder MS(APCI) 872(M + H) |
| 245(1) | 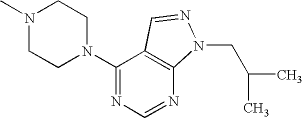 | Amorphous powder MS(APCI) 852(M + H) |
| 246(1) | 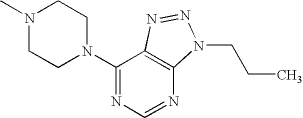 | Amorphous powder MS(APCI) 839(M + H) |

TABLE 10
(No. 7)

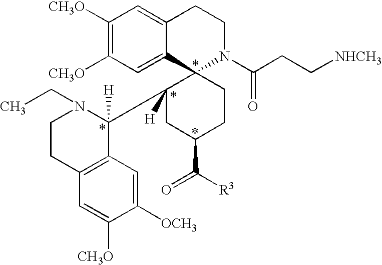

| Ex. No. | R³ | Physicochemical properties etc. |
|---|---|---|
| 247(1) | 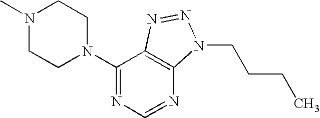 | Amorphous powder MS(APCI) 853(M + H) |
| 248(1) | 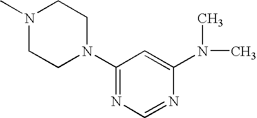 | Amorphous powder MS(APCI) 799.5(M + H) |
| 249(1) | 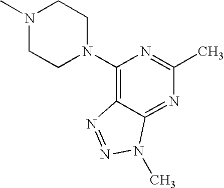 | Amorphous powder MS(APCI) 825.7(M + H) |
| 250(1) | 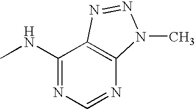 | Amorphous powder MS(APCI) 742.5(M + H) |

TABLE 10
(No. 8)

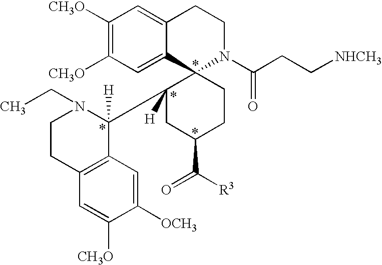

| | | Physicochemical |
|---|---|---|
| Ex. No. | R³ | properties etc. |
| 251(1) | 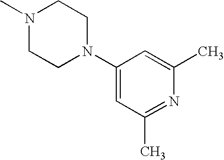 | M.p. 150-153° C. MS(APCI) 783.5(M + H) |
| 252(1) | 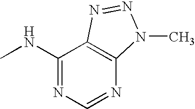 | Optically-active isomer Amorphous powder MS(APCI) 811(M + H) [a]_{D} −70.09°(c1.0, CHCl₃) |
| 253(1) | 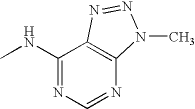 | Optically-active isomer Amorphous powder MS(APCI) 811(M + H) [a]_{D} +66.9°(c1.0, CHCl₃) |

2) The compounds obtained in the above Examples 214(1) to 253(1) are treated in the same manner as described in Example 2(2) to give the following compounds (salt).

Example 214(2): 2.5 Fumaric acid salt of the compound obtained in Example 214(1); Amorphous powder, MS(APCI)m/z: 755.6(M+H), IR(neat+chloroform)cm⁻¹: 1701, 1629, 1593

Example 215(2): 2 Fumaric acid salt of the compound obtained in Example 215(1); Amorphous powder, MS(APCI)m/z: 756.4(M+H), IR(neat+chloroform)cm⁻¹: 1705, 1629

Example 216(2): 2 Fumaric acid salt of the compound obtained in Example 216(1)

Example 217(2): 2 Fumaric acid salt of the compound obtained in Example 217(1)

Example 218(2): 2 Fumaric acid salt of the compound obtained in Example 218(1)

Example 219(2): 2 Fumaric acid salt of the compound obtained in Example 219(1); Amorphous powder, MS(APCI)m/z: 827.7(M+H), IR(neat+chloroform)cm⁻¹: 1739, 1704, 1635

Example 220(2): 2 Fumaric acid salt of the compound obtained in Example 220(1)

Example 221(2a): 2 Fumaric acid salt of the compound obtained in Example 221(1a)

Example 221(2b): 2 Fumaric acid salt of the compound obtained in Example 221(1b)

Example 222(2): 2 Fumaric acid salt of the compound obtained in Example 222(1); Powder, MS(APCI)m/z: 770.6(M+H), IR(Nujol)cm⁻¹: 3353, 3135, 1615

Example 223(2): 2 Fumaric acid salt of the compound obtained in Example 223(1)

Example 224(2): 2 Fumaric acid salt of the compound obtained in Example 224(1)

Example 225(2): 2 Fumaric acid salt of the compound obtained in Example 225(1)

Example 226(2): 2 Fumaric acid salt of the compound obtained in Example 226(1)

Example 227(2): 2 Fumaric acid salt of the compound obtained in Example 227(1)

Example 228(2): 2 Fumaric acid salt of the compound obtained in Example 228(1)

Example 229(2): 2 Fumaric acid salt of the compound obtained in Example 229(1); Amorphous powder, MS(APCI)m/z: 809.4(M+H), IR(neat+chloroform)cm⁻¹: 1704, 1632

Example 230(2): 2 Fumaric acid salt of the compound obtained in Example 230(1); Amorphous powder, MS(APCI)m/z: 809.4(M+H), IR(neat+chloroform)cm⁻¹: 1704, 1631, 1589

Example 231(2): 2 Fumaric acid salt of the compound obtained in Example 231(1)

Example 232(2): 2 Fumaric acid salt of the compound obtained in Example 232(1); Amorphous powder, MS(APCI)m/z: 809(M+H), IR(neat+chloroform)cm⁻¹: 1703, 1631

Example 233(2): 2 Fumaric acid salt of the compound obtained in Example 233(1); Amorphous powder, MS(APCI)m/z: 838(M+H), IR(neat+chloroform)cm⁻¹: 1705, 1633, 1571

Example 234(2): 2 Fumaric acid salt of the compound obtained in Example 234(1); Amorphous powder, MS(APCI)m/z: 852(M+H), IR(neat+chloroform)cm⁻¹: 1703, 1633, 1586

Example 235(2): 2 Fumaric acid salt of the compound obtained in Example 235(1); Amorphous powder, MS(APCI)m/z: 873(M+H), IR(Nujol)cm⁻¹: 3396, 1654

Example 236(2): 2 Fumaric acid salt of the compound obtained in Example 236(1); Amorphous powder, MS(APCI)m/z: 838(M+H), IR(Nujol)cm⁻¹: 3395, 1631

Example 237(2): 2 Fumaric acid salt of the compound obtained in Example 237(1); Amorphous powder, MS(APCI)m/z: 880(M+H), IR(Nujol)cm⁻¹: 3405, 1632

Example 238(2): 2 Fumaric acid salt of the compound obtained in Example 238(1); Amorphous powder, MS(APCI)m/z: 900(M+H), IR(Nujol)cm⁻¹: 3399, 1634

Example 239(2): 2 Fumaric acid salt of the compound obtained in Example 239(1); Amorphous powder, MS(APCI)m/z: 741.5(M+H), IR(neat+chloroform)cm⁻¹: 3129, 1703, 1627

Example 240(2): 2 Fumaric acid salt of the compound obtained in Example 240(1); Amorphous powder, MS(APCI)m/z: 866(M+H), IR(Nujol)cm⁻¹: 3409, 1633

Example 241(2): 2 Fumaric acid salt of the compound obtained in Example 241(1); Amorphous powder, MS(APCI)m/z: 811(M+H), IR(Nujol)cm⁻¹: 3132, 1635

Example 242(2): 2 Fumaric acid salt of the compound obtained in Example 242(1); Amorphous powder, MS(APCI)m/z: 851(M+H), IR(neat+chloroform)cm⁻¹: 3126, 1631

Example 243(2): 2 Fumaric acid salt of the compound obtained in Example 243(1); Amorphous powder, MS(APCI)m/z: 824(M+H), IR(neat+chloroform)cm⁻¹: 3130, 1633

Example 244(2): 2 Fumaric acid salt of the compound obtained in Example 244(1); Amorphous powder, MS(APCI)m/z: 872(M+H), IR(Nujol)cm⁻¹: 3398

Example 245(2): 2 Fumaric acid salt of the compound obtained in Example 245(1); Amorphous powder, MS(APCI)m/z: 852(M+H), IR(Nujol)cm⁻¹: 3329, 1637

Example 246(2): 2 Fumaric acid salt of the compound obtained in Example 246(1); Amorphous powder, MS(APCI)m/z: 839(M+H), IR(Nujol)cm⁻¹: 1638

Example 247(2): 2 Fumaric acid salt of the compound obtained in Example 247(1)

Example 248(2): 1 Fumaric acid salt of the compound obtained in Example 248(1)

Example 249(2): 1 Fumaric acid salt of the compound obtained in Example 249(1); Amorphous powder, MS(APCI)m/z: 825.7(M+H), IR(Nujol)cm⁻¹: 1636, 1597, 1576

Example 250(2): 1 Fumaric acid salt of the compound obtained in Example 250(1)

Example 251(2): 1 Fumaric acid salt of the compound obtained in Example 251(1); M.p.216-220° C. (Decomp.), MS(APCI)m/z: 783.5(M+H), IR(Nujol)cm⁻¹: 1652, 1634

Example 252(2): 1 Fumaric acid salt of the compound obtained in Example 252(1); Amorphous powder, MS(APCI)m/z: 811(M+H), [α]D −39.79° (c1.0 ethanol)

Example 253(2): 1 Fumaric acid salt of the compound obtained in Example 253(1); Amorphous powder, MS(APCI)m/z: 811(M+H), [α]D +39° (c1.0 ethanol)

### Example 254

1) To a solution of (1R*,2R*(S*),4R*)-2′-[3-(N-benzyloxycarbonyl-N-methylaminolpropionyl]-3 ′,4′-dihydro-6′,7′-dimethoxy-2-(2-ethyl-1,2,3,4-tetrahydro-6,7-dimethoxy-1-isoquinolyl)-4-[4-(4-pyrimidyl)piperazin-1-yl]carbonyl-spiro[cyclohexane-1,1′(2′H)-isoquinoline] (Compound obtained in Example 85(1)) (330 mg) in acetic acid (6 mL) is added dropwise a solution of hydrobromic acid-acetic acid (2 mL) in acetic acid (6 mL) and the mixture is stirred at room temperature for 2 hours. To the reaction mixture is added diisopropyl ether and the supernatant is removed. To the residual fraction is added saturated aqueous sodium hydrogencarbonate solution and the mixture is extracted with chloroform. The organic layer is dried over sodium sulfate and concentrated. The residue is purified by column chromatography on silica gel (Solvent; chloroform:methanol:28% aqueous ammonia=200:10:1) to give (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(4-pyridyl)-piperazine-1-yl]carbonyl-spiro[cyclohexane-1,1′(2′H)-isoquinoline] (110 mg, 39%) as an amorphous powder.

MS(APCI)m/z: 756(M+H), IR(Nujol)cm⁻¹: 1704, 1636, 1587.

2) The compound obtained in the above step (1) is treated in the same manner as described in Example 2(2) to give 2 fumaric acid salt thereof as an amorphous powder.

MS(APCI)m/z: 756.6(M+H), IR(neat+chloroform)cm⁻¹: 1704, 1634, 1589.

### Example 255

1) The corresponding materials are treated in the same manner as described in Example 254(1) to give the compounds as shown in the following table (Table.11).

TABLE 11

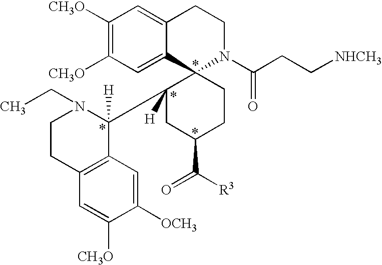

| Ex. No. | R³ | Physicochemical properties etc. |
|---|---|---|
| 255(1) | 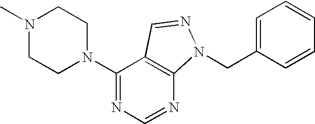 | Amorphous powder MS(APCI) 886(M + H) |

2) The compounds obtained in the above Example 254(1) is treated in the same manner as described in Example 2(2) to give 2 fumaric acid salt thereof as an amorphous powder.

MS(APCI)m/z: 886(M+H), IR(Nujol): 3395, 1632.

### Example 256

1) (1R*,2R*(S*),4R*)-2′-Ethyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (Compound obtained in Reference example 2(5)) (255 mg),4-piperazinyl-1-methylpyridinuimbromide hydrobromide (Compound obtained in Reference example 18(2)) (221 mg) and triethylamine (235 mg) are dissolved in dimethylformamide (3 mL) and diethylcyanophosphonate (0.12 mL) is added to the mixture. The mixture is stirred at room temperature for 4 hours. The reaction mixture is concentrated and the residue is purified by column chromatography on NH-silica gel (Solvent; chloroform:methanol=25:1) and product is treated with ion-exchange resin IRA-400(C1) (Organo Ltd.). The treated residue is lyophilized to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(1-methyl-4-pyridinio)-piperazine-1-yl]carbonyl-spiro[cyclohexane-1,1′(2′H)-isoquinoline]chloride (190 mg) as an amorphous powder.

MS(ESI)m/z: 712.3(M+), IR(Nujol)cm⁻¹: 1653, 1633.

### Examples 257 to 261

The corresponding materials are treated in the same manner as described in Example 256 to give compounds as shown in the following table (Table.12).

TABLE 12

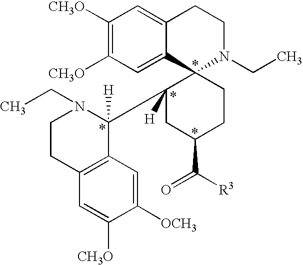

| Ex. No. | R³ | Physicochemical properties etc. |
|---|---|---|
| 257 | 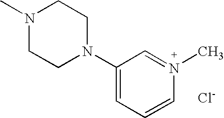 | Lyophilized powder MS(ESI) 712.3(M+) |
| 258 | 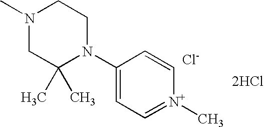 | Lyophilized powder MS(ESI) 740(M+) |
| 259 | 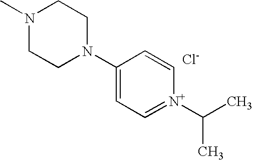 | Lyophilized powder MS(ESI) 740(M+) |
| 260 | 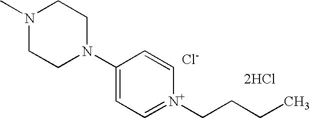 | Lyophilized powder MS(ESI) 754(M+) |
| 261 | 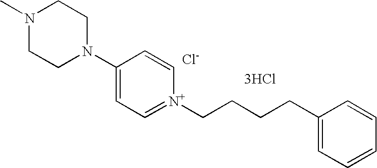 | Lyophilized powder MS(ESI) 830(M+) |

### Example 262

1) (1R,2R(S),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-methoxycarbonyl-4-pyridyl)piperazin-1-yl]carbonyl-spiro[cyclohexane-1,1′(2′H)-isoquinoline) (Compound obtained in Example 57(1))(370 mg), sodium hydroxide (121.1 mg), methanol (6.3 mL) and water (3.2 mL) are admixed and the mixture is stirred at room temperature for 16 hours. The reaction mixture is concentrated and the residue is dissolved in 10% sodium hydroxide solution. The solution is washed with ethyl acetate. The aqueous layer is neutralized with hydrochloric acid and extracted with chloroform. The organic layer is washed with saturated aqueous NaCl solution and dried over sodium sulfate, and then concentrated to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-carboxyl-4-pyridyl)-piperazine-1-yl]carbonyl-spiro[cyclohexane-1,1′(2′H)-isoquinoline] (317 mg, 87%) as an amorphous powder.

MS(FAB)m/z: 742(M+H), IR(Nujol)cm⁻¹: 1638.

2) The compound obtained in the above step (1) (198 mg) is dissolved in ethanol (5 mL) and water (1 mL), and sodium hydroxide (11 mg) is added to the mixture. The mixture is stirred for 30 minutes. The reaction mixture is concentrated and the residue is recrystallized from methanol and isopropyl alcohol to give sodium (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-carboxyl-4-pyridyl)piperazine-1-yl]carbonyl-spiro[cyclohexane-1,1′(2′H)-isoquinoline] sodium salt (117 mg, 57%) as crystals. M.p.217-220° C.

### Example 263

1) The corresponding materials are treated in the same manner as described in Example 262(1) to give the compound as shown in the following table (Table.13).

TABLE 13

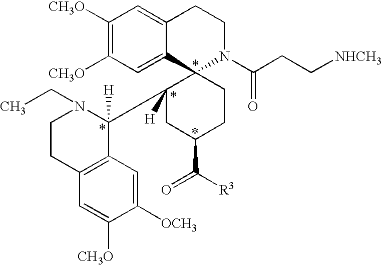

| Ex. No. | R³ | Physicochemical properties etc. |
|---|---|---|
| 263(1) | 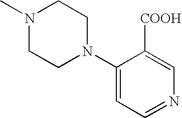 | Amorphous powder MS(APCI) 742(M + H) |

2) The compounds obtained in the above step (1) is treated in the same manner as described in Example 2(2) to give 2 fumaric acid salt thereof.

### Example 264

1) To a solution of (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-piperazinylcarbonyl-spiro[cyclohexane-1,1′(2′H)-isoquinoline] (Compound obtained in Example 55(1)) (151 mg) and triethylamine (30 mg) in methylene chloride (5 mL) is added N-methyl-N-(pyridin-2-yl)carbamoylchloride (50 mg) prepared from 2-(methylamino)-pyridine and phosgene, and the mixture is stirred at room temperature for 3 hours. The reaction mixture is concentrated. To the residue is added ethyl acetate and the mixture is washed with saturated aqueous sodium hydrogencarbonate and saturated aqueous NaCl solution, successively. The washed mixture is dried over sodium sulfate and concentrated. The residue is purified by column chromatography on silica gel (Solvent; chloroform:methanol:28% aqueous ammonia=200:100:5) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[[4-(N-(2-pyridyl)-N-methylcarbamoyl)-1-piperazinyl]-carbonyl]-spiro[cyclohexane-1,1′(2′H)-isoquinoline] (122 mg, 66%) as an amorphous powder.

MS(APCI)m/z: 755(M+H), IR(Nujol)cm⁻¹: 1659, 1640.

2) The compound obtained in the above step (1) is treated in the same manner as described in Example 2(2) to give 2 fumaric acid salt thereof as a lyopihilized amorphous powder.

### Example 265

1) (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-piperazinylcarbonyl-spiro[cyclohexane-1,1′(2′H)-isoquinoline] (Compound obtained in Example 55(1))(302 mg), pyrazolyl-1-carboxamidine (86 mg) and diisopropylethylamine (75 mg) are dissolved in dimethylformamide (10 mL) and the mixture is stirred at room temperature for 34 hours, and then further stirred at 80° C. for 14 hours after addition of pyrazolyl-1-carboxamidine (21 mg). The reaction mixture is concentrated and the residue is purified by column chromatography on NH-silica gel to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[[4-amidinopiperazin-1-yl]carbonyl]-spiro[cyclohexane-1,1′(2′H)-isoquinoline] (222 mg, 69%) as an amorphous powder.

MS(APCI)m/z: 663(M+H), IR(Nujol)cm⁻¹: 1639, 1609.

2) The compound obtained in the above step (1) is treated in the same manner as described in Example 2(2) to give 2 fumaric acid salt thereof as a lyophilized amorphous powder.

### Example 266

1) To a solution of (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-(4-nitroso-1-piperazinyl)-carbonyl-spiro[cyclohexane-1,1′(2′H)-isoquinoline] (Compound obtained in Example 73(1)) (978 mg) in 50% acetic acid (50 mL) is added zinc powder (590 mg) under ice-cooling and the mixture is stirred for 30 minutes at the same temperature. Consequently, the mixture is stirred for 2 hours at room temperature. The reaction mixture is basified with an aqueous 40% sodium hydroxide solution with ice cooling. To the mixture is added water and the mixture is extracted with chloroform. The extract is dried over sodium sulfate and concentrated. The residue is purified by column chromatography on NH-silica gel (Solvent; ethyl acetate) to give (1R*,2R*(S*),4R*)-2-ethyl-3′,4′-dihydro-6′,7′-dimethoxy-2-(2-ethyl-1,2,3,4-tetrahydro-6,7-dimethoxy-1-isoquinolyl)-4-(4-amino-1-piperazinyl)carbonyl-spiro[cyclohexane-1,1′(2′H)-isoquinoline] (912 mg, 95.3%) as an amorphous powder.

MS(APCI)m/z: 636(M+H), IR(Nujol)cm⁻¹: 1634.

2) The compound obtained in the above step (1) is treated in the same manner as described in Example 2(2) to give 2 fumaric acid salt thereof as a lyophilized amorphous powder.

MS(APCI)m/z: 636(M+H), IR(Nujol)cm⁻¹: 1705, 1636.

### Example 267

1) To a solution of (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-[4-(2-aminopyridin-4-yl)-1-piperazinyl]carbonyl-spiro[cyclohexane-1,1′(2′H)-isoquinoline] (Compound obtained in Example 52(1)) (230 mg) in pyridine (9 mL) is added acetyl chloride (60.8 mg) under ice-cooling. The mixture is stirred at the same temperature for 3.5 hours. The mixture is concentrated and ethyl acetate is added to the residue. The mixture is washed with saturated aqueous NaCl solution and dried over sodium sulfate. The mixture is concentrated and the residue is purified by column chromatography on silica gel (Solvent; chloroform:methanol:aqueous ammonia=150:5:1) to give (1R*,2R*(S*),4R*)-2′-ethyl-3′,4′-dihydro-6′,7′-dimethoxy-2-(2-ethyl-1,2,3,4 -tetrahydro-6,7-dimethoxy-1-isoquinolyl)-4-[4-(2-acetamino-4-pyridyl)-1-piperazinyl]carbonylspiro-[cyclohexane-1,1′(2′H)-isoquinoline] (189 mg, 78%) as an amorphous powder. MS(APCI)m/z: 755(M+H)

2) The compound obtained in the above step (1) is treated in the same manner as described in Example 2(2) to give 2 fumaric acid salt thereof as a lyophilized amorphous powder.

MS(APCI)m/z: 755(M+H), IR(Nujol)cm⁻¹: 1638.

### Example 268

1) To a solution of (1R*,2R*(S*),4R*)-2′-(4-pentenyl)-3′,4′-dihydro-6′,7′-dimethoxy-2-(2-ethyl-1,2,3,4-tetrahydro-6,7-dimethoxy-1-isoquinolyl)-4-[4-(4-pyridyl)-1-piperazinyl]carbonyl-spiro[cyclohexane-1,1′(2′H)-isoquinoline] (Compound obtained in Example 76(1)) (371 mg) in ethanol (30 mL) is added 10% palladium-carbon (300 mg) and the mixture is subjected to catalytic hydrogenation under atmospheric pressure at room temperature. The reaction mixture is filtered and the filtrate is concentrated. The residue is purified by column chromatography on NH-silica gel (Solvent; ethyl acetate) to give (1R*,2R*(S*),4R*)-2′-pentyl-3′,4′-dihydro-6′,7′-dimethoxy-2-(2-ethyl-1,2,3,4-tetrahydro-6,7-dimethoxy-1-isoquinolyl)-4-(4-(4-pyridyl)-1-piperazinyl]carbonyl-spiro[cyclohexane-1,1′(2′H)-isoquinoline] (315 mg, 85%) as an amorphous powder.

MS(APCI)m/z: 740(M+H), IR(Nujol)cm⁻¹: 1639, 1592.

2) The compound obtained in the above step (1) is treated in the same manner as described in Example 2(2) to give 2 fumaric acid salt thereof as a lyophilized amorphous powder.

### Example 269

1) To a solution of (1R*,2R*(S*),4R*)-2′-[3-(N-benzyloxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-methoxycarbonyl-4-pyridyl)-1-piperazinyl]carbonyl-spiro[cyclohexane-1,1′(2′H)-isoquinoline] (Compound obtained in Example 77(1))(250 mg) in tetrahydrofuran (5 mL) is added a suspension of calcium borohydride prepared from calcium chloride (59 mg) and sodium borohydride (40 mg) in tetrahydrofuran (S. Daluge et al, J. Org. Chem. 43, 2311, 1978) under ice-cooling. The mixture is stirred at 0° C. for 3 hours. To the reaction mixture is added ice-cooled water and the mixture is concentrated. The residue is extracted with ethyl acetate, dried over sodium sulfate, and concentrated. The residue is purified by thin-layer chromatography (chloroform:methanol=19:1) to give (1R*,2R*(S*),4R)-2′-3-(N-benzyloxycarbonyl-N-methylamino)-propionyl)-3′,4′-dihydro-6′,7′-dimethoxy-2-(2-ethyl-1,2,3,4-tetrahydro-6,7-dimethoxy-1-isoquinolyl)-4-[4-(2-hydroxymethyl-4-pyridyl)-1-piperazinyl]carbonyl-spiro[cyclohexane-1,1′(2′H)-isoquinoline] (150 mg, 57%) as an amorphous powder. MS(APCI)m/z: 919(M+H)

2) The compound obtained in the above step (1) is treated in the same manner as described in Example 200(1) to give (1R,2R(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-hydroxymethyl-4-pyridyl)-1-piperazinyl)carbonyl-spiro[cyclohexane-1,1′(2′H)-isoquinoline]

MS(APCI)m/z: 785.7(M+H).

3) The compound obtained in the above step (1) is treated in the same manner as described in Example 2(2) to give 2 fumaric acid salt thereof. MS(APCI)m/z: 785.7(M+H)

### Example 270

1) To a solution of (1R*,2R*(S*),4R*)-2′-[3-(N-methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1))(500 mg), acetaldehyde (63 μL) and acetic acid (93 μL) in methylene chloride (10 mL) is added sodium triacetoxyborohydride (179 mg) under ice-cooling and the mixture is stirred at room temperature for 5 hours. To the reaction mixture is added 10% hydrochloric acid and the organic layer is extracted with 10% hydrochloric acid. The extract is basified with potassium carbonate and extracted with chloroform. The organic layer is washed with water and saturated aqueous NaCl solution, dried over sodium sulfate and concentrated. The residue is purified by column chromatography on silica gel (Solvent; chloroform:methanol:28% aqueous ammonia=500:10:1) to give (1R*,2R*(S*),4R*)-2′-[3-(N-ethyl-N-methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)1H-pyrazolo[3,4-d]pyrimidin-4-yl]-1-piperazinyl]carbonyl-spiro[cyclohexane-1,1′(2′H)-isoquinoline] (428 mg, 83%). MS(APCI)m/z: 915.6(M+H), IR(Nujol)cm⁻¹: 1638.

2) The compound obtained in the above step (1) is treated in the same manner as described in Example 2(2) to give 1 fumaric acid salt thereof as an amorphous powder.

### Example 271

1) The corresponding materials are treated in the same manner as described in Example 270(1) to give a compound as shown in the following table (Table.14).

TABLE 14

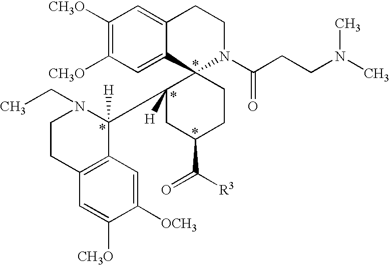

| Ex. No. | R³ | Physicochemical properties etc. |
|---|---|---|
| 271(1) | 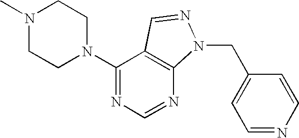 | Amorphous powder MS(APCI) 901.5(M + H) |

2) The compound obtained in the above step (1) is treated in the same manner as described in Example 2(2) to give 1 fumaric acid salt thereof.

### Example 272

1) To a solution of (1R*,2R*(S*),4R*)-2′-[3-(N-benzyloxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(1-tert-butoxycarbonyl-2-imidazolinyl)-1-piperazinyl]carbonyl-spiro[cyclohexane-1,1′(2′H)-isoquinoline] (Compound obtained in Example 144(1)) (126 mg) in methylene chloride (0.3 mL) is added trifluoroacetic acid (0.4 mL) and the mixture is stirred at room temperature for 24 hours. The reaction mixture is concentrated and the residue is dissolved in chloroform. The solution is washed with saturated aqueous sodium hydrogencarbonate solution and saturated aqueous NaCl solution. The mixture is dried over sodium sulfate and concentrated. The residue is purified by column chromatography on NH-silica gel (chloroform:methanol:=20:1) to give (1R*,2R*(S*),4R*)-2′-[3-(N-benzyloxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-imidazolinyl)-1-piperazinyl]carbonyl-spiro[cyclohexane-1,1′(2′H)-isoquinoline] (106 mg, 94%) as an amorphous powder. MS(APCI)m/z: 880(M+H), IR(Nujol)cm⁻: 3200-3500, 1700, 1635, 1612

2) The compound obtained in the above step (1) is treated in the same manner as described in Example 213 to give (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-imidazolinyl)-1-piperazinyl]carbonyl-spiro[cyclohexane-1,1′(2′H)-isoquinoline] (72 mg, 90%) as an amorphous powder.

MS(APCI)m/z: 746.6(M+H), IR(Nujol)cm⁻¹: 1635, 1611.

3) The compound obtained in the above step (2) is treated in the same manner as described in Example 2(2) to give 1 fumaric acid salt thereof as an amorphous powder.

MS(APCI)m/z: 746.7(M+H), IR(neat+chloroform)cm⁻¹: 3414, 1638, 1569.

### Example 273

1) A mixture of (1R*,2R*(S*),4R*)-2′-[3-(methylamino)-propionyl]-3′,4′-dihydro-6′,7′-dimethoxy-2-(2-ethyl-1,2,3,4-tetrahydro-6,7-dimethoxy-1-isoquinolyl)-4-[4-(1-benzyl-2-imidazolyl)-1-piperazinyl]carbonyl-spiro[cyclohexane-1,1′(2′H)-isoquinoline] (Compound obtained in Example 187(1)) (200 mg),10% palladium-carbon (200 mg), and ammonium formate (200 mg) in methanol (10 mL) is refluxed for 2 hours. After cooling, the reaction mixture is filtered and the filtrate is concentrated. The residue is dissolved in chloroform. The solution is dried over sodium sulfate and concentrated. The residue is purified by column chromatography on silica gel (Solvent; chloroform; methanol:28% aqueous ammonia=10:1:0.1) to give (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(2-imidazolyl)-1-piperazinyl]carbonyl-spiro[cyclohexane-1,1′(2′H)-isoquinoline] (91 mg, 51%) as an amorphous powder.

MS(APCI)m/z: 744(M+H), IR(Nujol)cm⁻¹: 3700-3100, 1634, 1567, 1512.

2) The compound obtained in the above step (1) is treated in the same manner as described in Example 2(2) to give 1 fumaric acid salt thereof as an amorphous powder.

MS(APCI)m/z: 744.6(M+H), IR(Nujol)cm⁻¹ : 3388, 3140, 1635, 1570.

### Example 274

1) A mixture of (1α,4β)-3′,4′-dihydro-6′,7′-dimethoxy-4-[4-(3-methyl-3H-1,2,3-triazolo[4,5-d]pyrimidin-7-yl)-1-piperazinyl]carbonyl-spiro[cyclohexane-1,1′(2′H)-isoquinoline] (Compound obtained in Example 161(1)) (223 mg), diisopropylethylamine (568 mg), and N-(3-bromoacetyl)-phthalimide (1.18 g) in dimethylacetamide (10 mL) is stirred at 100° C. for 1 hour, after addition of N-(3-bromopropyl)phthalimide (1.36 g), the mixture is further stirred at 120° C. for 5 hours. After cooling, ethyl acetate is added the reaction mixture and the mixture is washed with saturated aqueous NaCl solution. The mixture is dried over sodium sulfate and concentrated. The residue is purified by column chromatography on silica gel (Solvent; chloroform:methanol:=50:1) to give (1α,4β)-2′-(3-phthalimidopropyl)-3′,4′-dihydro-6′,7′-dimethoxy-4-[4-(3-methyl-3H-1,2,3-triazolo[4,5-d]pyrimidin-7-yl)-1-piperazinyl]carbonyl-spiro[cyclohexane-1,1′(2′H)-isoquinoline] (200 mg, 65%) as crystals. M.p. 229-230° C.,

MS(FAB)m/z: 694(M+H), IR(Nujol)cm⁻¹: 1711, 1651.

2) To a mixture of the compound obtained in the above step (1) (139 mg), ethanol (10 mL) and tetrahydrofuran (10 mL) is added hydrazine hydrate (150 mg) and the mixture is refluxed for 4 hours. After cooling the reaction mixture, the precipitates are removed by filtration and the filtrate is concentrated. The residue is purified by column chromatography on NH-silica gel (Solvent; chloroform:methanol:=5:1) and neutral column chromatography on silica gel (Solvent; chloroform:methanol:28% aqueous ammonia=200:10:1), successively, to give (1α,4β)-2′-(3-aminopropyl)-3′,4′-dihydro-6′,7′-dimethoxy-4-[4-[3-methyl-3H-1,2,3-triazolo[4,5-d]-pyrimidin-7-yl]-1-piperazinyl]-carbonyl-spiro[cyclohexane-1,1′(2′H)-isoquinoline] (118 mg, 100%) as an amorphous powder.

MS(APCI)m/z: 564(M+H), IR(neat+chloroform)cm⁻¹: 3370, 1643.

3) The compound obtained in the above step (2) is treated in the same manner as described in Example 2(2) to give 1 fumaric acid salt thereof as an amorphous powder.

MS(APCI)m/z: 564(M+H), IR(Nujol)cm⁻¹: 3387, 1633, 1598.

### Examples 275 to 277

The corresponding materials are treated in the same manner as described in Example 274(1) to give compounds as shown in the following table (Table.15).

TABLE 15

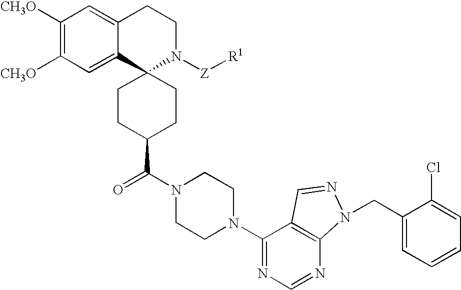

| Ex. No. | —Z—R¹ | Physicochemical properties etc. |
|---|---|---|
| 275 | 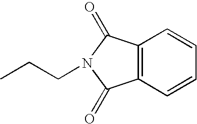 | Amorphous powder MS(APCI) 789.5(M + H) |
| 276 | 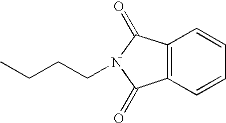 | Amorohous powder MS(APCI) 803.6(M + H) |
| 277 | 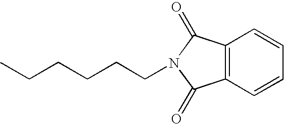 | Amorohous powder MS(APCI) 817(M + H) |

### Examples 278 to 288

1) The corresponding materials are treated in the same manner as described in Example 274(2) to give the following compounds as shown in the following tables (Table.16)

TABLE 16
(No. 1)

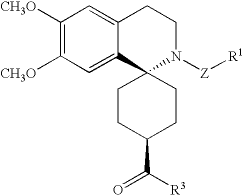

| Ex. No. | —Z—R¹ | R³ | Physicochemical properties etc. |
|---|---|---|---|
| 278(1) | 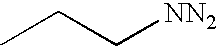 | 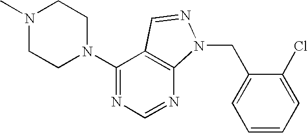 | Amorohous powder MS(APCI) 659(M + H) |
| 279(1) | 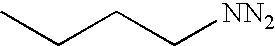 | 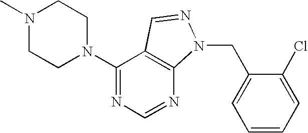 | Amorohous powder MS(APCI) 673.5(M + H) |
| 280(1) | 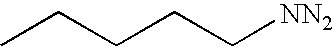 | 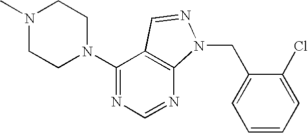 | Amorohous powder MS(APCI) 687(M + H) |
| 281(1) | 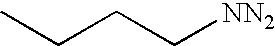 | 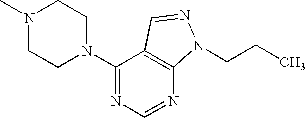 | Amorohous powder MS(APCI) 591(M + H) |
| 282(1) | 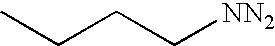 | 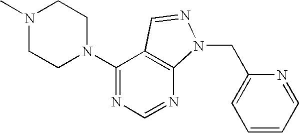 | Amorohous powder MS(APCI) 640(M + H) |
| 283(1) | 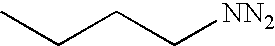 | 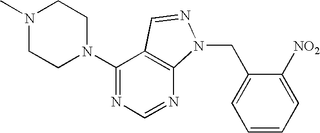 | Amorohous powder MS(APCI) 684.4(M + H) |
| 284(1) | 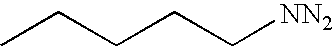 | 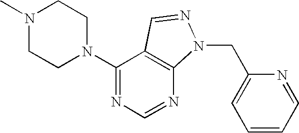 | Amorohous powder MS(APCI) 654.4(M + H) |

2) The compounds obtained in Examples 278 (1) to 288 (1) are treated in the same manner as described in Example 2(2) to give the following compounds (salt).

Example 278(2): 1 Fumaric acid salt of the compound obtained in Example 278(1); Amorphous powder, MS(APCI)m/z: 659.5(M+H), IR(Nujol)cm⁻¹: 1637, 1571

Example 279(2): 1 Fumaric acid salt of the compound obtained in Example 279(1); Amorphous powder, MS(APCI)m/z: 673.5(M+H), IR(Nujol)cm⁻¹: 1637, 1571

Example 280(2): 1 Fumaric acid salt of the compound obtained in Example 280(1); Amorphous powder, MS(APCI)m/z: 687(M+H), IR(neat+chloroform)cm⁻¹: 3416, 1635, 1571

Example 281(2): 1 Fumaric acid salt of the compound obtained in Example 281(1); Amorphous powder, MS(APCI)m/z: 591.5(M+H), IR(Nujol)cm⁻¹: 3381, 1629, 1573

Example 282(2): 1 Fumaric acid salt of the compound obtained in Example 282(1); Amorphous powder, MS(APCI)m/z: 640.4(M+H), IR(neat+chloroform)cm⁻¹: 3417, 1634, 1573

Example 283(2): 1 Fumaric acid salt of the compound obtained in Example 283(1); Amorphous powder, MS(APCI)m/z: 684(M+H), IR(Nujol)cm⁻¹: 3384, 1632, 1613, 1573

Example 284(2): 1 Fumaric acid salt of the compound obtained in Example 284(1); Amorphous powder, MS(APCI)m/z: 654(M+H), IR(Nujol)cm⁻¹: 3378, 1617, 1573

Example 285(2): 1 Fumaric acid salt of the compound obtained in Example 285(1); Amorphous powder, MS(APCI)m/z: 640(M+H), IR(Nujol)cm⁻¹: 3375, 1614, 1573

Example 286(2): 1 Fumaric acid salt of the compound obtained in Example 286(1); Amorphous powder, MS(APCI)m/z: 664(M+H), IR(Nujol)cm⁻¹: 3383, 2225, 1633, 1573

Example 287(2): 1 Fumaric acid salt of the compound obtained in Example 287(1); Amorphous powder, MS(APCI)m/z: 719(M+H), IR(Nujol)cm⁻¹: 3387, 1628, 1573

Example 288(2): 1 Fumaric acid salt of the compound obtained in

Example 288(1); Amorphous powder, MS(APCI)m/z: 654(M+H), IR(Nujol)cm⁻¹: 3385, 1625, 1573

TABLE 16
(No. 2)

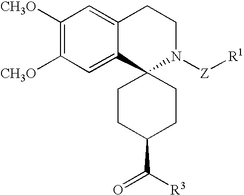

| Ex. No. | —Z—R¹ | R³ | Physicochemical properties etc. |
|---|---|---|---|
| 285(1) | 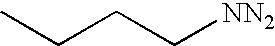 | 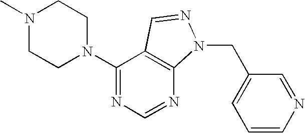 | Amorohous powder MS(APCI) 640(M + H) |
| 286(1) | 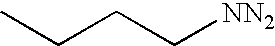 | 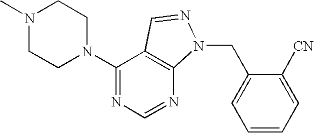 | Amorohous powder MS(APCI) 664(M + H) |
| 287(1) | 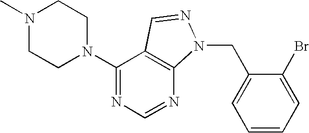 | 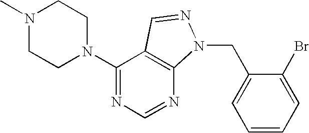 | Amorohous powder MS(APCI) 717, 719(M + H) |
| 288(1) | 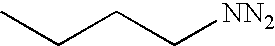 | 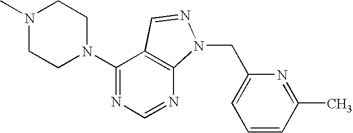 | Amorohous powder MS(ESI) 654(M + H) |

### Example 289

1) (1α,4β)-2′-(3-aminopropyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 279(1)) (160 mg) and aqueous 37% formaldehyde (0.5 mL) are added to a mixture of tetrahydrofuran (5 mL) and acetic acid (2 mL) and to the mixture is gradually added sodium borohydride (28.8 mg) under ice-cooling. The mixture is stirred at the same temperature for 1 hour and basified with saturated aqueous sodium hydrogencarbonate solution. The reaction mixture is extracted with chloroform. The organic layer is washed with saturated aqueous NaCl solution, dried over sodium sulfate, and concentrated. The residue is purified by column chromatography on silica gel (Solvent; chloroform:methanol:aqueous 28% ammonia=100:10:1) to give (1α,4β)-2′-(3-dimethylaminopropyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (104 mg, 62%) as an amorphous powder.

MS(APCI)m/z: 701(M+H), IR(Nujol)cm⁻¹: 1642, 1570.

2) The compound obtained in the above step (2) is treated in the same manner as described in Example 2(2) to give 1 fumaric acid salt thereof as an amorphous powder.

MS(APCI)m/z: 701.6(M+H), IR(Nujol)cm⁻¹: 3396, 1635, 1573.

### Examples 290 to 291

1) The corresponding materials (the compound obtained in Example 278(1) or 280(1)) are treated in the same manner as described in Example 289(1) to give the compounds as shown in the following table (Table.17).

TABLE 17

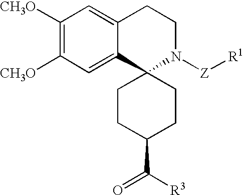

| Ex. | | | Physicochemical |
|---|---|---|---|
| No. | —Z—R¹ | R³ | properties etc. |
| 290 (1) | 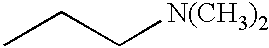 | 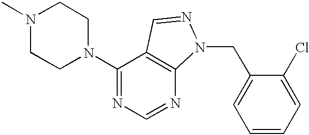 | Amorohous powder MS (APCI) 687.5 (M + H) |
| 291 (1) | 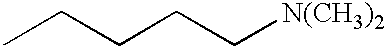 | 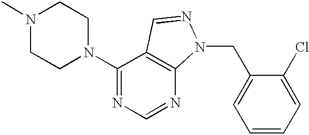 | Foam MS (APCI) 715 (M + H) |

2) The compounds obtained in the above Examples 290(1) and 291(1) are treated in the same manner as described in Example 2(2) to give the following compounds (salt). Example 290(2): 1 Fumaric acid salt of the compound obtained in Example 290(1); Powder, MS(APCI)m/z: 687.6(M+H), IR(Nujol)cm⁻¹: 1700, 1635

Example 291(2): 1 Fumaric acid salt of the compound obtained in Example 291(1); Amorphous powder, MS(APCI)m/z: 715.5(M+H), IR(neat+chloroform)cm⁻¹: 3425, 1637, 1571

### Example 292

1) To a suspension of (1α,4β)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 162(1))(283 mg) and triethylamine (140 mg) in methylene chloride is slowly added dropwise bromoacetyl chloride (217 mg) under ice-cooling and the mixture is stirred at the same temperature for 30 minutes. The reaction mixture is concentrated. The residue is dissolved in ethyl acetate, and the solution is washed with saturated aqueous sodium hydrogencarbonate solution and saturated aqueous NaCl solution, dried over sodium sulfate, and concentrated. The residue is purified by thin-layer chromatography on silica gel (Solvent; chloroform:methanol:=100:1) to give (1α,4β)-2′-bromoacetyl-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (357 mg, 100%) as an amorphous powder.

MS(APCI)m/z: 738(M+H), IR(Nujol)cm⁻¹: 1635.

2) The compound obtained in the above step (1) (100 mg) and 50% dimethylamine solution (1 mL) are added to acetonitrile (2 mL) and the mixture is stirred for 20 hours at room temperature. The reaction mixture is concentrated to remove solvent. The residue is purified by preparative column chromatography on silica gel (Solvent; chloroform:methanol:aqueous 28% ammonia=100:10:1) to give (1α,4β)-2′-(N,N-dimethylaminoacetyl)-3′,4′-dihydro-6′,7′-dimethoxy-4-[4-[1-(2-chlorophenylmethyl)-1H-pyrazolo[3,4-d]pyrimidin-4-yl)-1-piperazinyl]carbonyl-spiro[cyclohexan-1,1′(2′H)-isoquinoline] (57 mg) as amorphous.

MS(APCI)m/z: 701(M+H), IR(Nujol)cm⁻¹: 1635.

3) The compound obtained in the above step (2) is treated in the same manner as described in Example 2(2) to give 1 fumaric acid salt thereof as an amorphous powder.

MS(APCI)m/z: 701.5(M+H), IR(Nujol)cm⁻¹: 1639, 1572.

### Example 293

1) A solution of (1α,4β)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 162(1))(149 mg), 1,3-dibromoproapane (2.44 g) and diisopropylethylamine (310 mg) in dimethylacetamide (5 mL) is stirred at 120° C. for 1.5 hours. After cooling, saturated aqueous sodium hydrogencarbonate solution is added to the mixture and the mixture is extracted with ethyl acetate. The organic layer is washed with saturated aqueous NaCl solution, dried over sodium sulfate, concentrated. The residue is purified by column chromatography on silica gel (Solvent; chloroform:methanol=50:1) to give (1α,4β)-2′-(3-bromopropyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (122 mg, 69%) as an amorphous powder.

2) To a solution of the compound obtained in the above step (1) in acetonitrile (3 mL) is added 40% aqueous methylamine (1 mL) and the mixture is stirred at room temperature for 13 hours. The reaction mixture is concentrated and the residue is purified by preparative column chromatography on silica gel (Solvent; chloroform:methanol:28% aqueous ammonia=500:10:1) to give (1α,4β)-2′-(3-methylaminopropyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (78 mg, 68%) as an amorphous powder.

MS(APCI)m/z: 687(M+H), IR(Nujol)cm⁻¹: 1639.

3) The compound obtained in the above step (2) is treated in the same manner as described in Example 2(2) to give 1 fumaric acid salt thereof as an amorphous powder.

MS(APCI)m/z: 687(M+H), IR(Nujol)cm⁻¹: 3417, 1634, 1571.

### Example 294

1) A mixture of (1α,4β)-2′-[3-(dimethylamino)propionyl]-3′,4′-dihydro-6′,7′-dimethoxy-4-(4-(2-amino-4-pyridyl)-1-piperazinyl]carbonyl-spiro[cyclohexane-1,1′(2′H)-isoquinoline] (Compound obtained in Example 137(1))(250 mg) and benzaldehyde (141 mg) in toluene (25 mL) are refluxed for 6 hours under dehydrating condition. After cooling, the mixture is concentrated and the residue is dissolved in ethanol (10 mL). To the solution is added sodium borohydride (42 mg) under ice-cooling and the mixture is stirred overnight. The reaction mixture is concentrated and saturated aqueous sodium hydrogencarbonate is added to the residue. The mixture is extracted with ethyl acetate and the organic layer is washed with saturated aqueous NaCl solution, dried over sodium sulfate, and concentrated. The residue is purified by column chromatography on silica gel (Solvent; chloroform:methanol:aqueous 28% ammonia=100:10:1) to give (1α,4β)-2′-(3-dimethylaminopropionyl)-3′,4′-dihydro-6′,7′-dimethoxy-4-[4-(2-benzylamino-4-pyridyl)-1-piperazinyl]carbonyl-spiro[cyclohexane-1,1′(2′H)-isoquinoline] (77 mg, 29%) as an amorphous powder.

MS(APCI)m/z: 655(M+H), IR(Nujol)cm⁻¹: 3340, 1628.

2) The compound obtained in the above step (1) is treated in the same manner as described in Example 2(2) to give 1 fumaric acid salt thereof as an amorphous powder.

MS(APCI)m/z: 655(M+H), IR(neat+chloroform)cm⁻¹: 3408, 1633, 1608.

### Example 295

1) To a mixture of (1α,4β)-2′-(4-aminobutyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 280(1))(444 mg), sodium hydrogencarbonate (271 mg), dioxane (15 mL) and water (15 mL) is added dropwise benzyloxycarbonyl chloride (0.12 mL) under vigorously stirring and ice-cooling. The mixture is stirred at room temperature for 30 minutes and diluted with ethyl acetate. The reaction mixture is washed with water and saturated aqueous sodium hydrogencarbonate solution and dried over sodium sulfate. The mixture is concentrated and the residue is purified by column chromatography on silica gel (Solvent; chloroform:methanol:aqueous 28% ammonia=10:1:0.1) to give (1α,4β)-2′-[4-(benzyloxycarbonylamino)butyl]-3′,4′-dihydro-6′,7′-dimethoxy-4-[4-[1-(2-chlorophenylmethyl)-1Hpyrazolo[3,4-d]pyrimidin-4-yl]-1-piperazinyl]carbonyl-spiro[cyclohexane-1,1′(2′H)-isoquinoline] (479 mg, 90%). MS(APCI)m/z: 821(M+H), IR(Nujol)cm⁻¹: 1636, 1715

2) To a mixture of the compound obtained in the above step (1) (184 mg) in dimethylformamide (5 mL). and tetrahydrofuran (1 mL) is added sodium hydride (53.8 mg) under ice-cooling and the mixture is stirred for 1 hour. To the reaction mixture is added iodomethane (0.042 mL) and the mixture is stirred at room temperature for 3 hours. The reaction mixture is diluted with ethyl acetate, washed with water, saturated aqueous sodium hydrogencarbonate solution and saturated aqueous NaCl solution, dried over sodium sulfate, and concentrated. The residue is purified by column chromatography on silica gel (Solvent; chloroform:methanol=10:1) to give (1α,4β)-2′-[4-(N-benzyloxycarbonyl-N-methylamino)butyl]-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (132.8 mg, 71%)

MS(APCI)m/z: 835.5(M+H), IR(Nujol)cm⁻¹: 1637, 1698.

3) The compound obtained in the above step (2) (125.5 mg) is treated in the same manner as described in Example 165(1) to give (1α,4β)-2′-[4-(methylamino)butyl]-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (91.4 mg, 86.9%) as an amorphous powder.

MS(APCI)m/z: 701(M+H), IR(Nujol)cm⁻¹: 3419, 1639.

4) The compound obtained in the above step (3) is treated in the same manner as described in Example 2(2) to give 1 fumaric acid salt thereof.

MS(APCI)m/z: 701.5(M+H), IR(Nujol)cm⁻¹: 3425, 1641, 1572.

### Example 296

1) A mixture of (1α,4β)-2′-(3-aminopropyl)-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 279(1))(1.14 g) and diphenylcyanocarbonimidate (441 mg) in methylene chloride (6 mL) and isopropyl alcohol (18 mL) is stirred at room temperature for 18 hours. The reaction mixture is concentrated and the residue is purified by column chromatography on silica gel (Solvent; chloroform:methanol=20:1) to give (1α,4β)-2′-[3-(cyanimido-phenoxymethylenamino)propyl]-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (1.4 g, 100%).

MS(APCI)m/z: 817(M+H), IR(Nujol)cm⁻¹: 2185, 1734, 1635.

2) A mixture of the compound obtained in the above step (1) (463 mg), isopropyl alcohol (5 mL), and aqueous ammonia (5 mL) is heated in a sealed tube at 100° C. for 6 hours. After cooling, to the reaction mixture is added saturated aqueous sodium hydrogencarbonate solution and the mixture is extracted with chloroform. The extract is washed with saturated aqueous NaCl solution, dried over sodium sulfate, and concentrated. The residue is purified by column chromatography on silica gel (Solvent; chloroform:methanol:aqueous 28% ammonia=20:1:0.1) to give (1α,4β)-2′-[3-(2-cyano-guanidino)-propyl]-3′,4′-dihydro-6′,7′-dimethoxy-4-[4-[1-(2-chlorophenyl-methyl)-1H-pyrazolo[3,4-d]pyrimidin-4-yl]-1-piperazinyl]carbonyl-spiro[cyclohexane-1,1′(2′H)-isoquinoline] (396 mg, 94%).

MS(APCI)m/z: 740(M+H), IR(Nujol)cm⁻¹: 3325, 3175, 2169, 1635.

3) The compound obtained in the above step (2) is treated in the same manner as described in Example 2(2) to give 1 fumaric acid salt thereof as an amorphous powder.

MS(APCI)m/z: 740(M+H), IR(Nujol)cm⁻¹: 3333, 2174, 1705, 1635.

### Examples 297 to 303

1) The corresponding materials are treated in the same manner as described in Example 296(1) and (2)to give compounds as shown in the following tables (Table.18).

TABLE 18
(No.1)

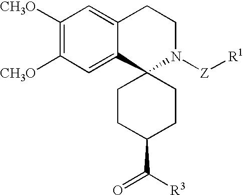

| Ex. | | | Physicochemica1 |
|---|---|---|---|
| No. | —Z—R¹ | R¹ | properties etc. |
| 297 (1) | 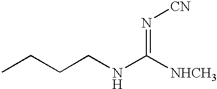 | 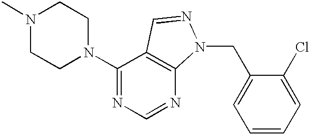 | Amorohous powder MS (APCI) 754 (M + H) |
| 298 (1) | 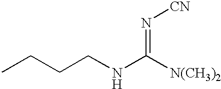 | 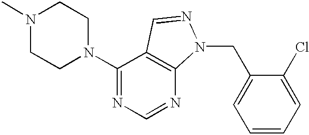 | Amorohous powder MS (APCI) 768 (M + H) |
| 299 (1) | 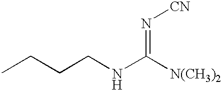 | 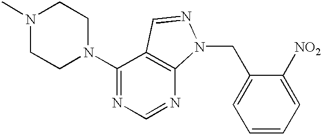 | Amorohous powder MS (ESI) 779 (M + H) |
| 300 (1) | 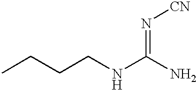 | 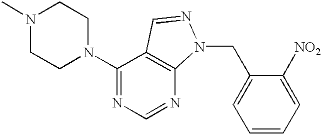 | Amorohous powder MS (ESI) 751 (M + H) |

TABLE 18
(No. 2)

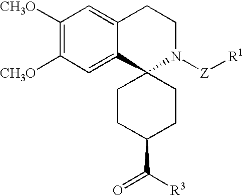

| Ex. | | | Physicochemical |
|---|---|---|---|
| No. | —Z—R¹ | R³ | properties etc. |
| 301 (1) | 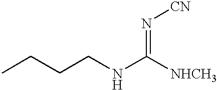 | 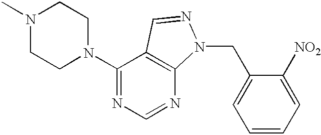 | Amorohous powder MS (APCI) 765 (M + H) |
| 302 (1) | 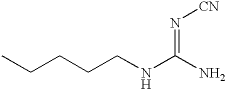 | 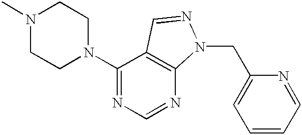 | Amorohous powder MS (APCI) 721.5 (M + H) |

TABLE 18
(No.3)

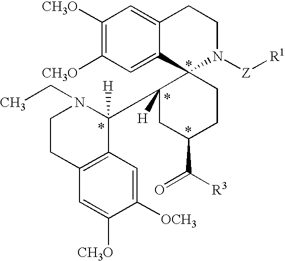

| Ex. | | | Physicochemical |
|---|---|---|---|
| No. | —Z—R¹ | R³ | properties etc. |
| 303 (1) | 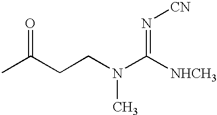 | 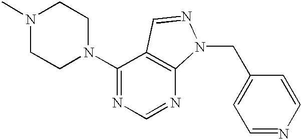 | Amorohous powder MS (APCI) 968 (M + H) |

2) The compound obtained in the above Examples 297(1) to 303(1) are treated in the same manner as described in Example 2(2) to give the following compounds (salt).

Example 297(2): 1 Fumaric acid salt of the compound obtained in Example 297(1); Amorphous powder, MS(APCI)m/z: 754(M+H), IR(Nujol)cm⁻¹: 3280, 2167, 1705

Example 298(2): 1 Fumaric acid salt of the compound obtained in Example 298(1); Amorphous powder, MS(APCI)m/z: 768(M+H), IR(Nujol)cm⁻¹: 2165, 1706, 1636

Example 299(2): 0.5 Fumaric acid salt of the compound obtained in Example 299(1); Amorphous powder, MS(APCI)m/z: 779(M+H), IR(Nujol)cm⁻¹: 2164, 1574

Example 300(2): 0.5 Fumaric acid salt of the compound obtained in Example 300(1); Amorphous powder, MS(APCI)m/z: 751(M+H), IR(Nujol)cm⁻¹: 2175, 1576

Example 301(2): 0.5 Fumaric acid salt of the compound obtained in Example 301(1); Amorphous powder, MS(APCI)m/z: 765(M+H), IR(Nujol)cm⁻¹: 2167, 1576

Example 302(2): 1 Fumaric acid salt of the compound obtained in Example 302(1); Amorphous powder, MS(APCI)m/z: 721.5(M+H), IR(Nujol)cm⁻¹: 3343, 2173, 1701

Example 303(2): 1 Fumaric acid salt of the compound obtained in Example 303(1); Amorphous powder, MS(APCI)m/z: 968.6(M+H), IR(Nujol)cm⁻¹: 2166, 1703, 1629

### Example 304

1) A solution of (1α,4β)-2′-(3-aminopropyl)-3′,4′-dihydro-6′,7′ dimethoxy-4-[4-[1-(2-chlorophenylmethyl)-1Hpyrazolo[3,4-d]-pyrimidin-4-yl]-1-piperazinyl]carbonyl-spiro[cyclohexane-1,1′(2′H)-isoquinoline] (Compound obtained in Example 279(1))(528 mg) and 1,1-bis(methylthio)-2-nitroethylene (260 mg) in tetrahydrofuran (5 mL) and isopropyl alcohol (10 mL) is stirred at room temperature for 22 hours and refluxed for 3 hours. The reaction mixture is concentrated and the residue is purified by column chromatography on silica gel (Solvent; chloroform:methanol=50:1) to give (1α,4β)-2′-[3-(1-methylthio-2-nitrovinylamino)propyl]-3′,4′-dihydro-6′,7′-dimethoxy-4-[4-[1-(2-chlorophenylmethyl)-1H-pyrazolo[3,4-d]pyrimidin-4-yl)-1-piperazinyl]carbonyl-spiro[cyclohexane-1,1′(2′H)-isoquinoline] (574 mg, 93%).

MS(ESI)m/z: 790.4(M+H), IR(Nujol)cm⁻¹: 1638, 3480.

2) A solution of the compound obtained in the above step (1) (148 mg)in dioxane (3 mL), isopropyl alcohol (3 mL) and dimethylamine (1 mL) is stirred at room temperature for 18 hours. The reaction mixture is concentrated and the residue is purified by column chromatography on NH-silica gel (Solvent; chloroform) to give (1α,4β)-2′-[3-(1-dimethylamino-2-nitrovinylamino)propyl]-3′,4′-dihydro-6′,7′-dimethoxy-4-[4-[1-(2-chlorophenyl-methyl)-1H-pyrazolo[3,4-d]pyrimidin-4-yl]-1-piperazinyl]carbonyl-spiro[cyclohexane-1,1′(2′H)-isoquinoline] (124 mg, 84%)

MS(APCI)m/z: 787.4(M+H), IR(Nujol)cm⁻¹: 1637, 3463.

3) The compound obtained in the above step (2) is treated in the same manner as described in Example 2(2) to give 1 fumaric acid salt thereof as an amorphous powder.

MS(ESI)m/z: 809(M+Na),787(M+H), IR(Nujol)cm⁻¹: 1699, 1617, 1570.

### Example 305 to 312

1) The corresponding materials are treated in the same manner as described in Example 304(1) and (2) to give the compounds as shown in the following table (Table.19).

TABLE 19

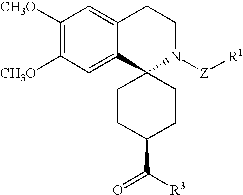

| Ex. | | | Physicochemical |
|---|---|---|---|
| No. | —Z—R¹ | R³ | properties etc. |
| 305 (1) | 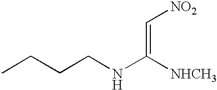 | 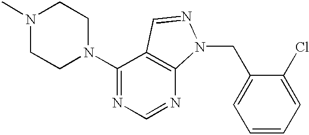 | Amorohous powder MS (ESI) 773 (M + H) |
| 306 (1) | 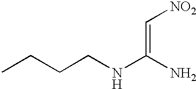 | 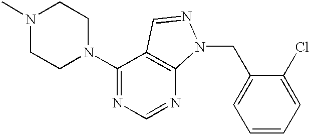 | Amorohous powder MS (ESI) 759 (M + H) |
| 307 (1) | 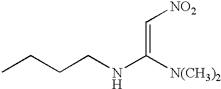 | 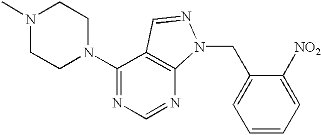 | Amorohous powder MS (ESI) 798 (M + H) |
| 308 (1) | 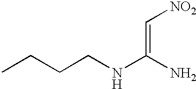 | 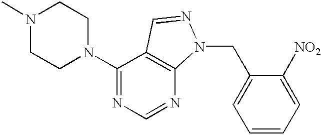 | Amorohous powder MS (ESI) 770 (M + H) |
| 309 (1) | 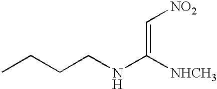 | 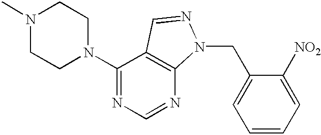 | Amorohous powder MS (ESI) 784 (M + H) |

2) The compounds obtained in the above steps (1) are treated in the same manner as described in Example 2(2) to give the following compounds (salt).

Example 305(2): 1 Fumaric acid salt of the compound obtained in Example 305(1); Powder, MS(ESI)m/z: 795(M+Na), 773 (M+H), IR(Nujol)cm⁻¹: 1701, 1623, 1573

Example 306(2): 1 Fumaric acid salt of the compound obtained in Example 306(1); Amorphous powder, MS(ESI)m/z: 781(M+Na), 759(M+H), IR(Nujol)cm⁻¹: 1699, 1614, 1574

Example 307(2): 2 Citric acid salt of the compound obtained in Example 307(1); Powder, MS(ESI)m/z: 796.5(M−H), IR(Nujol)cm⁻¹: 1724, 1665, 1610

Example 308(2): 2 Citric acid salt of the compound obtained in Example 308(1); Amorphous powder, MS(ESI)m/z: 792(M+Na), 770(M+H), IR(Nujol)cm⁻¹: 1720, 1610

Example 309(2): 2 Citric acid salt of the compound obtained in Example 309(1); Powder, MS(ESI)m/z: 782(M−H), IR(Nujol)cm⁻¹: 1725, 1612

Example 310(2): Citric acid salt of the compound obtained in Example 307(1)

Example 311(2): Citric acid salt of the compound obtained in Example 308(1)

Example 312(2): Citric acid salt of the compound obtained in Example 309(1)

### Example 313

1) To a solution of (1R*,2R*(S*),4R*)-2′-[3-(methylamino)propionyl)-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (10.0 g) and triethylamine (1.71 g) in methylene chloride (100 mL) is added dropwise a solution of propanoyloxy-methylcarbonochloridate (F. J. Lundet et al, Synthesis 1159, 1990) (2.50 g) in methylene chloride (30 mL) under ice-cooling. The mixture is stirred at the same temperature for 1 hour and further stirred at room temperature for 1 hour. The reaction mixture is washed with saturated aqueous sodium hydrogencarbonate solution and saturated aqueous NaCl solution, dried over sodium sulfate and concentrated. The residue is purified by column chromatography on silica gel (Solvent; chloroform methanol=50:1) to give (1R*,2R*(S*),4R*)-2′-[3-[N-(propionyloxymethyloxycarbonyl)-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (6.90 g, 60%) as crystals. M.p. 160-161° C.

MS(APCI)m/z: 1017.5(M+H), IR(Nujol)cm⁻¹: 1755, 1721, 1641.

2) The compound obtained in the above step (1) is treated in the same manner as described in Example 2(2) to give a 2 fumaric acid salt thereof as an amorphous powder.

MS(APCI)m/z: 1017(M+H), IR(Nujol)cm⁻¹: 3397, 1751, 1708, 1635.

### Example 314

1) To a solution of (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (0.55 g) and triethylamine (0.1 mL) in methylene chloride (8 mL) is added dropwise a solution of 2-(chloromethyloxycarbonyloxy)-pyridine (128 mg) in methylene chloride (2 mL) under ice-cooling. The mixture is stirred at 4° C. for 20 minutes. To the reaction mixture is added ethyl acetate. The mixture is washed with water and saturated aqueous NaCl solution, dried over sodium sulfate and concentrated.

2) A mixture of the compound obtained in the above step (1), cesium carbonate (1.11 g), molecular shieves 3A (MS3A) (1.11 g), and propionic acid (0.185 mL) in acetonitrile (15 mL) is refluxed for 6 hours. After cooling, ethyl acetate is added to the reaction mixture and the mixture is filtered through Celite to remove the precipitates. The filtrate is washed with saturated aqueous NaCl solution, dried over sodium sulfate, and concentrated. The residue is purified by column chromatography on NH-silica gel (solvent; ethyl acetate) to give (1R*,2R*(S*),4R*)-2′-[3-[N-(propionyloxymethyloxycarbonyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (430 mg, 68%; compound obtained in Example 313(1)) as crystals. M.p. 160-161° C.

### Example 315

1) To a mixture of (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[3-methyl-3H-1,2,3-triazolo[4,5-d]pyrimidin-7-yl]-1-piperazinyl]carbonyl-spiro[cyclohexane-1,1′(2′H)-isoquinoline] (Compound obtained in Example 241(1)) (250 mg), cesium carbonate (512 mg), and molecular shieves3A (M3A) (460 mg) in acetonitrile (8 mL)is added chloromethyl chloroformate (0.03 mL) under ice-cooling, and the mixture is stirred at room temperature for 30 minutes. To the reaction mixture is added pivalic acid (126 mg) and the mixture is refluxed for 4 hours. After cooling, the reaction mixture is filtered to remove inorganic materials and the residue on the filter is washed with ethyl acetate. The combined filtrate is concentrated and the residue is dissolved in ethyl acetate. The solution is washed with water, saturated aqueous sodium hydrogencarbonate solution and saturated aqueous NaCl solution, dried over sodium sulfate, and concentrated. The residue is purified by column chromatography on NH-silica gel (Solvent; ethyl acetate) to give (1R*,2R*(S*),4R*)-2′-[3-[N-(pivaloyloxy-methyloxycarbonyl)-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[3-methyl-3H-1,2,3-triazolo[4,5-d]pyrimidin-7-yl]-1-piperazinyl]carbonyl-spiro[cyclohexane-1,1′(2′H)-isoquinoline] (215 mg, 72%) as an amorphous powder.

MS(APCI)m/z: 969(M+H), IR(Nujol)cm⁻¹: 1746, 1721, 1639, 1597.

2) The compound obtained in the above step (1) is treated in the same manner as described in Example 2(2) to give a 2 fumaric acid salt thereof as an amorphous powder.

MS(APCI)m/z: 969(M+H), IR(Nujol)cm⁻¹: 3133, 1713, 1635.

### Examples 316 to 361

1) The corresponding materials are treated in the same manner as described in Example 313(1), 314(1) to (2) or 315(1) to give compounds as shown in the following tables (Table.20).

TABLE 20
(No. 1)

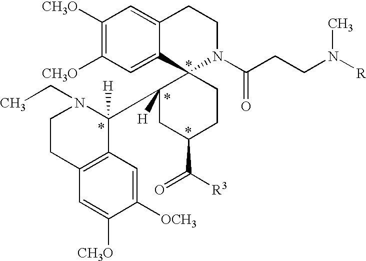

| Ex. | | | Physicochemical |
|---|---|---|---|
| No. | R | R³ | properties etc. |
| 316 (1) | 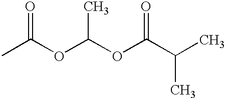 | 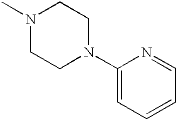 | Amorohous powder MS (APCI) 913 (M + H) |
| 317 (1) | 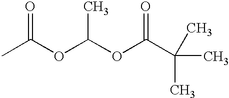 | 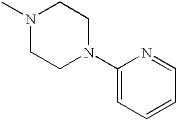 | Amorohous powder MS (ESI) 927 (M + H) |
| 318 (1) | 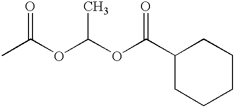 | 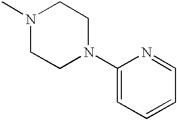 | Amorohous powder MS (ESI) 953 (M + H) |
| 319 (1) | 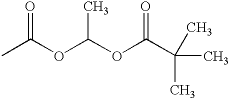 | 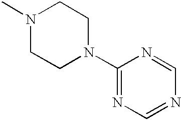 | Amorohous powder MS (APCI) 929 (M + H) |
| 320 (1) | 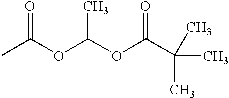 | 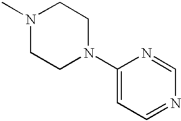 | Amorohous powder MS (APCI) 928 (M + H) |
| 321 (1) | 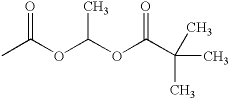 | 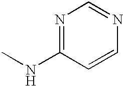 | Amorohous powder MS (APCI) 859 (M + H) |

TABLE 20
(No. 2)

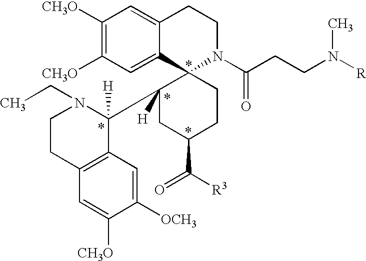

| Ex. | | | Physicochemical |
|---|---|---|---|
| No. | R | R³ | properties etc. |
| 322 (1) | 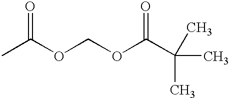 | 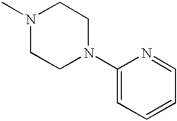 | Amorohous powder MS (APCI) 913 (M + H) |
| 323 (1) | 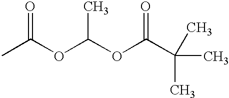 | 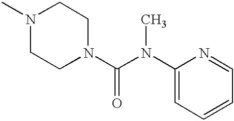 | Amorohous powder MS (APCI) 984.5 (M + H) |
| 324 (1) | 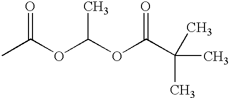 | 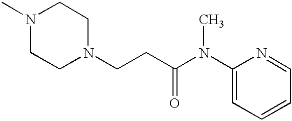 | Amorohous powder MS (APCI) 1012 (M + H) |
| 325 (1) | 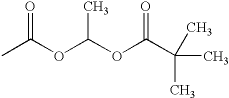 | 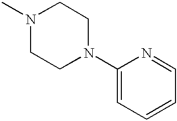 | Optically-active isomer (Amorohous powder) MS (APCI) 913 (M + H) [α]_{D} −46.2°(c 1.0, CHCl₃) |
| 326 (1) | 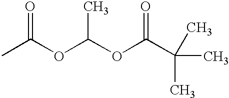 | 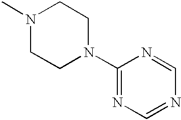 | Optically-active isomer (Amorohous powder) MS (APCI) 929 (M + H) [α]_{D} −47.8°(c 1.0, CHCl₃) |
| 327 (1) | 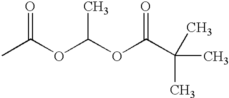 | 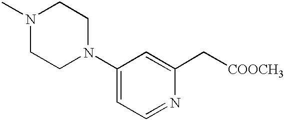 | Amorohous powder MS (APCI) 999 (M + H) |

TABLE 20
(No. 3)

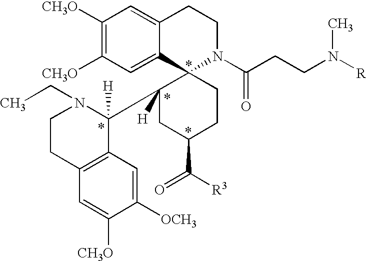

| Ex. | | | Physicochemical |
|---|---|---|---|
| No. | R | R³ | properties etc. |
| 328 (1) | 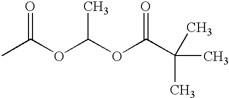 | 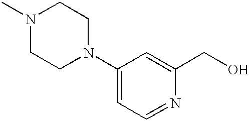 | Amorohous powder MS (APCI) 957 (M + H) |
| 329 (1) | 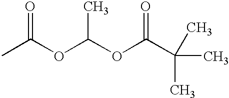 | 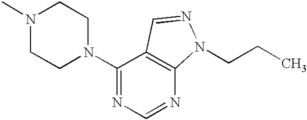 | Amorohous powder MS (APCI) 1010 (M + H) |
| 330 (1) | 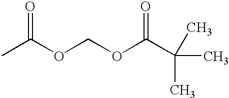 | 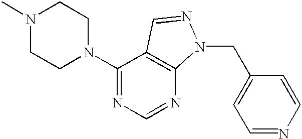 | Amorohous powder MS (APCI) 1045 (M + H) |
| 331 (1) | 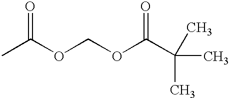 | 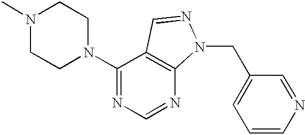 | Amorohous powder MS (APCI) 1045 (M + H) |
| 332 (1) | 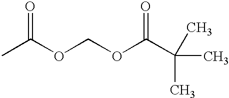 | 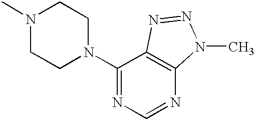 | Amorohous powder MS (APCI) 969 (M + H) [α]_{D} +58.0°(c 0.5, CHCl₃) |
| 333 (1) | 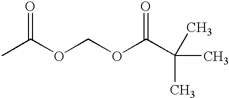 | 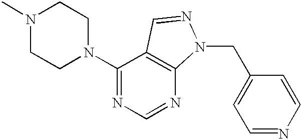 | Optically-active isomer (Amorohous powder) MS (APCI) 1045 (M + H) |

TABLE 20
(No. 4)

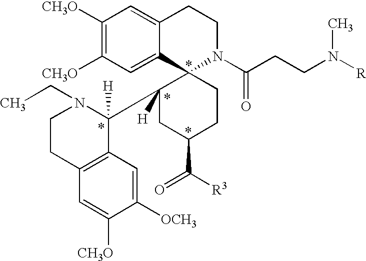

| Ex. | R | R³ | Physicochemical |
|---|---|---|---|
| No. | R | R³ | properties etc. |
| 334 (1) | 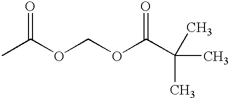 | 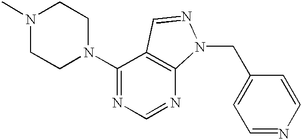 | Optically-active isomer (Amorohous powder) MS (APCI) 1045 (M + H) [α]_{D} −50.4°(c 1.0, CHCl₃) |
| 335 (1) | 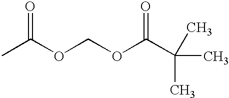 | 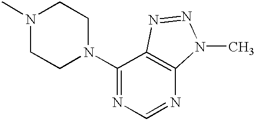 | Optically-active isomer (Amorohous powder) MS (APCI) 969 (M + H) [α]_{D} −59.4°(c 1.0, CHCl₃) |
| 336 (1) | 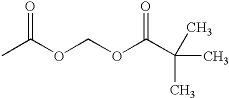 | 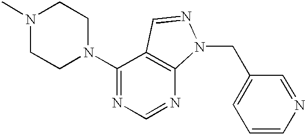 | Optically-active isomer (Amorohous powder) MS (APCI) 1045 (M + H) [α]_{D} −48.1°(c 1.0, CHCl₃) |
| 337 (1) | 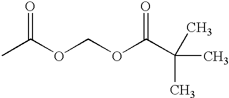 | 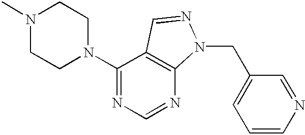 | Optically-active isomer (Amorohous powder) MS (APCI) 1045.6 (M + H) |
| 338 (1) | 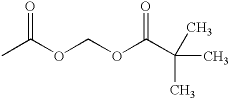 | 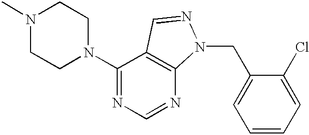 | Amorohous powder MS (APCI) 1078.5 (M + H) |

TABLE 20
(No. 5)

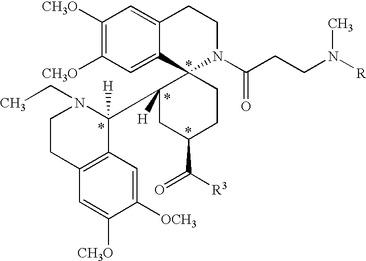

| Ex. | | | Physicochemical |
|---|---|---|---|
| No. | R | R³ | properties etc. |
| 339 (1) | 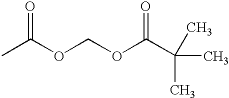 | 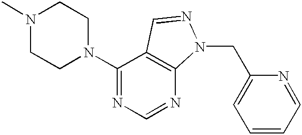 | Optically-active isomer (Amorohous powder) MS (APCI) 1045 (M + H) |
| 340 (1) | 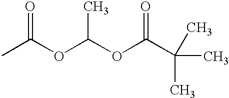 | 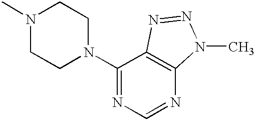 | Amorohous powder MS (APCI) 983.6 (M + H) |
| 341 (1) | 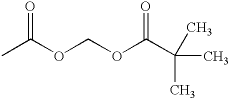 | 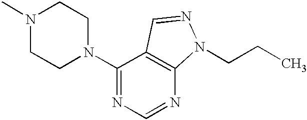 | Amorohous powder MS (APCI) 996.6 (M + H) |
| 342 (1) | 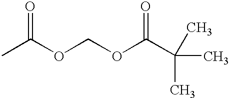 | 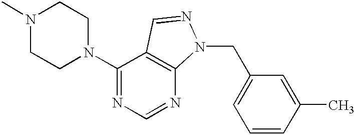 | Amorohous powder MS (APCI) 1058.7 (M + H) |
| 343 (1) | 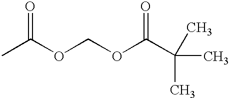 | 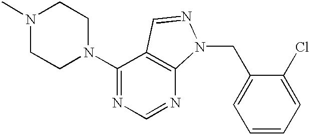 | Amorohous powder MS (APCI) 1078.6 (M + H) |
| 344 (1) | 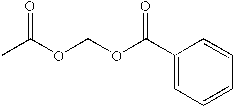 | 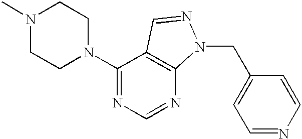 | Amorohous powder MS (APCI) 1065.5 (M + H) |

TABLE 20
(No. 6)

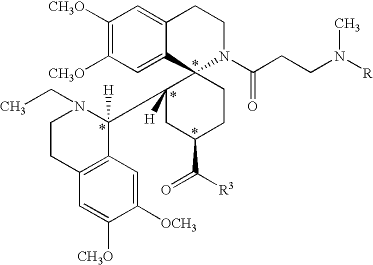

| Ex. | | | Physicochemical |
|---|---|---|---|
| No. | R | R³ | properties etc. |
| 345 (1) | 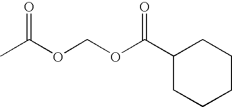 | 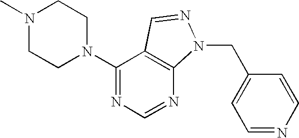 | Amorohous powder MS(APCI)1071.5(M + H) |
| 346 (1) | 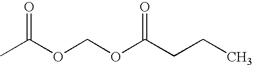 | 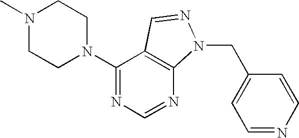 | Amorohous powder MS(APCI)1031(M + H) |
| 347 (1) | 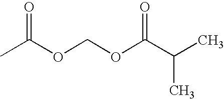 | 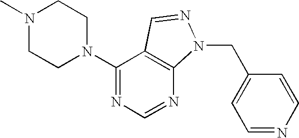 | Amorohous powder MS(APCI)1031(M + H) |
| 348 (1) | 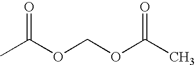 | 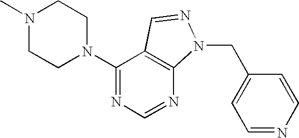 | Amorohous powder MS(APCI)1003(M + H) |
| 349 (1) | 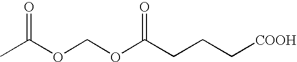 | 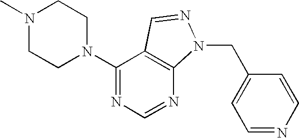 | Amorohous powder MS(ESI)1075.4(M + H) |
| 350 (1) | 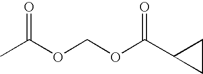 | 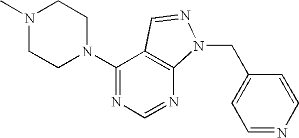 | Amorohous powder MS(APCI)1029(M + H) |

TABLE 20
(No. 7)

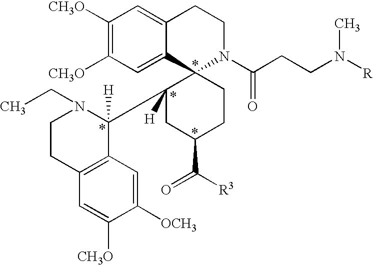

| Ex. | | | Physicochemical |
|---|---|---|---|
| No. | R | R³ | properties etc. |
| 351 (1) | 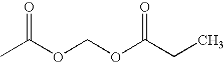 | 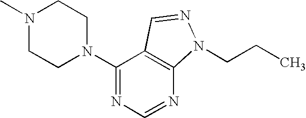 | Amorohous powder MS(APCI)968.6(M + H) |
| 352 (1) | 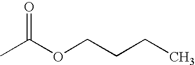 | 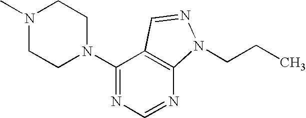 | Amorohous powder MS(APCI)938.6(M + H) |
| 353 (1) | 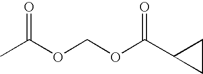 | 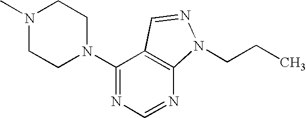 | Amorohous powder MS(APCI)980(M + H) |
| 354 (1) | 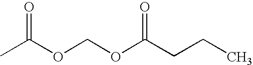 | 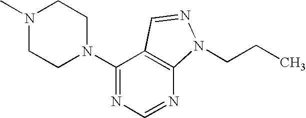 | Amorohous powder MS(APCI)982(M + H) |
| 355 (1) | 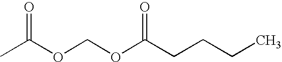 | 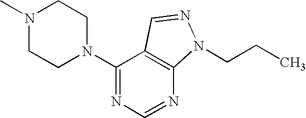 | Amorohous powder MS(APCI)996(M + H) |
| 356 (1) | 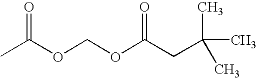 | 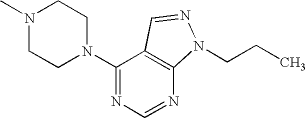 | Amorohous powder MS(APCI)1010.6(M + H) |

TABLE 20
(No. 8)

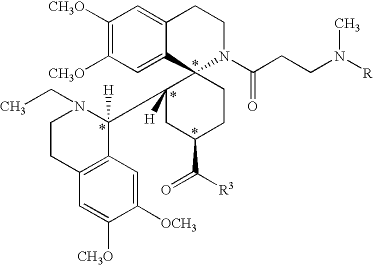

| Ex. | | | Physicochemical |
|---|---|---|---|
| No. | R | R³ | properties etc. |
| 357 (1) | 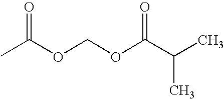 | 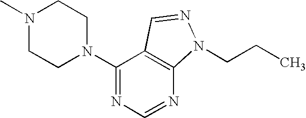 | Amorohous powder MS(APCI)982.6(M + H) |
| 358 (1) | 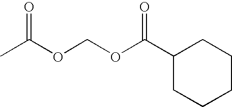 | 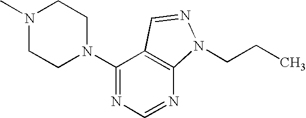 | Amorohous powder MS(APCI)1022.6(M + H) |
| 359 (1) | 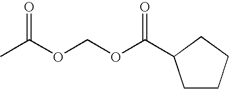 | 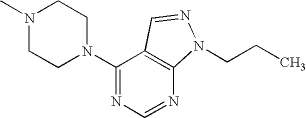 | Amorohous powder MS(APCI)1008(M + H) |
| 360 (1) | 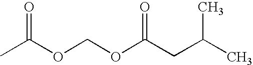 | 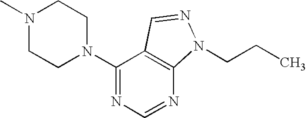 | Amorohous powder MS(APCI)996.7(M + H) |

TABLE 20
(No. 9)

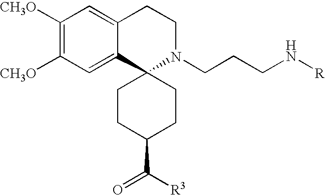

| Ex. | | | Physicochemical |
|---|---|---|---|
| No. | R | R³ | properties etc. |
| 361 (1) | 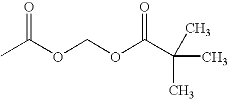 | 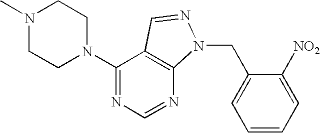 | Amorohous powder MS(APCI)842(M + H) |

2) The compound obtained in Example 316(1) to 361(1) is treated in the same manner as described in Example 2(2) to give the following compounds (salt).

Example 316(2): 2 Fumaric acid salt of the compound obtained in Example 316(1)

Example 317(2): 2 Fumaric acid salt of the compound obtained in Example 317(1)

Example 318(2): 2 Fumaric acid salt of the compound obtained in Example 318(1)

Example 319(2): 2 Fumaric acid salt of the compound obtained in Example 319(1)

Example 320(2): 2 Fumaric acid salt of the compound obtained in Example 320(1); Amorphous powder, MS(APCI)m/z: 928.6(M+H), IR(Nujol)cm−1: 3131, 1707, 1636

Example 321(2): 2 Fumaric acid salt of the compound obtained in Example 321(1); Amorphous powder, MS(APCI)m/z: 859.5(M+H), IR(Nujol)cm⁻¹: 3135, 1705, 1657

Example 322(2): 2 Fumaric acid salt of the compound obtained in Example 322(1); Amorphous powder, MS(APCI)m/z: 913.5(M+H), IR(Nujol)cm⁻¹: 1747, 1714, 1667, 1637

Example 323(2): 2 Fumaric acid salt of the compound obtained in Example 323(1); Amorphous powder, MS(APCI)m/z: 984(M+H), IR(Nujol)cm⁻¹: 3131, 1710, 1637

Example 324(2): 2 Fumaric acid salt of the compound obtained in Example 324(1)

Example 325(2): 2 Fumaric acid salt of the compound obtained in Example 325(1); Amorphous powder, [α]D −33.8° (c1.0, ethanol)

Example 326(2): 2 Fumaric acid salt of the compound obtained in Example 326(1); Amorphous powder, [α]D −34.39° (c1.0, ethanol), MS(APCI)m/z: 929.4(M+H), IR(Nujol)cm⁻¹: 3133, 1708, 1635

Example 327(2): 2 Fumaric acid salt of the compound obtained in Example 327(1)

Example 328(2): 2 Fumaric acid salt of the compound obtained in Example 328(1); Amorphous powder, MS(APCI)m/z: 957(M+H), IR(Nujol)cm⁻¹: 3131, 1706, 1637

Example 329(2): 2 Fumaric acid salt of the compound obtained in Example 329(1); Amorphous powder, MS(APCI)m/z: 1010(M+H), IR(Nujol)cm⁻¹: 3135, 1708, 1634

Example 330(2): 2 Fumaric acid salt of the compound obtained in Example 330(1); Amorphous powder, MS(APCI)m/z: 1045(M+H), IR(Nujol)cm⁻¹: 3133, 1709, 1633

Example 331(2): 2 Fumaric acid salt of the compound obtained in Example 331(1)

Example 332(2): 2 Fumaric acid salt of the compound obtained in Example 332(1); Amorphous powder, [α]D +40.2° (c 1.0, ethanol), MS(APCI)m/z: 969(M+H), IR(Nujol)cm⁻¹: 3081, 1749, 1713, 1666

Example 333(2): 2 Fumaric acid salt of the compound obtained in Example 333(1); Amorphous powder, [α]D +29° (c 0.5, ethanol), MS(APCI)m/z: 1045(M+H)

Example 334(2): 2 Fumaric acid salt of the compound obtained in Example 334(1); Amorphous powder, [α]D −38.8° (c 1.0 ethanol), MS(APCI)m/z: 1045.5(M+H), IR(Nujol)cm⁻¹: 1745, 1714, 1665, 1639

Example 335(2): 2 Fumaric acid salt of the compound obtained in Example 335(1); Amorphous powder, [α]D −43.0° (c 1.0, ethanol), MS(APCI)m/z: 969.5(M+H), IR(Nujol)cm⁻¹: 3084, 1714, 1637

Example 336(2): 2 Fumaric acid salt of the compound obtained in Example 336(1)

Example 337(2): 2 Fumaric acid salt of the compound obtained in Example 337(1); Amorphous powder, [α]D −37.24° (c 1.0, ethanol), MS(APCI)m/z: 1045.5(M+H), IR(Nujol)cm⁻¹: 3129, 1710, 1635

Example 338(2): 2 Fumaric acid salt of the compound obtained in Example 338(1); Amorphous powder, [α]D +40.2° (c 1.0, ethanol), MS(APCI)m/z: 1078(M+H)

Example 339(2): 2 Fumaric acid salt of the compound obtained in Example 339(1); Amorphous powder, [α]D +17.9° (c 1.0, ethanol), MS(APCI)m/z: 1045(M+H)

Example 340(2): 2 Fumaric acid salt of the compound obtained in Example 340(1)

Example 341(2): 2 Fumaric acid salt of the compound obtained in Example 341(1); Amorphous powder, MS(APCI)m/z: 996.6(M+H), IR(Nujol)cm⁻¹: 3133, 1711, 1633

Example 342(2): 2 Fumaric acid salt of the compound obtained in Example 342(1)

Example 343(2): 2 Fumaric acid salt of the compound obtained in Example 343(1); Amorphous powder, MS(APCI)m/z: 1078.7(M+H), IR(Nujol)cm⁻¹: 3130, 1711, 1633

Example 344(2): 2 Fumaric acid salt of the compound obtained in Example 344(1); Amorphous powder, MS(APCI)m/z: 1065.5(M+H), IR(Nujol)cm⁻¹: 3128, 1710, 1633

Example 345(2): 2 Fumaric acid salt of the compound obtained in Example 345(1); Amorphous powder, MS(APCI)m/z: 1071.5(M+H), IR(Nujol)cm⁻¹: 3130, 1710, 1633

Example 346(2): 2 Fumaric acid salt of the compound obtained in Example 346(1); Amorphous powder, MS(APCI)m/z: 1031(M+H), IR(neat+chloroform)cm⁻¹: 1715, 1635

Example 347(2): 2 Fumaric acid salt of the compound obtained in Example 347(1); Amorphous powder, MS(APCI)m/z: 1031(M+H), IR(Nujol)cm⁻¹: 1707, 1653, 1636

Example 348(2): 2 Fumaric acid salt of the compound obtained in Example 348(1); Amorphous powder, MS(APCI)m/z: 1003(M+H), IR(Nujol)cm⁻¹: 3415, 1757, 1707, 1651

Example 350(2): 2 Fumaric acid salt of the compound obtained in Example 350(1); Amorphous powder, MS(APCI)m/z: 1029(M+H), IR(Nujol)cm⁻¹: 3409, 1710, 1634

Example 351(2): 2 Fumaric acid salt of the compound obtained in Example 351(1); Amorphous powder, MS(FAB)m/z: 968(M+H), IR(Nujol)cm⁻¹: 3429, 1752, 1711, 1633

Example 352(2): 2 Fumaric acid salt of the compound obtained in Example 352(1); Amorphous powder, MS(APCI)m/z: 938.7(M+H), IR(Nujol)cm⁻¹: 3132, 1700, 1635

Example 353(2): 2 Fumaric acid salt of the compound obtained in Example 353(1); Amorphous powder, MS(APCI)m/z: 980(M+H), IR(Nujol)cm⁻¹: 1712, 1634

Example 354(2): 2 Fumaric acid salt of the compound obtained in Example 354(1); Amorphous powder, MS(APCI)m/z: 982(M+H), IR(Nujol)cm⁻¹: 1711, 1635

Example 355(2): 2 Fumaric acid salt of the compound obtained in Example 355(1); Amorphous powder, MS(APCI)m/z: 996(M+H), IR(Nujol)cm⁻¹: 3419, 1711, 1635

Example 356(2): 2 Fumaric acid salt of the compound obtained in Example 356(1); Amorphous powder, MS(APCI)m/z: 1010(M+H), IR(Nujol)cm⁻¹: 1710, 1631

Example 357(2): 2 Fumaric acid salt of the compound obtained in Example 357(1); Amorphous powder, MS(APCI)m/z: 982(M+H), IR(Nujol)cm⁻¹: 3129, 1710, 1635

Example 358(2): 2 Fumaric acid salt of the compound obtained in Example 358(1); Amorphous powder, MS(APCI)m/z: 1022.6(M+H), IR(Nujol)cm⁻¹: 3128, 1711, 1635

Example 359(2): 2 Fumaric acid salt of the compound obtained in Example 359(1); Amorphous powder, MS(APCI)m/z: 1008.8(M+H), IR(Nujol)cm⁻¹: 3425, 1711, 1633

Example 360(2): 2 Fumaric acid salt of the compound obtainer in Example 360(1); Amorphous powder, MS(APCI)m/z: 996.7(M+H), IR(Nujol)cm⁻¹: 3431, 1713, 1634

Example 361(2): 2 Citric acid salt of the compound obtained in Example 361(1); Amorphous powder, MS(APCI)m/z: 842(M+H), IR(Nujol)cm⁻¹: 3362, 2610, 1732, 1634

### Example 362

1) To a solution of (1R*,2R*(S*),4R*)-2′-[3-(methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (200 mg) and triethylamine (57 mg) in methylene chloride (8 mL) is added dropwise a solution of 2-(chloromethyloxy-carbonyloxy)pyridine (133 mL) in methylene chloride (2 mL) under ice-cooling and the mixture is stirred at 4° C. for 30 minutes. To the reaction mixture is added ethyl acetate and the mixture is washed with water and saturated aqueous NaCl solution, dried over sodium sulfate, and concentrated. A solution of thus-obtained residue in dimethylformamide (7 mL) is added to a suspension of tetrabutyl ammonium iodide (166 mg), and cesium carbonate (367 mg) in ethanol (52 mg) and the mixture is vigorously stirred under bubbling of CO₂ gas at room temperature for 2 hours. Cesium carbonate (367 mg) and ethanol (208 mg) are added to the mixture and the mixture is further stirred at 50-60° C. for 4 hours. To the reaction mixture is added ethyl acetate and the mixture is washed with water and saturated aqueous NaCl solution, dried over sodium sulfate, and concentrated. The residue is purified by column chromatography on silica gel (solvent; chloroform:methanol=100:1) to give (1R*,2R*(S*),4R)-2′-(3-[N-(ethoxycarbonyloxy-methyloxy-carbonyl)-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (167 mg, 43%) as an amorphous powder.

MS(APCI)m/z: 1033.5(M+H).

2) The compound obtained in the above step (1) is treated in the same manner as described in Example 2(2) to give a 2 fumaric acid salt thereof.

MS(APCI)m/z: 1033.6(M+H), IR(Nujol)cm⁻¹: 3131, 1757, 1710.

### Examples 363 to 367

1) The corresponding materials are treated in the same manner as described in Example 362(1) to give the compounds as shown in the following table (Table.21).

TABLE 21

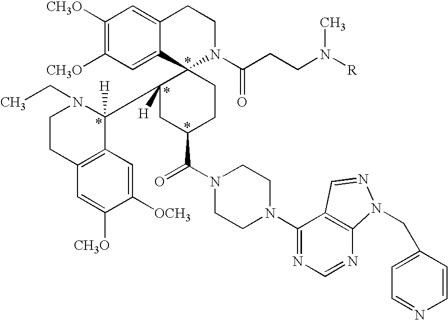

| Ex. | | Physicochemical |
|---|---|---|
| No. | R | properties etc. |
| 363 (1) | 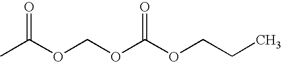 | Amorohous powder MS(APCI) 1047.5(M + H) |
| 364 (1) | 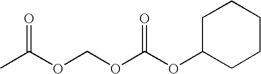 | Amorohous powder MS(APCI) 1087.5(M + H) |
| 365 (1) | 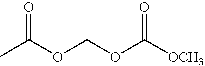 | Amorohous powder MS(APCI) 1019.5(M + H) |
| 366 (1) | 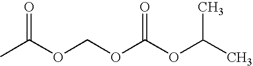 | Amorohous powder MS(APCI) 1047.5(M + H) |
| 367 (1) | 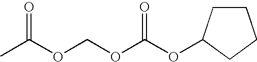 | Amorohous powder MS(APCI) 1073.5(M + H) |

2) The compound obtained in Example 363(1) to 367(1) is treated in the same manner as described in Example 2(2) to give the following compounds (salt).

Example 363(2): 2 Fumaric acid salt of the compound obtained in Example 363(1); Amorphous powder, MS(APCI)m/z: 1047.6(M+H), IR(Nujol)cm⁻¹: 3410, 3129, 1758, 1710

Example 364(2): 2 Fumaric acid salt of the compound obtained in Example 364(1); Amorphous powder, MS(APCI)m/z: 1087.5(M+H), IR(Nujol)cm⁻¹: 3408, 3129, 1752, 1710

Example 365(2): 2 Fumaric acid salt of the compound obtained in Example 365(1); Amorphous powder, MS(APCI)m/z: 1019.4(M+H), IR(Nujol)cm⁻¹: 3409, 3131, 1761, 1709

Example 366(2): 2 Fumaric acid salt of the compound obtained in Example 366(1); Amorphous powder, MS(APCI)m/z: 1047.5(M+H), IR(Nujol)cm⁻¹: 3127, 1751, 1715

Example 367(2): 2 Fumaric acid salt of the compound obtained in Example 367(1); Amorphous powder, MS(APCI)m/z: 1073.5(M+H), IR(Nujol)cm⁻¹: 3128, 1753, 1710

### Example 368

1) To a solution of triphosgene (446 mg) in methylene chloride (10 mL) is slowly added dropwise a solution of 4-hydroxymethyl-5-methyl-1,3-dioxol-2-one (M. Alpegiani et al, Synthetic Communication 22, 1277, 1992) (583 mg) in methylene chloride (5 mL) under ice-cooling and the mixture is stirred at the same temperature for 2.5 hours. The excess amount of phosgene is removed under reduced pressure. To the reaction mixture is added dropwise a solution of (1R*,2R*(S*),4R*)-2′-(3-(methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (1.00 g) and diisopropylethylamine (291 mg) in methylene chloride (5 mL) under ice-cooling for 5 minutes. water is added to the mixture and the mixture is concentrated. The residue is dissolved in ethyl acetate and washed with water and saturated aqueous sodium hydrogencarbonate solution, successively. The organic layer is dried over sodium sulfate and concentrated. The residue is purified by column chromatography on silica gel (solvent; chloroform:methanol=30:1) to give (1R*,2R*(S*),4R*)-2′-[3-[N-[(5-methyl-2-oxo-1,3-dioxol-4-yl)-methyloxycarbonyl]-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (273 mg, 23%) as an amorphous powder.

MS(APCI)m/e: 1043.5(M+H), IR(Nujol)cm⁻¹: 1817, 1736, 1703, 1635.

2) The compound obtained in the above step (1) is treated in the same manner as described in Example 2(2) to give a 2 fumaric acid salt thereof as an amorphous powder.

MS(APCI)m/e: 1043.5(M+H), IR(Nujol)cm⁻¹: 3131, 1815, 1704.

### Examples 369 to 377

1) The corresponding materials are treated in the same manner as described in Example 368(1) to give the compound as shown in the following tables (Table.22).

TABLE 22
(No. 1)

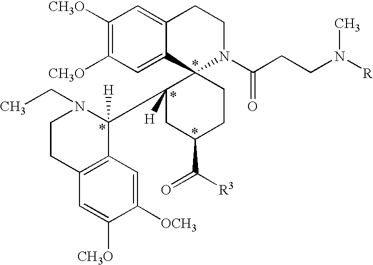

| Ex. | | | Physicochemical |
|---|---|---|---|
| No. | R | R³ | properties etc. |
| 369 (1) | 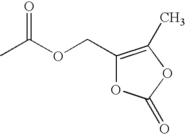 | 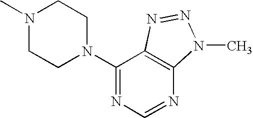 | Optically-active isomer (Amorohous powder) MS(APCI)967(M + H) [α]_{D} +59.0°(c 1.0, CHCl₃) |
| 370 (1) | 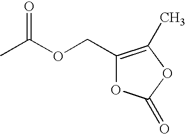 | 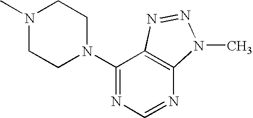 | Optically-active isomer (Amorohous powder) MS(APCI)967.6(M + H) |
| 371 (1) | 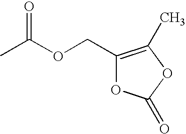 | 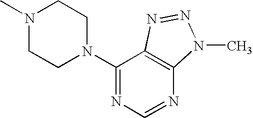 | M.P. 201-202° C. |
| 372 (1) | 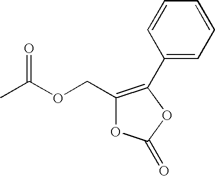 | 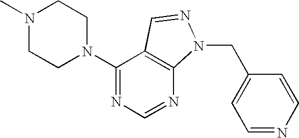 | Amorohous powder MS(APCI)1105.6(M + H) |
| 373 (1) | 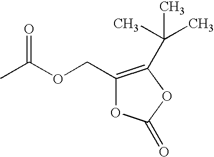 | 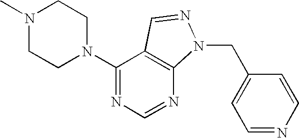 | Amorohous powder MS(APCI)1085.6(M + H) |

TABLE 22
(No. 2)

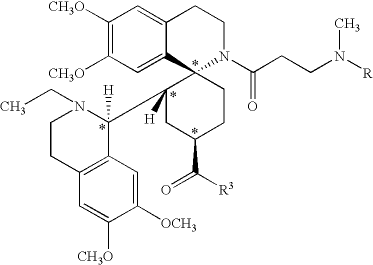

| Ex. | | | Physicochemical |
|---|---|---|---|
| No. | R | R³ | properties etc. |
| 374 (1) | 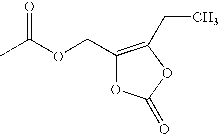 | 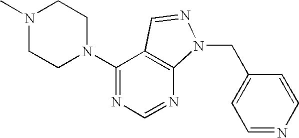 | Amorohous powder MS(APCI)1057(M + H) |
| 375 (1) | 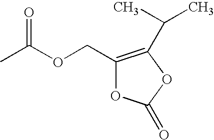 | 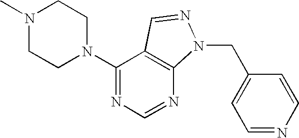 | Amorohous powder MS(APCI)1071(M + H) |
| 376 (1) | 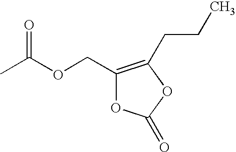 | 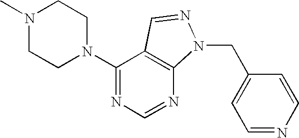 | Amorohous powder MS(APCI)1071(M + H) |
| 377 (1) | 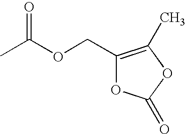 | 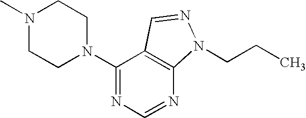 | Amorohous powder MS(APCI)994.8(M + H) |

2) The compound obtained in Example 369(1) to 377(1) is treated in the same manner as described in Example 2(2) to give the following compounds (salt).

Example 369(2): 2 Fumaric acid salt of the compound obtained in Example 369(1); Amorphous powder, [α]D +48.19° (c1.0, ethanol), MS(APCI)m/z: 967(M+H), IR(Nujol)cm⁻¹: 3133, 1817, 1706

Example 370(2): 2 Fumaric acid salt of the compound obtained in Example 370(1); Amorphous powder, [α]D −49.4° (c1.0, eternal), MS(APCI)m/z: 967.6(M+H), IR(Nujol)cm⁻¹: 3133, 1818, 1706

Example 371(2): 2 Fumaric acid salt of the compound obtained in Example 371(1); Amorphous powder, MS(APCI)m/z: 967(M+H), IR(Nujol)cm⁻¹: 3133, 1817, 1705

Example 372(2): 2 Fumaric acid salt of the compound obtained in Example 372(1); Amorphous powder, MS(APCI)m/z: 1105.5(M+H), IR(Nujol)cm⁻¹: 3404, 1821, 1759, 1704, 1651

Example 373(2): 2 Fumaric acid salt of the compound obtained in Example 373(1); Amorphous powder, MS(APCI)m/z: 1085.6(M+H), IR(Nujol)cm⁻¹: 3409, 3132, 1811, 1703

Example 374(2): 2 Fumaric acid salt of the compound obtained in Example 374(1); Amorphous powder, MS(APCI)m/z: 1057(M+H), IR(Nujol)cm⁻¹: 1819, 1703, 1652

Example 375(2): 2 Fumaric acid salt of the compound obtained in Example 375(1); Amorphous powder, MS(APCI)m/z: 1071(M+H), IR(Nujol)cm⁻¹: 3391, 1816, 1701, 1635

Example 376(2): 2 Fumaric acid salt of the compound obtained in Example 376(1); Amorphous powder, MS(APCI)m/z: 1071(M+H), IR(Nujol)cm⁻¹: 3407, 1703, 1636

Example 377(2): 2 Fumaric acid salt of the compound obtained in Example 377(1); Amorphous powder, MS(APCI)m/z: 994.7(M+H), IR(neat+chloroform)cm⁻¹: 1820, 1704, 1667, 1639

### Example 378

1) A solution of 4-Acetoxybenzylalcohol (337 mg), di-(2-pyridyl)carbonate (A. K. Ghosh et al, Tetrahedron Letters 32(34), 4251, 1991) (292 mg) and triethylamine (205 mg) in methylene chloride (5 mL) is stirred at room temperature overnight. To the reaction mixture is added (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1))(665 mg) and the mixture is stirred at room temperature for 3 hours. The reaction mixture is concentrated and ethyl acetate is added to the residue. The solution is washed with saturated aqueous sodium hydrogen carbonate solution and saturated aqueous NaCl solution, successively dried over sodium sulfate, and concentrated. The residue is purified by column chromatography on silica gel (solvent; chloroform:methanol=50:1) to give (1R*,2R*(S*),4R*)-2′-[3-[N-(4-acetoxybenzyloxycarbonyl)-N-methylamino]propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (697 mg, 86.1%) as an amorphous powder. MS(APCI)m/e: 1079.6(M+H)

2) The compound obtained in the above step (1) is treated in the same manner as described in Example 2(2) to give a 2 fumaric acid salt thereof as an amorphous powder.

MS(APCI)m/e: 1079.6(M+H), IR(Nujol)cm⁻¹: 1759, 1698, 1650.

### Examples 379 to 385

1) The corresponding materials are treated in the same manner as described in Example 378(1) to give the compound as shown in the following tables (Table.23).

TABLE 23
(No. 1)

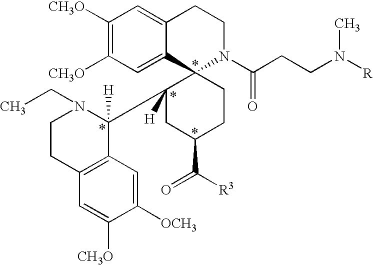

| Ex. | | | Physicochemical |
|---|---|---|---|
| No. | R | R³ | properties etc. |
| 379 (1) | 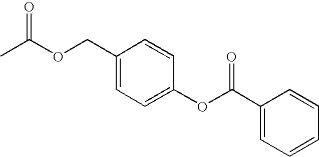 | 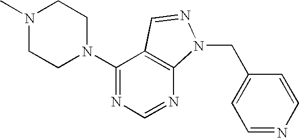 | Amorohous powder MS(APCI)1141.5(M + H) |
| 380 (1) | 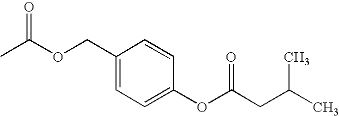 | 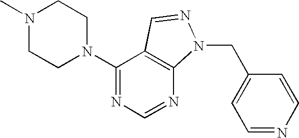 | Amorohous powder MS(APCI)1121.6(M + H) |
| 381 (1) | 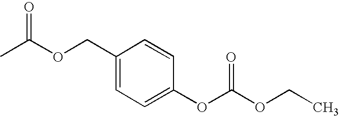 | 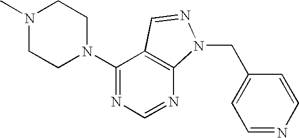 | Amorohous powder MS(APCI)1109.6(M + H) |
| 382 (1) | 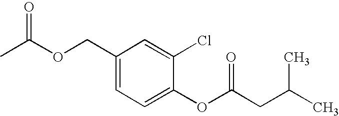 | 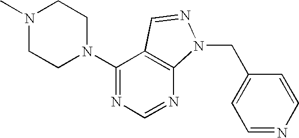 | Amorohous powder MS(APCI)1155.7(M + H) |
| 383 (1) | 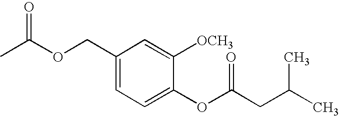 | 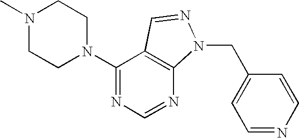 | Amorohous powder MS(APCI)1151.7(M + H) |

TABLE 23
(No. 2)

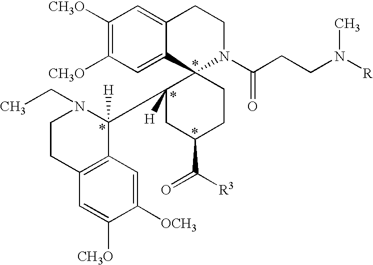

| Ex. | | | Physicochemical |
|---|---|---|---|
| No. | R | R³ | properties etc. |
| 384 (1) | 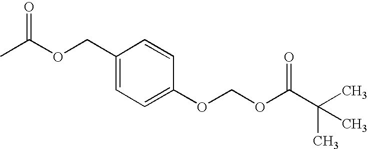 | 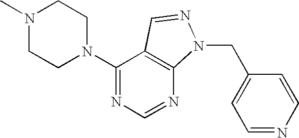 | Amorohous powder MS(APCI)1151.6(M + H) |
| 385 (1) | 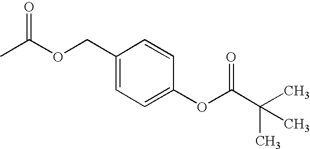 | 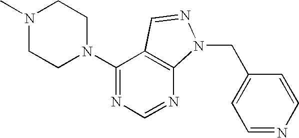 | Amorohous powder MS(APCI)1121.7(M + H) |

2) The compound obtained in Example 379(1) to 385(1) is 5 treated in the same manner as described in Example 2(2) to give the following compounds (salt).

Example 379(2): 2 Fumaric acid salt of the compound obtained in Example 379(1); Amorphous powder, MS(APCI)m/z: 1141.5(M+H), IR(Nujol)cm⁻¹: 1698, 1634

Example 380(2): 2 Fumaric acid salt of the compound obtained in Example 380(1); Amorphous powder, MS(APCI)m/z: 1121.6(M+H), IR(Nujol)cm⁻¹: 3125, 1751, 1700, 1635

Example 381(2): 2 Fumaric acid salt of the compound obtained in Example 381(1); Amorphous powder, MS(APCI)m/z: 1109.6(M+H), IR(neat+chloroform)cm⁻¹: 1759, 1700, 1637

Example 382(2): 2 Fumaric acid salt of the compound obtained in Example 382(1); Amorphous powder, MS(APCI)m/z: 1155.6(M+H), IR(Nujol)cm⁻¹: 3409, 3127, 1762, 1701, 1635

Example 383(2): 2 Fumaric acid salt of the compound obtained in Example 383(1); Amorphous powder, MS(APCI)m/z: 1151.7(M+H), IR(neat+chloroform)cm⁻¹: 1757, 1693, 1640

Example 384(2): 2 Fumaric acid salt of the compound obtained in Example 384(1); Amorphous powder, MS(APCI)m/z: 1151.6(M+H), IR(Nujol)cm⁻¹: 3129, 1711, 1635

Example 385(2): 2 Fumaric acid salt of the compound obtained in Example 385(1); Amorphous powder, MS(APCI)m/z: 1121.7(M+H), IR(neat+chloroform)cm⁻¹: 1746, 1693

### Example 386

1) A solution of (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(1-propyl-1H-pyrazolo[3,4-d]pyrimidin-4-yl)-1-piperazinyl]-carbonyl-spiro[cyclohexane-1,1′(2′H)-isoquinoline] (Compound obtained in Example 236(1)) (300 mg) and triethylamine (0.074 mL) in methylene chloride (1.5 mL) are added dropwise to a solution of 2-benzoyloxymethylbenzoyl chloride (108 mg) in methylene chloride (7 mL) under ice-cooling and the mixture is stirred at the same temperature for 30 minutes. The reaction mixture is poured into ice-water and extracted with methylene chloride. The organic layer is dried over sodium sulfate and concentrated. The residue is purified by column chromatography on silica gel (solvent; chloroform:methanol=4:1) to give (1R*,2R*(S*),4R*)-2′-[3-[N-(2-benzoyloxymethylbenzoyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-[1-propyl-1H-pyrazolo[3,4-d]pyrimidin-4-yl]-1-piperazinyl]carbonyl-spiro[cyclohexane-1,1′(2′H)-isoquinoline] (380 mg, 99%) as an amorphous powder.

MS (APCI) m/e: 1076 (M+H).

2) The compound obtained in the above step (1) is treated 10 in the same manner as described in Example 2(2) to give a 2 fumaric acid salt thereof as an amorphous powder.

MS(APCI)m/z: 1076(M+H), IR(Nujol)cm⁻¹: 1714, 1631.

### Examples 387 to 399

1) The corresponding materials are treated in the same manner as described in Example 386(1) to give the compound as shown in the following tables (Table.24).

TABLE 24
(No. 1)

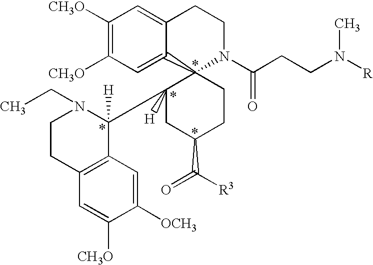

| Ex. | | | Physicochemical |
|---|---|---|---|
| No. | R | R³ | properties etc. |
| 387 (1) | 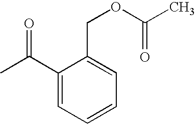 | 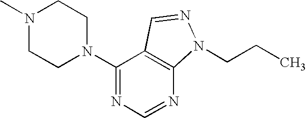 | Amorohous powder. MS(APCI)1014(M + H) |
| 388 (1) | 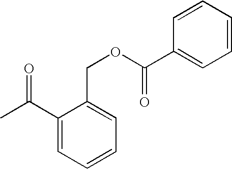 | 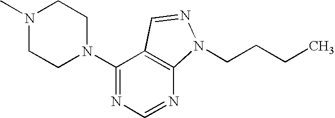 | Amorohous powder. MS(APCI)1090(M + H) |
| 389 (1) | 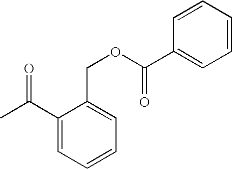 | 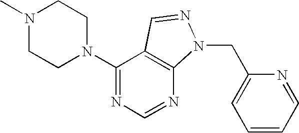 | M.p. 110p14 112° C. MS(APCI)1125(M + H) |
| 390 (1) | 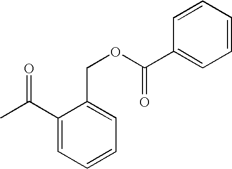 | 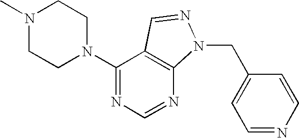 | Amorohous powder. MS(APCI)1125(M + H) |

TABLE 24
(No. 2)

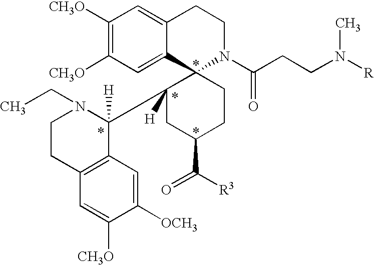

| Ex. | | | Physicochemical |
|---|---|---|---|
| No. | R | R³ | Properties etc. |
| 391 (1) | 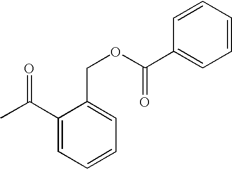 | 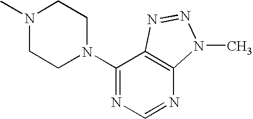 | M.p. 110-112° C. MS (APCI) 1049 (M + H) |
| 392 (1) | 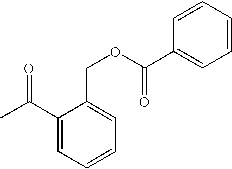 | 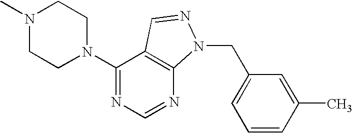 | Amorohous powder. MS (APCI) 1138 (M + H) |
| 393 (1) | 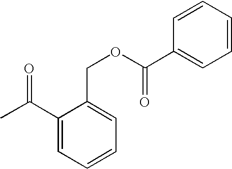 | 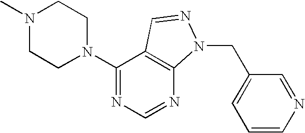 | Amorohous powder. MS (APCI) 1125 (M + H) |
| 394 (1) | 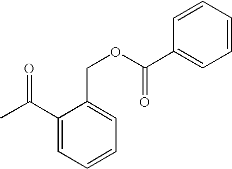 | 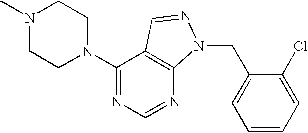 | Amorohous powder. MS (APCI) 1158.5 (M + H) |
| 395 (1) | 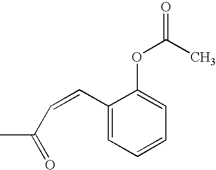 | 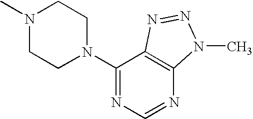 | Amorohous powder. MS (APCI) 999.5 (M + H) |
| 396 (1) | 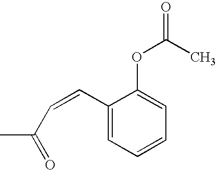 | 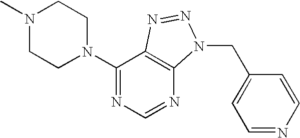 | Amorohous powder. MS (APCI) 1075.6 (M + H) |

TABLE 24
uz,27/31 (No. 3)

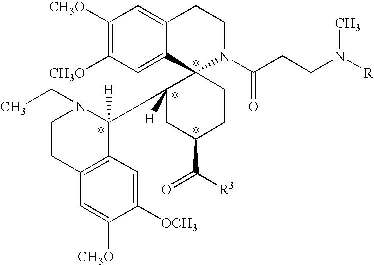

| Ex. | | | Physicochemical |
|---|---|---|---|
| No. | R | R3 | properties etc. |
| 397 (1) | 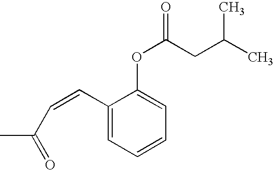 | 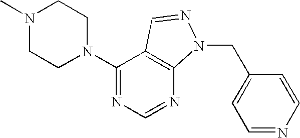 | Amorohous powder. MS (APCI) 1117.6 (M + H) |
| 398 (1) | 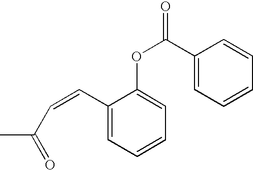 | 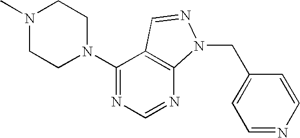 | Amorohous powder. MS (APCI) 1137.6 (M + H) |
| 399 (1) | 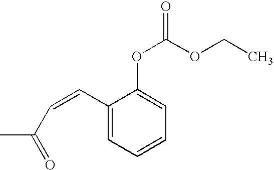 | 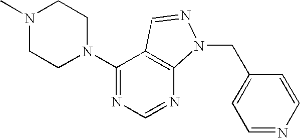 | Amorohous powder. MS (APCI) 1105 (M + H) |

2) The compound obtained in Example 387(1) to 399(1) is treated in the same manner as described in Example 2(2) to give the following compounds (salt).

Example 387(2): 2 Fumaric acid salt of the compound obtained in Example 387(1); Amorphous powder, MS(APCI)m/z: 1014(M+H), IR(Nujol)cm⁻¹: 3129, 1711, 1631

Example 388(2): 2 Fumaric acid salt of the compound obtained in Example 388(1); Amorphous powder, MS(APCI)m/z: 1090(M+H), IR(Nujol)cm⁻¹: 1715, 1629, 1572

Example 389(2): 2 Fumaric acid salt of the compound obtained in Example 389(1); Amorphous powder, MS(APCI)m/z: 1125(M+H), IR(Nujol)cm⁻¹: 3123, 1713, 1628, 1573

Example 390(2): 2 Fumaric acid salt of the compound obtained in Example 390(1); Amorphous powder, MS(APCI)m/z: 1125(M+H), IR(Nujol)cm⁻¹: 3124, 1713, 1630

Example 391(2): 2 Fumaric acid salt of the compound obtained in Example 391(1); Amorphous powder, MS(APCI)m/z: 1049(M+H), IR(Nujol)cm⁻¹: 3421, 1715, 1629

Example 392(2): 2 Fumaric acid salt of the compound obtained in Example 392(1); Amorphous powder, MS(APCI)m/z: 1138(M+H), IR(Nujol)cm⁻¹: 3431, 1716, 1632

Example 393(2): 2 Fumaric acid salt of the compound obtained in Example 393(1); Amorphous powder, MS(APCI)m/z: 1125(M+H), IR(Nujol)cm⁻¹: 3474, 1722, 1633

Example 394(2): 2 Fumaric acid salt of the compound obtained in Example 394(1); Amorphous powder, MS(APCI)m/z: 1158.5(M+H), IR(Nujol)cm⁻¹: 3447, 1721, 1635

Example 395(2): 2 Fumaric acid salt of the compound obtained in Example 395(1); Amorphous powder, MS(APCI)m/z: 999.5(M+H), IR(Nujol)cm⁻¹: 3432, 1760, 1635

Example 396(2): 2 Fumaric acid salt of the compound obtained in Example 396(1); Amorphous powder, MS(APCI)m/z: 1075.6(M+H), IR(Nujol)cm⁻¹: 3414, 1759, 1707

Example 397(2): 2 Fumaric acid salt of the compound obtained in Example 397(1); Amorphous powder, MS(APCI)m/z: 1117.7(M+H), IR(Nujol)cm⁻¹: 3412, 3129, 1751, 1706

Example 398(2): 2 Fumaric acid salt of the compound obtained in Example 398(1); Amorphous powder, MS(APCI)m/z: 1137.6(M+H), IR(Nujol)cm⁻¹: 3413, 1707

Example 399(2): 2 Fumaric acid salt of the compound obtained in Example 399(1); Amorphous powder, MS(APCI)m/z: 1105.7(M+H), IR(neat+chloroform)cm⁻¹: 1759, 1705, 1681

### Example 400

1) To a solution of (1R*,2R*(S*),4R*)-2′-[3-(methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (200 mg) in formic acid (2 mL) is added dropwise acetic anhydride (0.21 mL) under ice-cooling and the mixture is stirred at room temperature for 3 hours. The reaction mixture is concentrated and the residue is diluted with ethyl acetate. The solution is poured into 10% aqueous solution of potassium carbonate and the aqueous layer is extracted with ethyl acetate. The organic layer is washed with water and saturated aqueous NaCl solution, successively dried over sodium sulfate, and concentrated. The residue is purified by column chromatography on silica gel (solvent; chloroform:methanol:28% aqueous ammonia solution=30:1:0.1) to give (1R*,2R*(S*),4R*)-2′-[3-(N-formyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (179 mg, 87%) as an amorphous powder.

MS(APCI)m/z: 915(M+H), IR(Nujol)cm⁻¹: 1732, 1668.

2) The compound obtained in the above step (1) is treated in the same manner as described in Example 2(2) to give 1 fumaric acid salt thereof. MS(APCI)m/z: 915(M+H), IR(Nujol)cm⁻¹: 3411, 1704, 1659

### Examples 401

1) To a mixture of a solution of (1R*,2R*(S*),4R*)-2′-[3-(methylamino)propionyl]-3′,4′-dihydro-6′,7′-dimethoxy-2-[2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (200 mg) in ethyl acetate (2 mL) and a solution of potassium carbonate (62 mg)in water (2 mL) is added ethyl chloroformate (21 μL) under ice-cooling. The mixture is stirred at the same temperature for 1.5 hours. The reaction mixture is diluted with ethyl acetate and washed with water and saturated aqueous NaCl solution, successively dried over sodium sulfate, and concentrated. The residue is purified by column chromatography on silica gel (Solvent; chloroform:methanol:aqueous 28% ammonia=30:1:0.1) to give (1R*,2R*(S*),4R*)-2′-[3-(N-ethoxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-(1-4-pyridylmethyl)-1H-pyrazolo[3,4-d]pyrimidin-4-yl]-1-piperazinyl]carbonyl-spiro[cyclohexane-1,1′(2′H)-isoquinoline] (212 mg, 98%) as an amorphous powder.

MS(APCI)m/z: 959.5(M+H), IR(Nujol)cm⁻¹: 1694.

2) The compound obtained in the above step (1) is treated in the same manner as described in Example 2(2) to give a 1 fumaric acid salt thereof as an amorphous powder.

MS(APCI)m/z: 959(M+H), IR(Nujol)cm⁻¹: 1691, 1632.

### Examples 402

1) To a solution of (1R*,2R*(S*),4R*)-2′-[3-(methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-propyl-1H-pyrazolo[3,4-d]pyrimidin-4-yl]-1-piperazinyl]carbonyl-spiro[cyclohexane-1,1′(2′H)-isoquinoline] (Compound obtained in Example 236(1)) (500 mg) and triethylamine (0.13 mL) in methylene chloride (10 mL) is added butyl chloroformate (83 μL) under ice-cooling and the mixture is stirred at the same temperature for 1 hours. The reaction mixture is concentrated. Ethyl acetate is added to the residue and washed with water and saturated aqueous NaCl solution, successively dried over sodium sulfate, and concentrated. The residue is purified by column chromatography on silica gel (Solvent; chloroform:methanol=20:1) to give (1R*,2R*(S*),4R*)-2′-[3-(N-butoxycarbonyl-N-methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-propyl-1H-pyrazolo[3,4-d]pyrimidin-4-yl]-1-piperazinyl]carbonyl-spiro[cyclohexane-1,1′(2′H)-isoquinoline] (560 mg, 100%) as an amorphous powder.

MS(APCI)m/z: 938.6(M+H), IR(Nujol)cm⁻¹: 1697, 1637.

2) The compound obtained in the above step (1) is treated in the same manner as described in Example 2(2) to give a 2 fumaric acid salt thereof as an amorphous powder.

MS(APCI)m/z: 938.7(M+H), IR(Nujol)cm⁻¹: 3428, 3132, 1700, 1635.

### Example 403

1) A mixture of (1R*,2R*(S*),4R*)-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (Compound obtained in Example 165(1)) (800 mg), t-butoxycarbonylglycin (190 mg), triethylamine (0.38 mL), 1-ethyl-3-(3-dimehylaminopropyl)carbodiimide hydrochloride (233 mg), and 1-hydroxybenzotriazole (183 mg) in methylene chloride (20 mL) is stirred at room temperature for 13 hours. The reaction mixture is diluted with chloroform and washed with saturated aqueous sodium hydrogencarbonate solution and saturated aqueous NaCl solution, successively. The organic layer is dried over sodium sulfate and concentrated. The residue is purified by column chromatography on silica gel (solvent; chloroform:methanol:aqueous 28% ammonia; 50:1:0.1) to give (1R*,2R*(S*),4R)-2′-[3-[N-(tert-butoxycarbonylamino-acetyl)-N-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo [3,4-d]pyrimidin-4-yl]-1-piperazinyl]carbonyl-spiro[cyclohexane-1,1′(2′H)-isoquinoline] (978 mg, 100%) as an amorphous powder.

MS(APCI)m/z: 1044.5(M+H), IR(Nujol)cm⁻¹: 1711.

2) To a solution of the compound obtained in the above step (1) in methylene chloride (5 mL) is added trifluoroacetic acid (5 mL) and the mixture is stirred at room temperature for 15 hours. The reaction mixture is concentrated and the residue is basified with 10% aqueous potassium carbonate solution. The mixture is extracted with chloroform and the extract is washed with saturated aqueous NaCl solution, dried over sodium sulfate, and concentrated. The residue is purified by column chromatography on silica gel (solvent; chloroform:methanol:aqueous 28% ammonia; 20:1:0.1) to give (1R*,2R*(S*),4R*)-2′-[3-(N-aminoacetyl-N-methylamino)-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline] (846 mg, 96%) as an amorphous powder.

MS(APCI)m/z: 944.5(M+H), IR(Nujol)cm⁻¹: 3373, 1635.

3) The compound obtained in the above step (2) is treated in the same manner as described in Example 2(2) to give 1 fumaric acid salt thereof as an amorphous powder.

MS(APCI)m/z: 944(M+H), IR(nujol)cm⁻¹: 3398, 1633, 1573.

### Example 404

(1α,4β)-3′,4′-Dihydro-6′,7′-dimethoxy-4-carboxy-spiro[cyclohexane-1,1′(2′H)-isoquinoline] (Compound obtained in Reference example 12(4)) is treated in the same manner as described in Example 52(1) to give (1α,4β)-3′,4′-dihydro-6′,7′-dimethoxy-4-[4-[1-(3-nitrobenzyl)-1H-pyrazolo[3,4-d]pyrimidin-4-yl]-1-piperazinyl]carbonyl-spiro[cyclohexane-1,1′(2′H)-isoquinoline] as an amorphous powder. MS(APCI)m/z: 627(M+H)

### Example 405

1) To a solution of (1α,4β)-3′,4′-dihydro-6′,7′-dimethoxy-4-benzyloxycarbonyl-spiro[cyclohexane-1,1′(2′H)-isoquinoline] (Compound obtained in Reference example 16(1)) (1.226 g) and triethylamine (2.915 g) in methylene chloride (10 mL) is added dropwise a solution of chloroacetyl chloride (0.51 mL) in methylene chloride (10 mL) under ice-cooling and the mixture is stirred at the same temperature for 2 hours. The reaction mixture is poured into saturated aqueous sodium hydrogencarbonate solution and extracted with chloroform. The extract is washed with saturated aqueous NaCl solution, dried over sodium sulfate, and concentrated The residue is purified by column chromatography on silica gel (solvent; chloroform:methanol=50:1) to give (1α,4β)-2′-chloroacetyl-3′,4′-dihydro-6′,7′-dimethoxy-4-benzyloxycarbonyl-spiro[cyclohexane-1,1′(2′H)-isoquinoline] (1.35 g) as an oil.

MS(APCI)m/z: 472(M+H), IR(neat)cm⁻¹: 1727, 1661.

2) The compound obtained in the above step (1) (1.334 g) is dissolved in acetonitrile (20 ml) and 50% aqueous dimethylamine solution (10 mL) is added thereto. The mixture is stirred at room temperature overnight. The reaction mixture is concentrated and the residue is purified by column chromatography on silica gel (solvent; chloroform:methanol=20:1) to give (1α,4β)-2′-dimethylamino-acetyl-3′,4′-dihydro-6′,7′-dimethoxy-4-benzyloxycarbonyl-spiro[cyclohexane-1,1′(2′H)-isoquinoline] (1.34 g) as an oil.

MS(APCI)m/z: 481(M+H), IR(neat)cm⁻¹: 1731, 1658, 1651, 1633.

3) The compound obtained in the above step (2) (1.32 g) is dissolved in methanol (20 ml) and 10% palladium-carbon (263 mg) is added thereto. The mixture is subjected to catalytic hydrogenation under atmospheric pressure at room temperature for 24 hours. The reaction mixture is filtered and the filtrate is concentrated to give (1α,4β)-2′-dimethylaminoacetyl-3′,4′-dihydro-6′,7′-dimethoxy-4-carboxy-spiro[cyclohexane-1,1′(2′H)-isoquinoline] (0.99 g).

M.p.233-235° C., MS(APCI)m/z: 391(M+H), IR(Nujol)cm⁻¹: 1671.

4) The compound obtained in the above step (3) is treated in the same manner as described in Example 52(1) to give (1α,4β)-2′-dimethylaminoacetyl-3′,4′-dihydro-6′,7′-dimethoxy-4-[4-[1-(3-methylbenzyl)-1H-pyrazolo[3,4-d]pyrimidin-4-yl]-1-piperazinyl]carbonyl-spiro[cyclohexane-1,1′(2′H)-isoquinoline] as a crystalline powder.

M.p.116-118° C., MS(APCI)m/z: 681(M+H).

5) The compound obtained in the above step (4) is treated in the same manner as described in Example 2(2) to give 1 fumaric acid salt thereof.

MS(APCI)m/z: 681(M+H), IR(Nujol)cm⁻¹: 3407, 1650, 1634.

### Example 406

1) To a solution of (1α,4β)-3′,4′-dihydro-6′,7′-dimethoxy-4-benzyloxycarbonyl-spiro[cyclohexane-1,1′(2′H)-isoquinoline] (Compound obtained in Reference example 16(1)) (2.77 g) in tetrahydrofuran (30 mL) are added aqueous formaldehyde solution (30 mL) and sodium triacetoxyborohydride (4.45 g) under ice-cooling and the reaction mixture is stirred at room temperature for 2 hours. The reaction mixture is poured into saturated aqueous sodium hydrogencarbonate solution and extracted with ethyl acetate. The extract is washed with water and dried over sodium sulfate. The solution is concentrated and the residue is purified by column chromatography on silica gel (solvent; chloroform:methanol=20:1) to give (1α,4β)-2′-methyl-3′,4′-dihydro-6′,7′-dimethoxy-4-benzyloxycarbonyl-spiro[cyclohexane-1,1′(2′H)-isoquinoline] (2.76 g) as an amorphous powder solid.

MS(APCI)m/z: 410(M+H), IR(neat)cm⁻¹: 1727, 1608.

2) To a solution of the compound obtained in the above step (1) (2.76 g) in ethanol (30 ml) is added palladium hydroxide (400 mg). The mixture is subjected to catalytic hydrogenation under 3 atm pressure of hydrogen. To the reaction mixture is added water, and the mixture is filtered. The filtrate is concentrated to give (1α,4β)-2′-methyl-3′,4′-dihydro-6′,7′-dimethoxy-4-carboxy-spiro-[cyclohexane-1,1′(2′H)-isoquinoline] (1.87 g) as crystals.

M.p. 199° C., MS(APCI)m/z: 320(M+H), IR(Nujol)cm⁻¹: 3385, 1722, 1611.

3) The compound obtained in the above step (2) is treated in the same manner as described in Example 52(1) to give (1α,4β)-2′-methyl-3′,4′-dihydro-6′,7′-dimethoxy-4-[4-(3-methyl)-3H-1,2,3-triazolo[4,5-d]pyrimidin-7-yl]-1-piperazinyl]carbonyl-spiro[cyclohexane-1,1′(2′H)-isoquinoline] as crystalline powder.

M.p.189-191° C., MS(APCI)m/z: 521(M+H).

### Example 407

1) A mixture of 3′,4′-dihydro-6′,7′-dihydroxy-4,4-diethoxy-carbonyl-spiro[cyclohexane-1,1′(2′H)-isoquinoline] (Compound obtained in Reference example 13(1)) (14.767 g), potassium carbonate (27.03 g), and iodoethane (15.64 mL) in dimethylacetamide (60 mL) is stirred under ice-cooling for 30 minutes, at room temperature for 1 hour and at 100° C. for 3 hours. Consequently, the mixture is stirred overnight at room temperature. To the reaction mixture is added water and the mixture is extracted with ethyl acetate. The extract is washed with water, saturated aqueous sodium hydrogencarbonate and saturated aqueous NaCl solution, successively. The organic layer is dried over sodium sulfate and concentrated. The residue is purified by column chromatography on silica gel (solvent; ethyl acetate:n-hexan=1:1) to give 2′-ethyl-3′,4′-dihydro-6′,7′-diethoxy-4,4-diethoxycarbonyl-spiro[cyclohexane-1,1′(2′H)-isoquinoline] (17.81 g) as an oil.

MS(APCI)m/z: 462(M+H), IR(neat)cm⁻¹: 1730.

2) A mixture of the compound obtained in the above step (1) (17.79 g), sodium hydroxide (7.75 g), ethanol (30 mL) and water (30 mL) is stirred at room temperature for 5 hours and then refluxed for 13 hours. The reaction mixture is cooled with ice and adjusted its pH to 1-2 with 10% hydrochloric acid. The mixture is concentrated and to the residue is added pyridine (200 mL). The mixture is refluxed for 3 hours. After cooling, the mixture is concentrated. The trace of pyridine is removed by azeotropic distillation with toluene. The residue is dissolved in water. The solution is neutralized with 10% aqueous sodium hydroxide solution and NaCl is added to the mixture. The mixture is extracted with chloroform and the extract is washed with saturated aqueous NaCl solution, dried over sodium sulfate, and concentrated. The residue is crystallized from isopropyl alcohol and ethyl acetate to give (1α,4β)-2′-ethyl-3′,4′-dihydro-6′,7′-diethoxy-4-carboxy-spiro-[cyclohexane-1,1′(2′H)-isoquinoline] (3.26 g)

M.p. 203-213° C., MS(APCI)m/z: 362(M+H), IR(neat)cm⁻¹: 1715, 1735.

3) The compound obtained in the above step (2) is treated in the same manner as described in Example 52(1) to give (1α,4β)-2′-ethyl-3′,4′-dihydro-6′,7′-diethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline] as an amorphous powder.

MS(APCI)m/z: 639(M+H).

### Example 408

1) A mixture of 3,4-dihydro-6,7-dimethoxy-2-(3-dibenzyl-aminopropyl)-1′-[4-(3-methyl-3H-1,2,3-triazolo[4,5-d]pyrimidin-7-yl)piperazin-1-yl]carbonyl-spiro[isoquinoline-1(2H),4′-piperidine] (Compound obtained in Reference example 23(7)) (579 mg),5% palladium-carbon (116 mg), and ammonium formate (980 mg) in tetrahydrofuran (10 mL) and methanol (20 mL) is refluxed for 2.5 hours. The reaction mixture is filtered and the filtrate is concentrated. To the residue is added a 10% aqueous sodium carbonate solution and the mixture is extracted with chloroform. The extract is washed with saturated aqueous NaCl solution, dried over sodium sulfate, and concentrated. The residue is purified by column chromatography on neutral silica gel (solvent; chloroform:methanol:aqueous 28% ammonia=20:1:0.1) to give 3,4-dihydro-6,7-dimethoxy-2-(3-aminopropyl)-1′-[4-(3-methyl-3H-1,2,3-triazolo[4,5-d]pyrimidin-7-yl)piperazin-1-yl)carbonyl-spiro[isoquinoline-1(2H),4′-piperidine] (357 mg, 81%).

MS(APCI)m/z: 565(M+H), IR(neat)cm⁻¹: 3373, 1639.

2) A solution of the compound obtained in the above step (1) (139 mg) in dimethylformamide (3 mL) is added triethylamine (75 mg) and N-t-butoxycarbonyl-N′-benzyloxycarbonyl-1H-pyrazol-1-carboxamidine (cf. WO00/78723) and the mixture is stirred at room temperature for 1 hour. The reaction mixture is diluted with ethyl acetate and the mixture is washed with water and saturated aqueous NaCl solution. The organic layer is dried over sodium sulfate and concentrated. The residue is purified by column chromatography on silica gel (solvent; chloroform:methanol=100:1) to give 3,4-dihydro-6,7-dimethoxy-2-[3(N-t-butoxycarbonyl-N′-benzyloxycarbonyl)-guanidinopropyl]-1′-[4-(3-methyl-3H-1,2,3-triazolo[4,5-d]pyrimidin-7-yl)piperazin-1-yl]carbonyl-spiro[isoquinoline-1(2H),4′-piperidine] (190 mg, 92%).

MS(APCI)m/z: 841(M+H), IR(Nujol)cm⁻¹: 3325, 1722.

3) To a solution of the compound obtained in the above step (2) (175 mg) in methylene chloride (1 mL) is added trifluoroacetic acid (1 mL) and the mixture is stirred at room temperature for 2 hours. The reaction mixture is concentrated and to the residue is added chloroform. The mixture is washed with 10% aqueous sodium carbonate solution and saturated aqueous NaCl solution, successively. The organic layer is dried over sodium sulfate and concentrated. The residue is purified by column chromatography on silica gel (solvent; chloroform:methanol:aqueous ammonia=20:1:0.1) to give 3,4-dihydro-6,7-dimethoxy-2-(3-(N-benzyloxycarbonyl)guanidine-propyl]-1′-[4-(3-methyl-3H-1,2,3-triazolo[4,5-d]pyrimidin-7-yl)piperazin-1-yl]carbonyl-spiro[isoquinolin-1(2H),4′-piperidine] (162 mg; quantitatively).

MS(APCI)m/z: 841(M+H), IR(Nujol)cm⁻¹: 3375, 1634.

4) A mixture of the compound obtained in the above step (2) (150 mg) and 10% palladium-carbon (30 mg) in methanol (5 mL) is subjected to catalytic hydrogenation under 3-4 atm pressure. The reaction mixture is filtered. To the filtrate is added hydrochloric acid-ethanol. The mixture is concentrated. The residue is triturated with ethanol-methanol and collected to give 3,4-dihydro-6,7-dimethoxy-2-(3-guanidinopropyl)-1′-[4-(3-methyl-3H-1,2,3-triazolo[4,5-d]pyrimidin-7-yl)piperazin-1-yl]carbonyl-spiro[isoquinoline-1(2H),4′-piperidine hydrochloride (111 mg) as an amorphous powder.

MS(APCI)m/z: 607(M+H), IR(Nujol)cm⁻¹: 3327, 1650.

### Examples 409 to 447

The corresponding materials are treated in the same manner as described in Example 52(1) to give the compounds as shown in the following tables (Table.25).

TABLE 25
(No. 1)

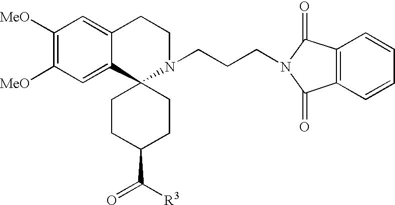

| Ex. | | Physicochemical |
|---|---|---|
| No. | R³ | properties etc. |
| 409 | 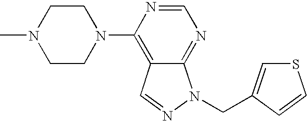 | Amorphous powder MS (APCI) 775 (M + H) |
| 410 | 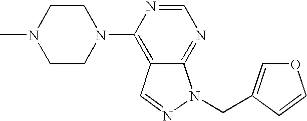 | Amorphous powder MS (APCI) 759 (M + H) |
| 411 | 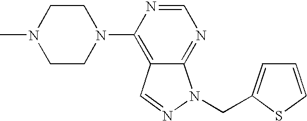 | Amorphous powder MS (APCI) 775 (M + H) |
| 412 | 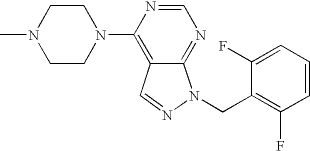 | Amorphous powder MS (APCI) 805 (M + H) |
| 413 | 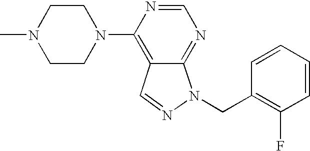 | Amorphous powder MS (APCI) 787 (M + H) |
| 414 | 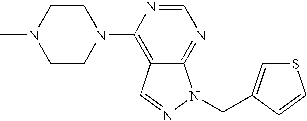 | Amorphous powder MS (APCI) 814 (M + H) |
| 415 | 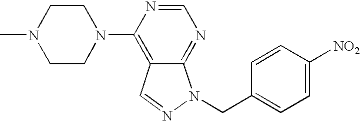 | Amorphous powder MS (APCI) 814 (M + H) |
| 416 | 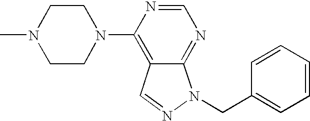 | Amorphous powder MS (APCI) 769 (M + H) |

TABLE 25
(No. 2)

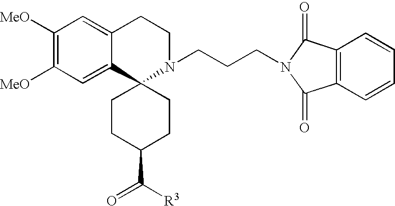

| Ex. | | Physicocheinical |
|---|---|---|
| No. | R³ | properties etc. |
| 417 | 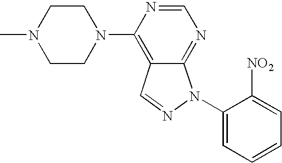 | Amorphous powder MS (APCI) 800/801 (M + H) |
| 418 | 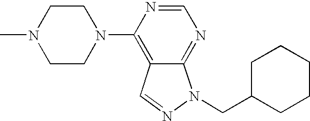 | crude product |
| 419 | 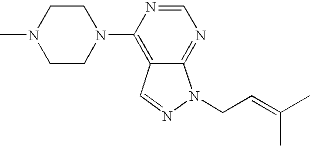 | crude product |
| 420 | 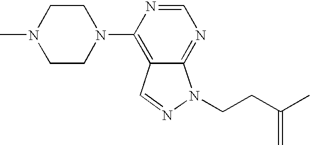 | crude product |
| 421 | 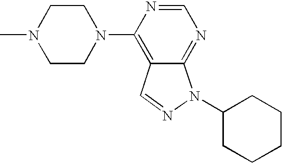 | crude product |
| 422 | 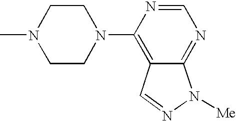 | crude product |
| 423 | 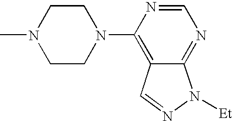 | crude product |
| 424 | 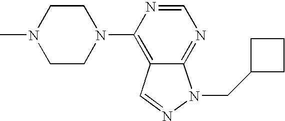 | crude product |

TABLE 25
(No. 3)

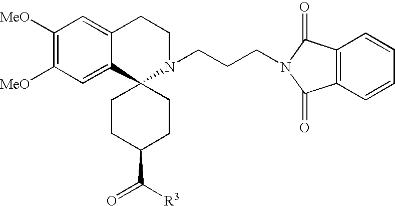

| | | Physico- |
|---|---|---|
| | | chemical |
| Ex. | | properties |
| No. | R³ | etc. |
| 425 | 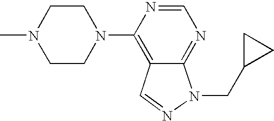 | crude product |
| 426 | 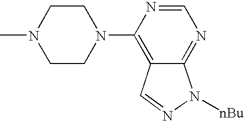 | crude product |
| 427 | 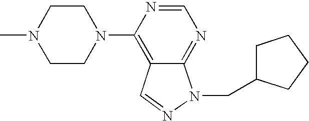 | crude product |
| 428 | 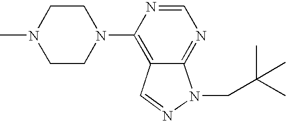 | crude product |
| 429 | 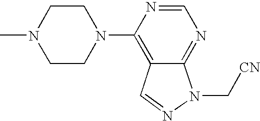 | crude product |
| 430 | 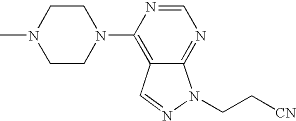 | crude product |
| 431 | 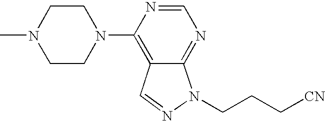 | crude product |
| 432 | 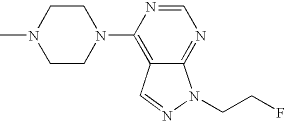 | crude product |
| 433 | 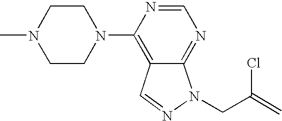 | crude product |

TABLE 25
(No. 4)

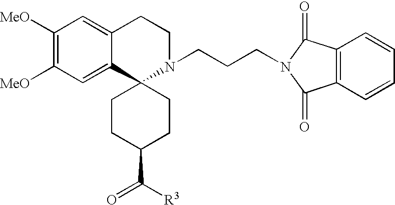

| Ex. | | Physicochemical |
|---|---|---|
| No. | R³ | properties etc. |
| 434 | 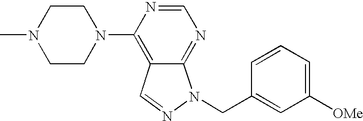 | crude product |
| 435 | 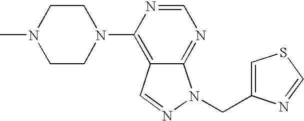 | crude product |
| 436 | 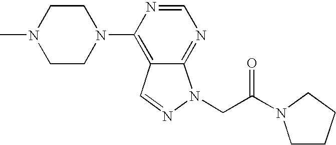 | crude product |
| 437 | 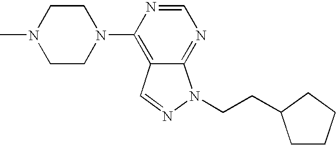 | crude product |
| 438 | 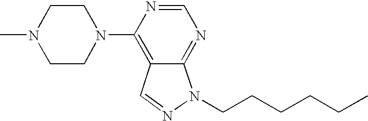 | crude product |
| 439 | 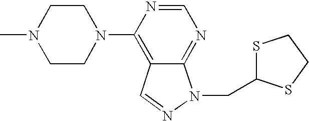 | crude product |
| 440 | 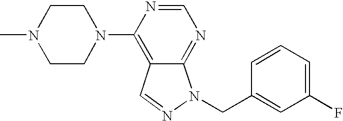 | crude product |
| 441 | 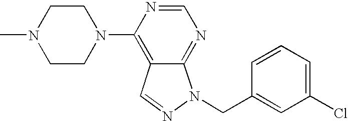 | crude product |
| 442 | 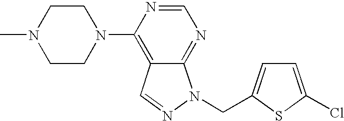 | Amorphous powder MS (APCI) 809 (M + H) IR (Nujol) 1769, 1707, 1638 |

TABLE 25
(No. 5)

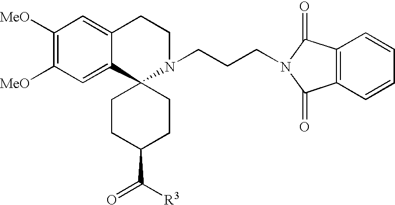

| Ex. | | Physicochemical |
|---|---|---|
| No. | R³ | properties etc. |
| 443 | 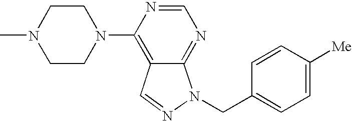 | Amorphous powder MS (APCI) 783 (M + H) IR (Nujol) 1769, 1709, 1639 |
| 444 | 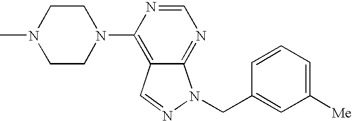 | Amorphous powder MS (APCI) 783 (M + H) IR (Nujol) 1769, 1709, 1639 |
| 445 | 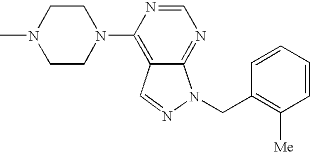 | Amorphous powder MS (APCI) 783 (M + H) IR (Nujol) 1769, 1709, 1639 |
| 446 | 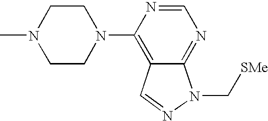 | crude product |
| 447 | 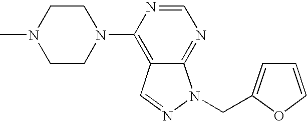 | Amorphous powder MS (APCI) 759 (M + H) |

### Examples 448 to 472

1) The corresponding materials are treated in the same manner as described in Example 274(2) to give the compounds as shown in the following tables (Table.26).

TABLE 26
(No. 1)

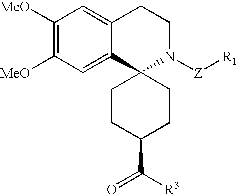

| Ex. | | | Physicochemical |
|---|---|---|---|
| No. | —Z—R¹ | R³ | properties etc. |
| 448 (1) | 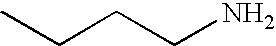 | 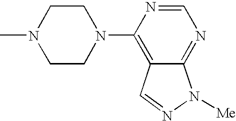 | Amorphous powder MS (APCI) 563 (M + H) |
| 449 (1) | 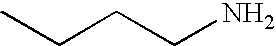 | 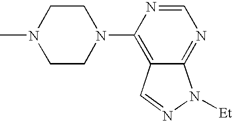 | Amorphous powder MS (APCI) 577 (M + H) |
| 450 (1) | 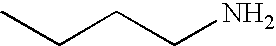 | 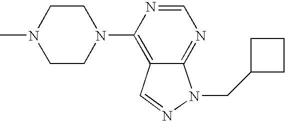 | Amorphous powder MS (APCI) 617 (M + H) |
| 451 (1) | 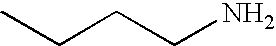 | 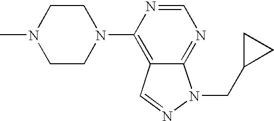 | Amorphous powder MS (APCI) 603 (M + H) |
| 452 (1) | 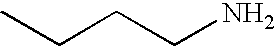 | 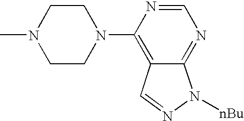 | Amorphous powder MS (APCI) 605 (M + H) |
| 453 (1) | 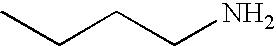 | 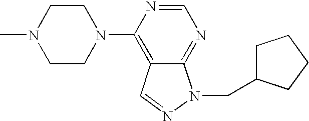 | Amorphous powder MS (APCI) 631 (M + H) |
| 454 (1) | 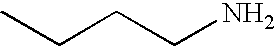 | 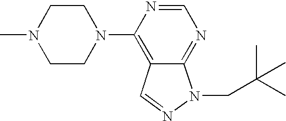 | Amorphous powder MS (APCI) 619 (M + H) |

TABLE 26
(No. 2)

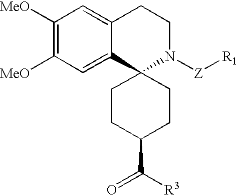

| Ex. | | | Physicochemical |
|---|---|---|---|
| No. | —Z—R¹ | R³ | properties etc. |
| 455 (1) | 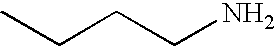 | 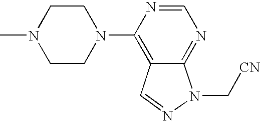 | Amorphous powder MS (APCI) 588 (M + H) |
| 456 (1) | 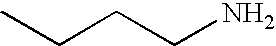 | 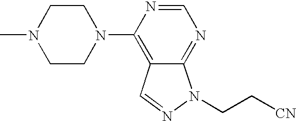 | Amorphous powder MS (APCI) 602 (M + H) |
| 457 (1) | 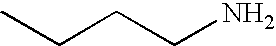 | 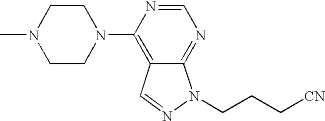 | Amorphous powder MS (APCI) 616 (M + H) |
| 458 (1) | 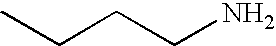 | 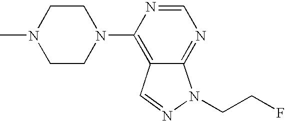 | Amorphous powder MS (APCI) 595 (M + H) |
| 459 (1) | 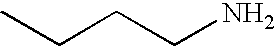 | 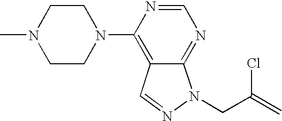 | Amorphous powder MS (APCI) 623 (M + H) |
| 460 (1) | 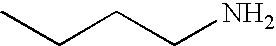 | 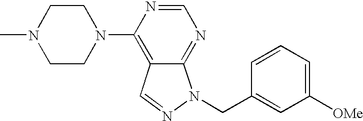 | Amorphous powder MS (APCI) 669 (M + H) |
| 461 (1) | 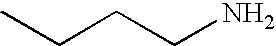 | 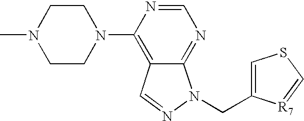 | Amorphous powder MS (APCI) 646 (M + H) |

TABLE 26
(No. 3)

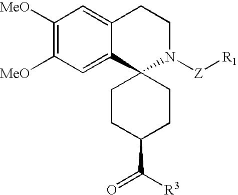

| Ex. | | | Physicochemical |
|---|---|---|---|
| No. | —Z—R¹ | R³ | properties etc. |
| 462 (1) | 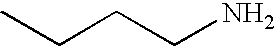 | 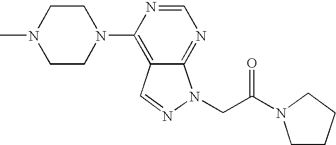 | Amorphous powder MS (APCI) 660 (M + H) |
| 463 (1) | 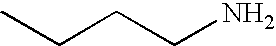 | 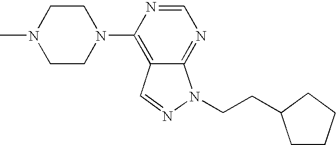 | Amorphous powder MS (APCI) 645 (M + H) |
| 464 (1) | 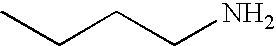 | 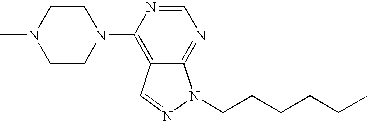 | Amorphous powder MS (APCI) 633 (M + H) |
| 465 (1) | 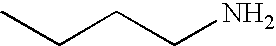 | 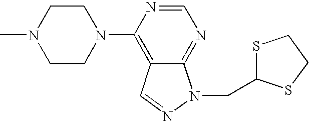 | Amorphous powder MS (APCI) 667 (M + H) |
| 466 (1) | 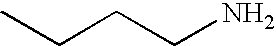 | 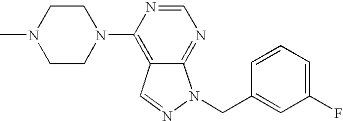 | Amorphous powder MS (APCI) 657 (M + H) |
| 467 (1) | 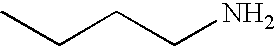 | 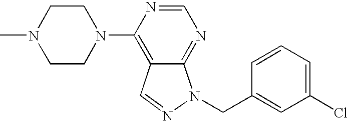 | Amorphous powder MS (APCI) 673 (M + H) |

TABLE 26
(No. 4)

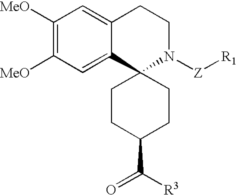

| Ex. No. | —Z—R¹ | R³ | Physicochemical properties etc. |
|---|---|---|---|
| 468(1) | 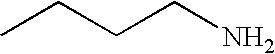 | 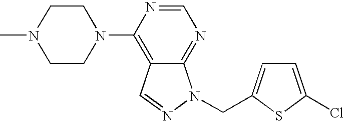 | Amorphous powder MS(APCI) 679(M + H) IR(Nujol) 1637 |
| 469(1) | 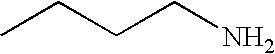 | 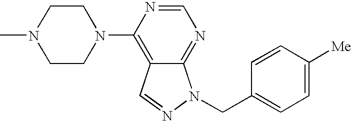 | Amorphous powder MS(APCI) 653(M + H) IR(Nujol) 1633 |
| 470(1) | 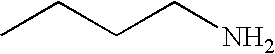 | 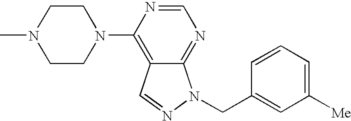 | Amorphous powder MS(APCI) 653(M + H) IR(Nujol) 1635 |
| 471(1) | 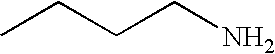 | 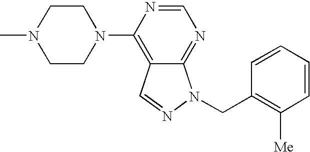 | Amorphous powder MS(APCI) 653(M + H) IR(Nujol) 1636 |
| 472(1) | 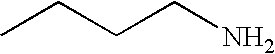 | 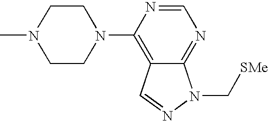 | Amorphous powder MS(APCI) 609(M + H) IR(Nujol) 1637 |

2) The compounds obtained in Examples 468(1) to 472(1) are treated in the same manner as described in Example 2(2) to give the following compounds (salt).

Example 468(2): 1 Fumaric acid salt of the compound obtained in Example 468(1); MS(APCI)m/z: 679(M+H), IR(Nujol)cm⁻¹: 1633

Example 469(2): 1 Fumaric acid salt of the compound obtained in Example 469(1); MS(APCI)m/z: 653(M+H), IR(Nujol) cm⁻¹: 1633

Example 470(2): 1 Fumaric acid salt of the compound obtained in Example 470(1); MS(APCI)m/z: 653(M+H), IR(Nujol)cm⁻¹: 1634

Example 471(2): 1 Fumaric acid salt of the compound obtained in Example 471(1); MS(APCI)m/z: 653(M+H), IR(Nujol)cm⁻¹: 1632

Example 472(2): 1 Fumaric acid salt of the compound obtained in Example 472(1); MS(APCI)m/z: 609(M+H), IR(Nujol)cm⁻¹: 1629

### Examples 473 to 486

The corresponding materials are treated in the same manner as described in Example 274(2) and 2(2) to give the compounds as shown in the following tables (Table.27).

TABLE 27
(No. 1)

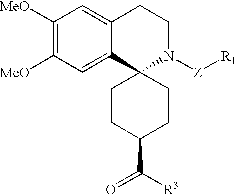

| Ex. No. | —Z—R¹ | R³ | Physicochemical properties etc. |
|---|---|---|---|
| 473 * | 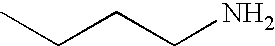 | 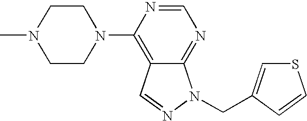 | Amorphous powder MS(APCI) 645(M + H) |
| 474 * | 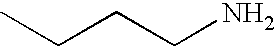 | 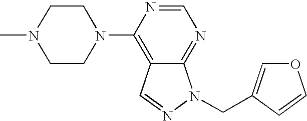 | Amorphous powder MS(APCI) 629(M + H) |
| 475 * | 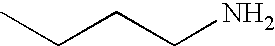 | 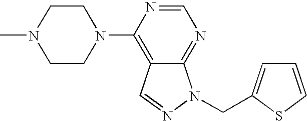 | Amorphous powder MS(APCI) 645(M + H) |
| 476 * | 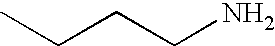 | 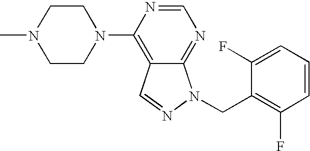 | Amorphous powder MS(APCI) 675(M + H) |
| 477 * | 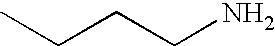 | 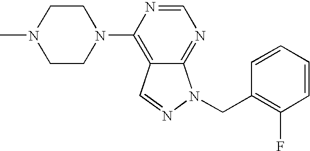 | Amorphous powder MS(APCI) 657(M + H) |
| 478 * | 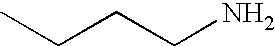 | 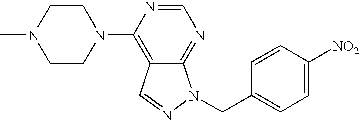 | Amorphous powder MS(APCI) 684(M + H) |
| 479 * | 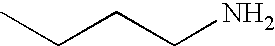 | 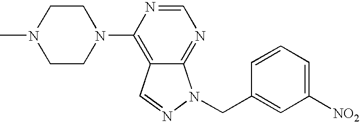 | Amorphous powder MS(APCI) 684(M + H) |

*: 1 Fumaric acid salt

TABLE 27
(No. 2)

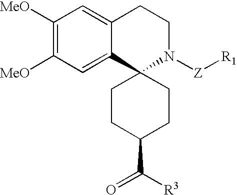

| Ex. No. | —Z—R¹ | R³ | Physicochemical properties etc. |
|---|---|---|---|
| 480 * | 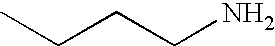 | 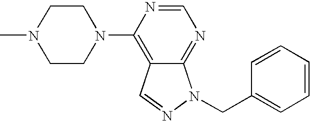 | Amorphous powder MS(APCI) 639(M + H) |
| 481 * | 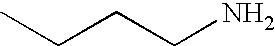 | 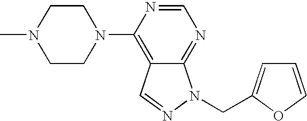 | Amorphous powder MS(APCI) 629(M + H) |
| 482 * | 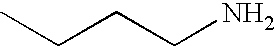 | 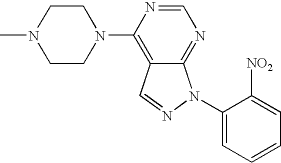 | Amorphous powder MS(APCI) 670(M + H) |
| 483 * | 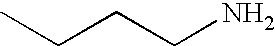 | 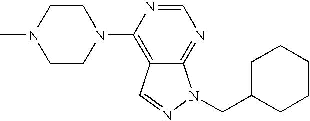 | Amorphous powder MS(APCI) 645(M + H) |
| 484 * | 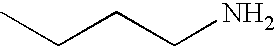 | 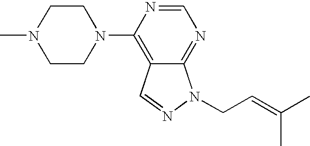 | Amorphous powder MS(APCI) 617(M + H) |
| 485 * | 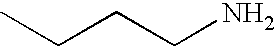 | 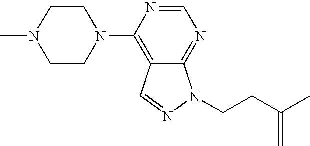 | Amorphous powder MS(APCI) 617(M + H) |
| 486* | 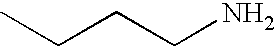 | 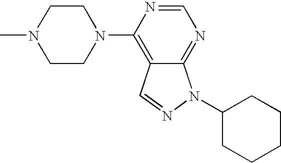 | Amorphous powder MS(APCI) 631(M + H) |

*: 1 Fumaric acid salt

### Examples 487 to 488

The corresponding materials are treated in the same manner as described in Example 293(1) and (2) to give the compounds as shown in the following table (Table.28).

TABLE 28

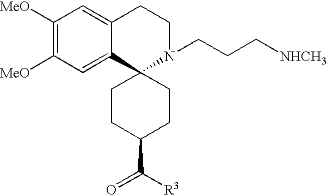

| Ex. No. | R³ | Physicochemical properties etc. |
|---|---|---|
| 487(1) | 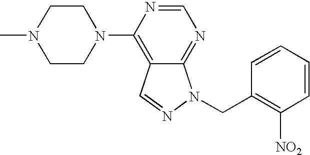 | Amorphous powder MS(APCI) 698(M + H) |
| 488(1) | 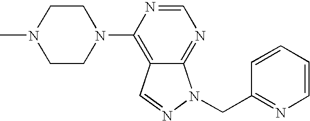 | Amorphous powder MS(APCI) 654(M + H) |

2) The compound obtained in Example 488(1) is treated in the same manner as described in Example 2(2) to give 1 Fumaric acid salt thereof. MS(APCI)m/z: 654(M+H), IR(Nujol)cm⁻¹: 3373, 1628

### Example 489

To a solution of (1α,4β)-2′-(3-aminopropyl)-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (200 mg) and cyclohexanone (0.3 mL) in methylene chloride is added sodium triacetoxyborohydride (94 mg) and the mixture is stirred at room temperature for 13 hours. The reaction mixture is diluted with ethyl acetate and the solution is washed with saturated aqueous sodium hydrogencarbonate, water and saturated aqueous NaCl solution, successively. The organic layer is dried over sodium sulfate and concentrated. The residue is purified by column chromatography on silica gel (solvent; chloroform:methanol:aqueous ammonia=20:1:0.1) to give (1α,4β)-2′-(3-cyclohexyl-aminopropyl)-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′H)-isoquinoline] (201 mg) as an amorphous powder. MS(APCI)m/z: 766(M+H), IR(Nujol)cm⁻¹: 1640, 1571

### Examples 490 to 503

1) The corresponding materials are treated in the same manner as described in Example 489 to give the compounds as shown in the following tables (Table.29).

TABLE 29
(No. 1)

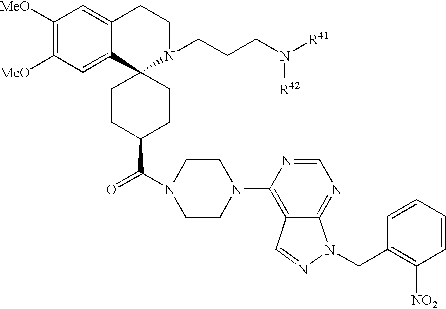

| Ex. No. | R⁴¹ | R⁴² | Physicochemical properties etc. |
|---|---|---|---|
| 490(1) | H | isopropyl | Amorphous powder |
| | | | MS(APCI) 726(M + H) |
| 491(1) | n-Pr | n-propyl | Amorphous powder |
| | | | MS(APCI) 768(M + H) |
| 492(1) | H | neopentyl | Amorphous powder |
| | | | MS(APCI) 754(M + H) |
| 493(1) | H | benzyl | Amorphous powder |
| | | | MS(APCI) 774(M + H) |
| 494(1) | CH₃ | neopentyl | Amorphous powder |
| | | | MS(APCI) 768(M + H) |
| 495(1) | CH₃ | isopropyl | Amorphous powder |
| | | | MS(APCI) 740(M + H) |
| 496(1) | CH₃ | Benzyl | Amorphous powder |
| | | | MS(APCI) 788(M + H) |
| 497(1) | CH₃ | cyclohexyl | Amorphous powder |
| | | | MS(APCI) 780(M + H) |
| 498(1) | CH₃ | n-hexyl | Amorphous powder |
| | | | MS(APCI) 782(M + H) |
| 499(1) | CH₃ | n-propyl | Amorphous powder |
| | | | MS(APCI) 740(M + H) |

TABLE 29
(No. 2)

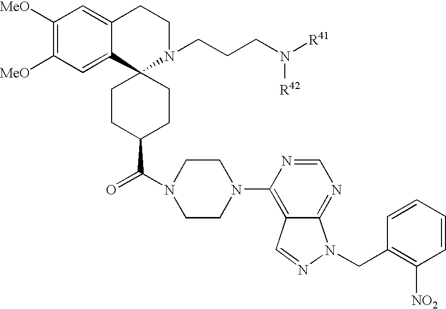

| Ex. No. | R⁴¹ | R⁴² | Physicochemical properties etc. |
|---|---|---|---|
| 500(1) | CH₃ | n-butyl | Amorphous powder |
| | | | MS(APCI) 754(M + H) |
| 501(1) | CH₃ | n-pentyl | Amorphous powder |
| | | | MS(APCI) 768.4(M + H) |
| 502(1) | CH₃ | Isobutyl | Amorphous powder |
| | | | MS(APCI) 754.4(M + H) |
| 503(1) | CH₃ | ethyl | Amorphous powder |
| | | | MS(APCI) 726(M + H) |

2) The compounds obtained in Examples 490(1) to 503(1) are treated in the same manner as described in Example 2(2) to give the following compounds (salt).

Example 490(2): 1 Fumaric acid salt of the compound obtained in Example 490(1); Amorphous powder, MS(APCI)m/z: 726(M+H), IR(Nujol)cm⁻¹: 1573

Example 491(2): 1 Fumaric acid salt of the compound obtained in Example 491(1); Amorphous powder, MS(APCI)m/z: 768(M+H), IR(Nujol)cm⁻¹: 1643

Example 494 (2): 1 Fumaric acid salt of the compound obtained in Example 494(1); Amorphous powder, MS(APCI)m/z: 768(M+H), IR(Nujol)cm⁻¹: 1635

Example 495(2): 1 Fumaric acid salt of the compound obtained in Example 495(1); Amorphous powder, MS(APCI)m/z: 740(M+H), IR(Nujol)cm⁻¹: 1634

Example 496(2): 1 Fumaric acid salt of the compound obtained in Example 496(1); Amorphous powder, MS(APCI)m/z: 788(M+H), IR(Nujol)cm⁻¹: 1704

Example 497(2): 1 Fumaric acid salt of the compound obtained in Example 497(1); Amorphous powder, MS(APCI)m/z: 780(M+H), IR(Nujol)cm⁻¹: 1635

Example 498(2): 1 Fumaric acid salt of the compound obtained in Example 498(1); Amorphous powder, MS(APCI)m/z: 782(M+H), IR(Nujol)cm⁻¹: 1705

Example 499(2): 1 Fumaric acid salt of the compound obtained in Example 499(1); Amorphous powder, MS(APCI)m/z: 740(M+H), IR(Nujol)cm⁻¹: 1635

Example 500(2): 1 Fumaric acid salt of the compound obtained in Example 500(1), Amorphous powder, MS(APCI)m/z: 754(M+H), IR(Nujol)cm⁻¹: 1635

Example 501(2): 1 Fumaric acid salt of the compound obtained in Example 501(1); Amorphous powder, MS(APCI)m/z: 768.4(M+H), IR(Nujol)cm⁻¹: 1635

Example 502(2): 1 Fumaric acid salt of the compound obtained in Example 502(1); Amorphous powder, MS(APCI)m/z: 754.4(M+H), IR(Nujol)cm⁻¹: 1635

Example 503(2): 1 Fumaric acid salt of the compound obtained in Example 503(1); Amorphous powder, MS(APCI)m/z: 726(M+H), IR(Nujol)cm⁻¹: 1574

### Examples 504 to 636

1) The corresponding materials are treated in the same manner as described in Example 52(1) to give the compounds as shown in the following tables (Table.30).

TABLE 30
(No. 1)

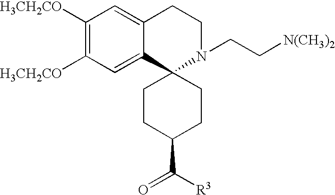

| Ex. No. | R³ | Physicochemical properties etc. |
|---|---|---|
| 504(1) | 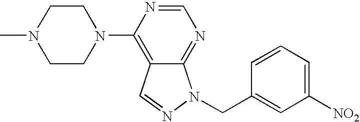 | Amorphous powder MS(APCI) 726(M + H) |
| 505(1) | 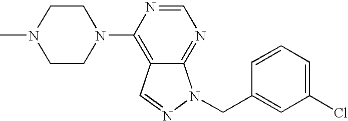 | Amorphous powder MS(APCI) 715(M + H) |
| 506(1) | 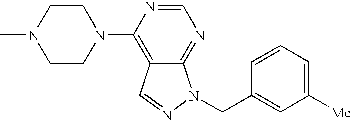 | Amorphous powder MS(APCI) 695(M + H) |
| 507(1) | 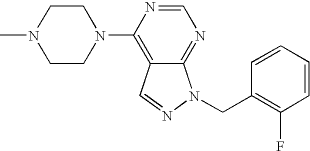 | Amorphous powder MS(APCI) 699(M + H) |
| 508(1) | 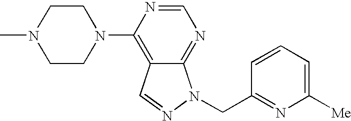 | Amorphous powder MS(APCI) 696(M + H) |
| 509(1) | 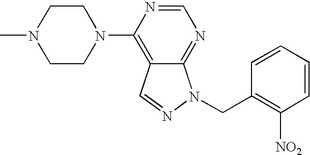 | Amorphous powder MS(APCI) 726(M + H) |
| 510(1) | 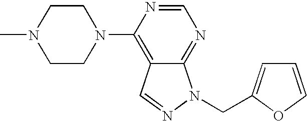 | Amorphous powder MS(APCI) 671(M + H) |

TABLE 30
(No. 2)

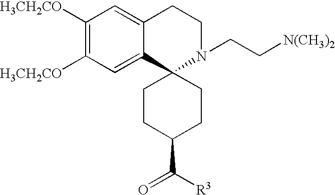

| Ex. No. | R³ | Physicochemical properties etc. |
|---|---|---|
| 511(1) | 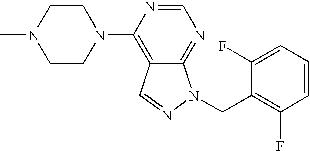 | Amorphous powder MS(APCI) 717(M + H) |
| 512(1) | 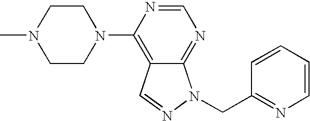 | Amorphous powder MS(APCI) 682(M + H) |
| 513(1) | 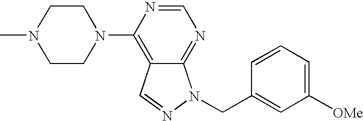 | Amorphous powder MS(APCI) 711(M + H) |

TABLE 30
(No. 3)

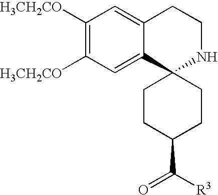

| Ex. No. | R³ | Physicochemical properties etc. |
|---|---|---|
| 514(1) | 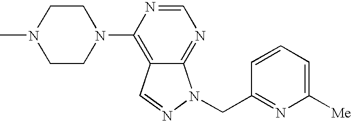 | Amorphous powder MS(APCI) 625(M + H) |
| 515(1) | 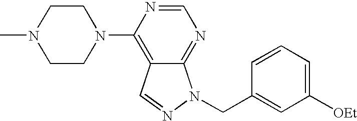 | Amorphous powder MS(APCI) 653(M + H) |
| 516(1) | 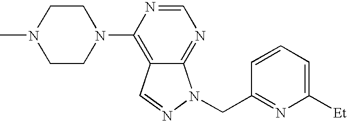 | Amorphous powder MS(APCI) 639(M + H) |
| 517(1) | 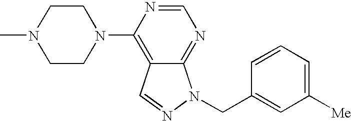 | Amorphous powder MS(APCI) 624(M + H) |
| 518(1) | 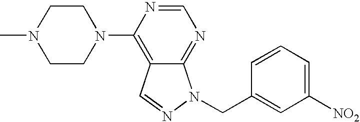 | Amorphous powder MS(APCI) 655(M + H) |
| 519(1) | 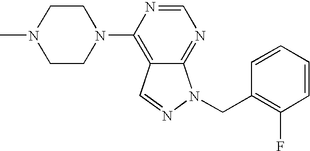 | Amorphous powder MS(APCI) 628(M + H) |
| 520(1) | 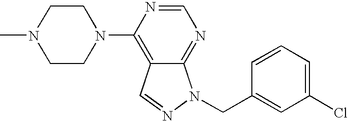 | Amorphous powder MS(APCI) 644(M + H) |

TABLE 30
(No. 4)

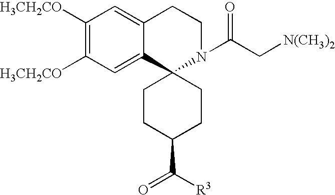

| Ex. No. | R³ | Physicochemical properties etc. |
|---|---|---|
| 521(1) | 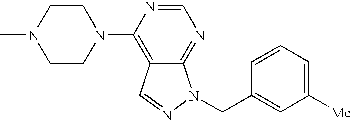 | Amorphous powder MS(APCI) 709(M + H) |
| 522(1) | 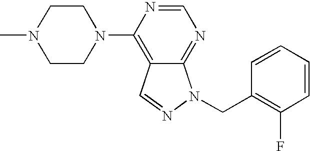 | Amorphous powder MS(APCI) 713(M + H) |
| 523(1) | 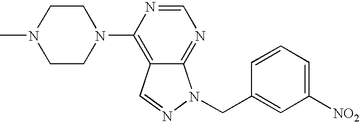 | Amorphous powder MS(APCI) 740(M + H) |
| 524(1) | 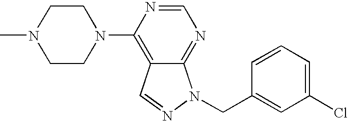 | Amorphous powder MS(APCI) 729(M + H) |
| 525(1) | 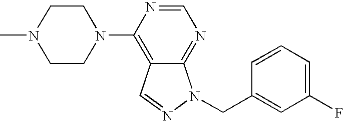 | Amorphous powder MS(APCI) 713(M + H) |
| 526(1) | 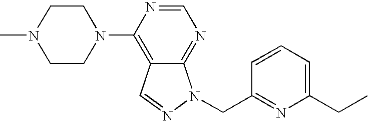 | Amorphous powder MS(APCI) 724(M + H) |

TABLE 30 (No.5)

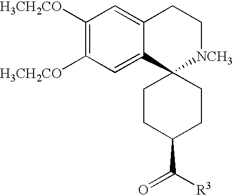

| | | Physicochemical |
|---|---|---|
| Ex. No. | R³ | properties etc. |
| 527 (1) | 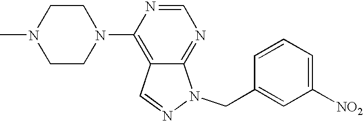 | Amorphous powder MS (APCI) 669 (M + H) |
| 528 (1) | 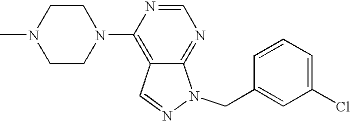 | Amorphous powder MS (APCI) 660 (M + H) |
| 529 (1) | 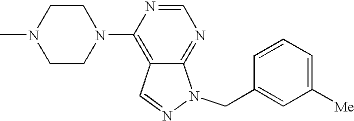 | Amorphous powder MS (APCI) 638(M + H) |
| 530 (1) | 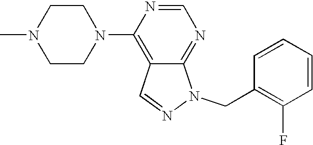 | Amorphous powder MS (APCI) 642(M + H) |
| 531 (1) | 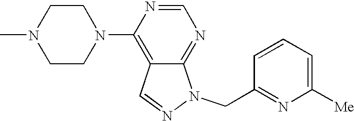 | Amorphous powder MS (APCI) 639 (M + H) |
| 532 (1) | 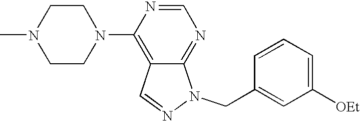 | Amorphous powder MS (APCI) 668 (M + H) |
| 533 (1) | 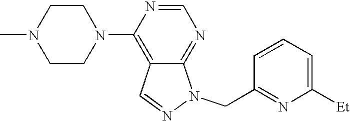 | Amorphous powder MS (APCI) 653 (M + H) |

TABLE 30 (No.6)

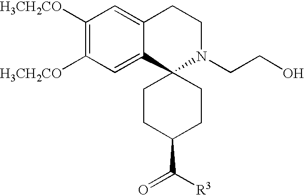

| Ex. | | Physicochemical |
|---|---|---|
| No. | R³ | properties etc. |
| 534 (1) | 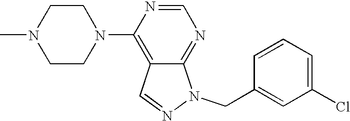 | Amorphous powder MS(APCI) 688, 690 (M + H) |
| 535 (1) | 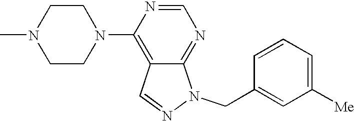 | Amorphous powder MS(APCI) 668 (M + H) |
| 536 (1) | 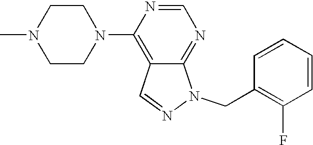 | Amorphous powder MS(APCI) 672 (M + H) |
| 537 (1) | 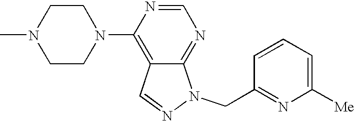 | Amorphous powder MS(APCI) 669 (M + H) |
| 538 (1) | 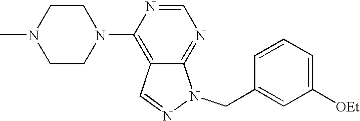 | Amorphous powder MS(APCI) 698 (M + H) |

TABLE 30 (No.7)

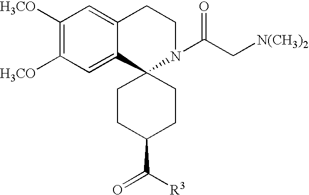

| Ex. | | Physicochemical |
|---|---|---|
| No. | R³ | properties etc. |
| 540 (1) | 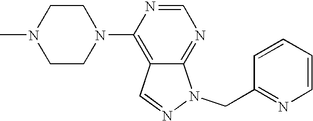 | Amorphous powder MS(APCI) 668 (M + H) |
| 541 (1) | 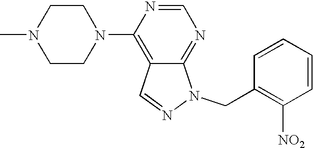 | Amorphous powder MS(APCI) 712 (M + H) |
| 542 (1) | 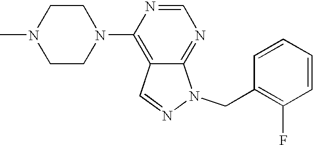 | Amorphous powder MS(APCI) 685 (M + H) |
| 543 (1) | 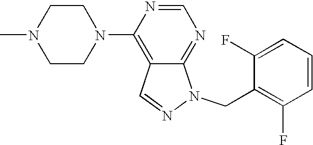 | Amorphous powder MS(APCI) 703 (M + H) |
| 544 (1) | 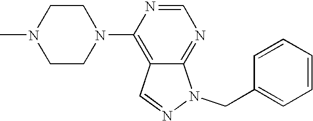 | Amorphous powder MS(APCI) 667 (M + H) |
| 545 (1) | 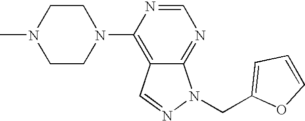 | Amorphous powder MS(APCI) 657 (M + H) |
| 546 (1) | 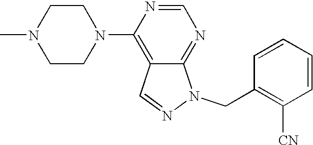 | Amorphous powder MS(APCI) 692 (M + H) |
| 547 (1) | 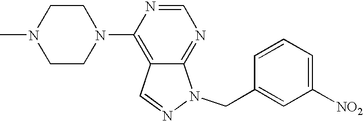 | Amorphous powder MS(APCI) 712 (M + H) |
| 548 (1) | 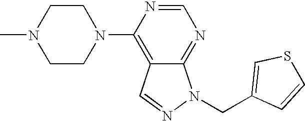 | Amorphous powder MS(APCI) 673 (M + H) |

TABLE 30 (No.8)

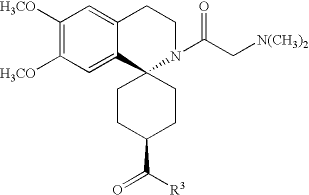

| Ex. | | Physicochemical |
|---|---|---|
| No. | R³ | properties etc. |
| 549 (1) | 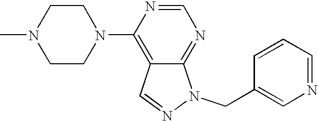 | Amorphous powder MS(APCI) 668 (M + H) |
| 550 (1) | 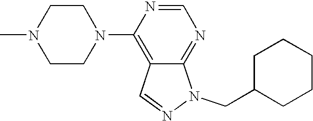 | Amorphous powder MS(APCI) 673 (M + H) |
| 551 (1) | 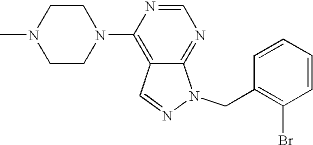 | Amorphous powder MS(APCI) 745/747 (M + H) |
| 552 (1) | 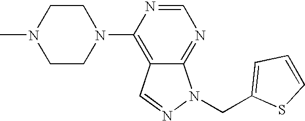 | Amorphous powder MS(APCI) 673 (M + H) |
| 553 (1) | 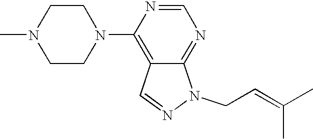 | Amorphous powder MS(APCI) 645 (M + H) |
| 554 (1) | 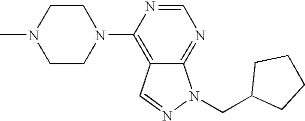 | Amorphous powder MS(APCI) 659 (M + H) |
| 555 (1) | 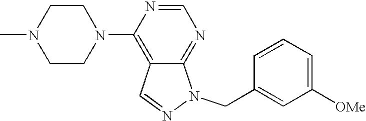 | Amorphous powder MS(APCI) 697 (M + H) |
| 556 (1) | 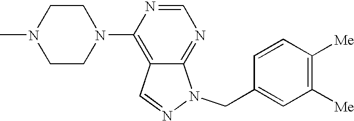 | Amorphous powder MS(APCI) 695 (M + H) IR (Nujol) 1634 |
| 557 (1) | 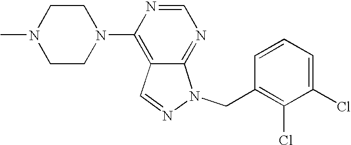 | Amorphous powder MS(APCI) 736 (M + H) IR (Nujol) 1636 |

TABLE 30 (No.9)

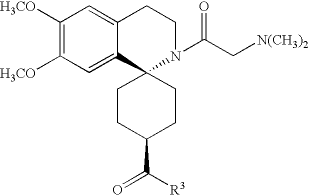

| Ex. | | Physicochemical |
|---|---|---|
| No. | R³ | properties etc. |
| 558 (1) | 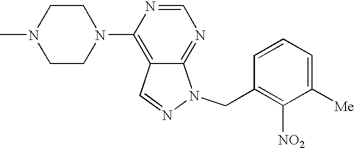 | Amorphous powder MS(APCI) 726 (M + H) IR (Nujol) 1636 |
| 559 (1) | 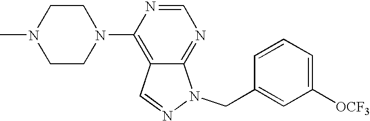 | Amorphous powder MS(APCI) 751 (M + H) IR (Nujol) 1637 |
| 560 (1) | 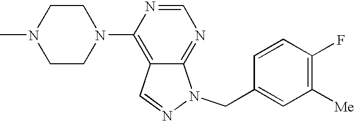 | Amorphous powder MS(APCI) 699 (M + H) IR (Nujol) 1636 |
| 561 (1) | 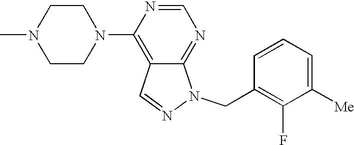 | Amorphous powder MS(APCI) 699 (M + H) IR (Nujol) 1636 |
| 562 (1) | 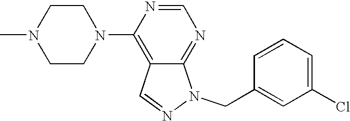 | Amorphous powder MS(APCI) 701 (M + H) IR (Nujol) 1633 |
| 563 (1) | 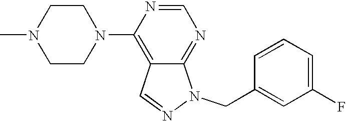 | Amorphous powder MS(APCI) 685 (M + H) IR (Nujol) 1633 |
| 564 (1) | 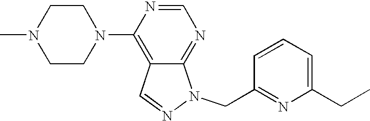 | Amorphous powder MS(APCI) 696 (M + H) |

TABLE 30 (No.10)

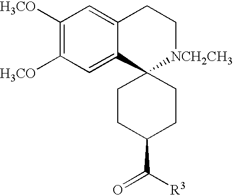

| Ex. | | Physicochemical |
|---|---|---|
| No. | R³ | properties etc. |
| 565 (1) | 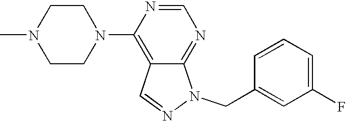 | Amorphous powder MS(APCI) 628 (M + H) |
| 566 (1) | 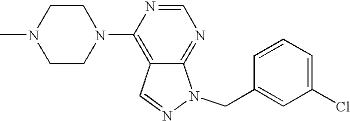 | Amorphous powder MS(APCI) 644 (M + H) |
| 567 (1) | 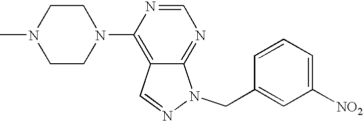 | Amorphous powder MS(APCI) 655 (M + H) |
| 568 (1) | 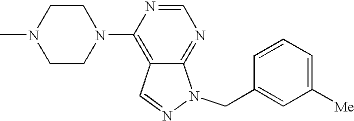 | Amorphous powder MS(APCI) 624 (M + H) |
| 569 (1) | 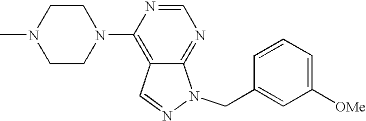 | Amorphous powder MS(APCI) 640 (M + H) |
| 570 (1) | 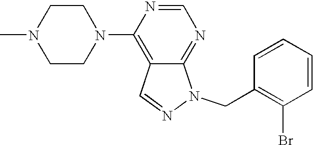 | Amorphous powder MS(APCI) 688/690 (M + H) |
| 571 (1) | 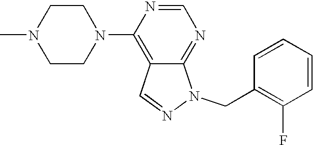 | Amorphous powder MS(APCI) 628 (M + H) |
| 572 (1) | 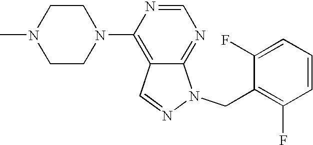 | Amorphous powder MS(APCI) 646 (M + H) |

TABLE 30 (No.11)

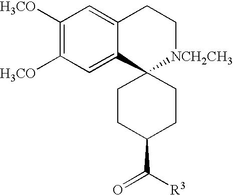

| Ex. | | Physicochemical |
|---|---|---|
| No. | R³ | properties etc. |
| 573 (1) | 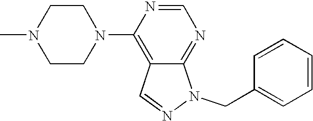 | Amorphous powder MS(APCI) 610 (M + H) |
| 574 (1) | 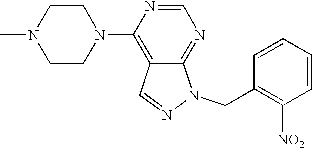 | Amorphous powder MS(APCI) 655 (M + H) |
| 575 (1) | 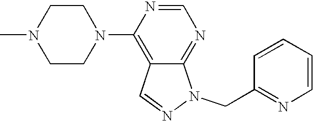 | Amorphous powder MS(APCI) 611 (M + H) |
| 576 (1) | 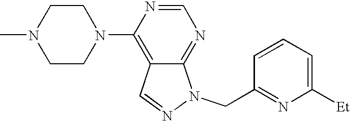 | Amorphous powder MS(APCI) 639 (M + H) |

TABLE 30 (No.12)

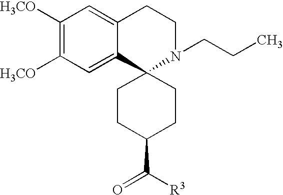

| Ex. | | Physicochemical |
|---|---|---|
| No. | R³ | properties etc. |
| 577 (1) | 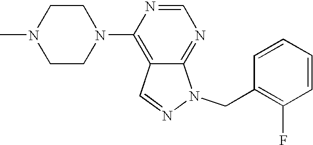 | Amorphous powder MS(APCI) 642 (M + H) IR (Nujol) 1642 |
| 578 (1) | 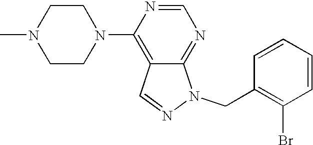 | Amorphous powder MS(APCI) 702,704 (M + H) IR (Nujol) 1641 |
| 579 (1) | 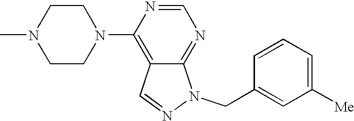 | Amorphous powder MS(APCI) 638 (M + H) IR (Nujol) 1642 |
| 580 (1) | 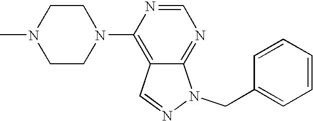 | Amorphous powder MS(APCI) 624 (M + H) IR (Nujol) 1641 |
| 581 (1) | 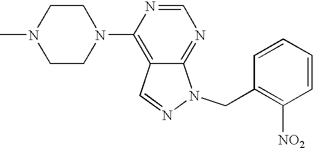 | Amorphous powder MS(APCI) 669 (M + H) |
| 582 (1) | 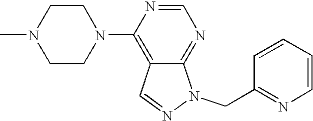 | Amorphous powder MS(APCI) 625 (M + H) |

TABLE 30 (No.13)

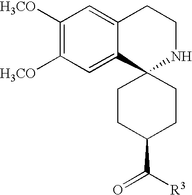

| Ex. | | Physicochemical |
|---|---|---|
| No. | R³ | properties etc. |
| 583(1) | 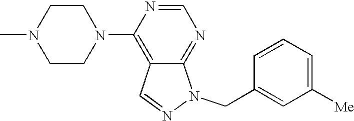 | Amorphous powder MS(APCI) 596 (M + H) IR (Nujol) 1637 |
| 584(1) | 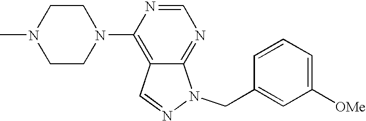 | Amorphous powder MS(APCI) 612 (M + H) IR (Nujol) 1637 |
| 585(1) | 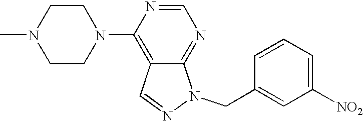 | Amorphous powder MS(APCI) 627 (M + H) IR (Nujol) 1636 |
| 586(1) | 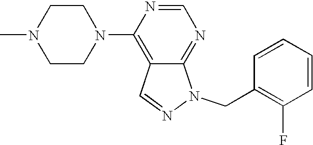 | Amorphous powder MS(APCI) 600 (M + H) IR (Nujol) 1637 |
| 587(1) | 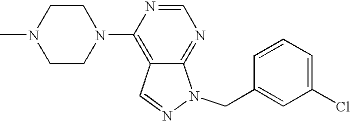 | Amorphous powder MS(APCI) 616 (M + H) IR (Nujol) 1637 |
| 588(1) | 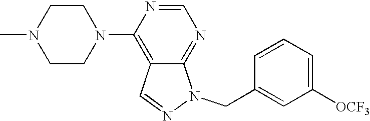 | Amorphous powder MS(APCI) 666 (M + H) IR (Nujol) 1642 |
| 589(1) | 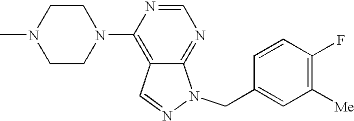 | Amorphous powder MS(APCI) 614 (M + H) IR (Nujol) 1639 |
| 590(1) | 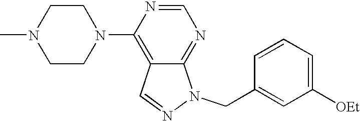 | Amorphous powder MS(APCI) 711 (M + H) IR (Nujol) 1633 |

TABLE 30 (No.14)

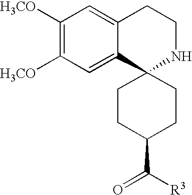

| Ex. | | Physicochemical |
|---|---|---|
| No. | R³ | properties etc. |
| 591(1) | 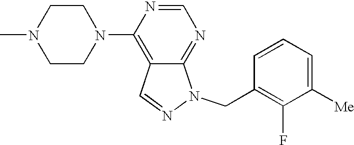 | Amorphous powder MS(APCI) 614 (M + H) IR (Nujol) 1640 |
| 592(1) | 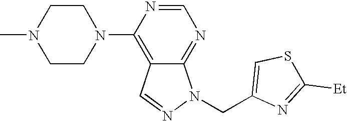 | Amorphous powder MS(APCI) 617 (M + H) IR (Nujol) 1637 |
| 593(1) | 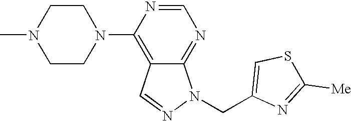 | Amorphous powder MS(APCI) 603 (M + H) IR (Nujol) 1637 |
| 594(1) | 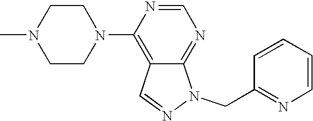 | Amorphous powder MS(APCI) 583 (M + H) IR (Nujol) 1633 |
| 595(1) | 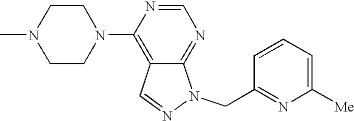 | Amorphous powder MS(APCI) 597 (M + H) |
| 596(1) | 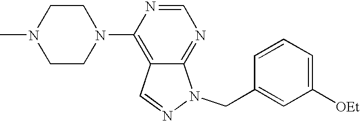 | Amorphous powder MS(APCI) 626 (M + H) |
| 597(1) | 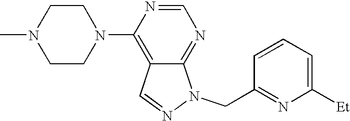 | Amorphous powder MS(APCI) 611 (M + H) |

TABLE 30
(No. 15)

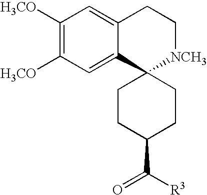

| Ex. | | Physicochemical |
|---|---|---|
| No. | R³ | properties etc. |
| 598 (1) | 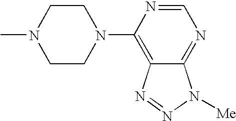 | M.p. 189-191° C. MS (APCI) 521 (M + H) |
| 599 (1) | 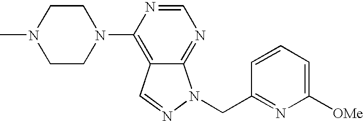 | Amorphous powder MS (APCI) 610 (M + H) |
| 600 (1) | 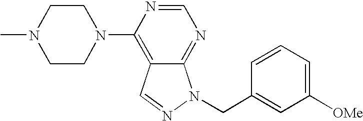 | Amorphous powder MS (APCI) 626 (M + H) |
| 601 (1) | 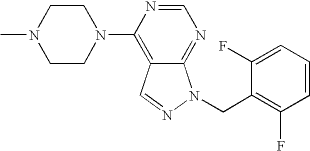 | Amorphous powder MS (APCI) 632 (M + H) |
| 602 (1) | 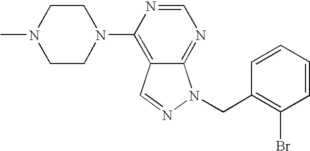 | Amorphous powder MS (APCI) 674/676 (M + H) |
| 603 (1) | 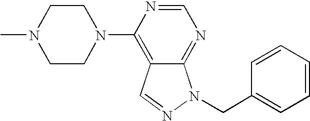 | Amorphous powder MS (APCI) 596 (M + H) |
| 604 (1) | 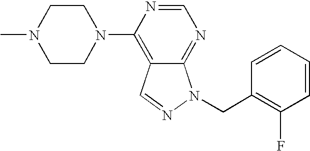 | Amorphous powder MS (APCI) 614 (M + H) |
| 605 (1) | 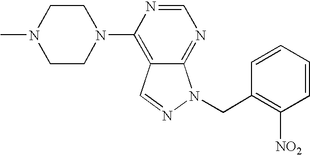 | Amorphous powder MS (APCI) 641 (M + H) |

TABLE 30
(No. 16)

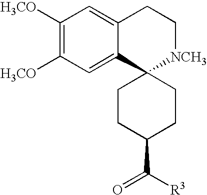

| Ex. | | Physicochemical |
|---|---|---|
| No. | R³ | properties etc. |
| 606 (1) | 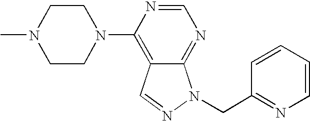 | Amorphous powder MS (APCI) 597 (M + H) |
| 607 (1) | 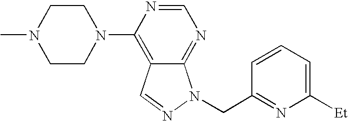 | Amorphous powder MS (APCI) 625 (M + H) |

TABLE 30
(No.17)

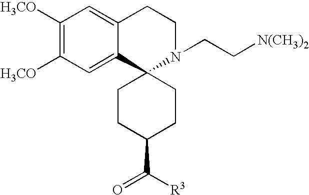

| Ex. | | Physicocheniical |
|---|---|---|
| No. | R³ | properties etc. |
| 608 (1) | 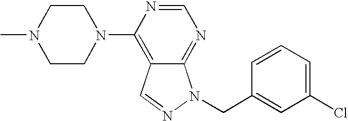 | Amorphous powder MS (APCI) 687 (M + H) |
| 609 (1) | 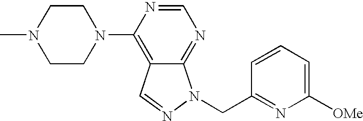 | Amorphous powder MS (APCI) 668 (M + H) |
| 610 (1) | 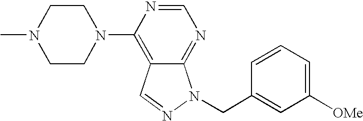 | Amorphous powder MS (APCI) 683 (M + H) |
| 611 (1) | 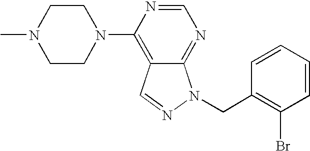 | Amorphous powder MS (APCI) 731/733 (M + H) |
| 612 (1) | 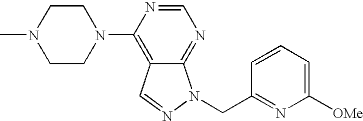 | Amorphous powder MS (APCI) 667 (M + H) |
| 613 (1) | 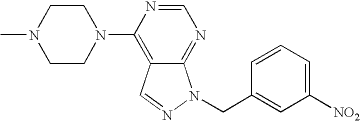 | Amorphous powder MS (APCI) 698 (M + H) |
| 614 (1) | 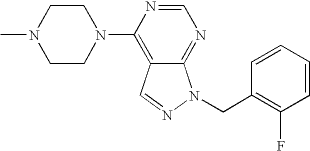 | Amorphous powder MS (APCI) 671 (M + H) |

TABLE 30
(No.18)

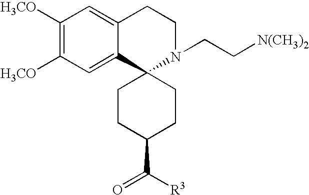

| Ex. | | Physicochemical |
|---|---|---|
| No. | R³ | properties etc. |
| 615 (1) | 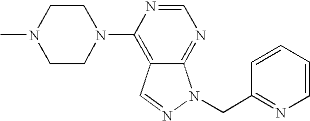 | Amorphous powder MS (APCI) 654 (M + H) |
| 616 (1) | 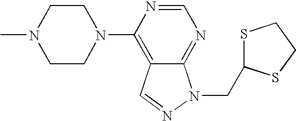 | Amorphous powder MS (APCI) 681 (M + H) |
| 617 (1) | 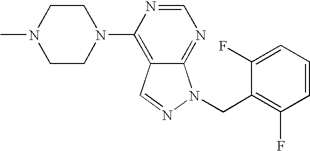 | Amorphous powder MS (APCI) 689 (M + H) |
| 618 (1) | 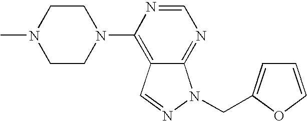 | Amorphous powder MS (APCI) 643 (M + H) |
| 619 (1) | 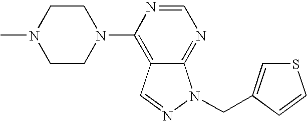 | Amorphous powder MS (APCI) 659 (M + H) |
| 620 (1) | 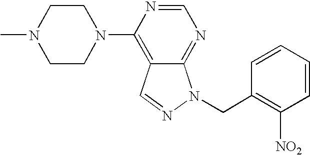 | Amorphous powder MS (APCI) 698 (M + H) |
| 621 (1) | 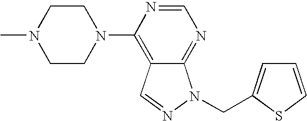 | Amorphous powder MS (APCI) 659 (M + H) |
| 622 (1) | 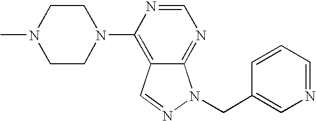 | Amorphous powder MS (APCI) 654 (M + H) |

TABLE 30
(No. 19)

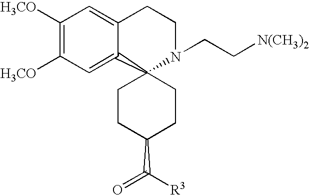

| Ex. | | Physicochemical |
|---|---|---|
| No. | R³ | properties etc. |
| 623 (1) | 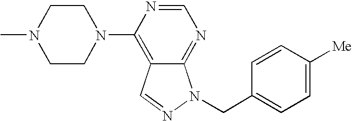 | Amorphous powder MS (APCI) 667 (M + H) |
| 624 (1) | 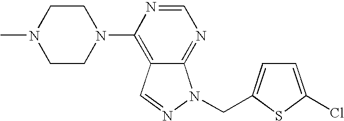 | Amorphous powder MS (APCI) 693 (M + H) |
| 625 (1) | 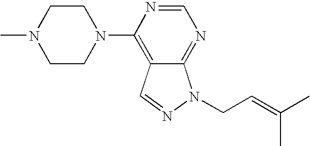 | Amorphous powder MS (APCI) 631 (M + H) |
| 626 (1) | 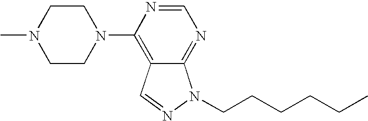 | Amorphous powder MS (APCI) 647 (M + H) |
| 627 (1) | 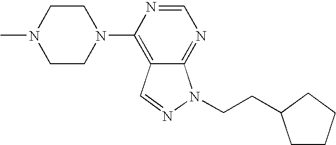 | Amorphous powder MS (APCI) 659 (M + H) |
| 628 (1) | 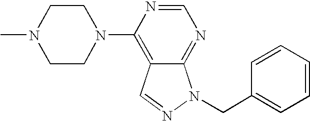 | Amorphous powder MS (APCI) 653 (M + H) |
| 629 (1) | 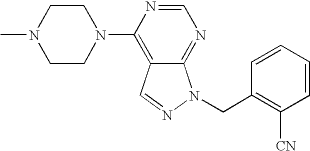 | Amorphous powder MS (APCI) 678 (M + H) |
| 630 (1) | 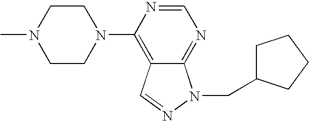 | Amorphous powder MS (APCI) 645 (M + H) |

TABLE 30
(No. 20)

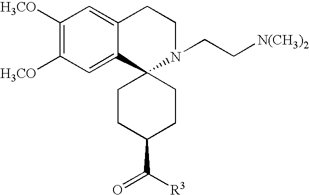

| Ex. | | Physicochemical |
|---|---|---|
| No. | R³ | properties etc. |
| 631 (1) | 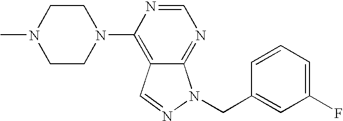 | Amorphous powder MS (APCI) 671 (M + H) |
| 632 (1) | 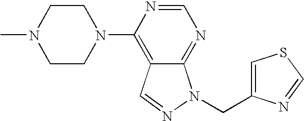 | Amorphous powder MS (APCI) 660 (M + H) |

TABLE 30
(No. 21)

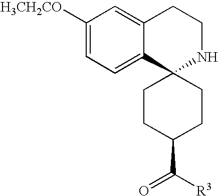

| Ex. | | Physicochemical |
|---|---|---|
| No. | R³ | properties etc. |
| 633 (1) | 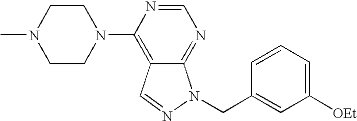 | Amorphous powder MS (APCI) 610 (M + H) |
| 634 (1) | 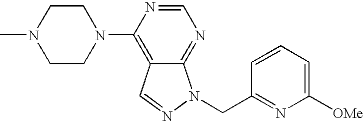 | Amorphous powder MS (APCI) 580 (M + H) |
| 635 (1) | 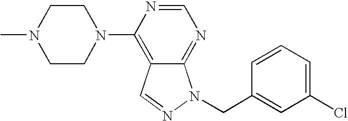 | Amorphous powder MS (APCI) 600/602 (M + H) |
| 636 (1) | 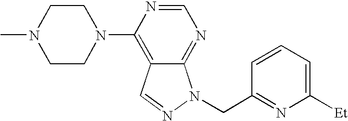 | Amorphous powder MS (APCI) 595 (M + H) |

2) The compounds obtained in Examples 504(1) to 636(1) are treated in the same manner as described in Example 2(2) to give the following compounds (salt).

Example 539(2): 1 Fumaric acid salt of the compound obtained in Example 539(1); MS(APCI)m/z: 681(M+H), IR(Nujol)cm⁻¹: 3407, 1650, 1634

Example 540(2): 1 Fumaric acid salt of the compound obtained in Example 540(1); Amorphous powder, MS(APCI)m/z: 668(M+H), IR(Nujol)cm⁻¹: 1638, 1573

Example 541(2): 1 Fumaric acid salt of the compound obtained in Example 541(1); Amorphous powder, MS(APCI)m/z: 712(M+H), IR(Nujol)cm⁻¹: 1641, 1573

Example 542(2): 1 Fumaric acid salt of the compound obtained in Example 542(1); Amorphous powder, MS(APCI)m/z: 685(M+H), IR(Nujol)cm⁻¹: 1638, 1573

Example 543(2): 1 Fumaric acid salt of the compound obtained in Example 543(1); Amorphous powder, MS(APCI)m/z: 703(M+H), IR(Nujol)cm⁻¹: 1643, 1627

Example 544 (2): 1 Fumaric acid salt of the compound obtained in Example 544(1); Amorphous powder, MS(APCI)m/z: 667(M+H), IR(Nujol)cm⁻¹: 1643, 1573

Example 545(2): 1 Fumaric acid salt of the compound obtained in Example 545(1); Amorphous powder, MS(APCI)m/z: 657(M+H), IR(Nujol)cm⁻¹: 1639, 1574

Example 546(2): 1 Fumaric acid salt of the compound obtained in Example 546(1); Amorphous powder, MS(APCI)m/z: 692(M+H), IR(Nujol)cm⁻¹: 1642, 1573

Example 547(2): 1 Fumaric acid salt of the compound obtained in Example 547(1); Amorphous powder, MS(APCI)m/z: 712(M+H), IR(Nujol)cm⁻¹: 1641, 1573

Example 548(2): 1 Fumaric acid salt of the compound obtained in Example 548(1); Amorphous powder, MS(APCI)m/z: 673(M+H), IR(Nujol)cm⁻¹: 1641, 1574

Example 549(2): 1 Fumaric acid salt of the compound obtained in Example 549(1); Amorphous powder, MS(APCI)m/z: 668(M+H), IR(Nujol)cm⁻¹: 1640, 1573

Example 550(2): 1 Fumaric acid salt of the compound obtained in Example 550(1); Amorphous powder, MS(APCI)m/z: 673(M+H), IR(Nujol)cm⁻¹: 1643, 1573

Example 551(2): 1 Fumaric acid salt of the compound obtained in Example 551(1); Amorphous powder, MS(APCI)m/z: 745/747(M+H), IR(Nujol)cm⁻¹: 1640, 1573

Example 552(2): 1 Fumaric acid salt of the compound obtained in Example 552(1); Amorphous powder, MS(APCI)m/z: 673(M+H), IR(Nujol)cm⁻¹: 1703, 1641

Example 553(2): 1 Fumaric acid salt of the compound obtained in Example 553(1); Amorphous powder, MS(APCI)m/z: 645(M+H), IR(Nujol)cm⁻¹: 1641, 1573

Example 554(2): 1 Fumaric acid salt of the compound obtained in Example 554(1); Amorphous powder, MS(APCI)m/z: 659(M+H), IR(Nujol)cm⁻¹: 1699, 1641

Example 555(2): 1 Fumaric acid salt of the compound obtained in Example 555(1); Amorphous powder, MS(APCI)m/z: 697(M+H)

Example 574(2): 1 Fumaric acid salt of the compound obtained in Example 574(1); Amorphous powder, MS(APCI)m/z: 655(M+H), IR(Nujol)cm⁻¹: 1705, 1634

Example 575(2): 1 Fumaric acid salt of the compound obtained in Example 575(1); Amorphous powder, MS(APCI)m/z: 611(M+H), IR(Nujol)cm⁻¹: 1704, 1634

Example 581(2): 1 Fumaric acid salt of the compound obtained in Example 581(1); Amorphous powder, MS(ESI)m/z: 669(M+H), IR(Nujol)cm⁻¹: 1705, 1636

Example 582(2): 1 Fumaric acid salt of the compound obtained in Example 582(1); Amorphous powder, MS(ESI)m/z: 625(M+H), IR(Nujol)cm⁻¹: 1704, 1635

Example 594(2): 1 Fumaric acid salt of the compound obtained in Example 594(1); Amorphous powder, MS(APCI)m/z: 583(M+H), IR(Nujol)cm⁻¹: 1703, 1633

Example 605(2): 1 Fumaric acid salt of the compound obtained in Example 605(1); Amorphous powder, MS(APCI)m/z: 641(M+H), IR(Nujol)cm⁻¹: 1705, 1636

Example 606(2): 1 Fumaric acid salt of the compound obtained in Example 606(1); Amorphous powder, MS(APCI)m/z: 597(M+H), IR(Nujol)cm⁻¹: 1704, 1634

### Example 637

To a mixture of (1α,4β)-3′,4′-dihydro-6′-ethoxy-4-[4-[1-(3-ethoxyphenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 633(1)) (150 mg), tetrahydrofuran (3 mL) and acetic acid (0.3 mL) is added aqueous formaldehyde (41 μL) and sodium triacetoxyborohydride (78 mg) and the mixture is stirred at room temperature for 2 hours. The reaction mixture is poured into saturated aqueous sodium hydrogencarbonate solution and extracted with ethyl acetate. The extract is washed with water and saturated aqueous NaCl solution. The organic layer is dried over sodium sulfate and concentrated. The residue is purified by column chromatography on NH-silica gel (solvent; chloroform: methanol=20:1) and lyophilized to give (1α,4β)-3′,4′-dihydro-6′-ethoxy-2′-methyl-4-[4-[1-(3-ethoxyphenylmethyl)-1H-pyrazolo[3,4-d]pyrimidin-4-yl]-1-piperazinyl]carbonyl-spiro[cyclohexane-1,1′(2′H)-isoquinoline] (133 mg, 86.7%).

MS(APCI)m/z: 624(M+H), IR(Nujol)cm⁻¹: 3445, 1640, 1570, 1460, 1375, 1000.

### Examples 638 to 640

The corresponding materials are treated in the same manner as described in Example 637 to give the compounds as shown in the following table (Table.31).

TABLE 32

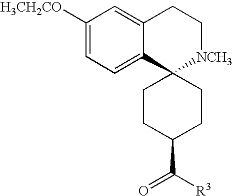

| Ex. | | Physicochemical |
|---|---|---|
| No. | R³ | properties etc. |
| 638 | 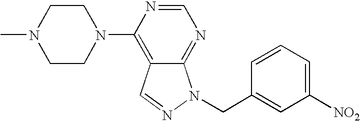 | Amorphous powder MS (APCI) 625 (M + H) |
| 639 | 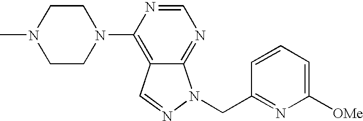 | Amorphous powder MS (APCI) 594 (M + H) |
| 640 | 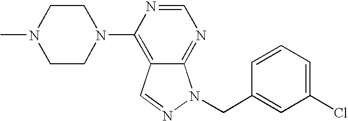 | Amorphous powder MS (APCI) 614/616 (M + H) |

### Example 641

To a solution of (1α,4β)-3′,4′-dihydro-6′-ethoxy-4-[4-[1-(3-ethoxyphenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 633(1)) (150 mg) in acetonitrile (3 mL) are added diisopropylethylamine (129 μL) and chloroacetyl chloride (39 μL) under ice-cooling and the mixture is stirred at room temperature for 2 hours. To the reaction mixture is added aqueous dimethylamine (1 mL) and the mixture is stirred at room temperature for 2 hours. The reaction mixture is diluted with ethyl acetate and washed with water, saturated aqueous sodium hydrogencarbonate solution, and saturated aqueous NaCl solution. The organic layer is dried over sodium sulfate and concentrated. The residue is purified by column chromatography on silica gel (solvent; chloroform→chloroform:methanol=9:1) and lyophilized with tert-butyl alcohol to give (1α,4β)-3′,4′-dihydro-6′-ethoxy-2′-dimethylaminoacetyl-4-[4-[1-(3-ethoxyphenylmethyl)-1H-pyrazolo[3,4-d]pyrimidin-4-yl]-1-piperazinyl]carbonyl-spiro[cyclohexane-1,1′(2′H)-isoquinoline] (94 mg,55%) as an amorphous powder.

MS(APCI)m/z: 695(M+H), IR(Nujol)cm⁻¹: 3465, 1635, 1570, 1460, 1000.

### Examples 642-647

The corresponding materials are treated in the same manner as described in Example 641 to give the compounds as shown in the following table (Table.32).

TABLE 32

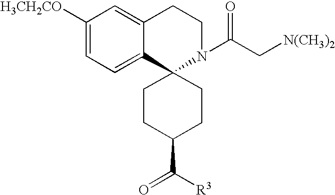

| Ex. | | Physicochemical |
|---|---|---|
| No. | R³ | properties etc. |
| 642 | 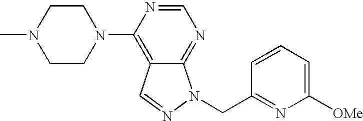 | Amorphous powder MS (APCI) 665 (M + H) |
| 643 | 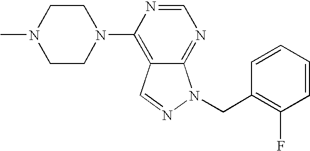 | Amorphous powder MS (APCI) 669 (M + H) |
| 644 | 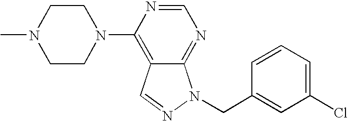 | Amorphous powder MS (APCI) 685/687 (M + H) |
| 645 | 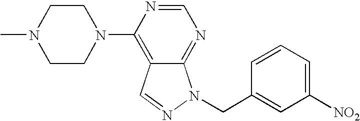 | Amorphous powder MS (APCI) 696 (M + H) |
| 646 | 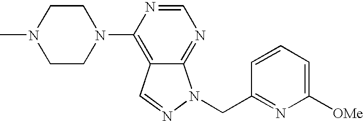 | Amorphous powder MS (APCI) 666 (M + H) |
| 647 | 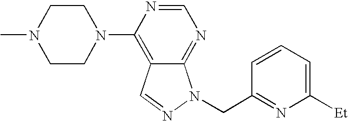 | Amorphous powder MS (APCI) 680 (M + H) |

### Example 648

To a mixture of (1α,4β)-3′,4′-dihydro-6′-ethoxy-4-[4-[1-(6-ethyl-2-pyridylmethyl)-1H-pyrazolo[3,4-d]pyrimidin-4-yl]-1-piperazinyl]carbonyl-spiro[cyclohexane-1,1′(2′H)-isoquinoline] (compound obtained in Example 636(1)) (150 mg) in dimethylacetamide (5 mL) are added potassium carbonate (174 mg) and iodoethane (101 μL) and the mixture is stirred at 100° C. for 2 hours. After cooling ethyl acetate is added to the mixture. The mixture is washed with water and saturated aqueous NaCl solution, successively. The organic layer is dried and concentrated. The residue is purified by column chromatography on NH-silica gel (solvent; ethyl acetate:n-hexan=1:1→4:1) and lyophilized with tert-butyl alcohol to give (1α,4β)-3′,4′-dihydro-6′-ethoxy-2′-ethyl-4-[4-[1-(6-ethyl-2-pyridylmethyl)-1H-pyrazolo[3,4-d]pyrimidin-4-yl]-1-piperazinyl]carbonyl-spiro[cyclohexane-1,1′(2′H)-isoquinoline] (124 mg, 79%) as an amorphous powder.

MS(APCI)m/z: 623(M+H), IR(Nujol)cm⁻¹: 1640, 1570.

### Examples 649

The corresponding materials are treated in the same manner as described in Example 648 to give (1α,4β)-3′,4′-dihydro-6′-ethoxy-2′-ethyl-4-[4-[1-(6-ethyl-2-pyridylmethyl)-1H-pyrazolo[3,4-d]pyrimidin-4-yl]-1-piperazinyl]carbonyl-spiro[cyclohexane-1,1′(2′H)-isoquinoline] as an amorphous powder.

MS(APCI)m/z: 623(M+H).

### Examples 650 to 661

The corresponding materials are treated in the same manner as described in Example 52(1) to give the compounds as shown in the following table (Table.33).

TABLE 33
(No. 1)

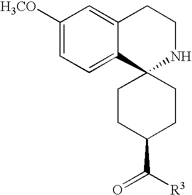

| Ex. | | Physicochemical |
|---|---|---|
| No. | R³ | properties etc. |
| 650 | 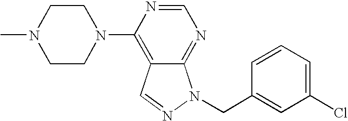 | Amorphous powder MS (APCI) 586/588 (M + H) |
| 651 | 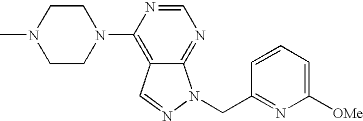 | Amorphous powder MS (APCI) 566 (M + H) |
| 652 | 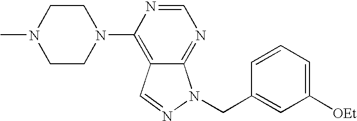 | Amorphous powder MS (APCI) 596 (M + H) |
| 653 | 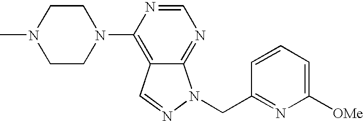 | Amorphous powder MS (APCI) 567 (M + H) |
| 654 | 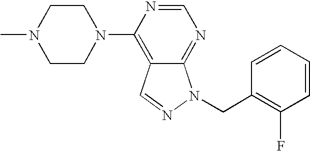 | Amorphous powder MS (APCI) 570 (M + H) |
| 655 | 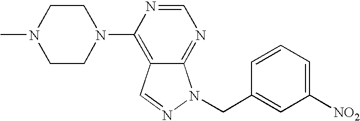 | Amorphous powder MS (APCI) 597 (M + H) |

TABLE 33
(No. 2)

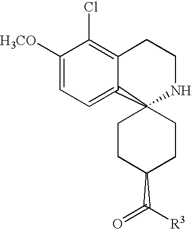

| Ex. No. | R³ | Physicochernical properties etc. |
|---|---|---|
| 656 | 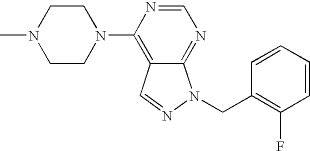 | Amorphous powder MS(APCI) 604/606(M + H) |
| 657 | 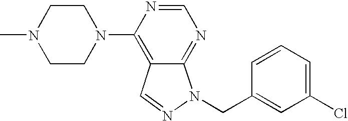 | Amorphous powder MS(APCI) 620/622(M + H) |
| 658 | 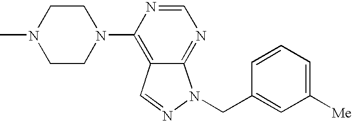 | Amorphous powder MS(APCI) 600/602(M + H) |
| 659 | 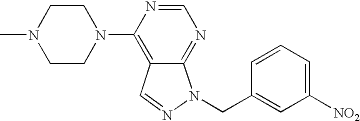 | Amorphous powder MS(APCI) 631/633(M + H) |
| 660 | 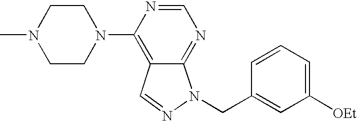 | Amorphous powder MS(APCI) 630/632(M + H) |
| 661 | 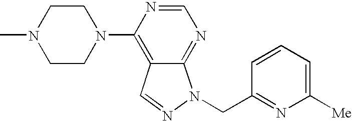 | Amorphous powder MS(APCI) 601/603(M + H) |

### Examples 662 to 667

The corresponding materials are treated in the same manner as described in Example 648 to give the compounds as shown in the following table (Table.34).

TABLE 34

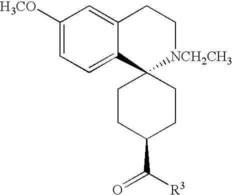

| EX. No. | R³ | Physicochemical properties etc. |
|---|---|---|
| 662 | 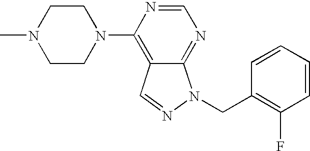 | Amorphous powder MS(APCI) 598(M + H) |
| 663 | 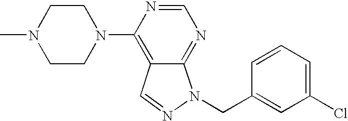 | Amorphous powder MS(APCI) 614/616(M + H) |
| 664 | 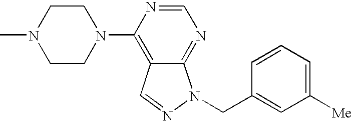 | Amorphous powder MS(APCI) 594(M + H) |
| 665 | 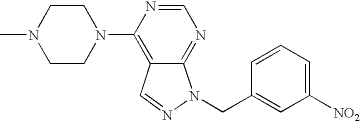 | Amorphous powder MS(APCI) 625(M + H) |
| 666 | 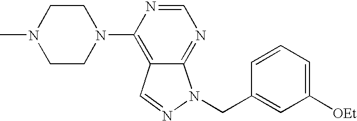 | Amorphous powder MS(APCI) 624(M + H) |
| 667 | 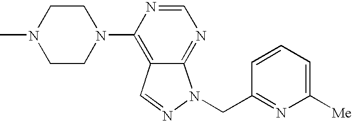 | Amorphous powder MS(APCI) 595(M + H) |

### Examples 668 to 673

The corresponding materials are treated in the same manner as described in Example 637 to give the compounds as shown in the following table (Table.35).

TABLE 35

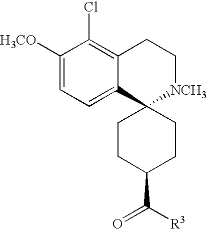

| EX. No. | R³ | Physicochemical properties etc. |
|---|---|---|
| 668 | 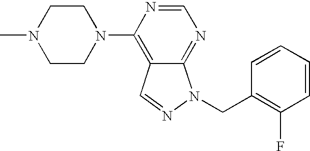 | Amorphous powder MS(APCI) 618/620(M + H) |
| 669 | 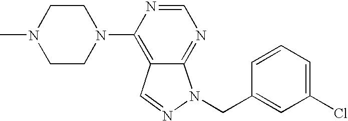 | Amorphous powder MS(APCI) 634/636(M + H) |
| 670 | 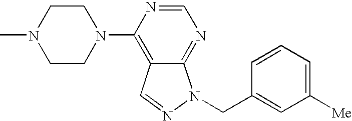 | Amorphous powder MS(APCI) 614/616(M + H) |
| 671 | 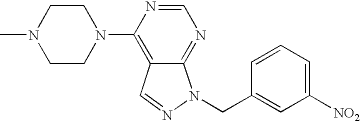 | Amorphous powder MS(APCI) 645/647(M + H) |
| 672 | 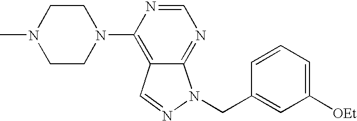 | Amorphous powder MS(APCI) 644/646(M + H) |
| 673 | 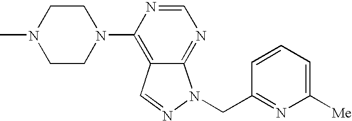 | Amorphous powder MS(APCI) 629/631(M + H) |

### Examples 674 to 685

The corresponding materials are treated in the same manner as described in Example 641 to give the compounds as shown in the following tables (Table.36).

TABLE 36
(No. 1)

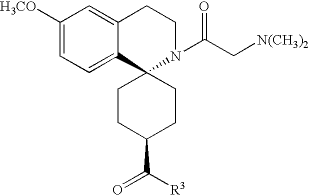

| EX. No. | R³ | Physicochemical properties etc. |
|---|---|---|
| 674 | 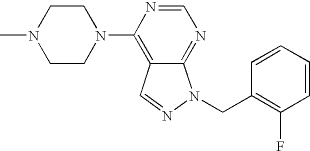 | Amorphous powder MS(APCI) 655(M + H) |
| 675 | 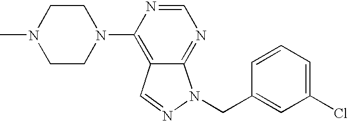 | Amorphous powder MS(APCI) 671/673(M + H) |
| 676 | 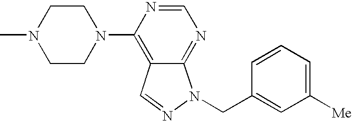 | Amorphous powder MS(APCI) 651(M + H) |
| 677 | 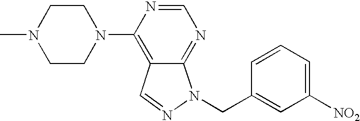 | Amorphous powder MS(APCI) 682(M + H) |
| 678 | 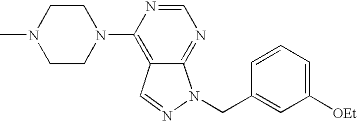 | Amorphous powder MS(APCI) 681(M + H) |
| 679 | 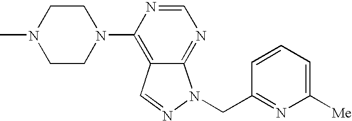 | Amorphous powder MS(APCI) 652(M + H) |

TABLE 36
(No. 2)

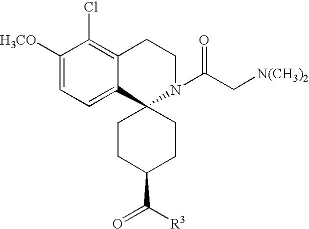

| EX. No. | R³ | Physicochemical properties etc. |
|---|---|---|
| 680 | 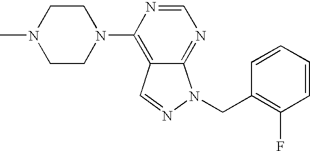 | Amorphous powder MS(APCI) 689/691(M + H) |
| 681 | 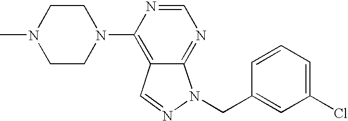 | Amorphous powder MS(APCI) 705/707(M + H) |
| 682 | 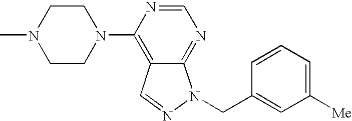 | Amorphous powder MS(APCI) 685/687(M + H) |
| 683 | 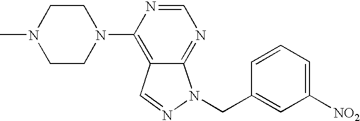 | Amorphous powder MS(APCI) 716/718(M + H) |
| 684 | 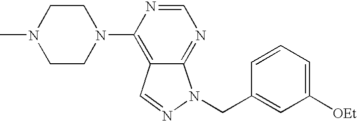 | Amorphous powder MS(APCI) 715/717(M + H) |
| 685 | 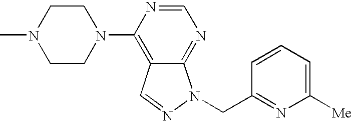 | Amorphous powder MS(APCI) 686/688(M + H) |

### Example 686

1) A mixture of (1α,4β)-2′-(3-aminopropyl)-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (100 mg), chloroform (2 mL), water (1 mL), sodium hydrogencarbonate (37 mg) methyl chloroformate (17 μL) is stirred under ice-cooling for 1 hour. To the reaction mixture is added chloroform and the mixture is washed with water. The organic layer is dried over sodium sulfate and concentrated. The residue is purified by column chromatography on silica gel (solvent; chloroform:methanol:aqueous ammonia=20:1:0.1) to give (1α,4β)-2′-(3-methoxycarbonyl-aminopropyl)-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′)-isoquinoline] (94 mg) as an amorphous powder.

MS(APCI)m/z: 742(M+H), IR(Nujol)cm⁻¹: 3328, 1718, 1637.

2) The compound obtained in the above step (1) is treated in the same manner as described in Example 2(2) to give 1 fumaric acid salt thereof.

MS(APCI)m/z: 742(M+H), IR(Nujol)cm⁻¹: 1713, 1635.

### Examples 687 to 695

The corresponding materials are treated in the same manner as described in Example 686(1) to give the compounds as shown in the following tables (Table.37).

TABLE 37
(No. 1)

| Ex. No. | R | R³ | Physicochemical properties etc. |
|---|---|---|---|
| 687(1) | COOMe | 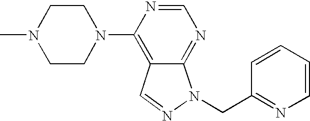 | Amorphous powder MS(APCI) 698(M + H) |
| 688(1) | COOEt | 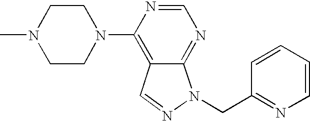 | Amorphous powder MS(APCI) 712(M + H) |
| 689(1) | COOEt | 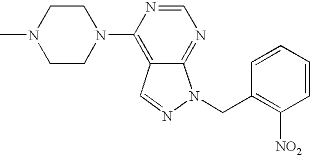 | Amorphous powder MS(APCI) 756(M + H) |
| 690(1) | COOEt | 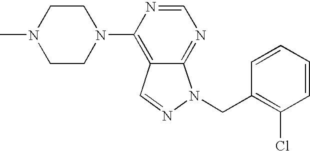 | Amorphous powder MS(APCI) 699(M + H) |
| 691(1) | COOEt | 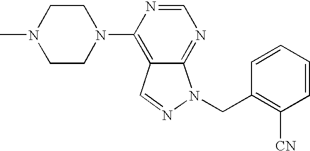 | Amorphous powder MS(APCI) 736(M + H) |
| 692(1) | COOEt | 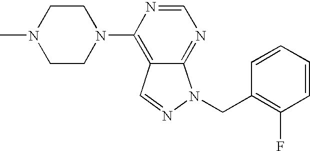 | Amorphous powder MS(APCI) 729(M + H) |
| 693(1) | COOn-Bu | 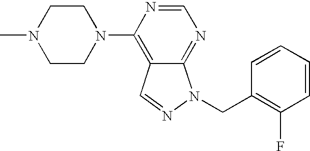 | Amorphous powder MS(APCI) 757(M + H) IR(Nujol) 1711, 1637 |

TABLE 37
(No. 2)

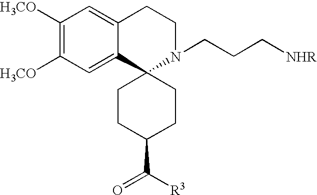

| Ex. No. | R | R³ | Physicochemical properties etc. |
|---|---|---|---|
| 694(1) | COOEt | 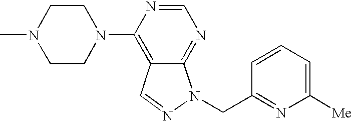 | Amorphous powder MS(APCI) 726(M + H) |
| 695(1) | COOEt | 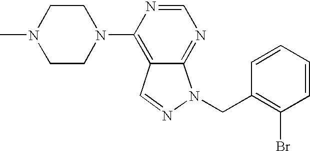 | Amorphous powder MS(APCI) 789 M + H) IR(Nujol) 1701, 1636 |

2) The compound obtained in the above steps (1) are treated in the same manner as described in Example 2(2) to give the following compounds (salt).

Example 687(2): 1 Fumaric acid salt of the compound obtained in Example 687(1); Amorphous powder, MS(APCI)m/z: 698(M+H), IR(Nujol)cm⁻¹: 1702, 1636

Example 693(2): 2 Fumaric acid salt of the compound obtained in Example 693(1); Amorphous powder, MS(APCI)m/z: 757(M+H), IR(Nujol)cm⁻¹: 1707, 1635, 1607

### Example 696

1) (1α,4β)-2′-(3-Aminopropyl)-3′,4′-dihydro-6′,7′-dimethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline] (compound obtained in Example 282(1)) (250 mg) is dissolved in methylene chloride and to the solution are added ethoxyacetic acid (55 μL), 1-ethyl-3-(3-dimethylaminopropyl)carbodiimide hydrochloride (112 mg),1-hydroxybenzotriazole (78 mg) and triethylamine (81 μL). The mixture is stirred at room temperature for 18 hours. To the reaction mixture is added saturated aqueous sodium hydrogencarbonate solution (20 mL) and the mixture is extracted with ethyl acetate. The extract is washed with water and saturated aqueous NaCl solution, dried over sodium sulfate, and concentrated. The residue is purified by column chromatography on silica gel (solvent; chloroform:methanol:aqueous ammonia=190:10:1) to give (1α,4β)-2′-[3-(ethoxyacetamido)propyl]-3′,4′-dihydro-6′,7′-dimethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline] (179 mg) as an amorphous powder.

MS(APCI)m/z: 726(M+H), IR(Nujol)cm⁻¹: 3415, 1653, 1637.

### Examples 697 to 709

1) The corresponding materials are treated in the same manner as described in Example 696 to give the compounds as shown in the following tables (Table.38).

TABLE 38
(No. 1)

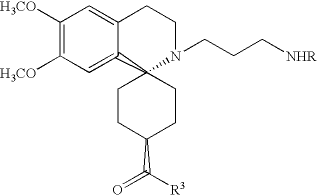

| Ex. No. | R | R³ | Physicochemical properties etc. |
|---|---|---|---|
| 697(1) | 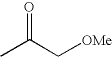 | 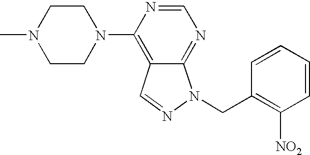 | Amorphous powder MS(APCI) 756(M + H) |
| 698(1) | 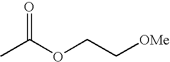 | 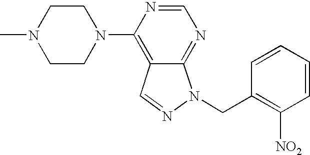 | Amorphous powder MS(APCI) 786(M + H) |
| 699(1) | 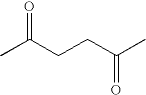 | 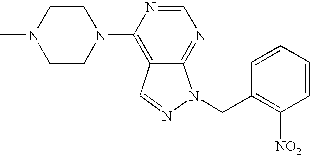 | Amorphous powder MS(APCI) 782(M + H) |
| 700(1) | 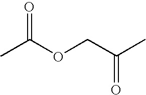 | 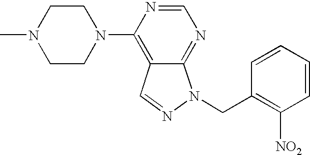 | Amorphous powder MS(APCI) 784(M + H) |
| 701(1) | 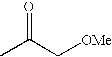 | 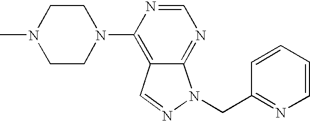 | Amorphous powder MS(APCI) 712(M + H) |
| 702(1) | 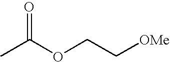 | 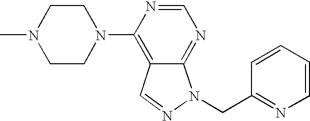 | Amorphous powder MS(APCI) 742(M + H) |
| 703(1) | 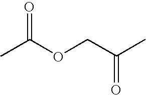 | 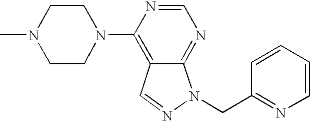 | Amorphous powder MS(APCI) 740(M + H) |

TABLE 38
(No. 2)

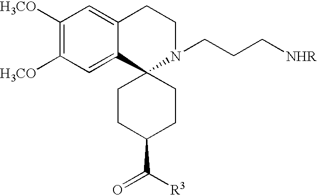

| Ex. No. | R | R³ | Physicochemical properties etc. |
|---|---|---|---|
| 704(1) | 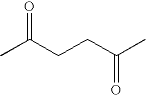 | 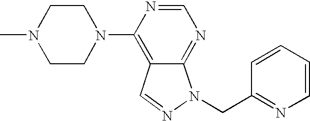 | Amorphous powder MS(APCI) 738(M + H) |
| 705(1) | 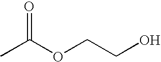 | 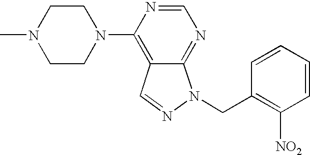 | Amorphous powder MS(APCI) 772(M + H) |
| 706(1) | 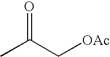 | 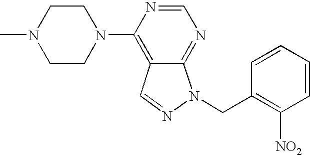 | Amorphous powder MS(APCI) 784(M + H) |
| 707(1) | 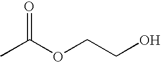 | 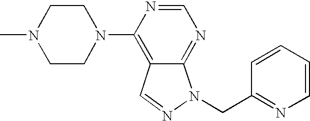 | Amorphous powder MS(APCI) 728(M + H) |
| 708(1) | 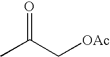 | 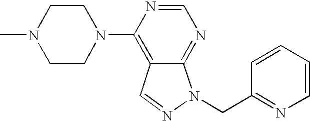 | Amorphous powder MS(APCI) 740(M + H) |
| 709(1) | 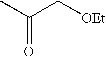 | 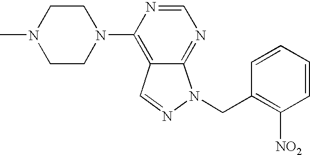 | Amorphous powder MS(APCI) 770(M + H) |

2) The compound obtained in the above steps (1) are treated in the same manner as described in Example 2(2) to give the following compounds (salt).

Example 697(2): 1 Fumaric acid salt of the compound obtained in Example 697(1); Amorphous powder, MS(APCI)m/z: 756(M+H), IR(Nujol)cm⁻¹: 1705, 1636

Example 698(2): 1 Fumaric acid salt of the compound MS(APCI)m/z: 786(M+H), IR(Nujol)cm⁻¹: 1711, 1636

Example 699(2): 1 Fumaric acid salt of the compound obtained in Example 699(1); Amorphous powder, MS(APCI)m/z: 782(M+H), IR(Nujol)cm⁻¹: 1667, 1643

Example 700(2): 1 Fumaric acid salt of the compound obtained in Example 700(1); Amorphous powder, MS(APCI)m/z: 784(M+H), IR(Nujol)cm⁻¹: 1718, 1635

Example 701(2): 1 Fumaric acid salt of the compound obtained in Example 701(1); Amorphous powder, MS(APCI)m/z: 712(M+H), IR(Nujol)cm⁻¹: 1701, 1635

Example 702(2): 1 Fumaric acid salt of the compound obtained in Example 702(1); Amorphous powder, MS(APCI)m/z: 742(M+H), IR(Nujol)cm⁻¹: 1711, 1634

Example 703(2): 1 Fumaric acid salt of the compound obtained in Example 703(1); Amorphous powder, MS(APCI)m/z: 740(M+H), IR(Nujol)cm⁻¹: 1744, 1715

Example 704(2): 1 Fumaric acid salt of the compound obtained in Example 704(1); Amorphous powder, MS(APCI)m/z: 738(M+H), IR(Nujol)cm⁻¹: 1636, 1573

### Example 710

1) To a solution of (1α,4β)-2′-[3-(2-hydroxyethoxy)carbonylaminopropyl]-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′H)-isoquinoline] (compound obtained in Example 704(1)) (95 mg) in methylene chloride (2 mL) is added triethylamine (19 mg) and triphosgene (18 mg) with ice-cooling and the mixture is stirred for 1 hour. To the reaction mixture is added for 1 hour. The reaction mixture is diluted with ethyl acetate and the mixture is washed with water and saturated aqueous NaCl solution, successively. The organic layer is dried over sodium sulfate and concentrated. The residue is purified by column chromatography on silica gel (solvent; chloroform:methanol:aqueous ammonia=20:1:0.1) to give (α,4β)-2′-[3-(2-dimethylaminocarboxyethoxy)-carbonylaminopropyl]-3′,4′-dihydro-6′,7′-dimethoxy-4-[4-[1-(2-nitrobenzyl)-1H-pyrazolo[3,4-d]pyrimidin-4-yl]-1-piperazinyl]carbonyl-spiro[cyclohexane-1,1′(2′H)-isoquinoline] (34 mg) as an amorphous powder.

MS(APCI)m/z: 843(M+H), IR(Nujol)cm⁻¹: 1702, 1637.

2) The compound obtained in the above step (1) is treated in the same manner as described in Example 2(2) to give 1 fumaric acid salt thereof.

MS(APCI)m/z: 843(M+H), IR(Nujol)cm⁻¹: 1704, 1633.

### Example 711

To a solution of (1α,4β)-2′-(3-acetoxyacetylamino-propyl)-3′,4′-dihydro-6′,7′-dimethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline] (compound obtained in Example 707(1)) (172 mg) in methanol (2 mL) is added 4M aqueous sodium hydroxide solution (0.17 mL) and the mixture is stirred for 1 hour. The reaction mixture is diluted with ethyl acetate and washed with saturated aqueous NaCl solution. The organic layer is dried over sodium sulfate and concentrated to give (1α,4β)-2′-(3-hydroxyacetylaminopropyl)-3′,4′-dihydro-6′,7′-dimethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline] (122 mg) as an amorphous powder.

MS(APCI)m/z: 698(M+H), IR(Nujol)cm⁻¹: 1649, 1573.

### Example 712 to 713

1) The corresponding materials are treated in the same manner as described in Example 710 to give the compounds as shown in the following tables (Table.39).

TABLE 39

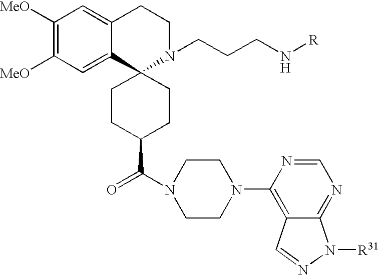

| Ex. No. | R | R³¹ | Physicochemical properties etc. |
|---|---|---|---|
| 712(1) | 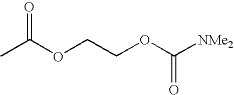 | 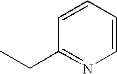 | Amorphous powder MS(APCI) 799(M + H) |
| 713(1) | 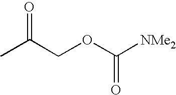 | 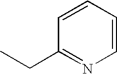 | Amorphous powder MS(APCI) 769(M + H) |

2) The compound obtained in the above steps (1) are treated in the same manner as described in Example 2(2) to give the following compounds (salt).

Example 712(2): 1 Fumaric acid salt of the compound obtained in Example 712(1); Amorphous powder, MS(APCI)m/z: 799(M+H), IR(Nujol)cm⁻¹1698, 1632

Example 713(2): 1 Fumaric acid salt of the compound obtained in Example 713()i; Amorphous powder, MS(APCI)m/z: 769(M+H), IR(Nujol)cm⁻¹: 1704, 1633

### Example 714 to 733

1) The corresponding materials are treated in the same manner as described in Example 296(1) and (2) to give the compounds as shown in the following tables (Table.40).

TABLE 40
(No. 1)

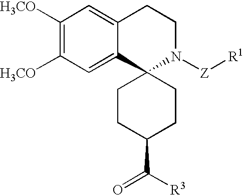

| Ex. | | | Physiochemical |
|---|---|---|---|
| No. | -Z-R¹ | R³ | properties etc. |
| 714 (1) | 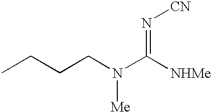 | 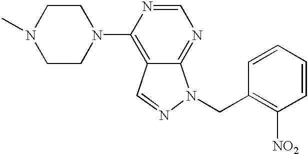 | Amorphous powder MS(APCI) 779(M + H) |
| 715 (1) | 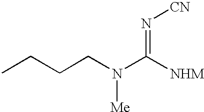 | 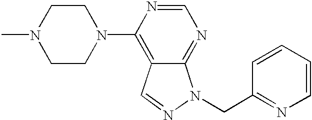 | Amorphous powder MS(APCI) 735(M + H) |
| 716 (1) | 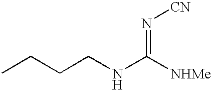 | 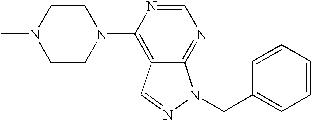 | Amorphous powder MS(APCI) 720(M + H) |
| 717 (1) | 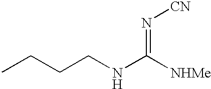 | 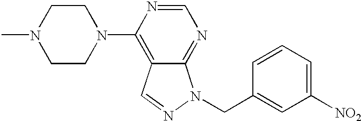 | Amorphous powder MS(APCI) 765(M + H) |
| 718 (1) | 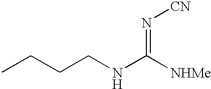 | 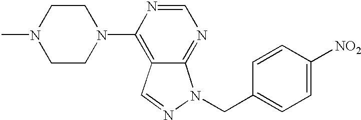 | Amorphous powder MS(APCI) 765(M + H) |
| 719 (1) | 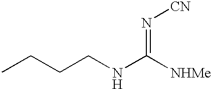 | 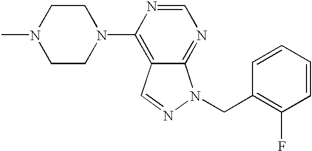 | Amorphous powder MS(APCI) 738(M + H) |
| 720 (1) | 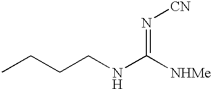 | 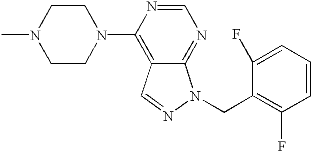 | Amorphous powder MS(APCI) 714 |

TABLE 40
(No. 2)

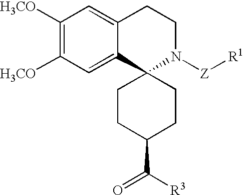

| Ex. | | | Physiochemical |
|---|---|---|---|
| No. | -X-R¹ | R³ | properties etc. |
| 721 (1) | 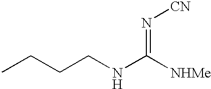 | 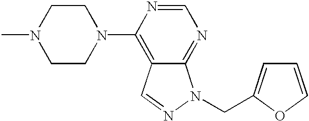 | Amorphous powder MS(APCI) 710(M + H) |
| 722 (1) | 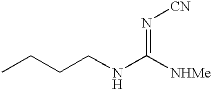 | 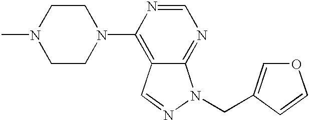 | Amorphous powder MS(APCI) 710(M + H) |
| 723 (1) | 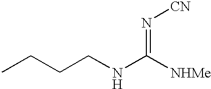 | 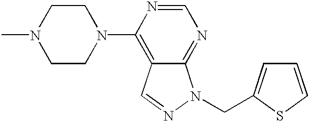 | Amorphous powder MS(APCI) 726(M + H) |
| 724 (1) | 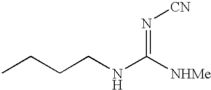 | 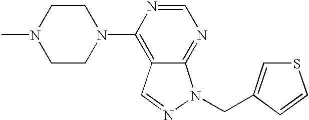 | Amorphous powder MS(APCI) 726(M + H) |
| 725 (1) | 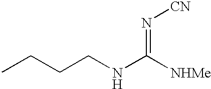 | 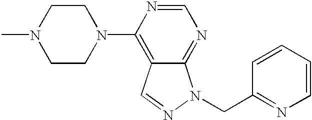 | Amorphous powder MS(APCI) 665 |
| 726 (1) | 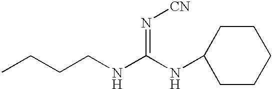 | 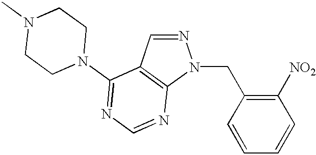 | Amorphous powder MS(APCI) 833(M + H) |
| 727 (1) | 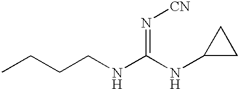 | 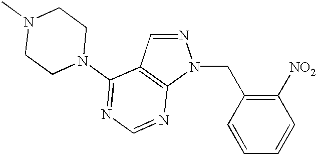 | Amorphous powder MS(APCI) 791(M + H) |

TABLE 40
(No. 3)

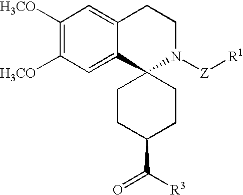

| Ex. | | | Physiochemical |
|---|---|---|---|
| No. | -Z-R¹ | R³ | properties etc. |
| 728 (1) | 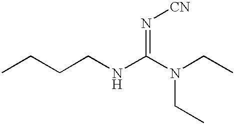 | 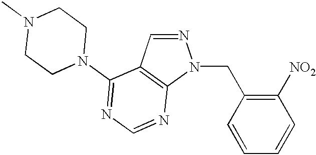 | Amorphous powder MS(APCI) 807(M + H) |
| 729 (1) | 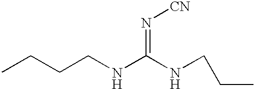 | 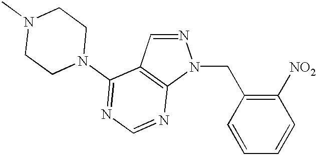 | Amorphous powder MS(APCI) 793(M + H) |
| 730 (1) | 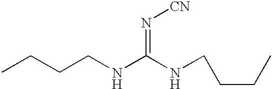 | 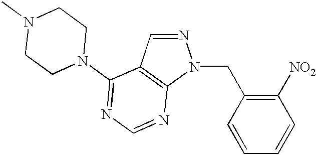 | Amorphous powder MS(APCI) 807(M + H) |
| 731 (1) | 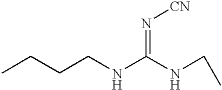 | 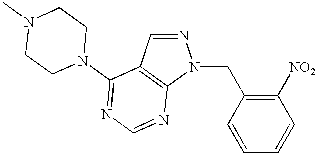 | Amorphous powder MS(APCI) 779(M + H) |
| 732 (1) | 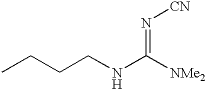 | 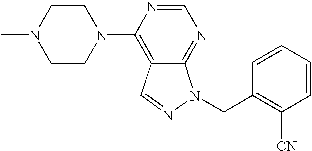 | Amorphous powder MS(APCI) 759(M + H) IR (Nujol) 2161, 1635 |
| 733 (1) | 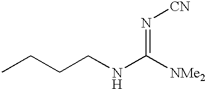 | 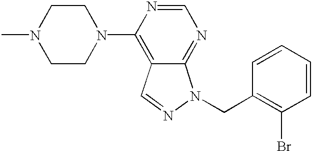 | Amorphous powder MS(APCI) 812, 814(M + H) IR (Nujol) 2165, 1637 |

2) The compound obtained in the above steps (1) are treated in the same manner as described in Example 2(2) to give the following compounds (salt).

Example 714 (2): 1 Fumaric acid salt of the compound obtained in Example 714(1); Amorphous powder, MS(APCI)m/z: 779(M+H), IR(Nujol)cm⁻¹: 2167, 1705, 1635

Example 715(2): 1 Fumaric acid salt of the compound obtained in Example 715(1); Amorphous powder, MS(APCI)m/z: 735(M+H), IR(Nujol)cm⁻¹: 2166, 1733, 1635

Example 716(2): 1 Fumaric acid salt of the compound obtained in Example 716(1); Amorphous powder, MS(ESI)m/z: 720(M+H), IR(Nujol)cm⁻¹: 2168

Example 717(2): 1 Fumaric acid salt of the compound obtained in Example 717(1); Amorphous powder, MS(ESI)m/z: 765(M+H), IR(Nujol)cm⁻¹: 2168

Example 718(2): 1 Fumaric acid salt of the compound obtained in Example 718(1); Amorphous powder, MS(ESI)m/z: 765(M+H), IR(Nujol)cm⁻¹: 2168

Example 719(2): 1 Fumaric acid salt of the compound obtained in Example 719(1); Amorphous powder, MS(ESI)m/z: 738(M+H), IR(Nujol)cm⁻¹: 2167

Example 720(2): 1 Fumaric acid salt of the compound obtained in Example 720(1); Amorphous powder, MS(ESI)m/z: 756(M+H), IR(Nujol)cm⁻¹: 2167

Example 721(2): 1 Fumaric acid salt of the compound obtained in Example 721(1); Amorphous powder, MS(APCI)m/z: 710(M+H), IR(Nujol)cm⁻¹: 2167

Example 722(2): 1 Fumaric acid salt of the compound obtained in Example 722(1); Amorphous powder, MS(APCI)m/z: 710(M+H), IR(Nujol)cm⁻¹: 2167

Example 723(2): 1 Fumaric acid salt of the compound obtained in Example 723(1); Amorphous powder, MS(APCI)m/z: 726(M+H), IR(Nujol)cm⁻¹: 2167

Example 724(2): 1 Fumaric acid salt of the compound obtained in Example 724(1); Amorphous powder, MS(APCI)m/z: 726(M+H), IR(Nujol)cm⁻¹: 2167

Example 725(2): 1 Fumaric acid salt of the compound obtained in Example 725(1); Amorphous powder, MS(APCI)m/z: 721(M+H), IR(Nujol)cm⁻¹: 2167

Example 726(2): 1 Fumaric acid salt of the compound obtained in Example 726(1); Amorphous powder, MS(APCI)m/z: 833(M+H), IR(Nujol)cm⁻¹3277, 2166, 1636, 1701

Example 727(2): 1 Fumaric acid salt of the compound obtained in Example 727 (1); Amorphous powder, MS(APCI)m/z: 791(M+H), IR(Nujol)cm⁻¹: 3271, 2169, 1699, 1682, 1635

Example 728(2): 1 Fumaric acid salt of the compound obtained in Example 728 (1); Amorphous powder, MS(APCI)m/z: 807(M+H), IR(Nujol)cm⁻¹: 3299, 2164, 1706, 1636, 1610

Example 729(2): 1 Fumaric acid salt of the compound obtained in Example 729(1); Amorphous powder, MS(APCI)m/z: 793(M+H), IR(Nujol)cm⁻¹: 3272, 2167, 1699, 1633,

Example 730(2): 1 Fumaric acid salt of the compound obtained in Example 730(1); Amorphous powder, MS(APCI)m/z: 807(M+H), IR(Nujol)cm⁻¹: 3278, 2166, 1705, 1631

Example 731(2): 1 Fumaric acid salt of the compound obtained in Example 731(1); Amorphous powder, MS(APCI)m/z: 779(M+H), IR(Nujol)cm⁻¹: 3287, 2167, 1705

Example 732(2): 1 Fumaric acid salt of the compound obtained in Example 732(1): Amorphous powder, MS(APCI)m/z: 759(M+H), IR(Nujol)cm⁻¹: 2167, 1708, 1637

Example 733(2): 1 Fumaric acid salt of the compound obtained in Example 733(1); Amorphous powder, MS(APCI)m/z: 812, 814(M+H), IR(Nujol)cm⁻¹: 2165, 1635

### Examples 734 to 737

1) The corresponding materials are treated in the same manner as described in Example 304(1) and (2) to give the compounds as shown in the following table (Table.41).

TABLE 41

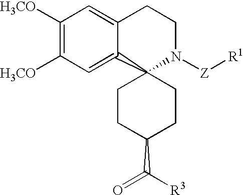

| Ex. | | | Physiochemical |
|---|---|---|---|
| No. | -Z-R¹ | R³ | properties etc. |
| 734 (1) | 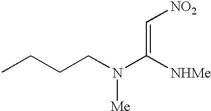 | 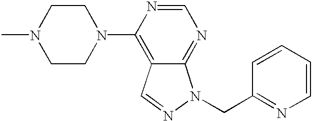 | Amorphous powder MS(APCI) 754(M + H) |
| 735 (1) | 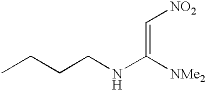 | 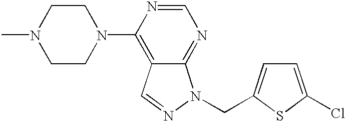 | Amorphous powder MS(APCI) 793(M + H) IR (Nujol) 1637 |
| 736 (1) | 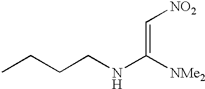 | 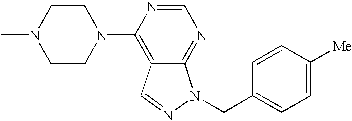 | Amorphous powder MS(APCI) 767(M + H) IR (Nujol) 1637 |
| 737 (1) | 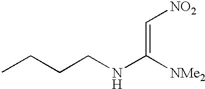 | 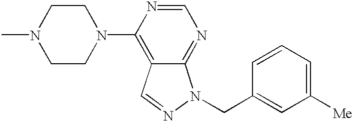 | Amorphous powder MS(APCI) 767(M + H) IR (Nujol) 1637 |

2) The compound obtained in the above steps (1) are treated in the same manner as described in Example 2(2) to give the following compounds (salt).

Example 734 (2): 1 Fumaric acid salt of the compound obtained in Example 734(1); Amorphous powder, MS(ESI)m/z: 754(M+H), IR(Nujol)cm⁻¹: 1700, 3395

Example 735(2): 1 Citric acid salt of the compound obtained in Example 735(1); MS(APCI)m/z: 793.5(M+H), IR(Nujol)cm⁻¹: 1719, 1613

Example 736(2): 1 Citric acid salt of the compound obtained in Example 736(1); MS(APCI)m/z: 767.5(M+H), IR(Nujol)cm⁻¹: 1721, 1613

Example 737(2): 1 Citric acid salt of the compound obtained in Example 737(1); MS(ESI)m/z: 767.5(M+H), IR(Nujol)cm⁻¹: 1719, 1611

### Example 738

1) To a solution of (1α,4β)-2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl)carbonyl-spiro[cyclohexane-1,1′(2′H)-isoquinoline] (compound obtained in Example 283(1)) (200 mg) in tetrahydrofuran (2 mL) are added cyclohexylthioisocyanate (44 μL) and the mixture is stirred at room temperature for 18 hours. The reaction mixture is concentrated and the residue is purified by column chromatography on silica gel (solvent; chloroform) to give (1α,4β)-2′-[(3-N-cyclohexylthioureido)propyl]-3′,4′-dihydro-6′,7′-dimethoxy-4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline] (240 mg, 100%) as amorphous powder.

MS(APCI)m/z: 825(M+H), IR(Nujol)cm⁻¹: 3331, 2175, 1716, 1699, 1683, 1636.

2) The compound obtained in the above step (1) is treated in the same manner a described in Example 2(2) to give 1 fumaric acid salt thereof as a colorless amorphous powder.

MS(APCI)m/z: 825(M+H), IR(Nujol)cm⁻¹: 3274, 1706, 1635.

### Examples 739 to 741

The corresponding materials are treated in the same manner as described in Example 738(1) to give the compounds as shown in the following table (Table.42).

TABLE 42

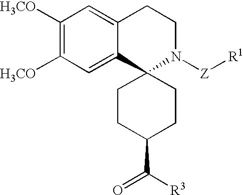

| Ex. | | | Physiochemical |
|---|---|---|---|
| No. | -Z-R¹ | R³ | properties etc. |
| 739 (1) | 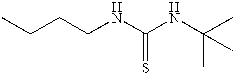 | 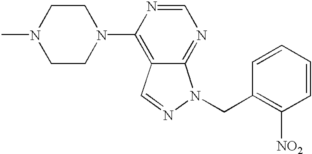 | Amorphous powder MS(APCI) 799(M + H) |
| 740 (1) | 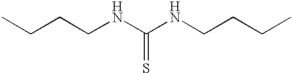 | 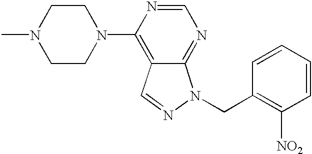 | Amorphous powder MS(APCI) 799(M + H) |
| 741 (1) | 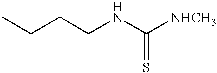 | 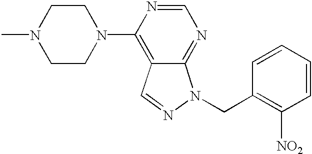 | Amorphous powder MS(APCI) 757(M + H) |

2) The compound obtained in the above steps (1) are treated in the same manner as described in Example 2(2) to give the following compounds (salt).

Example 739(2): 1 Fumaric acid salt of the compound obtained in Example 739(1); amorphous, MS(APCI)m/z: 799(M+H), IR(Nujol)cm⁻¹: 3343, 1706, 1636, 1612

Example 740(2): 1 Fumaric acid salt of the compound obtained in Example 740(1); amorphous, MS(APCI)m/z: 799(M+H), IR(Nujol)cm⁻¹: 3265, 1702, 1635, 1611

Example 741(2): 1 Fumaric acid salt of the compound obtained in Example 741(1); amorphous, MS(APCI)m/z: 757(M+H), IR(Nujol)cm⁻¹: 3341, 1707, 1635, 1612

### Example 742

1) To a solution of (1α,4β)-2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (200 mg) in tetrahydrofuran (5 mL) is added butylisocyanate (40 μL) and the mixture is stirred at room temperature for 18 hours. The reaction mixture is concentrated and the residue is purified by column chromatography on NH-silica gel (solvent; chloroform) to give (1α,4β)-2′-[3-(N-n-butylureido)-propyl]-3′,4′-dihydro-6′,7′-dimethoxy-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (201 mg, 88%) as amorphous powder.

MS(APCI)m/z: 783(M+H), IR(Nujol)cm⁻¹: 3347, 1716, 1636.

2) The compound obtained in the above step (1) is treated in the same manner a described in Example 2(2) to give 1 fumaric acid salt thereof as a colorless amorphous powder.

MS(APCI)m/z: 783(M+H), IR(Nujol)cm⁻¹: 3358, 1705, 1637.

### Example 743

1) The corresponding materials are treated in the same manner as described in Example 742(1) to give (1α,4β)-2′-[3-(N-ethylureido)-propyl]-3′,4′-dihydro-6′,7′-dimethoxy-4-[4-[1-(2-nitrophenyl-methyl)-1H-pyrazolo[3,4-d]pyrimidin-4-yl]-1-piperazinyl]carbonyl-spiro[cyclohexane-1,1′(2′H)-isoquinoline] (240 mg) as an amorphous powder.

MS(APCI)m/z: 755(M+H).

2) The compound obtained in the above step (1) is treated in the same manner a described in Example 2(2) to give 1 fumaric acid salt thereof as amorphous.

MS(APCI)m/z: 755(M+H), IR(Nujol)cm⁻¹: 3346, 1703, 1636, 1612.

### Example 744

1) To a solution of (1α,4β)-2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (200 mg) in methylene chloride (5 mL) are added acetyl chloride (25 μL) and triethylamine (60 μL) under ice-cooling and the mixture is stirred at room temperature for 18 hours. The reaction mixture is concentrated and the residue is purified by column chromatography on NH-silica gel (solvent; chloroform) to give (1α,4β)-2′-[3-(acetamido)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (191 mg, 90%) as amorphous powder.

MS(APCI)m/z: 726(M+H), IR(Nujol)cm⁻¹: 1698, 1636.

2) The compound obtained in the above step (1) is treated in the same manner a described in Example 2(2) to give 1 fumaric acid salt thereof as colorless amorphous powder.

MS(APCI)m/z: 726(M+H), IR(Nujol)cm⁻¹: 3273, 1705, 1635.

### Example 745

To a solution of (1α,4β)-2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (1.0 g) in methylene chloride (5 mL) and isopropanol (5 mL) is added 3,4-diethoxy-3-cyclobuten-1,2-dione (272 mg) and the mixture is stirred at room temperature for 24 hours. The reaction mixture is concentrated and the residue is purified by column chromatography on silica gel (solvent; chloroform:methanol:20:1) to give (1α,4β)-2′-[3-(3-ethoxy-3-cyclobutene-1,2-dion-4-yl)aminopropyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (1.13 g, 96%) as an amorphous powder.

MS(APCI)m/z: 808(M+H), IR(Nujol)cm⁻¹: 3213, 1801, 1705, 1607.

### Example 746

1) To a solution of (1α,4β)-2′-[3-(3-ethoxy-3-cyclobutene-1,2-dion-4-yl)aminopropyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 745) (200 mg) in tetrahydrofuran (2 mL) and isopropanol (4 mL) is added aqueous dimethylamine (2 mL) and the mixture is stirred at room temperature for 18 hours. The reaction mixture is concentrated and the residue is purified by column chromatography on NH-silica gel (solvent; chloroform) to give (1α,4β)-2′-[3-(3-dimethylamino-3-cyclobutene-1,2-dion-4-yl)aminopropyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (170 mg, 85%) as an amorphous powder.

MS(APCI)m/z: 807(M+H), IR(Nujol)cm⁻¹: 1793, 1668, 1636.

2) The compound obtained in the above step (1) is treated in the same manner a described in Example 2(2) to give 1 fumaric acid salt thereof as colorless amorphous powder.

MS(APCI)m/z: 807(M+H), IR(Nujol)cm⁻¹: 3387, 3246, 1795, 1707, 1637.

### Examples 747 to 748

The corresponding materials are treated in the same manner as described in Example 746(1) to give the compounds as shown in the following table (Table.43).

TABLE 43

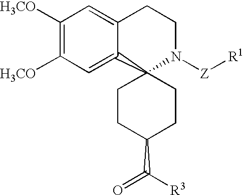

| Ex. | | | Physiochemical |
|---|---|---|---|
| No. | -Z-R¹ | R³ | properties etc. |
| 747 (1) | 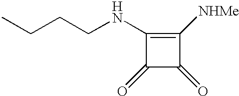 | 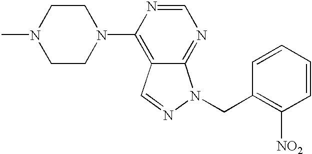 | Amorphous powder MS(APCI) 793(M + H) |
| 748 (1) | 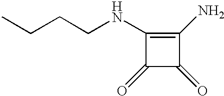 | 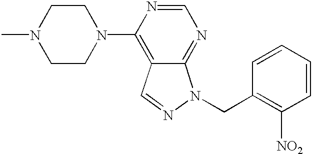 | Amorphous powder MS(APCI) 779(M + H) |

2) The compound obtained in the above steps (1) are treated in the same manner as described in Example 2(2) to give the following compounds (salt).

Example 747(2): 1 Fumaric acid salt of the compound obtained in Example 747(1); Amorphous powder, MS(APCI)m/z: 793(M+H), IR(Nujol)cm⁻¹: 3246, 1799, 1707

Example 748(2): 1 Fumaric acid salt of the compound obtained in Example 748(1); Amorphous powder, MS(APCI)m/z: 779(M+H), IR(Nujol)cm⁻¹: 3328, 3197, 1801, 1635

### Example 749

1) To a solution of (1α,4β)-2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 283(1)) (1.0 g) in tetrahydrofuran (2 mL) and isopropanol (10 mL) is added (bis(methylthio)methylene)propandinitrile ((CH₃S)₂C═C(CN)₂) (273 mg) and the mixture is stirred at room temperature for 8 hours. The reaction mixture is concentrated and the residue is purified by column chromatography on silica gel (solvent; chloroform:methanol; 30:1) to give (1α,4β)-2′-[3-(1-methylthio-2,2-dicyanoethylenamino)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (1.17 g, 99%) as an amorphous powder.

MS(APCI)m/z: 806(M+H), IR(Nujol)cm⁻¹: 2204, 1733, 1717, 1699, 1635.

### Example 750

1) To a solution of (1α,4β)-2′-[3-(1-methylthio-2,2-dicyanoethylenamino)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (compound obtained in Example 749) (200 mg) in acetonitrile (5 mL) is added aqueous dimethylamine (5 mL) and the mixture is heated in a sealed tube at 100° C. for 30 minute. The reaction mixture is concentrated and the residue is purified by column chromatography on silica gel (solvent; chloroform:methanol; 50:1) to give (1α,4β)-2′-[3-(1-amino-2,2-dicyanoethylenamino)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline] (268 mg, 72%) as an amorphous powder.

MS(APCI)m/z: 775(M+H), IR(Nujol)cm⁻¹: 2199, 2173, 1631.

2) The compound obtained in the above step (1) is treated in the same manner a described in Example 2 (2) to give 1 fumaric acid salt thereof as a colorless amorphous powder.

MS(APCI)m/z: 825(M+H), IR(Nujol)cm⁻¹: 2200, 2174, 1701, 1635.

### Examples 751 to 752

The corresponding materials are treated in the same manner as described in Example 750(1) to give the compounds as shown in the following table (Table.44).

TABLE 44

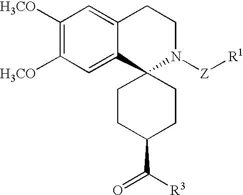

| Ex. | | | Physiochemical |
|---|---|---|---|
| No. | -Z-R¹ | R³ | properties etc. |
| 751 (1) | 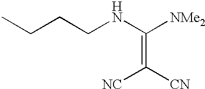 | 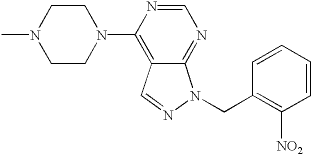 | Amorphous powder MS(APCI) 803(M + H) |
| 752 (1) | 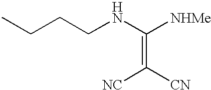 | 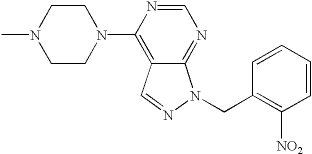 | Amorphous powder MS(APCI) 789(M + H) |

2) The compound obtained in the above steps (1) are treated in the same manner as described in Example 2(2) to give the following compounds (salt).

Example 751(2): 1 Fumaric acid salt of the compound obtained in Example 751(1); an amorphous powder, MS(APCI)m/z: 803(M+H), IR(Nujol)cm⁻¹: 3264, 2200, 2176, 1707, 1636

Example 752(2): 1 Fumaric acid salt of the compound obtained in Example 752(1); an amorphous powder, MS(APCI)m/z: 789(M+H), IR(Nujol)cm⁻¹: 3264, 2200, 2176, 1707, 1636

### Examples 753 to 779

1) (1α,4β)-2′-[3-(dimethylamino)propionyl]-3′,4′-dihydro-6′,7′-dimethoxy-4-carboxy-spiro[cyclohexane-1,1′(2′H)-isoquinoline] (compound obtained in Reference example 15(2)) and the corresponding starting materials are treated in the same manner as described in Example 52(1) to give the compounds as shown in the following table (Table.45).

TABLE 45
(No. 1)

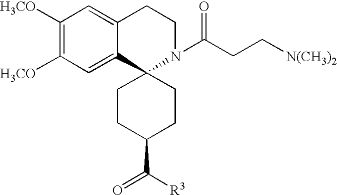

| Ex. | | Physiochemical |
|---|---|---|
| No. | R³ | properties etc. |
| 753 (1) | 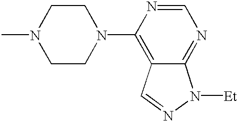 | Amorphous powder MS(APCI) 619(M + H) |
| 754 (1) | 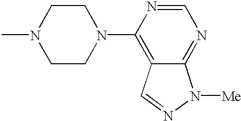 | Amorphous powder MS(APCI) 605(M + H) |
| 755 (1) | 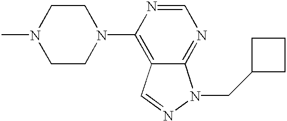 | Amorphous powder MS(APCI) 659(M + H) |
| 756 (1) | 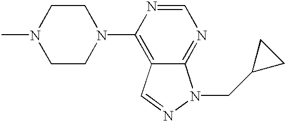 | Amorphous powder MS(APCI) 645(M + H) |
| 757 (1) | 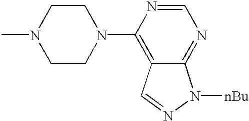 | Amorphous powder MS(APCI) 647(M + H) |
| 758 (1) | 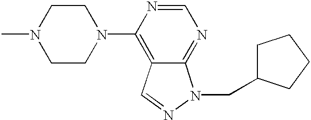 | Amorphous powder MS(APCI) 673(M + H) |
| 759 (1) | 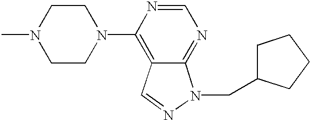 | Amorphous powder MS(APCI) 630(M + H) |
| 760 (1) | 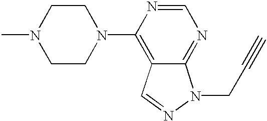 | Amorphous powder MS(APCI) 629(M + H) |
| 761 (1) | 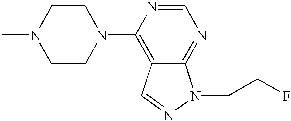 | Amorphous powder MS(APCI) 637(M + H) |

TABLE 45
(No. 2)

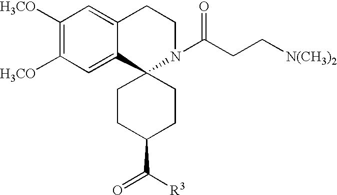

| Ex. | | Physiochemical |
|---|---|---|
| No. | R³ | properties etc. |
| 762 (1) | 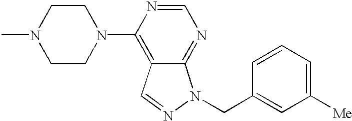 | Amorphous powder MS(APCI) 695(M + H) |
| 763 (1) | 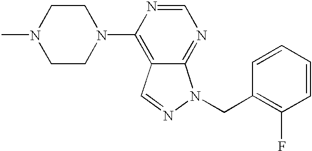 | Amorphous powder MS(APCI) 699(M + H) |
| 764 (1) | 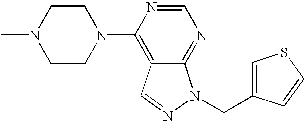 | Amorphous powder MS(APCI) 687(M + H) |
| 765 (1) | 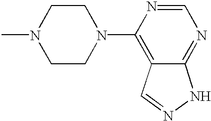 | Amorphous powder MS(APCI) 591(M + H) |
| 766 91) | 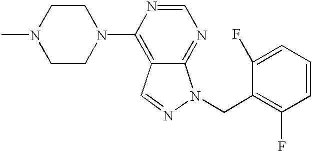 | Amorphous powder MS(APCI) 717(M + H) |
| 767 (1) | 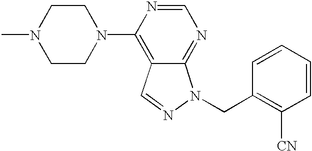 | Amorphous powder MS(APCI) 706(M + H) |
| 768 (1) | 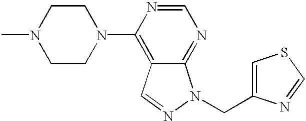 | Amorphous powder MS(APCI) 688(M + H) |

TABLE 45
(No. 3)

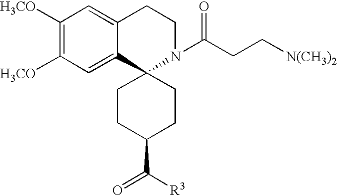

| Ex. | | Physiochemical |
|---|---|---|
| No. | R³ | properties etc. |
| 769 (1) | 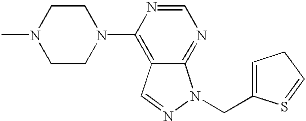 | Amorphous powder MS(APCI) 687(M + H) |
| 770 (1) | 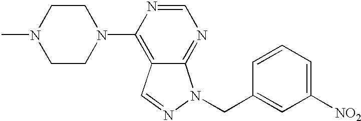 | Amorphous powder MS(APCI) 726(M + H) |
| 771 (1) | 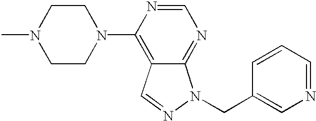 | Amorphous powder MS(APCI) 682(M + H) |
| 772 (1) | 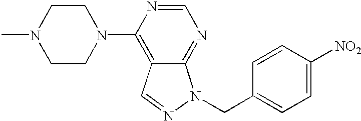 | Amorphous powder MS(APCI) 726(M + H) |
| 773 (1) | 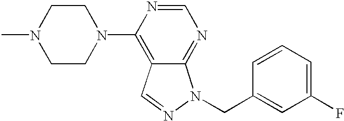 | Amorphous powder MS(APCI) 699(M + H) |
| 774 (1) | 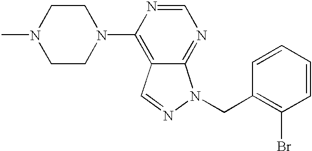 | Amorphous powder MS(APCI) 759/761 (M + H) |
| 775 (1) | 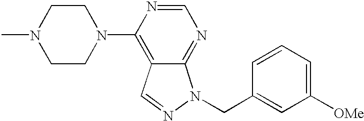 | Amorphous powder MS(APCI) 711(M + H) |
| 776 (1) | 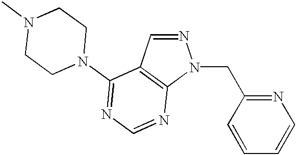 | Amorphous powder MS(APCI) 682(M + H) |

TABLE 45
(No.4)

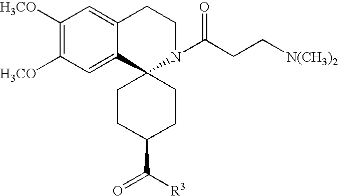

| Ex. | | Physicochemical |
|---|---|---|
| No. | R³ | properties etc. |
| 777 (1) | 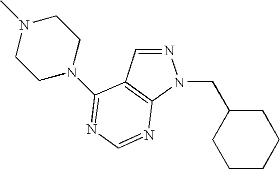 | Amorphous powder MS (APCI) 687 (M + H) |
| 778 (1) | 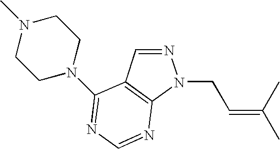 | Amorphous powder MS (APCI) 659 (M + H) |
| 779 (1) | 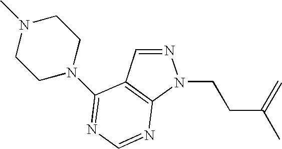 | Amorphous powder MS (APCI) 659 (M + H) |

2) The compounds obtained in the above steps (1) are treated in the same manner as described in Example 2(2) to give the following compounds (salt).

Example 776(2): 1 Fumaric acid salt of the compound obtained in Example 776(1); an amorphous powder, MS(APCI)m/z: 682(M+H), IR(Nujol)cm⁻¹: 3855, 3629, 3395

Example 777(2): 1 Fumaric acid salt of the compound obtained in Example 777(1); an amorphous powder, MS(APCI)m/z: 687(M+H), IR(Nujol)cm⁻¹: 3416, 1705, 1636

Example 778(2): 1 Fumaric acid salt of the compound obtained in Example 778(1); an amorphous powder, MS(APCI)m/z: 659(M+H), IR(Nujol)cm⁻¹: 3407, 1706, 1636

Example 779(2): 1 Fumaric acid salt of the compound obtained in Example 779(1); an amorphous powder, MS(APCI)m/z: 659(M+H), IR(Nujol)cm⁻¹: 3406, 1705, 1635

### Example 780

1) (1R*,2R*(S*),4R*)-2′-[3-(N-methyl-N-benzyloxycarbonyl-amino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (compound obtained in Reference example 1(10)) and 4,6-dimethyl-2-piperazinylpyridine are treated in the same manner as described in Example 52(1) to give (1R*,2R*(S*),4R*)-2′-[3-(N-methyl-N-benzyloxycarbonyl-amino)propionyl]-3′,4′-dihydro-6′,7′-dimethoxy-2-(2-ethyl-1,2,3,4-tetrahydro-6,7-dimethoxy-1-isoquinolyl)-4-[4-(4,6-dimethylpyridin-2-yl)-1-piperazinyl]carbonyl-spiro[cyclohexane-1,1′(2′H)-isoquinoline] as an amorphous powder.

MS(APCI)m/z: 917(M+H), IR(Nujol)cm⁻¹: 1701, 1635, 1605.

2) The compound obtained in the above step (1) is treated in the same manner as described in Example 165(1) to give (1R*,2R*(S*),4R*)-2′-[3-(N-methyl-amino)propionyl]-3′,4′-dihydro-6′,7′-dimethoxy-2-(2-ethyl-1,2,3,4-tetrahydro-6,7-dimethoxy-1-isoquinolyl)-4-[4-(4,6-dimethylpyridin-2-yl)-1-piperazinyl]carbonyl-spiro[cyclohexan-1,1′(2′H)-isoquinoline] as an amorphous powder.

MS(APCI)m/z: 783(M+H), IR(Nujol)cm⁻¹: 1635, 1605.

3) The compound obtained in the above step (2) is treated in the same manner as described in Example 2(2) to give 1 fumaric acid salt thereof.

MS(APCI)m/z: 783(M+H), IR(Nujol)cm⁻¹: 1634, 1606.

### Example 781

1) To a solution of (1α,4β)-2′-(3-aminopropyl)-3′,4′-dihydro-6′,7′-dimethoxy-4-[4-[1-(2-fluorobenzyl)-1H-pyrazolo[3,4-d]pyrimidin-4-yl]-1-piperazinyl]carbonyl-spiro[cyclohexane-1,1′(2′H)-isoquinoline] (compound obtained in Example 477) (500 mg) triethylamine (0.13 mL) in dichloromethane (15 mL) is added (5-methyl-1,3-dioxolen-2-on-4-yl)-methyl p-nitrophenyl carbonate (270 mg) (J. Alexander and L. Kans, U.S. Pat. No. 5,466,811) and the mixture is stirred with ice-cooling for 1.5 hours. To the reaction mixture is added water (10 mL) and the organic layer is separated. The organic layer is washed with saturated aqueous sodium hydrogencarbonate solution (10 mL), dried over sodium sulfate, concentrated. The residue is purified by column chromatography on silica gel (solvent; chloroform:methanol; 30:1→20:1) to give (1α,4β)-2′-[3-[N-(5-methyl-2-oxo-1,3-dioxol-4-yl)-methyloxycarbonylamino]propyl]-3′,4′-dihydro-6′,7′-dimethoxy-4-[4-[1-(2-fluorobenzyl)-1H-pyrazolo[3,4-d]pyrimidin-4-yl]-1-piperazinyl]carbonyl-spiro[cyclohexane-1,1′(2′H)-isoquinoline] (355 mg, 57%) as an amorphous powder. MS(APCI)m/z: 657 (parent compound form), IR(Nujol)cm⁻¹: 1816, 1717, 1633.

2) The compound obtained in the above step (1) is treated in the same manner a described in Example 2(2) to give 2 fumaric acid salt thereof as a colorless amorphous powder.

MS(APCI)m/z: 813(M+H), IR(Nujol)cm⁻¹: 1817, 1708, 1638.

### Examples 782 to 816

The corresponding starting materials are treated in the same manner as described in Example 52(1) to give the compounds as shown in the following tables (Table.46).

TABLE 46
(NO.1)

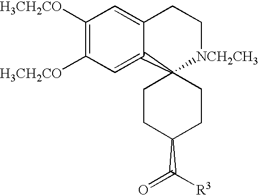

| Ex. | | Physicochemical |
|---|---|---|
| No | R³ | properties etc. |
| 782 | 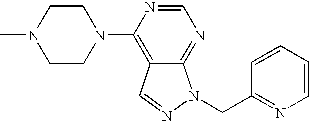 | Amorphous powder MS (APCI) 639 (M + H) |
| 783 | 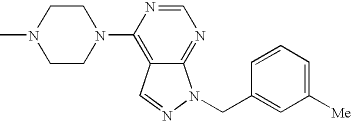 | Amorphous powder MS (APCI) 652 (M + H) |
| 784 | 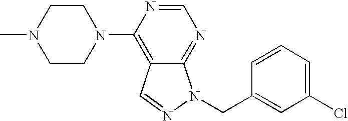 | Amorphous powder MS (APCI) 672 (M + H) |
| 785 | 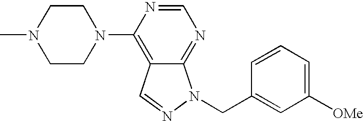 | Amorphous powder MS (APCI) 668 (M + H) |
| 786 | 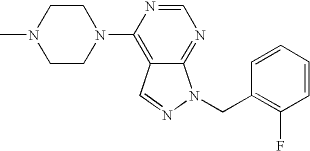 | Amorphous powder MS (APCI) 656 (M + H) |
| 787 | 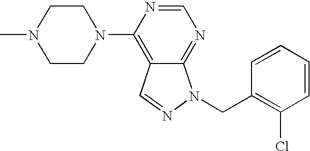 | Amorphous powder MS (APCI) 672 (M + H) |
| 788 | 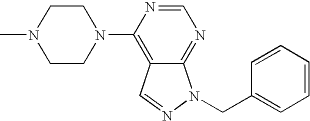 | Amorphous powder MS (APCI) 638 (M + H) |
| 789 | 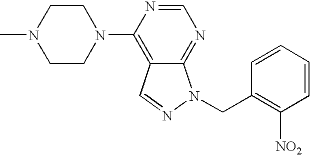 | Amorphous powder MS (APCI) 683 (M + H) |

TABLE 46
(No.2)

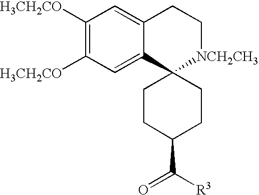

| Ex. | | Physicochemical |
|---|---|---|
| No. | R³ | properties etc. |
| 790 | 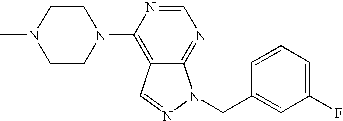 | Amorphous powder MS (APCI) 656 (M + H) |
| 791 | 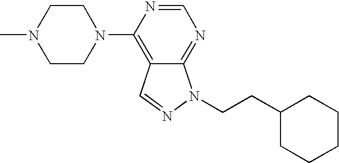 | Amorphous powder MS (APCI) 644 (M + H) |
| 792 | 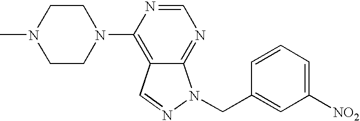 | Amorphous powder MS (APCI) 683 (M + H) |
| 793 | 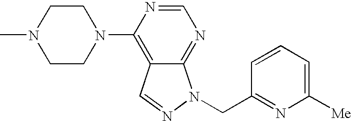 | Amorphous powder MS (APCI) 653 (M + H) |
| 794 | 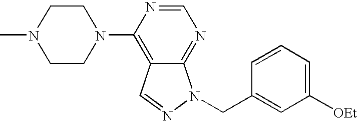 | Amorphous powder MS (APCI) 682 (M + H) |
| 795 | 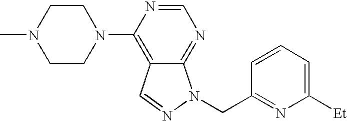 | Amorphous powder MS (APCI) 667 (M + H) |

TABLE 46
(No.3)

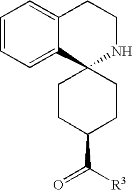

| Ex. | | Physicochemical |
|---|---|---|
| No. | R³ | properties etc. |
| 796 | 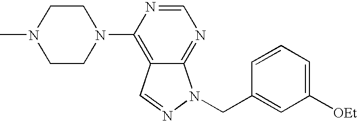 | Amorphous powder MS (APCI) 566 (M + H) |
| 797 | 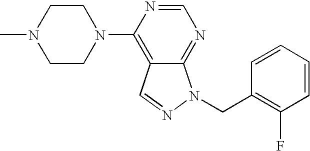 | Amorphous powder MS (APCI) 540 (M + H) |
| 798 | 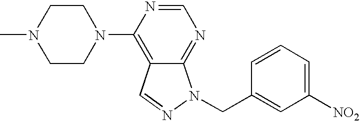 | Amorphous powder MS (APCI) 567 (M + H) |
| 799 | 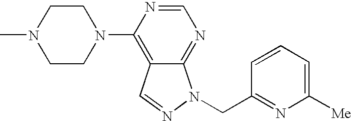 | Amorphous powder MS (APCI) 537 (M + H) |

TABLE 46
(No.4)

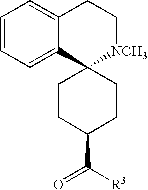

| Ex. | | Physicochemical |
|---|---|---|
| No. | R³ | properties etc. |
| 800 | 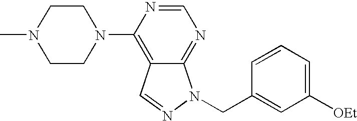 | Amorphous powder MS (APCI) 580 (M + H) |

TABLE 46
(No.5)

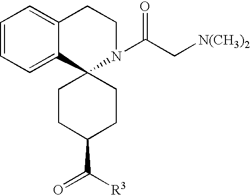

| Ex. | | Physicochemical |
|---|---|---|
| No. | R³ | properties etc. |
| 801 | 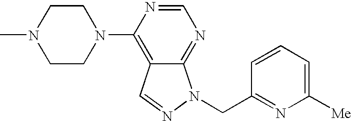 | Amorphous powder MS (APCI) 622 (M + H) |
| 802 | 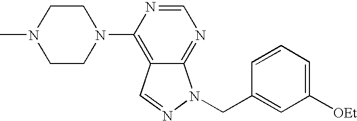 | Amorphous powder MS (APCI) 651 (M + H) |
| 803 | 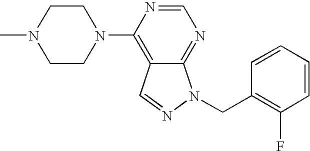 | Amorphous powder MS (APCI) 625 (M + H) |
| 804 | 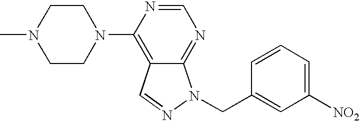 | Amorphous powder MS (APCI) 652 (M + H) |

TABLE 46
(No.6)

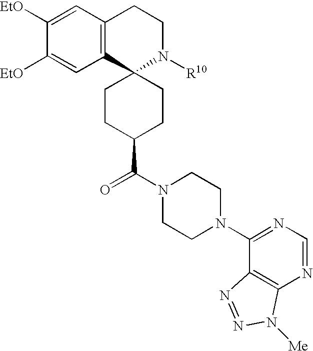

| Ex. | | Physicochemical |
|---|---|---|
| No. | R¹⁰ | properties etc. |
| 805 | —Et | Amorphous powder |
| | | MS (APCI) 563 (M + H) |
| 806 | —CH₂CH₂OH | Amorphous powder |
| | | MS (APCI) 579 (M + H) |

TABLE 46
(No.7)

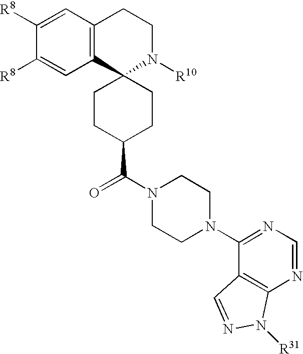

| Ex. | | | | Physicochemical |
|---|---|---|---|---|
| No. | R⁸ | R¹⁰ | R³¹ | properties etc. |
| 807 | MeO | H | 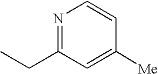 | Amorphous powder MS (APCI) 597 (M + H) |
| 808 | MeO | Me | 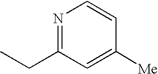 | Amorphous powder MS (APCI) 611 (M + H) |
| 809 | EtO | H | 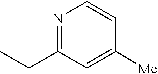 | Amorphous powder MS (APCI) 625 (M + H) |
| 810 | EtO | Me | 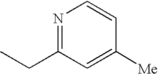 | Amorphous powder MS (APCI) 639 (M + H) |
| 811 | MeO | Me | 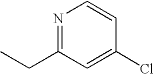 | Amorphous powder MS (APCI) 631 (M + H) |
| 812 | EtO | Me | 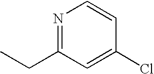 | Amorphous powder MS (APCI) 659 (M + H) |

TABLE 46
(No.8)

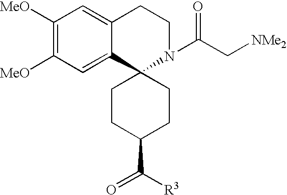

| Ex. | | Physicochemical |
|---|---|---|
| No. | R³ | properties etc. |
| 813 | 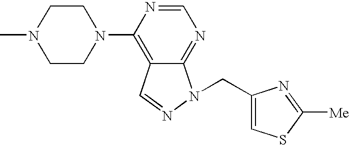 | Amorphous powder MS (APCI) 688 (M + H) IR (Nujol) 1633 |
| 814 | 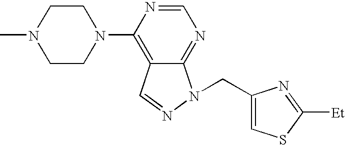 | Amorphous powder MS (APCI) 702 (M + H) IR (Nujol) 1634 |
| 815 | 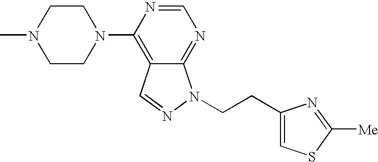 | Amorphous powder MS (APCI) 702 (M + H) IR (Nujol) 1633 |
| 816 | 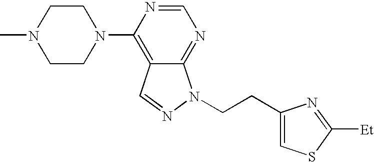 | Amorphous powder MS (APCI) 716 (M + H) IR (Nujol) 1633 |

### Examples 817 to 936

The corresponding materials are treated in the same manner as described in Example 52 (1) to give a compound as shown in the following tables (Table.47).

TABLE 47
(No.1)

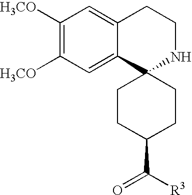

| Ex. | | Physicochemical. |
|---|---|---|
| No. | R³ | Properties etc. |
| 817 | 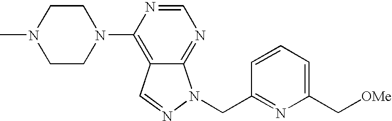 | Amorphous powder MS (APCI) 627 (M + H) |
| 818 | 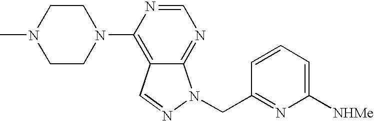 | Amorphous powder MS (APCI) 612 (M + H) |
| 819 | 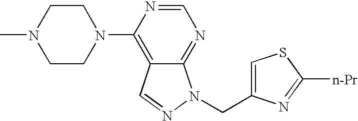 | Amorphous powder MS (APCI) 631 (M + H) |
| 820 | 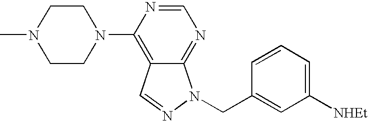 | Amorphous powder MS (APCI) 625 (M + H) |
| 821 | 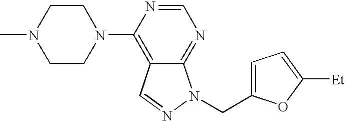 | Amorphous powder MS (APCI) 600 (M + H) |
| 822 | 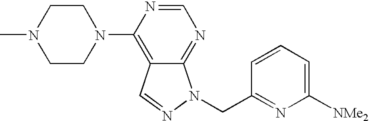 | Amorphous powder MS (APCI) 626 (M + H) |
| 823 | 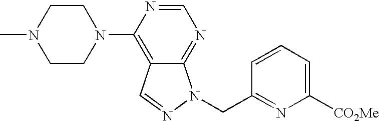 | Amorphous powder MS (APCI) 641 (M + H) |
| 824 | 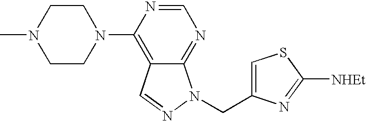 | Amorphous powder MS (APCI) 632 (M + H) |
| 824A | 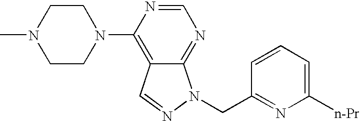 | Amorphous powder (APCI) 625 (M + H) |

TABLE 47
(No. 2)

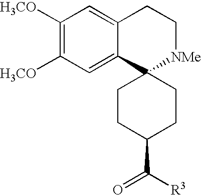

| Ex. | | Physicochemical |
|---|---|---|
| No. | R³ | Properties etc. |
| 825 | 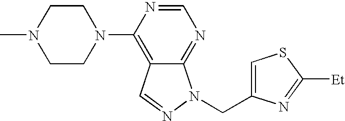 | Amorphous powder MS (APCI) 631 (M + H) |
| 826 | 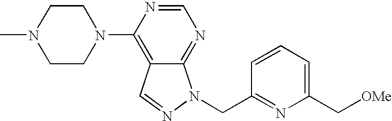 | Amorphous powder MS (APCI) 641 (M + H) |
| 827 | 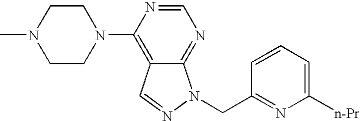 | Amorphous powder MS (APCI) 639 (M + H) |
| 828 | 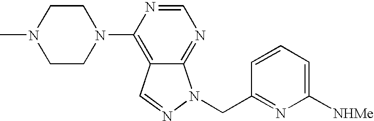 | Amorphous powder MS (APCI) 626 (M + H) |
| 829 | 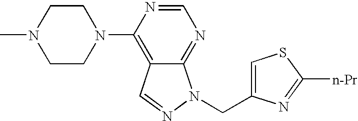 | Amorphous powder MS (APCI) 645 (M + H) |
| 830 | 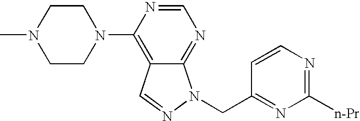 | Amorphous powder MS (APCI) 640 (M + H) |

TABLE 47
(No. 3)

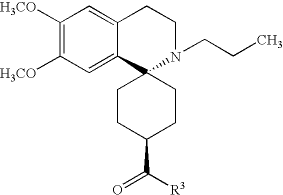

| Ex. | | Physicochemical |
|---|---|---|
| No. | R³ | Properties etc. |
| 831 | 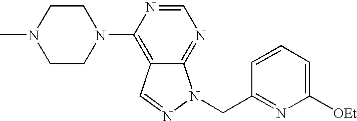 | Amorphous powder MS (APCI) 669 (M + H) |
| 832 | 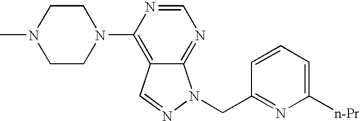 | Amorphous powder MS (APCI) 667 (M + H) |
| 833 | 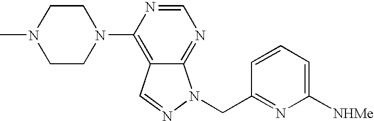 | Amorphous powder MS (APCI) 654 (M + H) |
| 834 | 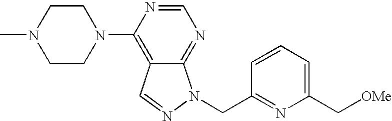 | Amorphous powder MS (APCI) 669 (M + H) |
| 835 | 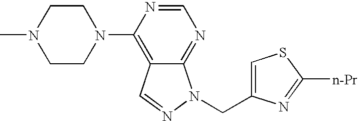 | Amorphous powder MS (APCI) 673 (M + H) |
| 836 | 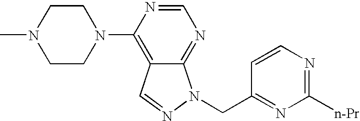 | Amorphous powder MS (APCI) 668 (M + H) |
| 837 | 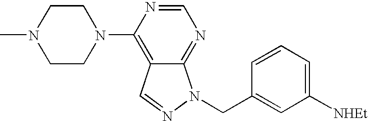 | Amorphous powder MS (APCI) 667 (M + H) |
| 838 | 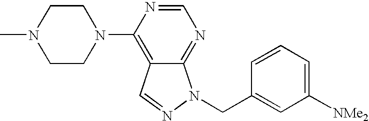 | Amorphous powder MS (APCI) 667 (M + H) |
| 839 | 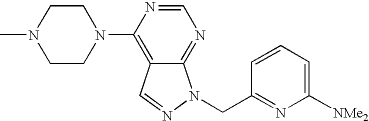 | Amorphous powder MS (APCI) 668 (M + H) |
| 840 | 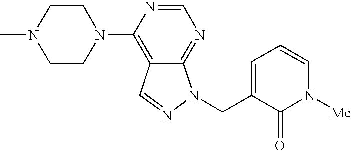 | Amorphous powder MS (APCI) 655 (M + H) |

TABLE 47
(No. 4)

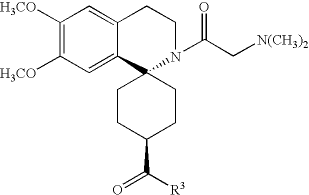

| Ex. | | Physicochemical |
|---|---|---|
| No. | R³ | Properties etc. |
| 841 | 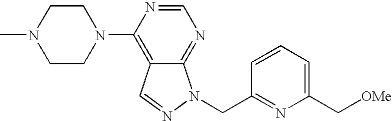 | Amorphous powder MS (APCI) 712 (M + H) |
| 842 | 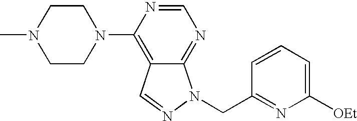 | Amorphous powder MS (APCI) 712 (M + H) |
| 843 | 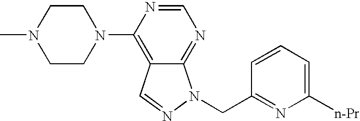 | Amorphous powder MS (APCI) 710 (M + H) |
| 844 | 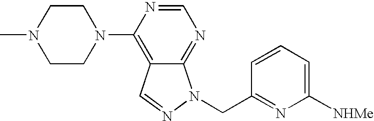 | Amorphous powder MS (APCI) 697 (M + H) |
| 845 | 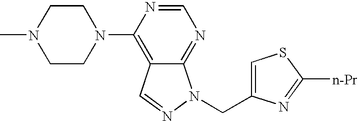 | Amorphous powder MS (APCI) 716 (M + H) |
| 846 | 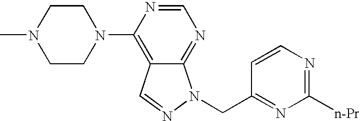 | Amorphous powder MS (APCI) 711 (M + H) |
| 847 | 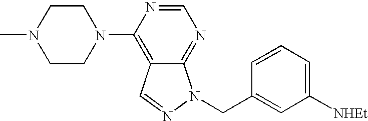 | Amorphous powder MS (APCI) 710 (M + H) |
| 848 | 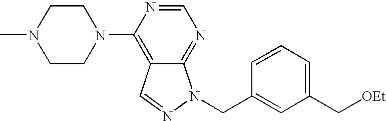 | Amorphous powder MS (APCI) 725 (M + H) |
| 849 | 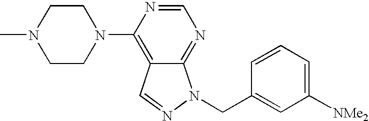 | Amorphous powder MS (APCI) 710 (M + H) |
| 850 | 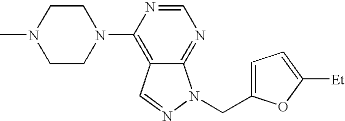 | Amorphous powder MS (APCI) 685 (M + H) |
| 851 | 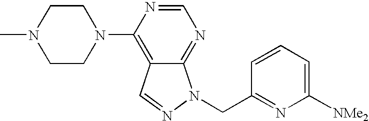 | Amorphous powder MS (APCI) 711 (M + H) |
| 852 | 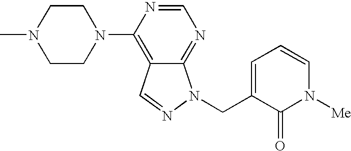 | Amorphous powder MS (APCI) 698 (M + H) |
| 853 | 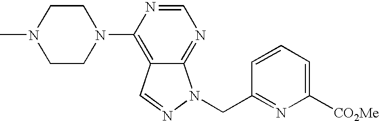 | Amorphous powder MS (APCI) 726 (M + H) |
| 854 | 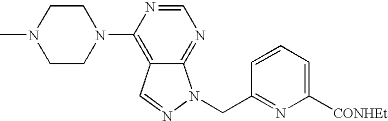 | Amorphous powder MS (APCI) 739 (M + H) |
| 855 | 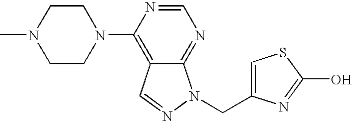 | Amorphous powder MS (APCI) 718 (M + H) |
| 856 | 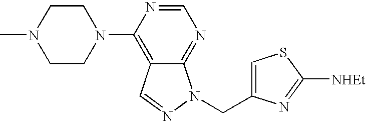 | Amorphous powder MS (APCI) 717 (M + H) |
| 857 | 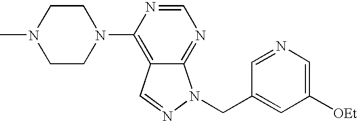 | Amorphous powder MS (APCI) 710 (M + H) |
| 858 | 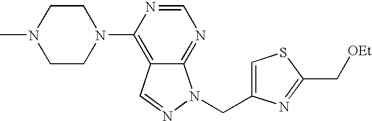 | Amorphous powder MS (APCI) 761 (M + H) |

TABLE 47
(No. 5)

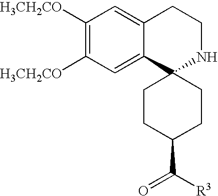

| Ex. | | Physicochemical |
|---|---|---|
| No. | R³ | Properties etc. |
| 859 | 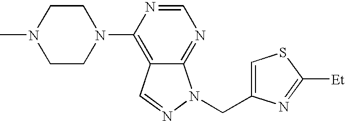 | Amorphous powder MS (APCI) 645 (M + H) |
| 860 | 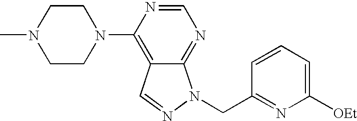 | Amorphous powder MS (APCI) 655 (M + H) |
| 861 | 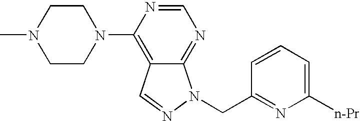 | Amorphous powder MS (APCI) 653 (M + H) |
| 862 | 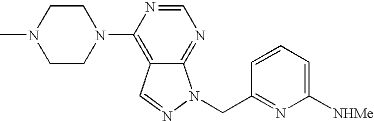 | Amorphous powder MS (APCI) 640 (M + H) |
| 863 | 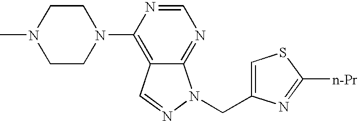 | Amorphous powder MS (APCI) 659 (M + H) |
| 864 | 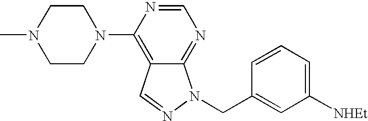 | Amorphous powder MS (APCI) 653 (M + H) |
| 865 | 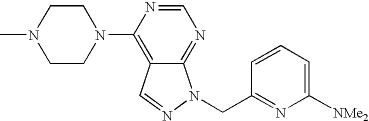 | Amorphous powder MS (APCI) 654 (M + H) |
| 866 | 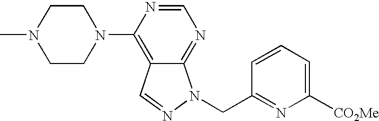 | Amorphous powder MS (APCI) 669 (M + H) |
| 867 | 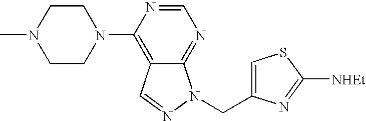 | Amorphous powder MS (APCI) 660 (M + H) |
| 868 | 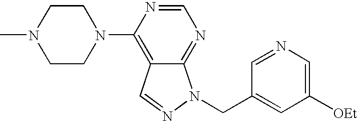 | Amorphous powder MS (APCI) 653 (M + H) |

TABLE 47
(No. 6)

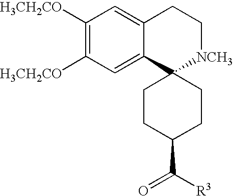

| Ex. | | Physicochemical |
|---|---|---|
| No. | R³ | Properties etc. |
| 869 | 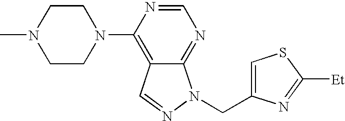 | Amorphous powder MS (APCI) 659 (M + H) |
| 870 | 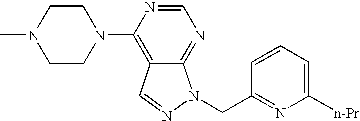 | Amorphous powder MS (APCI) 667 (M + H) |
| 871 | 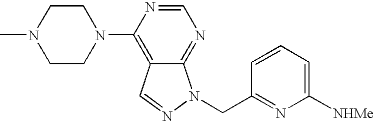 | Amorphous powder MS (APCI) 654 (M + H) |
| 872 | 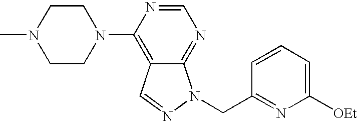 | Amorphous powder MS (APCI) 669 (M + H) |
| 873 | 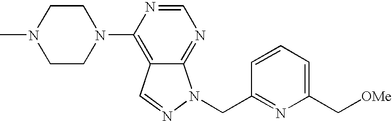 | Amorphous powder MS (APCI) 669 (M + H) |
| 874 | 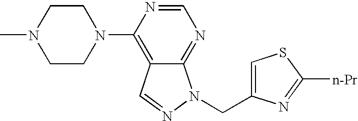 | Amorphous powder MS (APCI) 673 (M + H) |
| 875 | 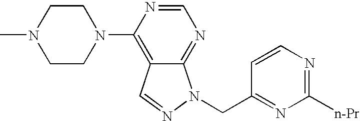 | Amorphous powder MS (APCI) 668 (M + H) |
| 876 | 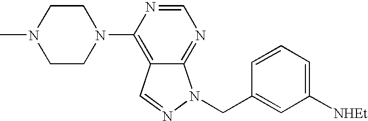 | Amorphous powder MS (APCI) 667 (M + H) |
| 877 | 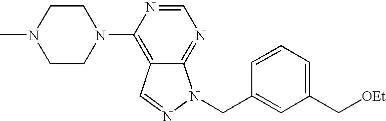 | Amorphous powder MS (APCI) 682 (M + H) |
| 878 | 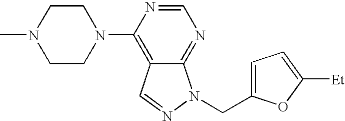 | Amorphous powder MS (APCI) 642 (M + H) |
| 879 | 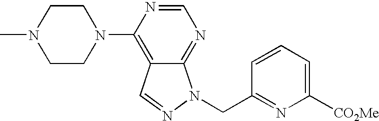 | Amorphous powder MS (APCI) 683 (M + H) |
| 880 | 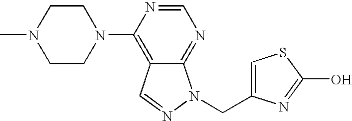 | Amorphous powder MS (APCI) 675 (M + H) |
| 881 | 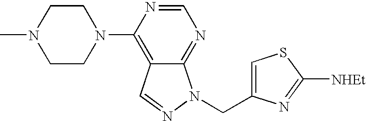 | Amorphous powder MS (APCI) 674 (M + H) |
| 882 | 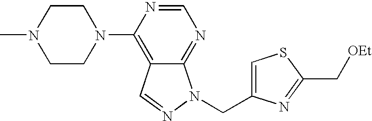 | Amorphous powder MS (APCI) 718 (M + H) |

TABLE 47
(No. 7)

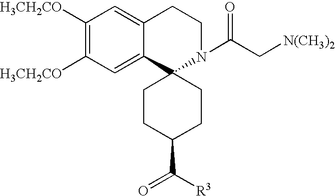

| Ex. | | Physicochemical |
|---|---|---|
| No. | R³ | Properties etc. |
| 883 | 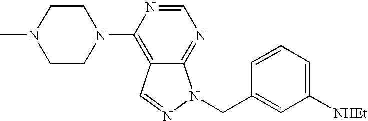 | Amorphous powder MS (APCI) 738 (M + H) |
| 884 | 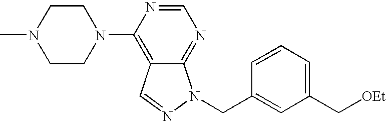 | Amorphous powder MS (APCI) 753 (M + H) |
| 885 | 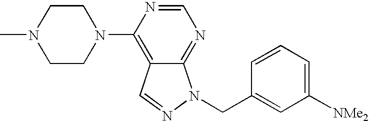 | Amorphous powder MS (APCI) 738 (M + H) |
| 886 | 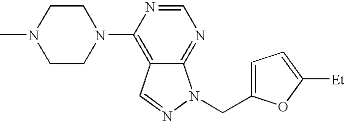 | Amorphous powder MS (APCI) 713 (M + H) |
| 887 | 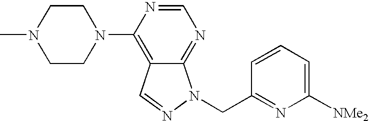 | Amorphous powder MS (APCI) 739 (M + H) |
| 888 | 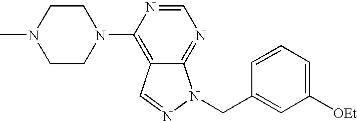 | Amorphous powder MS (APCI) 739 (M + H) |
| 889 | 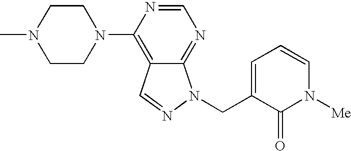 | Amorphous powder MS (APCI) 726 (M + H) |
| 890 | 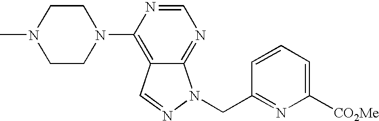 | Amorphous powder MS (APCI) 754 (M + H) |
| 891 | 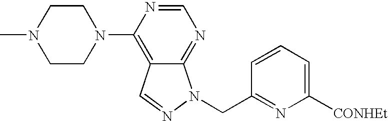 | Amorphous powder MS (APCI) 767 (M + H) |
| 892 | 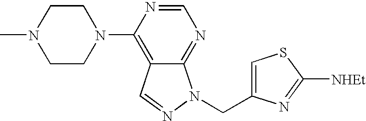 | Amorphous powder MS (APCI) 745 (M + H) |
| 893 | 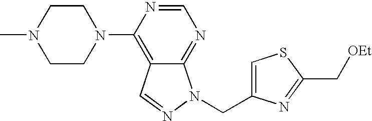 | Amorphous powder MS (APCI) 758 (M + H) |
| 894 | 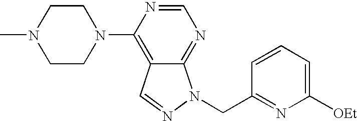 | Amorphous powder MS (APCI) 740 (M + H) |
| 895 | 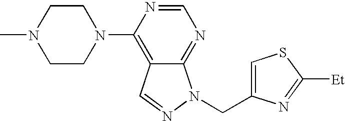 | Amorphous powder MS (APCI) 730 (M + H) |
| 896 | 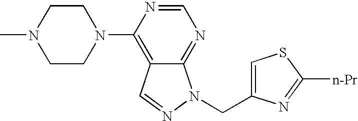 | Amorphous powder MS (APCI) 744 (M + H) |
| 897 | 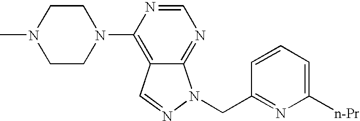 | Amorphous powder MS (APCI) 738 (M + H) |
| 898 | 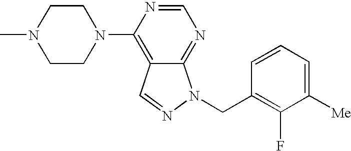 | Amorphous powder MS (APCI) 727 (M + H) |
| 899 | 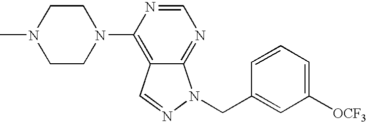 | Amorphous powder MS (APCI) 779 (M + H) |

TABLE 47
(No. 8

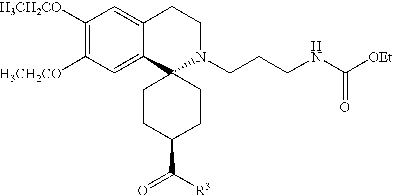

| Ex. | | Physicochemical |
|---|---|---|
| No. | R³ | Properties etc. |
| 900 | 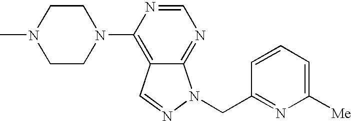 | Amorphous powder MS (APCI) 754 (M + H) |
| 901 | 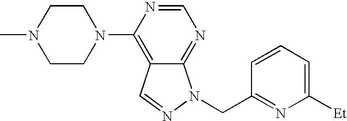 | Amorphous powder MS (APCI) 768 (M + H) |
| 902 | 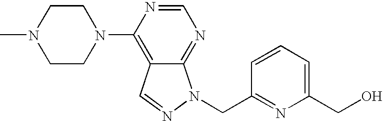 | Amorphous powder MS (APCI) 770 (M + H) |
| 903 | 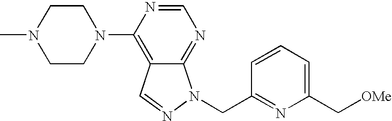 | Amorphous powder MS (APCI) 784 (M + H) |
| 904 | 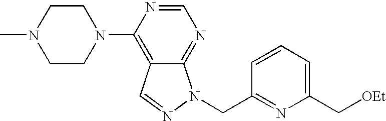 | Amorphous powder MS (APCI) 798 (M + H) |
| 905 | 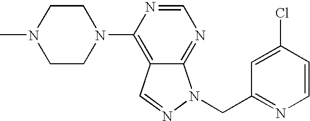 | Amorphous powder MS (APCI) 774 (M + H) |
| 906 | 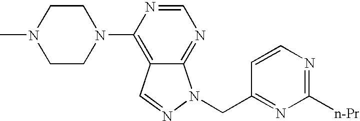 | Amorphous powder MS (APCI) 783 (M + H) |
| 907 | 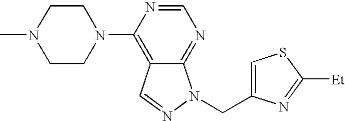 | Amorphous powder MS (APCI) 774 (M + H) |
| 908 | 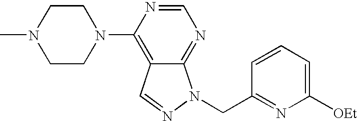 | Amorphous powder MS (APCI) 784 (M + H) |
| 909 | 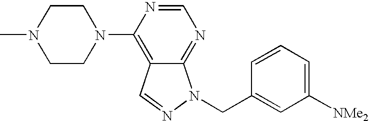 | Amorphous powder MS (APCI) 782 (M + H) |
| 910 | 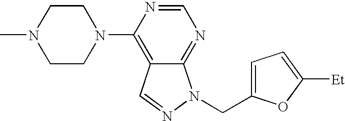 | Amorphous powder MS (APCI) 757 (M + H) |
| 911 | 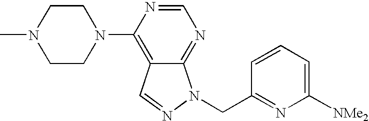 | Amorphous powder MS (APCI) 783 (M + H) |
| 912 | 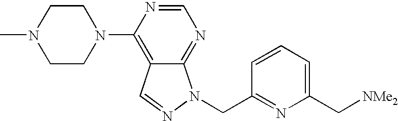 | Amorphous powder MS (APCI) 797 (M + H) |
| 913 | 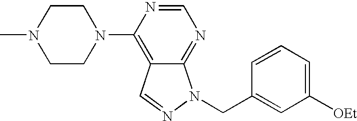 | Amorphous powder MS (APCI) 783 (M + H) |
| 914 | 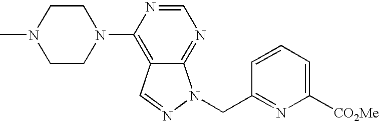 | Amorphous powder MS (APCI) 798 (M + H) |
| 915 | 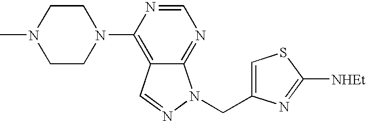 | Amorphous powder MS (APCI) 789 (M + H) |
| 916 | 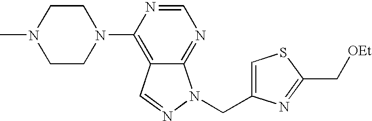 | Amorphous powder MS (APCI) 802 (M + H) |

TABLE 47
(No. 9)

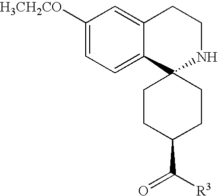

| Ex. | | Physicochemical |
|---|---|---|
| No. | R³ | Properties etc. |
| 917 | 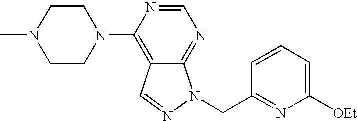 | Amorphous powder MS (APCI) 611 (M + H) |
| 918 | 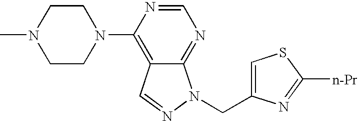 | Amorphous powder MS (APCI) 615 (M + H) |
| 919 | 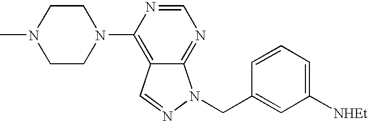 | Amorphous powder MS (APCI) 609 (M + H) |
| 920 | 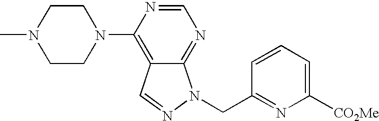 | Amorphous powder MS (APCI) 625 (M + H) |

TABLE 47
(No. 10)

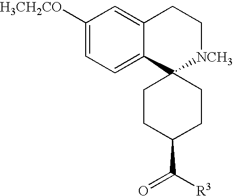

| Ex. | | Physicochemical |
|---|---|---|
| No. | R³ | Properties etc. |
| 921 | 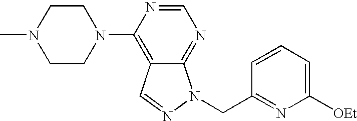 | Amorphous powder MS (APCI) 625 (M + H) |
| 922 | 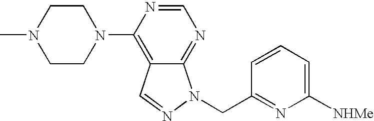 | Amorphous powder MS (APCI) 610 (M + H) |
| 923 | 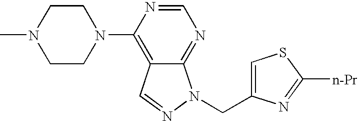 | Amorphous powder MS (APCI) 629 (M + H) |
| 924 | 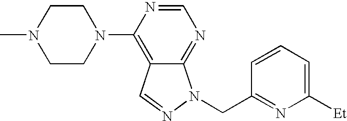 | Amorphous powder MS (APCI) 609 (M + H) |

TABLE 47
(No. 11)

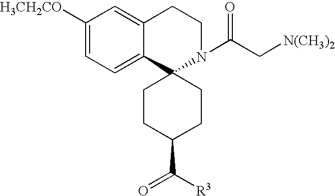

| Ex. | | Physicochemical |
|---|---|---|
| No. | R³ | Properties etc. |
| 925 | 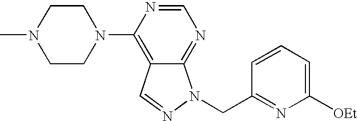 | Amorphous powder MS (APCI) 696 (M + H) |
| 926 | 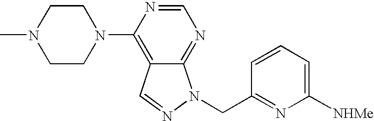 | Amorphous powder MS (APCI) 681 (M + H) |
| 927 | 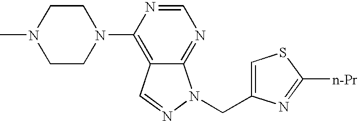 | Amorphous powder MS (APCI) 700 (M + H) |
| 928 | 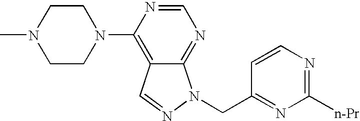 | Amorphous powder MS (APCI) 695 (M + H) |
| 929 | 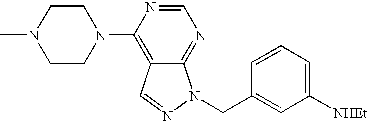 | Amorphous powder MS (APCI) 694 (M + H) |
| 930 | 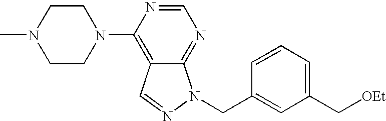 | Amorphous powder MS (APCI) 709 (M + H) |
| 931 | 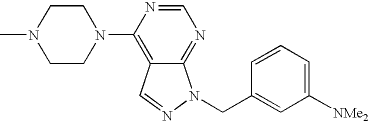 | Amorphous powder MS (APCI) 694 (M + H) |
| 932 | 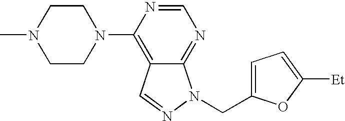 | Amorphous powder MS (APCI) 669 (M + H) |
| 933 | 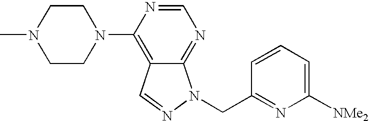 | Amorphous powder MS (APCI) 695 (M + H) |
| 934 | 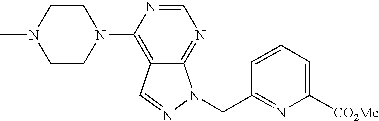 | Amorphous powder MS (APCI) 710 (M + H) |
| 935 | 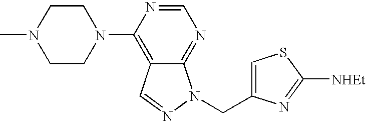 | Amorphous powder MS (APCI) 701 (M + H) |
| 936 | 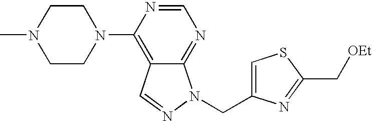 | Amorphous powder MS (APCI) 714 (M + H) |

### Example 937

A solution of (1α,4β)-3′,4′-dihydro-6′,7′-dimethoxy-4-[4-[1-(3-methylbenzyl)-1H-pyrazolo[3,4-d]pyrimidin-4-yl]-1-piperadinyl]carbonyl-spiro[cyclohexan-1,1′(2′H)-isoquinoline] (Compound obtained in Example 583(1)) (185 mg) and triethylamine (0.056 mL) in dichloromethane is added dropwise chloroacetyl chloride (0.030 mL) under ice-cooling and the mixture is stirred for 30 minutes. After addition of an aqueous 40% methylamine to the reaction mixture under ice-cooling, it is further stirred overnight at a room temperature. The reaction mixture is evaporated to concentrate and the residue is dissolved in ethylacetate. The mixture is washed with a saturated sodium hydrogencarbonate solution and a saturated NaCl solution, successively. The mixture is dried over sodium sulfate and evaporated to remove solvents. The residue is purified by silica gel column chromatography (solvents; chloroform:methanol:aqueous 28% ammonia=200:10:1) and lyophilized in the presence of tert-butanol to give (1α,4β)-3′,4′-dihydro-6′,7′-dimethoxy-2′-(N-methyl)aminoacetyl-4-[4-[1-(3-methylbenzyl)-1H-pyrazolo[3,4-d]pyrimidin-4-yl]-1-piperadinyl]carbonyl-spiro[cyclohexan-1,1′(2′H)-isoquinoline] as an amorphous powder. MS(APCI)m/z: 667(M+H)

### Examples 938 to 944

The corresponding compounds are treated in the same manner as described in Example 937 to give the compounds as shown in the following table (Table.48).

TABLE 48

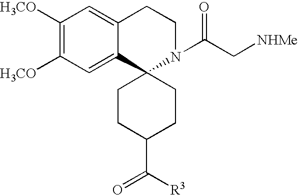

| Ex. No. | R³ | Physicochemical Properties etc. |
|---|---|---|
| 938 | 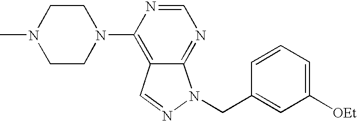 | Amorphous powder MS(APCI) 697(M + H) |
| 939 | 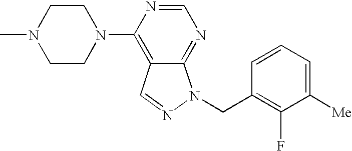 | Amorphous powder MS(APCI) 685(M + H) |
| 940 | 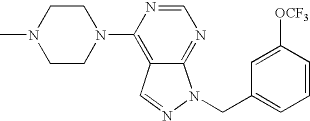 | Amorphous powder MS(APCI) 737(M + H) |
| 941 | 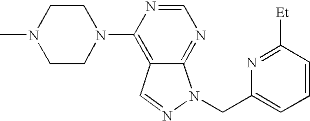 | Amorphous powder MS(APCI) 682(M + H) |
| 942 | 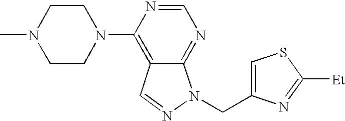 | Amorphous powder MS(APCI) 688(M + H) |
| 943 | 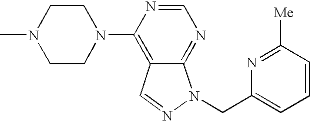 | Amorphous powder MS(APCI) 668(M + H) |
| 944 | 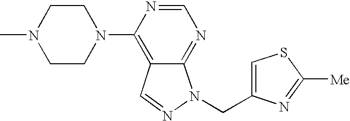 | Amorphous powder MS(APCI) 574(M + H) |

### Reference Example 1

1) To a solution of diethyl malonate (300 g) and tert-butyl acrylate (624 g) in tetrahydrofuran (1.5 L) and tert-butyl alcohol (1.5 L) is added potassium hydroxide (2.62 g) under ice-cooling and the mixture is stirred at room temperature overnight. The reaction mixture is concentrated and to the residue is added diethyl ether (4.5 L). The mixture is washed with water (1.5 L×2) and saturated aqueous NaCl solution (1.5 L), successively and dried over sodium sulfate. The solvent is concentrated under reduced pressure to give di-tert-butyl 4,4-bisethoxycarbonylpimelate as an oil.

2) A solution of the compound obtained in the above step (1) in formic acid (1.2 L) is stirred at room temperature overnight and refluxed for 2 hours. The reaction mixture is evaporated and to the residue is added diethyl ether (750 mL). The precipitates are collected and washed with diisopropyl ether (450 mL) to give 4,4-bis(ethoxycarbonyl)pimelic acid (557 g, 98%) as colorless crystals. M.p. 120° C.

3) To a suspension of the compound obtained in the above step (2) (100 g) and oxalyl chloride (104 g) in methylene chloride (400 mL) is added dimethylformamide (0.03 mL) and the mixture is stirred for 5 hours. The reaction mixture is concentrated. A solution of the residue in dimethoxyethane (200 mL) is added dropwise to a solution of homoveratrylamine (3,4-dimethoxyphenethylamine; 119 g) and potassium carbonate (114 g) in ethyl acetate (1.2 L) and water (1 L) under vigorously stirring at 0-5° C. over a period of 40 minutes. The mixture is stirring for 20 minutes under ice-cooling and at room temperature for 1 hour. The organic layer is separated and washed with saturated aqueous NaCl solution (0.5 L), 10% aqueous hydrochloric acid (1 L), saturated aqueous NaCl solution (0.5 L), saturated aqueous sodium hydrogencarbonate solution (1 L) and saturated aqueous NaCl solution, successively. The organic layer is dried over sodium sulfate and concentrated under reduced pressure. The resultant crystals are recrystallized from ethyl acetate to give N,N′-di-(3,4-dimethoxyphenethyl)-4,4-bis(ethoxycarbonyl)pimeloyl diamide (196 g) as colorless crystals. M.p. 85-86° C.

4) A solution of the compound obtained in the above step (3) (200 g) and phosphorus oxychloride (97.2 g) in acetonitrile (1 L) is refluxed for 3 hours. The reaction mixture is concentrated under reduced pressure. The residue is dissolved in 10% aqueous hydrochloric acid (400 mL) and the solution is washed with ethyl acetate (600 mL)). The ethyl acetate layer are extracted with 10% aqueous hydrochloric acid (100 mL). The aqueous layer is combined and diluted with water (500 mL). The solution is basified with potassium carbonate (500 g) under ice-cooling and extracted with chloroform (700 mL). The organic layer is washed with saturated aqueous NaCl solution (300 mL), dried over sodium sulfate and concentrated under reduced pressure. The residue is recrystallized from isopropanol by 5 times to give (1R*,2R*)-3′,4′-dihydro-6′,7′-dimethoxy-2-(3,4-dihydro-6,7-dimethoxy-1-isoquinolyl)-4,4-bisethoxycarbonyl-spiro[cyclohexane-1,1′(2′H)-isoquinoline] (184 g, 95%) as crystals. M.p. 116-118° C.

5) To a solution of the compound obtained in the above step (4) (178 g) and diisopropylethylamine (77.6 g) in dichloromethane (1.7 L) is added dropwise a solution of acetyl bromide (73.8 g) in dichloromethane (150 mL) at −5-0° C. for 2 hours. To the mixture is added water (1 L) and the organic layer is separated and washed with saturated aqueous sodium hydrogencarbonate solution and saturated aqueous NaCl solution, successively. The washed solution is dried over sodium sulfate and concentrated under reduced pressure. The resultant crystals are recrystallized from methylene chloride (100 mL) and ethyl acetate (1 L) to give (1R*2R*)-2′-acetyl-3′,4′-dihydro-6′,7′-dimethoxy-2-(3,4-dihydro-6,7-dimethoxy-1-isoquinolyl)-4,4-bisethoxycarbonyl-spiro[cyclohexane-1,1′(2′H)-isoquinoline] (184 g) as crystals.

M.p. 163-165° C.

6) To a suspension of the compound obtained in the above step (5) (340 g) in ethanol (1360 mL), 2-methoxyethanol (1360 mL), and tetrahydrofuran (1360 mL) is added portionwise sodium borohydride (30.4 g) at room temperature over a period of 1 hour and the mixture is stirred for 4 hours. To the reaction mixture is added sodium borohydride (10.1 g) at room temperature over a period of 20 minutes and the mixture is stirred for 30 minutes. The reaction mixture is evaporated under reduced pressure. To the residue is added dropwise water (3.4 L) at room temperature and the mixture is stirred overnight. The precipitates are collected and dried to give (1R*,2R*(S*))3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (271 g, 81%) as crystals.

M.p. 146-147° C.

7) To a solution of the compound obtained in the above step (6) (271 g) and triethylamine (66 g) in methylene chloride (1.3 L) is added dropwise a solution of acryloyl chloride (59 g) in methylene chloride (350 mL) at −5-0° C. for 3 hours. The mixture is stirred for 30 minutes. To the reaction mixture is added water (1 L) and the organic layer is separated. The organic layer is washed with saturated aqueous sodium hydrogencarbonate solution and saturated aqueous NaCl solution, successively and dried over sodium sulfate. The dried solution is concentrated under reduced pressure and the residue is dissolved in chloroform. The solution is filtered through NH-silica gel (540 g). The filtrate is concentrated and recrystallized from ethyl acetate to give (1R*,2R*(S*))-2′-acryloyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (208 g, 79%) as crystals.

M.p. 150-151° C.

8) A solution of the compound obtained in the above step (7) (181 g) and 40% methylamine (820 mL) in acetonitrile (2 L) is stirred at room temperature overnight and the reaction mixture is concentrated under reduced pressure. The residue is dissolved in ethyl acetate and the solution is washed with saturated aqueous sodium hydrogencarbonate solution and saturated aqueous NaCl solution. The organic layer is dried over sodium sulfate and concentrated. The residue is recrystallized from ethyl acetate and diisopropyl ether (2:1) to give (1R*,2R*(S*))-2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4,4-bisethoxycarbonyly-spiro[cyclohexane-1,1′(2′H)-isoquinoline] (141 g, 89%) as crystals.

M.p. 119-121° C.

9) To a suspension of the compound obtained in the above step (8) (119 g) in ethanol (675 mL) is added a solution of sodium hydroxide (67.3 g) in water (137 mL) and the mixture is refluxed for 45 minutes. After cooling with ice-water, the reaction mixture is diluted with water (600 mL). The mixture is evaporated to remove 700 mL of the solvent and to the residue is added dioxane (675 mL). To the mixture is added dropwise benzyl chloroformate (33.2 g) under ice-cooling and the mixture is stirred for 30 minutes. To the residue is added conc. HCl (ca. 130 mL) under ice-cooling to adjust its pH to 4 to 5. The mixture is evaporated to remove 400 mL of the solvent. The residual solution is stirred overnight at room temperature. The precipitates are collected and washed with water and ethyl ether and dried to give (1R*,2R*(S*))-2′-[3-(N-methyl-N-benzyloxy-carbonylamino)propionyl]-3′,4′-dihydro-6′,7′-dimethoxy-2-(2-ethyl-1,2,3,4-tetrahydro-6,7-dimethoxy-1-isoquinolyl)-4,4-biscarboxy-spiro[cyclohexane-1,1′(2′H)-isoquinoline] (156 g).

10) A solution of the compound obtained in the above step (9) (200 g) in pyridine (2 L) is refluxed for 2.5 hours. After cooling, the reaction mixture is concentrated under reduced pressure and the residue is dissolved in chloroform. The solution is washed with water (500 mL) and saturated aqueous NaCl solution (500 mL). The organic layer is dried over sodium sulfate and evaporated under reduced pressure. The residue is crystallized from acetone (1 L) under stirring. The precipitates are dissolved in hot chloroform and the solution is evaporated. The residue is dissolved in hot acetone (1 L) and stirred. The precipitated crystals are collected by filtration and dried to give (1R*,2R*(S*),4R*)-2′-[3-(N-methyl-N-benzyloxycarbonylamino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid as crystals (145 g, 77%; Compound 1(10a)).

M.p. 181-183° C.

The mother liquor obtained from the above step is collected and concentrated, and the residue is purified by column chromatography on silica gel to give (1R*,2R*(S*),4S*)-2′-[3-(N-methyl-N-benzyloxycarbonylamino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid as crystals (15 g, 8%; Compound 1(10b)). M.p. 118-120° C.

### Reference Example 2

1) To a solution of (1R*,2R*(S*))-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (3.0 g) and diisopropylethylamine (1.56 g) in 1,2-dichloroethane (50 mL) is added dropwise a solution of acetyl bromide (1.19 g) in dichloroethane at 0° C. for 3 hour and the mixture is stirred for 1 hour. And then, to the mixture is added silica gel (1.5 g) and the mixture is stirred overnight at room temperature. The reaction mixture is filtered and the silica gel on the filter is washed with chloroform-methanol (5:1). The combined filtrate is concentrated and the residue is dissolved in ethyl acetate and the solution is washed with saturated aqueous sodium hydrogencarbonate solution and saturated aqueous NaCl solution. The organic layer is dried over sodium sulfate and recrystallized from isopropyl alcohol-diisopropyl alcohol to give (1R*,2R*(S*))-2′-acetyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (2.6 g, 81%) as crystals.

The mother liquor obtained from the above step is concentrated and the resultant residue is purified by column chromatography on NH-silica gel (solvent; ethyl acetate:n-hexane=1:3→1:1) to give the same compound as above (374 mg, 12%).

M.p. 142-144° C.

2) To a suspension of the compound obtained in the above step (1) (50.0 g) in ethanol (600 mL) is added an aqueous sodium hydroxide (30.07 g/300 mL water) and the mixture is refluxed for 22 hours. After cooling, the reaction mixture is neutralized with cooled 10% hydrochloric acid and concentrated. The trace of water is removed by azeotropic distillation with toluene. The suspension of the obtained residue in pyridine (1 L) is refluxed for 2 hours. The reaction mixture is concentrated and the residue is purified by column chromatography on silica gel (chloroform:methanol=35:1→5:1) to give the more polar product, (1R*,2R*(S*),4R*)-2′-acetyl-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (30.6 g, 72%; compound 2(2a)), and the less polar product, (1R*,2R*(S*),4S*)-2′-acetyl-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (4.0 g, 95%; compound 2(2b)).

Compound 2 (2a): M.p. 202-205° C.

Compound 2(2b): An amorphous powder, MS(FAB): 567(M+H), IR(Nujol)cm⁻¹: 3360, 1720, 1640, 1610.

3) To a suspension of the compound obtained in the above step (2) (Compound 2(2a); 2.0 g) in tetrahydrofuran (100 mL) is slowly added portionwise lithium aluminum hydride (0.67 g) and the mixture is refluxed for 3.5 hours. To the reaction mixture is added saturated aqueous ammonium chloride solution (2.0 mL) under ice-cooling and the mixture is filtered to remove insoluble materials. The filtrate is concentrated under reduced pressure and the residue is purified by column chromatography on silica gel (solvent; chloroform:methanol:aqueous 28% ammonia=120:10:1→80:10:1) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-hydroxymethyl-spiro[cyclohexane-1,1′(2′H)-isoquinoline] (1.45 g, 77%) as an amorphous powder.

4) To a solution of oxalyl chloride (0.44 mL) in methylene chloride (45 mL) is added dropwise a solution of dimethyl sulfoxide (0.54 g) in methylene chloride (10 mL) at −78° C. and the mixture is stirred for 10 minutes. To the mixture is added dropwise a solution of the compound obtained in the above step (3) (1.37 g) in methylene chloride (15 mL) at −78° C. The mixture is stirred at −78° C. for 15 minutes and at −45-−30° C. for 2 hours. To the mixture is added dropwise triethylamine (2.58 mL) at −45° C. and the mixture is stirred at 0° C. for 1 hour. To the reaction mixture is added saturated aqueous ammonium chloride solution and the mixture is extracted with ethyl acetate. The extract is washed with a saturated NaCl solution, dried over sodium sulfate, and concentrated under reduced pressure to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-4-formyl-spiro[cyclohexane-1,1′(2′H)-isoquinoline] (1.30 g) as an amorphous powder.

5) To a solution of the compound obtained in the above step (4) (1.1 g) and 2-methyl-2-butene (11 mL) in tert-butyl alcohol (110 mL) is added dropwise a solution of sodium chlorate (1.7 g) in phosphate buffer (pH7, 50 mL) at room temperature and the mixture is stirred for 30 minutes. To the mixture is added water and the reaction mixture is extracted with chloroform (×4). The organic layer is dried over sodium sulfate and concentrated under reduced pressure. The residue is purified by column chromatography on silica gel (chloroform:methanol:aqueous ammonia=400:100:1) to give (1R*,2R*(S*),4R*)-2′-ethyl-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (0.85 g) as crystals. M.p. 124-126° C.

6) The compound obtained in Reference example 2(2b) is treated in the same manner as described in the above steps (3) and (4) to give (1R*,2R*(S*),4S*)-2′-ethyl-3′,4′-dihydro-6′,7′-dimethoxy-2-)2-ethyl-1,2,3,4-tetrahydro-6,7-dimethoxy-1-isoquinolyl)-4-formyl-spiro[cyclohexane-1,1′(2′H)-isoquinoline]. To a solution of the present compound (389 mg) in ethanol (6 mL) is added silver nitrate (369 mg) at 0° C. To the mixture is added dropwise an aqueous potassium hydroxide (264 mg in 3 mL of water) and the mixture is washed with water (3 mL). The mixture is stirred for 2 hours at room temperature and filtered with Celite. The residue on the filter is washed with ethanol and the combined filtrate is concentrated. To the residue is added water and the mixture is neutralized with 10% hydrochloric acid. The mixture is extracted with chloroform-methanol (4:1). The extract is dried over sodium sulfate and concentrated. The residue is purified by column chromatography on silica gel (chloroform:methanol:aqueous ammonia=200:50:2) to give (1R*,2R*(S*),4S*)-2′-ethyl-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (260 mg, 65%) as crystals. M.p. 136-140° C.

### Reference Example 3

1) To a suspension of (1R*,2R*(S*),4R*)-2′-[3-(N-methyl-N-benzyloxycarbonylamino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (compound obtained in Reference example 1(10a)) (40 g) in tetrahydrofuran (240 mL) is added dropwise a solution of oxalyl chloride (7.51 g) in tetrahydrofuran (40 mL) at room temperature over a period of 5 minutes. The mixture is stirred for 15 minutes, and the excess of oxalyl chloride is concentrated under reduced pressure. To the residue is added tetrahydrofuran (240 mL). A solution of triethylamine (6.53 g) in tetrahydrofuran (40 mL) is added dropwise to the mixture under ice-cooling. The mixture is stirred for 30 minutes and the precipitates (triethylammonium chloride) are removed by filtration. The filtrate is concentrated to give a corresponding acid chloride of the compound obtained in Reference example 1(10a) as an amorphous powder.

2) To a solution of (4R)-4-isopropyloxazolidin-2-one (8.33 g) in tetrahydrofuran (240 mL) is added dropwise 1.47M n-butyllithium in n-hexane at −60° C. over a period of 20 minutes. The mixture is stirred for 20 minutes at −60° C. A solution of the acid chloride obtained in the above step (1) in tetrahydrofuran (280 mL) is added dropwise to the mixture at −60° C. over a period of 40 minutes, at −60° C.-−5° C. for 1 hour, and under ice-cooling for 1 hour. To the mixture is added saturated aqueous ammonium chloride (100 mL) under ice-cooling and the mixture is stirred for 10 minutes. To the reaction mixture is added water (100 mL) and the organic layer is separated. The organic layer is washed with saturated aqueous NaCl solution, dried over sodium sulfate, and concentrated. The residue is separated by column chromatography on neutral silica gel (chloroform acetonitrile=5:1→2:1, chloroform:methanol=5:1) to give the less polar product (compound 3(2a)) and the more polar product (compound 3(2b)). The less polar product is further purified by column chromatography on NH-silica gel (Chromatorex NH DM1020; Fuji Silysia Kagaku KK) (ethyl acetate:n-hexane=1:1 to 2:1) to give (1R*,2R*(S*),4R*)-2′-[3-(N-methyl-N-benzyloxy-carbonylamino)propionyl]-3′,4′-dihydro-6′,7′-dimethoxy-2-(2-ethyl-1,2,3,4-tetrahydro-6,7-dimethoxy-1-isoquinolyl)-4-[((4R)-isopropyl-2-oxo-3-oxazolinyl)carbonyl]-spiro[cyclohexane-1,1′(2′H)-isoquinoline] (16.01 g, 34.8%; compound 3(2a)) as an amorphous powder. The more polar product is purified by column chromatography on alumina gel (chloroform:ethyl acetate:n-hexane=1:2:5) to give (1R*,2R*(S*),4S*)-2′-[3-(N-methyl-N-benzyoxylcarbonyl-amino)propionyl]-3′,4′-dihydro-6′,7′-dimethoxy-2-(2-ethyl-1,2,3,4-tetrahydro-6,7-dimethoxy-1-isoquinolyl)-4-[((4R)-isopropyl-2-oxo-3-oxazolinyl)carbonyl]-spiro[cyclohexane-1,1′(2′H)-isoquinoline] (13.05 g, 28%; compound 3(2b)) as an amorphous powder.

3) To a solution of the compound 3(2a) obtained in the above step (2) (13.05 g) in methanol (260 mL) is added dropwise an aqueous potassium hydroxide (9.94 g in 100 mL water) at room temperature over a period of 40 minutes. The mixture is stirred at room temperature for 20 minutes and refluxed for 40 minutes. After cooling, the reaction mixture is evaporated under reduced pressure. To the residue is added water (131 mL) and the mixture is washed with a mixture of diethyl ether (78 mL) and ethyl acetate (16 mL) (×4). The mixture is neutralized with 10% hydrochloric acid (ca. 12.5 mL) under ice cooling and then basified with 10% aqueoussodium hydroxide (18 mL). The mixture is washed twice with a mixture of diethyl ether (45 mL) and ethyl acetate (9 mL). The mixture is neutralized (pH7) with 10% hydrochloric acid under ice-cooling. The aqueous layer is saturated by addition of NaCl (60 g) and extracted with ethyl acetate (110 mL×3). The extract is washed with saturated aqueous NaCl solution and dried over sodium sulfate. The organic layer is concentrated under reduced pressure and the residue is recrystallized from diethyl ether, (93 mL) to give an optically active compound, (−)-(1R*,2R*(S*),4R*)-2′-[3-(N-methyl-N-benzyloxy-carbonylamino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (9.71 g, 24%; Compound 3(3a)) as crystals.

The compound 3(2b) is treated in the same manner as described above to give (+)-(1R*,2R*(S*),4R*)-2′-[3-(N-methyl-N-benzyloxy-carbonylamino)propionyl]-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid 7.64 g, 19%; Compound 3(3b)) as crystals.

Compound 3(3a): M.p. 144-145° C., [α]_{D}²⁵−26.5° (c1.0, chloroform), 99.9%ee (HPLC; SUMICHIRAL OA-3100 with 20 mM ammonium acetate/methanol).

Compound 3(3b): M.p. 146-147° C., [α]_{D}²⁵+26.5° (c1.0, chloroform), 99.6%ee (HPLC; The same condition as above).

### Reference Example 4

1) (1R*,2R*)-2′-acetyl-3′,4′-dihydro-6′,7′-dimethoxy-2-(3,4-dihydro-6,7-dimethoxy-1-isoquinolyl)-4,4-bisethoxycarbonyl-spiro[cyclohexane-1,1′(2′H)-isoquinoline] (compound obtained in Reference example 1(5)) (10.0 g) and iodomethane (100 mL) are refluxed for 7 hours. After cooling, the precipitates are collected and washed with ethyl acetate to give 2′-acetyl-3′,4′-dihydro-6′,7′-dimethoxy-2-(2-methyl-3,4-dihydro-6,7-dimethoxyisoquinolinium-1-yl)-4,4-bisethoxycarbonyl-spiro[cyclohexane-1,1′(2′H)-isoquinoline]iodide (10.2 g, 84%) as a mixture of diastereomers. M.p. 171-173° C. (Decomp.)

MS(ESI)m/z: 651 (M+), IR(Nujol)cm⁻¹: 1743, 1727, 1629, 1601.

2) A solution of the compound obtained in the above step (1) (9.0 g) in ethanol (180 mL) is added sodium borohydride (0.438 g) under ice-cooling. The mixture is stirred at the same temperature for 30 minutes and to the reaction mixture is added 10% hydrochloric acid. The reaction mixture is concentrated and to the residue is added ethyl acetate. The solution is washed with water, saturated aqueous sodium hydrogencarbonate solution and saturated aqueous NaCl solution. The organic layer is dried over sodium sulfate and concentrated. The residue is recrystallized from a mixture of ethyl acetate-isopropyl ether to give (1R*,2S*)-2′-acetyl-3′,4′-dihydro-6′,7′-dimethoxy-2-(2-methyl-1,2,3,4-tetrahydro-6,7-dimethoxy-1-isoquinolyl)-4,4-bisethoxycarbonyl-spiro[cyclohexane-1,1′(2′H)-isoquinoline] (7.38 g, 98%) as crystals. M.p. 165.5-167.5° C., MS(ESI)m/z: 653 (M+H), IR(Nujol)cm⁻¹: 1739, 1721

3) A mixture of the compound obtained in the above step (2) (7.0 g), sodium hydroxide (4.3 g), ethanol (86 mL) and water (43 mL) is refluxed for 2 days. After cooling, the reaction mixture is neutralized with 1M hydrochloric acid and concentrated. The residue is dissolved in pyridine (100 mL) and refluxed for 4 hours. The reaction mixture is concentrated and to the residue is added chloroform. The solution is washed with water and dried over sodium sulfate. The organic layer is concentrated and purified by column chromatography on silica gel (chloroform:methanol=5:1 to 1:1) to give (1R*,2S*(S*),4R*)-2′-acetyl-3′,4′-dihydro-6′,7′-dimethoxy-2-(2-methyl-1,2,3,4-tetrahydro-6,7-dimethoxy-1-isoquinolyl)-4-carboxy-spiro[cyclohexane-1,1′(2′H)-isoquinoline] (3.43 g, 58%; compound 4(3a)) and (1R*,2S*(R*),4S*)-2′-acetyl-3′,4′-dihydro-6′,7′-dimethoxy-2-(2-methyl-1,2,3,4-tetrahydro-6,7-dimethoxy-1-isoquinolyl)-4-carboxy-spiro[cyclohexane-1,1′(2′H)-isoquinoline] (0.96 g, 16%; compound 4(3b)) as crystals, respectively.

Compound 4(3a): M.p. 235-237° C. (dec.), MS(ESI)m/z: 553 (M+H), IR(Nujol)cm⁻¹: 1713, 1597.

Compound 4(3b): M.p. 218-220° C. (dec.), MS(ESI)m/z: 553 (M+H), IR(Nujol)cm⁻¹: 3400, 1732, 1710.

4) To a solution of the compound 4(3a) obtained in the above step (3) (1.0 g) in methylene chloride (30 mL) are added dropwise oxalyl chloride (0.3 mL) and dimethylformamide (1 drop) under ice-cooling and the mixture is stirred at room temperature for 2 hours. The reaction mixture is concentrated and to the residue is added cooled ethanol. The mixture is stirred at room temperature for 30 minutes and concentrated. To the residue is added ethyl acetate and the solution is washed with saturated aqueous sodium hydrogencarbonate solution and saturated aqueous NaCl solution, successively. The organic layer is dried over sodium sulfate and concentrated. The residue is purified by column chromatography on silica gel (chloroform:methanol=10:1) to give (1R*,2S*(R*),4R*)-2′-acetyl-3′,4′-dihydro-6′,7′-dimethoxy-2-(2-methyl-1,2,3,4-tetrahydro-6,7-dimethoxy-1-isoquinolyl)-4-ethoxycarbonyl-spiro[cyclohexane-1,1′(2′H)-isoquinoline] (1.08 g, 100%) as an amorphous powder. MS(ESI)m/z: 581 (M+H), IR(Nujol)cm⁻¹: 1726, 1628

5) To a suspension of lithium aluminum hydride (2.77 g) in tetrahydrofuran (40 mL) is added dropwise a solution of the compound obtained in the above step (4) (8.47 g) in tetrahydrofuran. (40 mL) under refluxing. The mixture is refluxed for 3 hours and to the mixture is added an aqueous sodium hydroxide (2.8 mL) and water (5.6 mL), successively. The mixture is stirred at room temperature for 2 hours and chloroform is added to the mixture. The reaction mixture s filtered to remove infiltrate. The filtrate is dried over sodium sulfate and concentrated to give (1R*,2S*(R*),4R*)-2′-ethyl-3′,4′-dihydro-6′,7′-dimethoxy-2-(2-methyl-1,2,3,4-tetrahydro-6,7-dimethoxy-1-isoquinolyl)-4-hydroxymethyl-spiro[cyclohexane-1,1′(2′H)-isoquinoline] (8.50 g) as a crude product. MS(APCI)m/z:525 (M+H), IR(Nujol)cm⁻¹: 3541

6) To a solution of oxalyl chloride (2.55 mL) in methylene chloride (70 mL) is added dropwise dimethylsulfoxide (2.8 mL) at −78 t and the mixture is stirred at the same temperature for 15 minutes. To the mixture is added dropwise a solution of the compound obtained in the above step (5) (7.65 g) in methylene chloride (30 mL) at −78° C. The mixture is stirred for 2 hours at a temperature of −45° C. to −35° C., and to the mixture is added dropwise triethylamine (14.9 mL) at −45° C. The mixture is stirred at 0° C. for 2 hours. To the reaction mixture is added ethyl acetate and the mixture is washed with saturated aqueous NaCl solution. The organic layer is dried over sodium sulfate and concentrated. After addition of methylene chloride-diisopropylether, insoluble materials are removed by filtration and crystallized from isopropyl ether to give (1R*,2S*(R*),4R*)-2′-ethyl-3′,4′-dihydro-6′,7′-dimethoxy-2-(2-methyl-1,2,3,4-tetrahydro-6,7-dimethoxy-1-isoquinolyl)-4-formyl-spiro[cyclohexane-1,1′(2′H)-isoquinoline] (5.25 g, 69%) as crystals.

M.p. 137-139° C., MS(FAB)m/z: 523(M+H), IR(Nujol)cm⁻¹: 1726.

7) To a solution of silver nitrate (975 mg) in water (10 mL) is added dropwise a solution of the compound obtained in the above step (6) (1.0 g), potassium hydroxide (773 mg), ethanol (40 mL) and water (10 mL) under ice-cooling. The mixture is stirred at room temperature for 3 hours and the reaction mixture is filtered to remove insoluble materials through Celite. The filtrate is neutralized with hydrochloric acid and concentrated. The residue is dissolved in chloroform and dried over sodium sulfate. The solution is concentrated and crystallized from ethanol-ethyl acetate to give (1R*,2S*(R*),4R*)-2′-ethyl-3′,4′-dihydro-6′,7′-dimethoxy-2-(2-methyl-1,2,3,4-tetrahydro-6,7-dimethoxy-1-isoquinolyl)-4-carboxy-spiro[cyclohexane-1,1′(2′H)-isoquinoline] (0.65 g) as crystals.

M.p. 197-199° C. (dec.), MS(APCI)m/z: 539(M+H), IR(Nujol)cm⁻¹: 3388, 1717.

8) The compound 4(3b) is treated in the same manner as described in the above steps (4) to (7) to give (1R*,2S*(R*),4S*)-2′-ethyl-3′,4′-dihydro-6′,7′-dimethoxy-2-(2-methyl-1,2,3,4-tetrahydro-6,7-dimethoxy-1-isoquinolyl)-4-carboxy-spiro[cyclohexane-1,1′(2′H)-isoquinoline].

### Reference Example 5

1) A mixture of (1R*,2R*(S*))-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (compound obtained in Reference example 1(6)) (5.0 g), paraformaldehyde (2.4 g), acetic acid (25 mL), trifluoroacetic acid (4.56 g) and tetrahydrofuran (50 mL) is stirred at room temperature for 1 hour. To the mixture is slowly added portionwise sodium borohydride (2.42 g) and the mixture is stirred at room temperature for two days. The reaction mixture is concentrated and to the residue is added ethyl acetate. The solution is washed with saturated aqueous sodium hydrogencarbonate solution and saturated aqueous NaCl solution, successively. The organic layer is dried over sodium sulfate and concentrated. The residue is purified by column chromatography on silica gel (chloroform:methanol=20:1) to give (1R*,2R*(S*))-2′-methyl-3′,4′-dihydro-6′,7′-dimethoxy-2-(2-ethyl-1,2,3,4-tetrahydro-6,7-dimethoxy-1-isoquinolyl)-4,4-bisethoxy-carbonyl-spiro[cyclohexane-1,1′(2′H)-isoquinoline] (2.82 g, 55.2%) as an amorphous powder. MS(APCI)m/z: 639(M+H), IR(Nujol)cm⁻¹: 1725.

2) A mixture of the compound obtained in the above step (1) (2.63 g),50% sodium hydroxide (9.9 mL) and ethanol (25 mL) is stirred at room temperature overnight. The reaction mixture is refluxed for 18 hours. After cooling, the reaction mixture is neutralized with hydrochloric acid and concentrated. The residue is extracted with hot chloroform and dried over sodium sulfate. The organic layer is concentrated to give (1R*,2R*(S*))-2′-methyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (2.83 g) as a crude product. MS(APCI)m/z: 583(M+H), IR(Nujol)cm⁻¹: 3388, 1732

3) A mixture of the compound obtained in the above step (2) (1.87 g), pyridine hydrochloride (371 mg) and pyridine (20 mL) is refluxed for 1.5 hours. After cooling, the reaction mixture is concentrated and the residue is purified by column chromatography on silica gel (chloroform:methanol=20:1) to give (1R*,2R*(S*),4R*)-2′-methyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (1.58 g, 91%; compound 5(3a)) and (1R*,2R*(S*),4S*)-2′-methyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (0.15 g, 8.6%; compound 5(3b)).

Compound 5(3a): MS(APCI)m/z: 539 (M+H),IR(neat+chloroform)cm⁻¹: 3400-3300, 1709.

Compound 5(3b): MS(APCI)m/z: 539(M+H), IR(Nujol)cm⁻¹: 3400, 1707.

### Reference Example 6

1) A solution of (1R*,2R*(S*))-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (compound obtained in Reference example 1(6))(3.0 g) and diisopropylethylamine (4.34 g) in methylene chloride (60 mL) is added a solution of propionyl bromide (2.16 mL) in methylene chloride (20 mL) under ice-cooling over a period of 2 hours. The mixture is stirred at room temperature overnight and to the reaction mixture is added water. The reaction mixture is concentrated. The residue is diluted with ethyl acetate and washed with saturated aqueous sodium hydrogencarbonate solution, water, and saturated aqueous NaCl solution. The organic layer is dried over sodium sulfate and concentrated. The residue is purified by column chromatography on NH-silica gel (ethyl acetate) to give (1R*,2R*(S*))-2′-propionyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (1.59 g, 48.6%) as crystals.

M.p. 138° C., MS(APCI)m/z: 681 (M+H), IR(Nujol)cm⁻¹: 1749, 1725, 1639.

2) To a solution of the compound obtained in the above step (1) (2.02 g) in tetrahydrofuran (80 mL) is added 10M borane-dimethylsulfide complex (3.00 mL) and the mixture is stirred at 60° C. for 3 hours. After cooling, to the reaction mixture is added dropwise ethanol and the mixture is stirred at room temperature for 1 hour. Then, to the mixture is added dropwise 5M hydrogen chloride-ethanol solution (15 mL) and the mixture is refluxed for 1.5 hours. After cooling, the reaction mixture is concentrated and to the residue is added saturated aqueous sodium hydrogencarbonate solution. The solution is extracted with chloroform and dried over sodium sulfate. The extract is concentrated and the residue is purified by column chromatography on silica gel (chloroform:methanol:aqueous 28% ammonia=20:1:0.1) to give (1R*,2R*(S*))-2′-n-propyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (966 mg, 48.8%) as an amorphous powder.

MS(APCI)m/z: 667 (M+H), IR(Nujol)cm⁻¹: 1727.

3) The compound obtained in the above step (2) (839 mg) is treated in the same manner a described in Reference example 1(9) and (10) to give (1R*,2R*(S*),4R*)-2′-n-propyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (361 mg, 63.7%; compound 6(3a)) and (1R*,2R*(S*),4S*)-2′-n-propyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (154 mg, 21%; compound 6(3b)).

Compound 6(3a): MS(APCI)m/z: 567 (M+H), IR(Nujol)cm⁻¹: 3600, 3530, 1699.

Compound 6(3b): MS(APCI)m/z: 567(M+H), IR(Nujol)cm⁻¹: 1715, 1699.

### Reference Example 7

1) A mixture of (1R*,2R*(S*))-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (compound obtained in Reference example 1(6)) (8.0 g) and benzyl chloroformate (16 mL) is stirred at 75° C. for 4 hours. After cooling, to the reaction mixture is added ethyl acetate. The mixture is washed with saturated aqueous sodium hydrogencarbonate solution and saturated aqueous NaCl solution, and concentrated. Washing and evaporation the solution are repeated for 4 times. The residue is purified by column chromatography on silica gel (n-hexane→n-hexane/ethyl acetate (1:1)→ethyl acetates→chloroform/methanol (10:1)) to give (1R*,2R*(S*))-2′-benzyloxycarbonyl-3′,4′-dihydro-6′,7′-dimethoxy-2-(2-ethyl-1,2,3,4-tetrahydro-6,7-dimethoxy-1-isoquinolyl)-4,4-bisethoxycarbonyl-spiro[cyclohexane-1,1′(2′H)-isoquinoline] (7.35 g, 75.7%) as an amorphous powder.

MS(APCI)m/z: 759(M+H), IR(Nujol)cm⁻¹: 1724, 1696.

2) The compound obtained in the above step (1) is treated in the same manner as described in Reference example 1(9) and (10) to give (1R*,2R*(S*),4R*)-2′-benzyloxycarbonyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (4.92 g, 77.6%; compound 7(2a)) and (1R*,2R*(S*),4S*)-2′-benzyloxy-carbonyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (1.20 g, 18.9%; compound 7(2b)) as an amorphous powder, respectively.

Compound 7(2a): MS(ESI)m/z: 659 (M+H), IR(Nujol)cm⁻¹: 1694.

Compound 7(2b): MS(ESI)m/z: 659(M+H), IR(Nujol)cm⁻¹: 1703.

3) A mixture of the compound 7(2a) obtained in the above step (2) (2.06 g),5M hydrogen chloride-ethanol solution (10 mL) and ethanol (20 mL) is stirred at room temperature for 24 hours. The reaction mixture is concentrated and the residue is neutralized with saturated aqueous sodium hydrogencarbonate. The mixture is extracted with ethyl acetate, and the extract is washed with saturated aqueous NaCl solution and dried over sodium sulfate. The organic layer is concentrated to give crude (1R*,2R*(s*),4R*)-2′-benzyloxy-carbonyl-3′,4′-dihydro-6′,7′-dimethoxy-2-(2-ethyl-1,2,3,4-tetrahydro-6,7-dimethoxy-1-isoquinolyl)-4-ethoxycarbonyl-spiro[cyclohexane-1,1′(2′H)-isoquinoline] (2.08 g, 97%) as an amorphous powder.

MS(APCI)m/z: 687(M+H), IR(Nujol)cm⁻¹: 1720, 1695.

4) The compound obtained in the above step (3) (2.06 g) and 10% palladium-carbon (2.06 g) are added to ethanol (40 mL) and the mixture is subjected to catalytic hydrogenation under atmospheric pressure. The catalyst is removed by filtration and the filtrate is concentrated. The residue is crystallized from diisopropylether to give (1R*,2R*(S*),4R*)-3′,4′-dihydro-6′,7′-dimethoxy-2-(2-ethyl-1,2,3,4-tetrahydro-6,7-dimethoxy-1-isoquinolyl)-4-ethoxycarbonyl-spiro[cyclohexane-1,1′(2′H)-isoquinoline] (1.23 g, 74%) as crystals. M.p. 140-142° C., MS(APCI)m/z: 553(M+H), IR(Nujol)cm⁻¹: 3345, 1720

5) To a solution of the compound obtained in the above step (4) (0.80 g) in methylene chloride (16 mL) is added dropwise acryloyl chloride (0.59 mL) and triethylamine (1.0 mL), successively, and the mixture is stirred at room temperature for 2 hours. The reaction mixture is washed with water, dried over sodium sulfate and concentrated. The residue is purified by column chromatography on silica gel (chloroform:methanol=35:1) to give (1R*,2R*(S*),4R*)-2′-acryloyl-3′,4′-dihydro-6′,7′-dimethoxy-2-(2-ethyl-1,2,3,4-tetrahydro-6,7-dimethoxy-1-isoquinolyl)-4-ethoxycarbonyl-spiro[cyclohexane-1,1′(2′H)-isoquinoline] (0.834 g, 95%) as an amorphous powder.

MS(APCI)m/z: 607(M+H) IR(Nujol)cm⁻¹: 1719, 1645 1609.

6) To a solution of the compound obtained in the above step (5) (0.91 g) in acetonitrile (10 mL) is added 50% aqueous dimethylamine (3 mL) and the mixture is stirred at room temperature for 12 hours. The reaction mixture is concentrated and the residue is purified by column chromatography on silica gel (chloroform:methanol:aqueous 28% ammonia=2000:40:10) to give (1R*,2R*(S*),4R*)-2′-[3-(dimethylamino)propionyl]-3′,4′-dihydro-6′,7′-dimethoxy-2-(2-ethyl-1,2,3,4-tetrahydro-6,7-dimethoxy-1-isoquinolyl)-4-ethoxycarbonyl-spiro[cyclohexane-1,1′(2′H)-isoquinoline] (0.81 g, 83%) as crystals. M.p. 154-156° C.

MS(APCI)m/z: 652(M+H), IR(Nujol)cm⁻¹: 1713, 1649.

7) The compound obtained in the above step (6) (790 mg) is suspended in a mixture of ethanol (10 mL) and methanol (3 mL) and to the suspension is added aqueous 10% sodium hydroxide (4 mL). The mixture is stirred at room temperature for 2 hours. The reaction mixture is neutralized with 10% hydrochloric acid and concentrated. The residue is extracted with chloroform and the extract is concentrated to give (1R*,2R*(S*),4R*)-2′-[3-(dimethylamino)propionyl]-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (850 mg, quantitativly) as an amorphous powder.

MS(APCI)m/z: 624(M+H), IR(Nujol)cm⁻¹: 1706, 1656, 1640.

8) The compound 7(2b) obtained in the above step (2) is treated in the same manner as described in the above steps (3) to (7) to give (1R*,2R*(S*),4S*)-2′-[3-(dimethylamino)propionyl]-3′,4′-dihydro -6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline].

### Reference Example 8

1) (1R*,2R*)-2′-Acetyl-3′,4′-dihydro-6′,7′-dimethoxy-2-(3,4-dihydro-6,7-dimethoxy-1-isoquinolyl)-4,4-bisethoxy-carbonyl-spiro[cyclohexane-1,1′(2′H)-isoquinoline] (10.0 g) is dissolved in a mixture of ethanol (630 mL) and acetic acid (70 mL), and to the solution is added platinum dioxide (PtO₂) (100 Mg). The mixture is subjected to catalytic hydrogenation under atmospheric pressure at room temperature. The reaction mixture is filtered to remove catalysts and the filtrate is concentrated. The residue is neutralized with saturated aqueous sodium hydrogencarbonate and extracted with ethyl acetate. The extract is washed with saturated aqueous NaCl solution, dried over sodium sulfate, and concentrated. The residue is crystallized from diethylether and recrystallized from ethanol to give (1R*,2R*(S*))-3′,4′-dihydro-6′,7′-dimethoxy-2-(2-acetyl-1,2,3,4-tetrahydro-6,7-dimethoxy-1-isoquinolyl)-4,4-bisethoxycarbonyl-spiro[cyclohexane-1,1′(2′H)-isoquinoline] (8.45 g) as crystals. M.p. 146-149° C.

MS(FAB)m/z: 639(M+H), IR(Nujol)cm⁻¹: 3280, 1740, 1725, 1620, 1610.

2) To a solution of the compound obtained in the above step (1) (4.0 g) and triethylamine (2.5 g) in methylene chloride (40 mL) is added dropwise acryloyl chloride (1.70 g) under ice-cooling and the mixture is stirred at room temperature for 2 hours. The reaction mixture is washed with an aqueous sodium hydroxide and dried over sodium sulfate. The organic layer is concentrated and the residue is crystallized from diisnropylether to give (1R*, 2R*(S*))-2′-acryloyl-3′,4′-dihydro-6′,7′-dimethoxy-2-(2-acetyl-1,2,3,4-tetrahydro-6,7-dimethoxy-1-isoquinolyl)-4,4-bisethoxycarbonyl-spiro[cyclohexane-1,1′(2′H)-isoquinoline](4.09 g, 94%) as crystals. M.p. 162-165° C., MS(APCI)m/z: 693(M+H)

3) To a solution of the compound obtained in the above step (2) (200 mg) in acetonitrle (2 mL) is added 50% aqueous dimethylamine (1 mL) and the mixture is stirred at room temperature overnight. The reaction mixture is concentrated and the residue is purified by column chromatography on silica gel (chloroform:methanol:ammonium hydroxide=20:1:0.1) to give (1R*,2R*(S*))-2′-[3-(dimethylamino)propionyl)-3′,4′-dihydro-6′,7′-dimethoxy-2-(2-acetyl-1,2,3,4-tetrahydro-6,7-dimethoxy-1-isoquinolyl)-4,4-bisethoxycarbonyl-spiro[cyclohexane-1,1′(2′H)-isoquinoline] (174 mg, 81%) as crystals. M.p. 154-156° C., MS(APCI)m/z: 738(M+H)

4) To a solution of the compound obtained in the above step (3) (2.0 g) in ethanol (20 mL) is added 10% aqueous sodium hydroxide (10 mL) and the mixture is refluxed for 18 hours. After cooling, the reaction mixture is concentrated and the residue is acidified with hydrochloric acid and washed with chloroform. The aqueous layer is neutralized with aqueous ammonia and concentrated. The residue is extracted with chloroform-methanol (5:1) and the extract is filtered to remove inorganic materials. The filtrate is concentrated to give crude (1R*,2R*(S*))-2′-[3-(dimethylamino)-propionyl]-3′, 4′-dihydro-6′,7′-dimethoxy-2-(2-acetyl-1,2,3,4-tetrahydro-6,7-dimethoxy-1-isoquinolyl)-4,4-biscarboxy-spiro[cyclohexane-1,1′(2′H)-isoquinoline] (2.42 g). MS(ESI)m/z: 680(M−H)

5) A mixture of the compound obtained in the above step (4) (2.4 g), pyridine (25 mL) and pyridine hydrochloride (0.813 g) is refluxed for 2 hours. After cooling, the reaction mixture is concentrated and to the residue is added chloroform. The solution is filtered to remove insoluble materials and the filtrate is concentrated. The residue is purified by column chromatography on NH-silica gel (chloroform to methanol) to give (1R*,2R*(S*))-2′-[3-(dimethylamino)propionyl]-3′,4′-dihydro-6′,7′-dimethoxy-2-(2-acetyl-1,2,3,4-tetrahydro-6,7-dimethoxy-1-isoquinolyl)-4-carboxy-spiro[cyclohexane-1,1′(2′H)-isoquinoline] (1.34 g, 78% from the compound obtained in the above step (3)) as an amorphous powder. MS(ESI)m/z: 636(M−H), IR(Nujol)cm⁻¹: 1640

### Reference Example 9

1) To a solution of diisopropylamine (4.44 g) in tetrahydrofuran (25 mL) is added dropwise 1.6M solution of 1.6M n-butyllithium (24.9 mL) in n-hexane under ice-cooling and the mixture is stirred for 30 minutes at the same temperature. The reaction mixture is added dropwise to a solution of (1R*,2R* (S*),4R*)-2′-acetyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (compound 2(2a) obtained in Reference example 2(2)) (4.52 g) in tetrahydrofuran (25 mL) at −78° C. The mixture is stirred at −20° C.-−30° C. for 2 hours and to the mixture is added dropwise allyl bromide (9.65 g) at −78° C. The mixture is stirred at 0° C. for 1 hour and neutralized with 10% hydrochloric acid at the same temperature. The reaction mixture is evaporateid to remove tetrahydrofuran and the residue is extracted with chloroform. The extract is dried over sodium sulfate and concentrated. The residue is purified by column chromatography on silica gel (chloroform:methanol=50:1 to 10:1) to give of (1R*,2R*(S*),4R*)-2′-(4-pentenoyl)-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (4.22 g) as foam.

MS(APCI)m/z: 607(M+H), IR(Nujol)cm⁻¹: 1609.

2) To a suspension of lithium aluminum hydride (1.23 g) in tetrahydrofuran (70 mL) is added dropwise a solution of the compound obtained in the above step (1) (3.94 g) in tetrahydrofuran (70 mL) under refluxing. The mixture is refluxed for 2 hours and to the reaction mixture is added water (1.2 mL), 10% aqueous sodium hydroxide (1.8 mL) and water (0.6 mL), successively. The mixture is stirred at room temperature overnight and dried over sodium sulfate. The mixture is concentrated and the residue is purified by column chromatography on silica gel (chloroform:methanol=10:1 to Chloroform:methanol:aqueous 28% ammonia=10:2:0.1) to give (1R*,2R*(S*),4R*)-2′-(4-pentenyl)-3′,4′-dihydro-6′,7′-dimethoxy-2-(2-ethyl-1,2,3,4-tetrahydro-6,7-dimethoxy-1-isoquinolyl)-4-hydroxymethyl-spiro[cyclohexane-1,1′(2′H)-isoquinoline] (2.75 g, 73%) as an amorphous powder.

MS(APCI)m/z: 579(M+H), IR(Nujol)cm⁻¹: 3525.

3) The compound obtained in the above step (2) is treated in the same manner as described in Reference example 2(4) to give (1R*,2R*(S*)4R*)-2′-(4-pentenyl)-3′,4′-dihydro-6′,7′-dimethoxy-2-(2-ethyl-1,2,3,4-tetrahydro-6,7-dimethoxy-1-isoquinolyl)-4-formyl-spiro[cyclohexane-1,1′(2′H)-isoquinoline].

MS(APCI)m/z: 577(M+H), IR(Nujol)cm⁻¹: 1721.

4) The compound obtained in the above step (3) is treated in the same manner as described in Reference example 2(5) to give (1R*,2R*(S*),4R*)-2′-(4-pentenyl)-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline].

MS(APCI)m/z: 593(M+H), IR(Nujol)cm⁻¹: 1702.

### Reference Example 10

1) To a solution of diisopropylamine (1.78 g) in tetrahydrofuran (19 mL) is added dropwise 1.6M solution of n-butyllithium in n-hexane (10.5 mL) under ice-cooling and the mixture is stirred at the same temperature. The reaction mixture is added dropwise to a solution of (1R*, 2R*(S*),4R*)-2′-acetyl-3′,4′-dihydro-6′,7′-dimethoxy-2-(2-ethyl-1,2,3,4-tetrahydro-6,7-dimethoxy-1-isoquinolyl)-spiro[cyclohexane-1,1′(2′H)-isoquinoline]-4-carboxylic acid (compound 2(2a) obtained in Reference example 2(2)) (1.90 g) in tetrahydrofuran (19 mL) at −78° C. and the mixture is stirred at 0° C. for 3 hours. The reaction mixture is neutralized with 1M hydrochloric acid and evaporated to remove tetrahydrofuran. The residue is extracted with chloroform and the extract is dried over sodium sulfate. The organic layer is concentrated and the residue is purified by column chromatography on silica gel (chloroform:methanol=20:1 to 5:1) to give (1R*,2R*(S*),4R*)-2′-(3-hydroxy)butyryl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (1.62 g, 79%) as an amorphous powder.

MS(ESI)m/z: 609(M+H), IR(Nujol)cm⁻¹: 1711, 1610.

2) To a solution of the compound obtained in the above step (1) (888 mg), ethanol (1 mL) and N,N-dimethylaminopyridine (35 mg) in methylene chloride (10 mL) is added 1-(3-dimethylamino-propyl)-3-ethylcarbodiimide hydrochloride (418 mg) and the mixture is stirred at room temperature for 3 hours. The reaction mixture is washed with saturated aqueous sodium hydrogencarbonate solution and saturated aqueous NaCl solution, successively. The organic layer is dried over sodium sulfate and concentrated. The residue is purified by column chromatography on silica gel (chloroform:methanol=50:1 to 20:1) to give (1R*,2R*(S*),4R*)-2′-(3-hydroxy)butyryl-3′,4′-dihydro-6′,7′-dimethoxy-2-(2-ethyl-1,2,3,4-tetrahydro-6,7-dimethoxy-1-isoquinolyl)-4-ethoxycarboxy-spiro[cyclohexane-1,1′(2′H)-isoquinoline](893 mg, 96%) as an amorphous powder.

MS(APCI)m/z: 639(M+H), IR(neat)cm⁻¹: 3465, 1720, 1613.

3) To a solution of the compound obtained in the above step (2) (692 mg) and triethylamine (219 mg) in methylene chloride (20 mL) is added methanesulfonyl chloride (167 μL) under ice-cooling and the mixture is stirred at the same temperature for 1 hour. The reaction mixture is concentrated. To the residue is added ethyl acetate and the solution is washed with saturated aqueous sodium hydrogencarbonate solution and saturated aqueous NaCl solution, successively. The organic layer is dried over sodium sulfate and concentrated. The residue is purified by column chromatography on NH-silica gel (ethyl acetate) to give (1R*,2R*(S*),4R*)-2′-(3-methanesulfonyloxy)butyryl-3′,4′-dihydro-6′,7′-dimethoxy-2-(2-ethyl-1,2,3,4-tetrahydro-6,7-dimethoxy-1-isoquinolyl)-4-ethoxycarboxy-spiro[cyclohexane-1,1′(2′H)-isoquinoline] (726 mg) as an amorphous powder.

MS(APCI)m/z: 717(M+H), IR(neat)cm⁻¹: 1719, 1639.

4) A suspension of lithium aluminum hydride (427 mg) in tetrahydrofuran (7 mL) is refluxed and to the mixture is added dropwise a solution of the compound obtained in the above step (3) in tetrahydrofuran (7 mL). The mixture is refluxed for 2 hours. To the reaction mixture is added water (0.3 mL), 10% aqueous sodium hydroxide (0.45 mL), and water (0.15 mL) under ice-cooling, successively and the mixture is stirred at room temperature for 3 hours. The solution is dried over sodium sulfate and concentrated. The above steps are repeated to accomplish the reaction to give (1R*,2R*(S*),4R*)-2′-n-butyl-3′,4′-dihydro-6′,7′-dimethoxy-2-(2-ethyl-1,2,3,4-tetrahydro-6,7-dimethoxy-1-isoquinolyl)-4-hydroxymethyl-spiro[cyclohexane-1,1′(2′H)-isoquinoline] (595 mg) as an oil.

MS(APCI)m/z: 567(M+H), IR(neat)cm⁻¹: 3524, 1609.

5) The compound obtained in the above step (4) is treated in the same manner as described in Reference example 2(4) to give (1R*,2R*(S*),4R*)-2′-n-butyl-3′,4′-dihydro-6′,7′-dimethoxy-2-(2-ethyl-1,2,3,4-tetrahydro-6,7-dimethoxy-1-isoquinolyl)-4-formyl-spiro[cyclohexane-1,1′(2′H)-isoquinoline]

MS(APCI)m/z: 565(M+H), IR(neat)cm⁻¹: 1719.

6) The compound obtained in the above step (5) is treated in the same manner as described in Reference example 2(5) to give (1R*,2R*(S*),4R*)-2′-n-butyl-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline]

MS(APCI)m/z: 581(M+H), IR(neat)cm⁻¹: 1695.

### Reference Example 11

1) To a solution of the compound obtained in Reference example 7(5) (1.0 g) in acetonitrile (20 mL) is added benzylamine (3.54 g) and the mixture is refluxed for 36 hours. The reaction mixture is concentrated and the residue is purified by column chromatography on silica gel (chloroform:methanol:aqueous 28% ammonia=2000:25:10) to give (1R*,2R*(S*),4R*)-2′-(3-benzylamino)propionyl-3′,4′-dihydro-6′,7′-dimethoxy-2-(2-ethyl-1,2,3,4-tetrahydro-6,7-dimethoxy-1-isoquinolyl)-4-ethoxycarbonyl-spiro[cyclohexane-1,1′(2′H)-isoquinoline] (0.60 g, 51%) as crystals. M.p. 83-85° C.

MS(APCI)m/z: 714(M+H), IR(Nujol)cm⁻¹: 1717, 1643.

2) To a solution of the compound obtained in the above step (1) (248 mg) and triethylamine (0.098 mL) in methylene chloride (5 mL) is added benzyl chloroformate (0.055 mL) and the mixture is stirred at room temperature for 2 hours. The reaction mixture is concentrated and to the residue is added ethyl acetate. The solution is washed with saturated aqueous sodium hydrogencarbonate solution and saturated aqueous NaCl solution, successively. The organic layer is dried over sodium sulfate and concentrated to give (1R*, 2R*(S*),4R*)-2′-[3-(N-benzyl-N-benzyloxycarbonylamino)propionyl]-3′,4′-dihydro-6′,7′-dimethoxy-2-(2-ethyl-1,2,3,4-tetrahydro-6,7-dimethoxy-1-isoauinolyl)-4-ethoxycarbonyl-spiro[cyclohexane-1,1′(2′H)-isoquinoline] (295 mg).

3) The compound obtained in the above step (2) (295 mg) is dissolved in a mixture of methanol (20 mL) and ethanol (20 mL) and to the solution is added an aqueous 10% sodium hydroxide (10 mL). The mixture is stirred at room temperature for 10 hours and at 60° C. for 1 hour. The reaction mixture is neutralized with 10% hydrochloric acid under ice-cooling. The mixture is evaporated to remove alcohol. The residue is extracted with chloroform, and the extract is dried over sodium sulfate and concentrated to give (1R*,2R*(S*),4R*)-2′-[3-(N-benzyl-N-benzyloxy-carbonylamino)propionyl]-3′,4′-dihydro-6′,7′-dimethoxy-2-(2-ethyl-1,2,3,4-tetrahydro-6,7-dimethoxy-1-isoquinolyl)-4-carboxy-spiro[cyclohexane-1,1′(2′H)-isoquinoline] (285 mg) as a crude product.

### Reference Example 12

1) A mixture of 3,4-dimethoxyphenethylamine (20.4 g),4,4-bisethoxycarbonylcyclohexanone (14.9 g) and polyphosphoric acid (200 g) is stirred at 130° C. for 40 minutes. After cooling, the reaction mixture is poured into saturated aqueous sodium hydrogencarbonate solution to neutralize it. The mixture is extracted with ethyl acetate and the extract is washed with saturated aqueous sodium hydrogencarbonate solution and saturated aqueous NaCl solution. The solution is dried over sodium sulfate and concentrated. The residue is triturated with ethyl ether and collected to give 3′,4′-dihydro-6′,7′-dimethoxy-4,4-bisethoxycarbonyl-spiro[cyclohexane-1,1′(2′H)-isoquinoline] (6.98 g, 31.5%) as crystals. M.p. 103-104° C., MS(APCI)m/z: 406(M+H), IR(Nujol)cm−1: 3306, 1735, 1715

2) To a solution of the compound obtained in the above step (1) (998 mg) and diusopropylethylamine (413 mg) in methylene chloride (20 mL) is added dropwise benzyl chloroformate (0.42 mL). The mixture is stirred at room temperature for 3 hours and refluxed for 3 hours. The reaction mixture is concentrated and to the residue is added ethyl acetate. The solution is washed with 10% hydrochloric acid and saturated aqueous NaCl solution, and dried over sodium sulfate. The organic layer is concentrated and the residue is purified by column chromatography on silica gel (chloroform:ethyl acetate=3:2) to give 2′-benzyloxycarbonyl-3′,4′-dihydro-6′,7′-dimethoxy-4,4-bisethoxycarbonyl-spiro[cyclohexane-1,1′(2′H)-isoquinolin] (1.34 g, 100%) as crystals.

M.p. 101-102° C., MS(APCI)m/z: 540(M+H), IR(Nujol)cm−1: 1727, 1702, 1609.

3) A mixture of the compound obtained in the above step (2) (75.0 g), sodium hydroxide (55.6 g), ethanol (400 mL) and water (400 mL) is stirred at room temperature for 16 hours and refluxed for 6 hours. After cooling, the reaction mixture is concentrated and acidified with conc. hydrochloric acid. The mixture is extracted with ethyl acetate. The extract is washed with saturated aqueous NaCl solution and dried over sodium sulfate. The organic layer is concentrated and the residue is recrystallized from ethyl acetate-diisopropyl ether to give 2′-benzyloxycarbonyl-3′,4′-dihydro-6′,7′-dimethoxy-4,4-biscarboxy-spiro[cyclohexane-1,1′(2′H)-isoquinolin] (72.0 g, 100%) as crystals. M.p. 181-182° C., MS(ESI)m/z: 481(M−H), IR(Nujol)cm−1: 1749, 1703, 1669, 1611

4) A mixture of the compound obtained in the above step (3) (72.0 g) and pyridine (500 mL) is refluxed for 5 hours. After cooling, the reaction mixture is concentrated and the residue is dissolved in methanol (500 mL). To the solution is added 10% palladium-carbon (3.0 g) and the mixture is subjected to catalytic hydrogenation under atmospheric pressure. After the reaction, the reaction mixture is filtered to remove catalysts and the filtrate is concentrated. To the residue is added pyridine (500 mL) and the mixture is refluxed for 16 hours. The reaction mixture is concentrated and the residue is crystallized from ethanol to give (1α,4β)-3′,4′-dihydro-6′,7′-dimethoxy-4-carboxy-spiro[cyclohexane-1,1′(2′H)-isoquinolin] (21.3 g, 50%) as crystals.

M.p. 238-241° C., MS(APCI)m/z: 306(M+H), IR(Nujol)cm−1: 1616.

### Reference Example 13

1) A solution of dopamine hydrochloride (54.0 g),4,4-bisethoxycarbonylcyclohexanone (69.0 g) and triethylamine (28.8 g) in ethanol (570 mL) is stirred at 50° C. for 20 hours. After cooling, the precipitated crystals are collected to give 3′,4′-dihydro-6′,7′-dihydroxy-4,4-bisethoxycarbonyl-spiro[cyclohexane-1,1′(2′H)-isoquinoline] (78.6 g) as crystals. M.p. 180-181° C., MS(APCI)m/z: 378(M+H), IR(Nujol)cm⁻¹: 3475, 1719, 1620

2) To a solution of the compound obtained in the above step (1) (68.0 g) and triethylamine (91.1 g) in methylene chloride (1000 mL) is added dropwise chlorotrimethylsilane (78.3 g) at 4° C. and the mixture is stirred at room temperature for 1 hour. To the reaction mixture is added 1M citric acid (500 mL). The organic layer is separated, washed with water, dried over sodium sulfate, and concentrated. The residue is triturated with ethyl acetate-n-hexane and collected to give 3′,4′-dihydro-6′,7′-di(trimethylsilyloxy)-4,4-bisethoxycarbonyl-spiro[cyclohexane-1,1′(2′H)-isoquinoline] (86.4 g) as an amorphous powder.

3) To the crude compound obtained in the above step (2) is added diisopropylethylamine (46.5 g) and methylene chloride (1000 mL) and to the mixture is added dropwise benzyl chloroformate (61.4 g) at 4° C. over a period of 30 minutes. The mixture is stirred for 20 hours at room temperature. The reaction mixture is concentrated and to the residue is added ethyl acetate. The solution is washed with 1M citric acid and water and dried over sodium sulfate. The organic layer is concentrated and to the residue is added acetonitrile (600 mL). To the mixture is added aqueous 47% hydrofluoric acid and the mixture is stirred at the same temperature for 30 minutes. The mixture is further stirred at room temperature for 30 minutes and neutralized with sodium hydrogencarbonate powder. The mixture is concentrated, poured into water (1 L), and extracted with ethyl acetate. The extract is washed with water and saturated aqueous NaCl solution, and dried over sodium sulfate. The washed extract is concentrated and the residue is purified by column chromatography on silica gel (n-hexane:ethyl acetate=4:1 to 1:1) to give 2′-benzyloxycarbonyl-3′,4′-dihydro-6′,7′-dihydroxy-4,4-bisethoxycarboxy-spiro[cyclohexane-1,1′(2′H)-isoquinoline] (73.0 g, 69%) as crystals. M.p. 132-133° C., MS(APCI)m/z: 512(M+H), IR(Nujol)cm−1: 3430, 3375, 1746, 1725, 1690, 1620

4) The compound obtained in the above step (3) (73.0 g) is slowly added portionwise to a suspension of 60% sodium hydride (12.6 g) in dimethylformamide (500 mL) and the mixture is stirred at room temperature for 30 minutes. To the reaction mixture is added dropwise methyl iodide (50.7 g) and the mixture is stirred at room temperature for 17 hours. The reaction mixture is concentrated and poured into an aqueous ammonium chloride. The solution is extracted with ethyl acetate, and the extract is washed with water and saturated aqueous NaCl solution. The organic layer is dried over sodium sulfate and concentrated. The residue is purified by column chromatography on silica gel (n-hexane:ethyl acetate=4:1) to give 2′-benzyloxycarbonyl-3′,4′-dihydro-6′,7′-dimethoxy-4,4-bisethoxycarbonyl-spiro[cyclohexane-1,1′(2′H)-isoquinoline] (75.0 g, 97%; the same compound as obtained in Reference example 12(2)) as crystals. M.p. 101-102° C., MS(APCI)m/z: 540(M+H), IR(Nujol)cm−1: 1727, 1702, 1609

### Reference Example 14

1) To a solution of the compound obtained in Reference example 12(1) (3.97 g) and triethylamine (1.52 g) in methylene chloride (80 mL) is added dropwise a solution of acryloyl chloride (1.09 g) in methylene chloride under ice-cooling and the mixture is stirred at the same temperature for 20 minutes. To the reaction mixture is added water (100 mL) and the mixture is stirred at room temperature for 1.5 hours. The organic layer is separated and dried over sodium sulfate. After addition of NH-silica gel (10 g), the mixture is filtered and the filtrate is concentrated to give 2′-acryloyl-3′,4′-dihydro-6′,7′-dimethoxy-4,4-bisethoxycarbonyl-spiro[cyclohexane-1,1′(2′H)-isoquinoline] (4.56 g) as crystals. M.p. 131-134.5° C., MS(APCI)m/z: 460(M+H), IR(Nujol)cm−1: 1725, 1661

2) To a solution of the compound obtained in the above step(1) (4.14 g) in acetonitrile (40 mL) is added aqueous 50% dimethylamine (15 mL) and the mixture is stirred at room temperature for 13 hours. The reaction mixture is concentrated and to the residue is added saturated aqueous NaCl solution. The mixture is extracted with ethyl acetate and the organic layer is further extracted with 5% hydrochloric acid. The aqueous layer is basified with potassium carbonate and extracted with ethyl acetate. The extract is washed with saturated aqueous NaCl solution, dried over sodium sulfate, and concentrated. The residue is purified by chromatography on silica gel (ethyl acetate-chloroform/methanol(50:1)) to give 2′-[3-(dimethylamino)-propionyl]-3′,4′-dihydro-6′,7′-dimethoxy-4,4-bisethoxycarboxy-spiro[cyclohexane-1,1′(2′ H)-isoquinoline] (4.13 g, 91%) as an oil.

MS(APCI)m/z: 505(M+H), IR(neat)cm−: 1728, 1657.

The present compound can be converted in a conventional manner to 2 hydrochloric acid salt thereof. M.p. 208-209.5° C.

3) To a solution of the compound obtained in the above step (2) (3.74 g) in ethanol (20 mL) is added an aqueous sodium hydroxide (2.96 g) in water (20 mL) and the mixture is refluxed for 2.5 hours. To the reaction mixture is added conc. HCl (6.8 mL) and the mixture is concentrated. To the residue is added methanol and insoluble materials are removed by decantation. The supernatant is concentrated and to the residue is added pyridine (30 mL). The mixture is refluxed for 4 hours. After cooling, the reaction mixture is concentrated. To the residue is added water and the solution is filtered to remove insoluble materials. The filtrate is purified by column chromatography on non-ionic adsorbent resin (HP-20; Mitsubishi Chemical) to give 2′-[3-(dimethylamino)propionyl]-3′,4′-dihydro-6′,7′-dimethoxy-4-carboxy-spiro[cyclohexane-1,1′(2′H)-isoquinoline] (2.88 g, 96%; a mixture of cis-/trans-form) as an amorphous powder.

MS(APCI)m/z: 405(M+H), IR(neat)cm−1:1712, 1652, 1609.

### Reference Example 15

1) The compound obtained in Reference example 12(4) is treated in the same manner as described in Reference example 14(1) to give (1α,4β)-2′-acryloyl-3′,4′-dihydro-6′,7′-dimethoxy-4-carboxy-spiro[cyclohexane-1,1′(2′H)-isoquinoline] as an amorphous powder.

MS(ESI): 358(M−H), IR(Nujol)cm−1:1718, 1635.

2) The compound obtained in the above step (1) is treated in the same manner as described in Reference example 14(2) to give (1α,4β)-2′-[3-(dimethylamino)propionyl]-3′,4′-dihydro-6′,7′-dimethoxy-4-carboxy-spiro[cyclohexane-1,1′(2′H)-isoquinoline] as an amorphous powder.

MS(ESI)m/z: 403(M−H), IR(Nujol)cm−1: 1700, 1637.

### Reference Example 1

1) A mixture of the compound obtained in Reference example 12(4) (1 g), benzylalcohol (531 mg),1-ethyl-3-(3-dimethylaminopropyl)carbodiimide hydrochloride (942 mg),1-hydroxybenzotriazole (664 mg), triethylamine (662 mg), N,N-dimethylaminopyridine (40 mg) and dimethylformamide (20 mL) is stirred for 20 hours at room temperature. The reaction mixture is extracted with ethyl acetate after addition of saturated aqueous sodium hydrogencarbonate. The extract is washed with saturated aqueous NaCl solution, dried over sodium sulfate, and concentrated. The residue is purified by column chromatography on NH-silica gel (n-hexane:ethyl acetate=1:1) to give (1α,4β)-3′,4′-dihydro-6′,7′-dimethoxy-4-benzyloxycarbonyl-spiro[cyclohexane-1,1′(2′H)-isoquinoline] (1.16 g) as crystals. M.p. 89-90° C., MS(APCI)m/z: 396(M+H), IR(Nujol)cm−1: 1713, 3317

2) A mixture of the compound obtained in the above step (1) (1.14 g), N-(3-bromopropyl)phthalimide (7.73 g), and diisopropylethylamine (3.73 g) in N,N-dimethylacetamide (10 mL) is stirred at 120° C. for 4 hours. After cooling, to the reaction mixture is added water and the mixture is extracted with ethyl acetate. The extract is washed with saturated aqueous sodium hydrogencarbonate solution and saturated aqueous NaCl solution, successively. The organic layer is dried and concentrated. The residue is purified by column chromatography on silica gel (n-hexane:ethyl:acetate=2:1) to give (1α,4β)-2′-(3-phthalimidopropyl)-3′,4′-dihydro-6′,7′-dimethoxy-4-benzyloxy-carbonyl-spiro[cyclohexane-1,1′(2′H)-isoquinoline] (1.41 g, 84%) MS(APCI)m/z: 583(M+H), IR(Nujol)cm−1: 1711, 1770

3) A mixture of the compound obtained in the above step (2) (5.15 g),10% palladium-carbon (70 mg), ammonium formate (27.87 g) and methanol (100 mL) is refluxed under heating for 6 hours. After cooling, the reaction mixture is filtered to remove catalysts and the filtrate is concentrated. To the residue is added water and the mixture is extracted with chloroforrii. The extract is washed with saturated aqueous NaCl solution, dried over sodium sulfate, and concentrated. The residue is purified by column chromatography on silica gel (chloroform:methanol=100:1 to 20:1) to give (1α,4β)-2′-(3-phthalimidopropyl)-3′,4′-dihydro-6′,7′-dimethoxy-4-carboxy-spiro[cyclohexane-1,1′(2′H)-isoquinoline] (3.98 g, 91%) MS(ESI)m/z: 491(M−H), IR(Nujol)cm⁻¹: 1709, 1770, 3370

### Reference Example 37

1) To a suspension of 4-hydroxypyrazolo[3,4-d]pyrimidine (allopurinol; 19.8 g) in N,N-dimethylaniline (59 mL) is added phosphorus oxychloride (296 mL) and the mixture is refluxed for 2.5 hours. After cooling, the excess of phosphorus oxychloride is removed and to the residue is added water. The mixture is extracted with ethyl acetate (×5). The extract is washed with saturated aqueous NaCl solution (200 mL×2), dried over sodium sulfate and concentrated. The residue is triturated with diethylether and collected to give 4-chloroallopurinol (16.7 g, 74%) as crude crystals. To a solution of the crude crystals (18 g) in dimethylformamide (100 mL) is added dropwise a solution of ethyl 1-piperazincarboxylate (14.7 g) and diisopropylethylamine (18.4 g) in dimethylformamide (10 mL) and the mixture is stirred at room temperature for 15 hours. To the reaction mixture is added water (300 mL) and the mixture is stirred at room temperature for 2 hours. The precipitated crystals are collected by filtration and the crystals are washed with water (200 mL), diisopropylether (50 mL), and ethyl acetate (200 mL) to give 4-(4-ethoxycarbonyl-1-piperazinyl)-1H-pyrazolo[3,4-d]pyrimidine (21.6 g, 82%) as crystals.

M.p. 245-247° C.

2) A mixture of allopurinol (10.88 g), ethyl piperazincarboxylate(37.96 g), 1,1,1,3,3,3-hexamethyl-disilazane (35.44 mL) and ammonium sulfate (1.06 g) is stirred at 140° C. for 29 hours. To the reaction mixture is added dropwise methanol (150 mL) under ice-cooling. The precipitates are collected and washed with methanol, saturated aqueous sodium hydrogencarbonate solution and water to give 4-(4-ethoxycarbonyl-1-piperazinyl)-1H-pyrazolo[3,4-d]pyrimidine (18.33 g, 82%) as crystals.

M.p. 247° C., MS(APCI)m/z: 277(M+H), IR(Nujol)cm⁻¹: 3196, 3115, 1699.

3) To a solution of the compound obtained in the above step (1) or (2) (20 g) and 4-chloromethylpyridine hydrochloride (13.1 g) in dimethylformamide (500 mL) is added lithium hydroxide (12.4 g) and the mixture is stirred at room temperature for 6 hours. To the reaction mixture is added water (1.6 L), saturated aqueous NaCl solution and saturated aqueous sodium hydrogencarbonate solution and the mixture is extracted with ethyl acetate (×3). The extract is washed twice with saturated aqueous NaCl solution, dried over sodium sulfate, and concentrated. The residue is recrystallized from isopropyl alcohol to give 1-(4-pyridylmethyl)-4-(4-ethoxycarbonyl-1-piperazinyl)-1H-pyrazolo[3,4-d]pyrimidine (17.7 g, 66%) as crystals. M.p. 123-124° C.

4) A suspension of the compound obtained in the above step (3) (20 g) and potassium hydroxide (20.6 g) in ethanol (300 mL) and water (60 mL) is refluxed for 3.5 hours. The reaction mixture is concentrated. To the reside is added water and the mixture is extracted with chloroform. The extract is washed with saturated aqueous NaCl solution, dried over sodium sulfate and concentrated. The residue is recrystallized from ethyl acetate-n-hexane to give 1-(4-pyridylmethyl)-4-(1-piperazinyl)-1H-pyrazolo[3,4-d]pyrimidine (12.2 g, 76%) as crystals. M.p. 121° C.

### Reference Example 18

1) To a solution of 4-bromopyridine (1.01 g) and iodomethane(5 mL) in acetonitrile (10 mL) is added dropwise triethylamine (0.81 mL) at room temperature and the mixture is stirred overnight. The precipitates are collected to give a mixture of triethylammmonium hyrdrochloride and 1-methyl-4-bromopyridinium iodide (1.35 g) as crude crystals.

2) To a solution of N-(benzyloxycarbonyl)piperazine (1.12 g) and triethylamine (525 mg) in acetonitrile (15 mL) is added dropwise a suspension of the compound obtained in the above step (1) (1.24 g) in acetonitrile (20 mL) at room temperature. The mixture is stirred for 2.5 hours and then concentrated. The residue is dissolved in water and the solution is treated with activated carbon powder. The solution is washed with diethylether and concentrated. The residue is dissolved in acetic acid (12 mL) and to the solution is added 25% hydrogen bromide-acetic acid (5 mL). The mixture is stirred at room temperature for 2 hours. To the reaction mixture is added dropwise diisopropylether (10 mL) and the precipitates are collected and washed with isopropyl alcohol and ethanol to give 4-piperazinyl-1-methylpyridimiumbromide hydrobromide (1.01 g, 62.3%) as crystals. M.p. 267-276° C. (dec.), MS(EIS)m/z: 178 (M⁺)

### Reference Example 19

1) A mixture of N-(2-bromoethyl)phthalimide (46.98 g), triethylamine (38.66 mL), acetonitrile (300 mL) and 1-benzyloxycarbonylpiperazine (48.88 g) is refluxed for 22 hours. After cooling, to the reaction mixture is added 1M hydrochloric acid (570 mL) and the mixture is refluxed for 3 hours. After cooling, the reaction mixture is basified with saturated aqueous sodium hydrogencarbonate solution and extracted with ethyl acetate. The extract is washed with water and saturated aqueous NaCl solution and dried over sodium sulfate. The organic layer is concentrated and the residue is recrystallized from ethanol to give N-(2-(4-benzyloxycarbonyl-1-piperazinyl)ethyl)phthalimide (35.09 g) as crystals.

M.p. 89.5-90.5° C.

2) A mixture of the compound obtained in the above step (1) (52.75 g), hydrazine hydrate (13.42 g) and ethanol (600 mL) is refluxed for 2 hours. The reaction mixture is filtered to remove the precipitated materials and the filtrate is concentrated. The residue is basified with 10% aqueous sodium hydroxide and extracted with ethyl acetate. The extract is dried over sodium sulfate and concentrated. To the residue is added hydrogen chloride in methanol and the mixture is concentrated. The residue is recrystallized from methanol-diethylether to give 1-(2-aminoethyl)-4-benzyloxy-carbonyl-piperazine 2 HCl (43.03 g) as crystals.

M.p. 174-174.5° C.

3) To the free base product of the compound obtained in the above step (2) (29.95 g; 2HCl salt) is added ethyl formate (300 mL). The mixture is refluxed for 19 hours. The reaction mixture is concentrated to give 1-(2-formylaminoethyl)-4-benzyloxycarbonyl-piperazine (25.67 g).

4) To a solution of the compound obtained in the above step (3) (25.67 g) in tetrahydrofuran (500 mL) is added borane-dimethylsulfide complex (35.2 mL) and the mixture is refluxed under heating for 15 hours. After cooling, to the reaction mixture is added dropwise methanol (100 mL) and the mixture is heated for 10 minutes. To the mixture is added dropwise 2M HCl-methanol (250 mL) and the mixture is refluxed under 3 hours. After cooling, the reaction mixture is concentrated and the residue is recrystallized from methanol-diethylether to give 1-(2-methylaminoethyl)-4-benzyloxycarbonyl-piperazine (28.63 g).

M.p. 190-190.5° C.

5) A mixture of the compound obtained in the above step (4) (3.5 g), picolinic acid (pyridine-2-carboxylic acid; 1.48 g), diethylcyanophosphonate (2.45 g), triethylamine (4.05 g) and dimethylformamide (35 mL) is stirred at room temperature for 5 hours. The reaction mixture is concentrated and to the residue is added ethyl acetate. The mixture is washed with saturated aqueous sodium hydrogencarbonate solution and saturated aqueous NaCl solution. The organic layer is dried over sodium sulfate and concentrated. The residue is purified by column chromatography on NH-silica gel (ethyl acetate:n-hexane=1:1) to give 1-(2-N-methyl-N-picolinoylaminoethyl)-4-benzyloxycarbonylpiperazine (3.64 g) as oil. MS(ESI)m/z: 383(M+H), IR(neat)cm⁻¹: 1699, 1634

6) A mixture of the compound obtained in the above step (5) (36 g) and ethanol (50 mL) is subjected to catalytic hydrogenation in the presence of 10% palladium-carbon (500 mg) under atmospheric pressure at room temperature. The reaction mixture is filtered and the filtrate is concentrated. The residue is recrystallized from methanol to give 1-(2-N-methyl-N-picolinoylaminoethyl)piperazine (3.74 g) as crystals. M.p. 225-227° C. (dec.)

7) A mixture of the compound obtained in the above step (5) (1.51 g), Lawesson's reagent (1.13 g) and dimethylformamide (30 mL) is refluxed for 6 hours. After cooling, the reaction mixture is concentrated and to the residue is added ethyl acetate. The solution is washed with saturated aqueous sodium hydrogencarbonate solution and saturated aqueous NaCl solution. The organic layer is dried over sodium sulfate and concentrated The residue is purified by column chromatography on silica gel (chloroform:methanol=80:1) to give 1-[2-(N-methyl-N-thiopicolinoylamino)ethyl]-4-benzyloxycarbonylpiperazine (1.36 g). MS(APCI)m/z: 399(M+H), IR(Nujol)cm⁻¹: 1700

8) A mixture of the compound obtained in the above step (7) (1.34 g), acetic acid (7 mL) and hydrobromide-acetic acid (13 mL) is stirred at room temperature for 3 hours. To the reaction mixture is added diisopropylether and the supernatant is discarded. The residue is dissolved in water and washed with ethyl acetate. The aqueous layer is basified with an aqueous ammonia and extracted with chloroform. The extract is dried over sodium sulfate and concentrated to give 1-[2-(N-methyl-N-thiopicolinoylamino)-ethyl]piperazine (923 mg) as oil. MS(APCI)m/z: 265(M+H), IR(neat)cm⁻¹: 3280

### Reference Example 20

1) To a solution of 2-aminopyridine (5.0 g) in tetrahydrofuran (31 mL) is added dropwise a solution of 1.6M n-butyllithium in n-hexane (1.6M) at −78° C. The mixture is stirred at 0° C. for 5 minutes. To the mixture is added dropwise ethyl bromoacetate (5.5 mL) at −78° C. The mixture is stirred at −20° C. for 20 minutes. After addition of saturated aqueous sodium hydrogencarbonate solution at −78° C., the reaction mixture is diluted with ethyl acetate. After warming to room temperature, the organic layer is washed with saturated aqueous sodium hydrogencarbonate solution and saturated aqueous NaCl solution, dried over sodium sulfate, and concentrated. The residue is purified by column chromatography on silica gel (n-hexane:ethyl acetate=1:1) and crystallized from diisopropylether to give 2-(bromoacetylamino)pyridine (4.72 g) as crystals.

M.p. 248-250° C. (dec.).

2) A solution of the compound obtained in the above step (1) (4.2 g),1-benzyloxycarbonylpiperazine (5.0 g) and triethylamine (2.52 g) in acetonitrile (50 mL) is stirred at room temperature for 4 hours. The reaction mixture is concentrated and to the residue is added ethyl acetate. The solution is successively washed with saturated aqueous sodium hydrogencarbonate solution, water and saturated aqueous NaCl solution. The organic layer is dried over sodium sulfate and concentrated. The residue is purified by column chromatography on silica gel (chloroform:methanol=100:1) and crystallized from diisopropylether to give 2-[(4-benzyloxycarbonylpiperazin-1-yl)acetylamino]pyridine (6.80 g) as crystals. M.p. 78-80° C.

3) To a solution of the compound obtained in the above step (2) (3.54 g) in tetrahydrofuran (50 mL) is added dropwise borane-dimethylsulfide complex in tetrahydrofuran (2M, 20 mL) and the mixture is stirred at room temperature for 18 hours. To the reaction mixture is added dropwise a solution of oxalic acid (7.2 g) in methanol (80 mL) under ice-cooling. The mixture is refluxed for 2 hours. After cooling, the reaction mixture is concentrated and to the residue is added ethyl acetate. The solution is washed with water and saturated aqueous NaCl solution. The organic layer is dried over sodium sulfate and concentrated. The residue is purified by column chromatography on silica gel (chloroform:methanol=100:1) to give 2-[2-(4-benazyloxycarbonylpiperazin-1-yl)-ethylamino]pyridine (3.42 g). MS(DI═EI)m/z: 340(M⁺), IR(neat)cm⁻¹: 3380, 1700, 1603

4) To a mixture of the compound obtained in the above step (3) (1.70 g), pyridine (593 mg) and methylene chloride (30 mL) is added dropwise a solution of acetyl chloride (470 mg) in methylenechloride (5 mL) under ice-cooling and the mixture is stirred at room temperature for 1 hour. The reaction mixture is concentrated and to the residue is added ethyl acetate. The solution is washed with saturated aqueous sodium hydrogencarbonate solution, water and saturated aqueous NaCl solution. The organic layer is dried over sodium sulfate and concentrated to give 2-[N-(2-(4-benzyloxycarbonylpiperazin-1-yl) ethyl)-N-acetylamino]-pyridine (1.92 g) as an oil.

5) A mixture of the compound obtained in the above step (4) (3.9 g) and ethanol (50 mL) is subjected to catalytic hydrogenation in the presence of 10% palladium-carbon (1.0 g) under atmospheric pressure at room temperature. The reaction mixture is filtered and the filtrate is concentrated. The residue is purified by column chromatography on silica gel (chloroform:methanol:aqueous 28% ammonia=80:20:1) to give 2-[N-(2-(piperazin-1-yl)ethyl)-N-acetylamino]pyridine (2.15 g) as an oil.

6) To a solution of the compound obtained in the above step (5) in methanol is two equivalent of fumaric acid. The mixture is concentrated and the residue is crystallized from ethanol-diisopropylether to give 2-[N-(2-(piperazin-1-yl)ethyl)-N-acetylamino]pyridine 2 fumarate (2.15 g). M.p. 153-155° C.

### Reference Example 21

A mixture of 3-bromopyridine (1.36 g), piperazine (3.71 g), sodium t-butoxide (1.24 g), trisdibenzylidenaceton-palladium complex (Pd₂(dba)₃; 158 mg), tri-o-toluylphosphine (105 mg), and toluene (60 mL) is stirred under argon gas atmosphere for 4 days under heating. The reaction mixture is concentrated and the residue is purified by column chromatography on silica gel (chloroform:methanol:aqueous ammonia=200:10:1). To the purified residue is added 5M HCl-ethanol and the mixture is concentrated. The residue is recrystallized from methanol-ethyl acetate to give 3-piperazinylpyridine 2 hydrochloride (759 mg) as crystals. M.p. 247-250° C. (dec.)

### Reference Example 22

1) To a solution of 4-chloro-5-amino-6-methylaminopyrimidine (24.59 g) in acetic acid (120 mL) and water (740 mL) is added dropwise a solution of sodium nitrite (21.39 g) in water (100 mL) at 3° C. and the mixture is stirred at the same temperature for 40 minutes. The reaction mixture is extracted with ethyl acetate. The aqueous layer is basified with saturated aqueous sodium hydrogencarbonate solution and extracted with ethyl acetate. The extract is washed with saturated aqueous sodium hydrogencarbonate solution, dried over sodium sulfate, and concentrated to give 7-chloro-3-methyl-3H-1,2,3-triazolo[4,5-d]pyrimidine (16.02 g) (Higashino T et al, Yakugaku-zassi 99, 1031(1979)). To the solution the above compound in dimethylacetamide (80 mL) are added dropwise 1-benzylpiperazine (17.48 g) and diisopropylamine (18.31 g) under ice-cooling, successively. The mixture is stirred at the same temperature for 1 hour. To the reaction mixture is added ethyl acetate and the mixture is washed with water, dried over sodium sulfate, and concentrated. The residue is crystallized from diisopropylether to give 7-(4-benzylpiperazin-1-yl)-3-methyl-3H-1,2,3-triazolo[4,5-d]pyrimidine (28.16 g, 58%) as crystals.

M.p. 91-92° C.

2) A mixture of the compound obtained in the above step (1) (28 g),10% palladium-carbon (5.6 g), ammonium formate (57 g) and methanol (560 mL) is refluxed for 1.5 hours. After cooling, the reaction mixture is filtered and the filtrate is concentrated. To the residue is added 10% aqueous potassium carbonate and the mixture is extracted with chloroform. The extract is washed with water and saturated aqueous NaCl solution, dried over sodium sulfate, and concentrated The residue is crystallized from diisopropylether to give 7-(1-piperazinyl)-3-methyl-3H-1,2,3-triazolo[4,5-d]pyrimidine (17.68 g, 89%) as crystals.

M.p. 95-96° C.

### Reference Example 23

1) A mixture of 3,4-Dihydroxyphenethylamine hydrochloride (1.0 g),1-carboethoxy-4-piperidone (1.083 g) and triethylamine (535 mg) in ethanol (15 mL) is refluxed for 13 hours. After cooling, to the reaction mixture is added HCl-ethanol and the mixture is concentrated. The residue is recrystallized from methanol-ethanol to give 1′-ethoxycarbonyl-3,4-dihydro-6,7-dihydroxy-spiro[isoquinoline-1(2H),4′-piperidine] hydrochloride (1.64 g, 91%) as crystals.

M.p. 285-286° C. (dec.).

2) To a mixture of the compound obtained in the above step (1) (5.0 g) in dimethylformamide (100 mL) is added potassium t-butoxide (5.401 g) under ice-cooling and the mixture is stirred for 10 minutes. To the reaction mixture is added dropwise methyl iodide (2 mL) and the mixture is stirred for 1.5 hours. The reaction mixture is poured into water and the mixture is extracted with ethyl acetate. The extract is washed with water and saturated aqueous NaCl solution, dried over sodium sulfate, and concentrated. The residue is purified by column chromatography on NH-silica gel (n-hexane:ethyl acetate=5:1 to 3:1) to give 1′-ethoxycarbonyl-3,4-dihydro-6,7-dimethoxy-spiro[isoquinoline-1(2H),4′-piperidine] (2.89 g).

MS(APCI)m/z: 335(M+H), IR(neat)cm⁻¹: 1694, 1609.

3) To a solution of the compound obtained in the above step (2) (1.0 g) in methylene chloride (20 mL) is added triethylamine (1.26 mL) and then acryloyl chloride (0.36 mL) under ice-cooling. The mixture is stirred for 2 hours. To the reaction mixture is added water and the mixture is extracted with chloroform. The extract is washed with 10% HCl, saturated aqueous sodium hydrogencarbonate solution, and saturated aqueous NaCl solution. The organic layer is dried over sodium sulfate and concentrated to give 1′-ethoxycarbonyl-2-acryloyl-3,4-dihydro-6,7-dimethoxy-spiro[isoquinoline-1(2H),4′-piperidine] (1.18 g).

4) To the compound obtained in the above step (3) is added benzylamine (5 mL) and the mixture is stirred at 150° C. for 3 hours. After cooling, the reaction mixture is diluted with ethyl acetate and then added 10% HCl. The precipitates are removed by filtration and the filtrate is basified with potassium carbonate. The mixture is extracted with ethyl acetate and the extract is washed with water and saturated aqueous NaCl solution. The organic layer is dried over sodium sulfate and concentrated . The residue is purified by column chromatography on silica gel (n-hexane:ethyl acetate=2:1) to give 1′-ethoxycarbonyl-3,4-dihydro-6,7-dimethoxy-2-(3-dibenzylaminopropionyl)-spiro[isoquinoline-1(2H),4′-piperidine] (1.30 g, 74%) as an oil. MS(APCI)m/z: 586(M+H), IR(neat)cm⁻¹: 1735, 1693

5) To a suspension of sodium borohydride (247 mg) in tetrahydrofuran (10 mL) is added trifluoroborane diethylether complex (1.1 mL) under ice-cooling and the mixture is stirred for 15 minutes. To the mixture is added dropwise a solution of the compound obtained in the above step (4) (1.27 g) in tetrahydrofuran (10 mL) under ice-cooling. The mixture is refluxed for 1.5 hours. To the reaction mixture is added 10% HCl (10 mL) and the mixture is refluxed for 2 hours and then stirred at room temperature for 12 hours. The organic solvent is removed and the resiUue is basified with potassium carbonate Th mixture is extracted with ethyl acetate and the extract is washed with saturated aqueous NaCl solution, dried over sodium sulfate, and concentrated. The residue is purified by column chromatography on NH-silica gel (n-hexane:ethyl acetate=3:1) to give 1′-ethoxycarbonyl-3,4-dihydro-6,7-dimethoxy-2-(3-dibenzylaminopropyl)-spiro[isoquinoline-1(2H),4′-piperidine] (717 mg, 58%) as an oil. MS(APCI)m/z: 572(M+H), IR(neat)cm⁻¹: 1697, 1694

6) A mixture of the compound obtained in the above step (5) (697 mg), ethylene glycol (7 mL) and 50% aqueous sodium hydroxide (7 mL) is refluxed for 3 hours. After cooling, the reaction mixture is diluted with water and extracted with chloroform. The extract is washed with saturated aqueous NaCl solution, dried over sodium sulfate, and concentrated. The residue is purified by column chromatography on silica gel (chloroform:methanol:aqueous ammonia=20:1:0.1) to give 3,4-dihydro-6,7-dimethoxy-2-(3-dibenzylaminopropyl)-spiro[isoquinoline-1(2H),4′-piperidine] (565 mg). MS(APCI)m/z: 500(M+H), IR(neat)cm⁻¹: 3327

7) To a solution of the compound obtained in the above step (6) (535 mg) and triphosgene (127 mg) in methylene chloride (10 mL) is added dropwise triethylamine (0.3 mL) under ice cooling. The mixture is stirred at the same temperature for 15 minutes. The reaction mixture is concentrated and to the residue is added methylene chloride (10 mL), triethylamine (0.3 mL) and 1-(3-methyl-3H-1,2,3-triazolo[4,5-dlpyrimidin-7-yl)piperazine (235 mg) and the mixture is stirred for 13 hours at room temperature. The reaction mixture is concentrated and the residue is diluted with ethyl acetate. The solution is washed with water, saturated aqueous sodium hydrogencarbonate solution and saturated aqueous NaCl solution. The organic layer is dried over sodium sulfate and concentrated. The residue is purified by column chromatography on silica gel (chloroform:methanol=100:1) to give 3,4-dihydro-6,7-dimethoxy-2-(3-dibenzylaminopropyl)-1′-[4-(3-methyl-3H-1,2,3-triazolo[4,5-d]pyrimidin-7-yl)piperazin-1-yl]carbonyl-spiro[isoquinoline-1(2H),4′-piperidine] (595 mg, 75%). MS(APCI): 745(M+H), IR(neat)cm⁻¹: 1643

### Reference Example 24

1) To a solution of the compound obtained in Reference example 13(3) (20.0 g) in dimethylformamide (140 mL) is added 60% sodium hydride (3.44 g) at 4° C. and the mixture is stirred at the same temperature for 30 minutes and then stirred at room temperature for 30 minutes. The reaction mixture is cooled to 4° C. and to the mixture is added dropwise iodoethane (15.2 g) and then stirred at the same temperature for 1 hour. The mixture is further stirred at room temperature for 17 hours and poured into saturated aqueous ammonium chloride solution. The mixture is extracted with ethyl acetate and the extract is washed with water. The organic layer is dried over sodium sulfate and concentrated. The residue is crystallized from diisopropylether-n-hexane to give 2′-benzyloxycarbonyl-3′,4′-dihydro-6′,7′-diethoxy-4,4-bisethoxycarbonyl-spiro[cyclohexane-1,1′(2′H)-isoquinoline] (19.7 g, 88.8%).

M.p. 88.4-89.1° C., MS(APCI): 568(M+H), IR(Nujol)cm⁻¹: 1725, 1703, 1611.

2) A mixture of the compound obtained in the above step (1) (19.7 g) and sodium hydroxide (13.9 g) in water (90 mL) and ethanol (90 mL) is stirred at room temperature for 15 hours and then refluxed for 5 hours. After cooling, the reaction mixture is concentrated, acidified with hydrochloric acid, and extracted with ethyl acetate. The extract is washed with water, dried over sodium sulfate, and concentrated. The residue is crystallized from diisopropylether-n-hexane to give 2′-benzyloxycarbonyl-3′,4′-dihydro-6′,7′-diethoxy-4,4-biscarboxy-spiro[cyclohexane-1,1′(2′H)-isoquinoline] (16.14 g, 90.9%).

M.p. 175-176° C., MS(ESI): 510(M−H), IR(Nujol)cm⁻¹: 1747, 1705, 1669, 1609.

3) A solution of the compound obtained in the above step (2) (16.13 g) in pyridine (120 mL) is refluxed for 15 hours. After cooling, the solvent is removed in vacuo and the residue is subjected to azeotropic distillation with toluene to remove pyridine completely to give 2′-benzyloxycarbonyl-3′,4′-dihydro-6′,7′-diethoxy-4-carboxy-spiro[cyclohexane-1,1′(2′H)-isoquinoline] as a crude product.

4) A mixture of the compound obtained in the above step (3), methanol (120 mL) and 10% palladium-carbon (700 mg) is subjected to catalytic hydrogenation under atmosphric pressure. The reaction mixture is filtered to remove catalysts and the filtrate is washed with hot methanol and hot dimethylformamide. The combined filtrate is concentrated. The residue is dissolved in pyridine and the solution is refluxed for 15 hours. After cooling, the reaction mixture is evaporated and the ressdue 1crystallized from ethanol to give (1α,4β)-3′,4′-dihydro-6′,7′-diethoxy-4-carboxy-spiro[cyclohexane-1,1′(2′H)-isoquinoline] (5.26 g, 50%) as crystals.

M.p. 197-198° C., MS(APCI): 334(M+H), IR(Nujol)cm⁻¹: 3549, 3314, 1642.

5) To a solution of the compound obtained in the above step (4) (2.2 g) in dimethylformamide (33 mL) are added cesium carbonate (2.58 g) and benzyl bromide (863 μL). The mixture is stirred at room temperature for 2 hours. The reaction mixture is concentrated and the residue is poured into water (100 mL) and then extracted with ethyl acetate. The extract is washed with water and saturated aqueous NaCl solution, dried over sodium sulfate, and concentrated. The residue is purified by column chromatography on silica gel (chloroform:methanol=20:1) to give (1α,4β)-3′,4′-dihydro-6′,7′-diethoxy-4-benzyloxy-carbonyl-spiro[cyclohexane-1,1′(2′H)-isoquinoline] (2.80 g, quantitativly) as a yellow oil.

MS(APCI): 424(M+H), IR(neat)cm⁻¹: 1727, 1607.

6) A mixture of the compound obtained in the above step (5) (2.80 g), N-(2-bromoethyl)phthalimide (23.5 g), diisopropylethylamine (12.0 g) and dimethylformamide (5 mL) is stirred at 130° C. for 3 hours. After cooling, to the reaction mixture is added ethyl acetate (50 mL) and the mixture is filtered to remove insoluble materials. The filtrate is concentrated and to the residue is added ethyl acetate and then washed with water and saturated aqueous NaCl solution. The organic layer is dried over sodium sulfate and concentrated. The residue is column chromatography on silica gel (chloroform:methanol:aqueous ammonia=19:1:0.1) to give (1α,4β)-2′-(2-phthalimidoethyl)-3′,4′-dihydro-6′,7′-diethoxy-4-benzyloxycarbonyl-spiro[cyclohexane-1,1′(2′H)-isoquinoline] (2.55 g, 82.7%) as a yellow oil.

MS(APCI): 597(M+H), IR(neat)cm⁻¹: 1773, 1714, 1607.

7) To a solution of the compound obtained in the above step (6) (2.55 g) in tetrahydrofuran (20 mL) and ethanol (20 mL) is added hydrazine hydrate (2.14 g) and the mixture is stirred at room temperature for 16 hours. The reaction mixture is poured into saturated aqueous sodium hydrogencarbonate solution and extracted with chloroform. The extract is washed with water and saturated aqueous NaCl solution. The organic layer is dried over sodium sulfate.and concentrated. The residue is purified by column chromatography on silica gel (chloroform:methanol aqueous ammonia=9:1:0.1) to give (1α,4β)-2′-(2-aminoethyl)-3′,4′-dihydro-6′,7′-diethoxy-4-benzyloxycarbonyl-spiro[cyclohexane-1,1′(2′H)-isoquinoline] (1.64 g, 82.3%) as a yellow oil.

MS(APCI): 467(M+H), IR(neat)cm⁻¹: 1727, 1684, 1653, 1606.

8) To a solution of the compound obtained in the above step (7) (1.64 g) in tetrahydrofuran (30 mL) is added aqueous formalin (3 mL) and then added dropwise sodium borohydride (665 mg) over a period of 30 minutes. The mixture is stirred at room temperature for 16 hours. The reaction mixture is concentrated and subjected to azeotropic distillation with toluene. To the distilled product is added saturated aqueous sodium hydrogencarbonate solution (100 mL) and the solution is extracted with sodium hydrogencarbonate solution and saturated aqueous NaCl solution. The organic layer is dried over sodium sulfate and concentrated. The residue is purified by column chromatography on silica gel (chloroform:methanol:aqueous ammonia=20:1:0.1) to give (1α,4β)-2′-(2-dimethylaminoethyl)-3′,4′-dihydro-6′,7′-diethoxy-4-benzyloxycarbonyl-spiro[cyclohexane-1,1′(2′H)-isoquinoline] (1.42 g, 81.8%) as a colorless oil.

MS(APCI): 495(M+H), IR(neat)cm⁻¹: 1729, 1606.

9) To a solution of the compound obtained in the above step (8) (1.42 g) in methanol (30 mL) is added 10% palladium-carbon (300 mg). The mixture is subjected to catalytic hydrogenation under atmosphric pressure. The reaction mixture is filtered to remove catalysts. The catalysts are washed with methanol and water. The combined filtrate is concentrated. The residue is crystallized from ethyl acetate-diethylether to give (1α,4β)-2′-(2-dimethylaminoethyl)-3′,4′-dihydro-6′,7′-diethoxy-4-carboxy-spiro[cyclohexane-1,1′(2′H)-isoquinoline] (1.09 g, 93.9%) as crystals.

M.p. 111.9-112° C., MS(APCI): 405(M+H), IR(Nujol)cm⁻¹: 3385, 1737, 1693, 1607.

### Reference Example 25

1) To a solution of the compound obtained in the Reference example 16(1) (1.226 g) and triethylamine (2.915 g) in methylene chloride (10 mL) is added dropwise a solution of chloroacetyl chloride (0.51 mL) in methylene chloride (10 mL) under ice-cooling. The mixture is stirred at the same temperature for 2 hours. The reaction mixture is poured into saturated aqueous sodium hydrogenchloride solution and extracted with chloroform. The extract is washed with saturated aqueous NaCl solution, dried over sodium sulfate, and concentrated. The residue is purified by column chromatography on silica gel (chloroform:methanol=50:1) to give (1α,4β)-2′-chloroacetyl-3′,4′-dihydro-6′,7′-dimethoxy-4-benzyloxycarbonyl-spiro[cyclohexane-1,1′(2′H)-isoquinoline] (1.35 g) as an oil.

MS(APCI): 472(M+H), IR(neat)cm⁻¹: 1727, 1661.

2) To a solution of the compound obtained in the above step (1) (1.334 g) in acetonitrile (20 mL) is added 50% dimethylamine (10 mL). The mixture is stirred at room temperature overnight. The reaction mixture is concentrated and the residue is purified by column chromatography on silica gel (chloroform:methanol=20:1) to give (1α,4β)-2′-dimethylaminoacetyl-3′,4′-dihydro-6′,7′-dimethoxy-4-benzyloxycarbonyl-spiro[cyclohexane-1,1′(2′H)-isoquinoline] (1.34 g) as an oil.

MS(APCI): 481(M+H), IR(neat)cm⁻¹: 1731, 1658, 1651, 1633.

3) To a solution of the compound obtained in the above step (2) (1.32 g) in methanol (20 mL) is added 10% palladium-carbon (263 mg). The mixture is subjected to catalytic hydrogenation under atmospheric pressure for 24 hours. The reaction mixture is filtered to remove catalysts and the filtrate is concentrated to give (1α,4β)-2′-dimethylaminoacetyl-3′,4′-dihydro-6′,7′-dimethoxy-4-carboxy-spiro[cyclohexane-1,1′(2′H)-isoquinoline] (0.99 g) as crystals.

M.p. 233-235° mC. (dec.), MS(APCI): 391(M+H), IR(Nujol)cm⁻¹: 1671.

### Reference Example 26

1) The compound obtained in the Reference example 24(5) (1.53 g) is treated in the same manner as described in Reference example 25(1) to give (1α,4β)-2′-chloroacetyl-3′,4′-dihydro-6′,7′-diethoxy-4-benzyloxycarbonyl-spiro[cyclohexane-1,1′(2′H)-isoquinoline] (1.86 g) as an oil.

MS(APCI): 500(M+H), IR(neat)cm⁻¹: 1729, 1661, 1644, 1608.

2) The compound obtained in the above step (1) (1.85 g) is treated in the same manner as described in Reference example 25(2) to give (1α,4β)-2′-dimethylaminoacetyl-3′,4′-dihydro-6′,7′-diethoxy-4-benzyloxycarbonyl-spiro[cyclohexane-1,1′(2′H)-isoquinoline] (1.84 g) as an oil.

MS(APCI): 509(M+H), IR(neat)cm⁻¹: 1729, 1656, 1635, 1609.

3) The compound obtained in the above step (2) (1.83 g) is treated in the same manner as described in Reference example 25(3) to give (1α,4β)-2′-dimethylaminoacetyl-3′,4′-dihydro-6′,7′-diethoxy-4-carboxy-spiro[cyclohexane-1,1′(2′H)-isoquinoline] (768 mg) as an amorphous powder.

MS(APCI): 419(M+H), IR(Nujol)cm⁻¹: 3397, 1651, 1610.

### Reference Example 27

1) To a solution of the compound obtained in the Reference example 24(5) (1.49 g) in a mixture of tetrahydrofuran (15 mL) and acetic acid (5 mL) are added an aqueous formalin (590 μL) and sodium triacetoxy borohydride (895 mg) under ice-cooling and the mixture is stirred at room temperature for 3 hours. To the reaction mixture is added saturated aqueous sodium hydrogencarbonate solution and the mixture is extracted with ethyl acetate. The extract is washed with water, dried over sodium sulfate, and then concentrated. The residue is purified by column chromatography on silica gel (chloroform:methanol=50:1) to give (1α,4β)-2′-methyl-3′,4′-dihydro-6′,7′-diethoxy-4-benzyloxycarbonyl-spiro[cyclohexane-1,1′(2′H)-isoquinoline] (1.6 g, quantitatively) as an oil.

MS(APCI): 438(M+H), IR(neat)cm⁻¹: 1727, 1608.

2) To a solution of the compound obtained in the above step (1) (1.59 g) in methanol (20 mL) is added 10% palladium-carbon (300 mg). The mixture is subjected to catalytic hydrogenation under atmospheric pressure. The reaction mixture is filtered to remove catalysts and the filtrate is concentrated to give (1α,4β)-2′-methyl-3′,4′-dihydro-6′,7′-diethoxy-4-carboxy-spiro[cyclohexane-1,1′(2′H)-isoquinoline] (1.1 g) as crystals. M.p. 235° C.

### Reference Example 22

1) To a solution of the compound obtained in the Reference example 24(5) (1.35 g) in dimethylacetamide (7 mL) are added 2-bromoethanol (7.0 mL) and diisopropylethylamine (16.7 mL) and the mixture is stirred at 100° C. for 3 hours. The reaction mixture is poured into water (200 mL) and extracted with ethyl acetate. The extract is washed with water, dried over sodium sulfate, and concentrated. The residue is purified by column chromatography on silica gel (chloroform:methanol=50:1) to give (1α,4β)-2′-(2-hydroxyethyl)-3′,4′-dihydro-6′,7′-diethoxy-4-benzyloxycarbonyl-spiro[cyclohexane-1,1′(2′H)-isoquinoline] (1.45 g, quantitativly) as an oil.

MS(APCI): 468(M+H), IR(neat)cm⁻¹: 3446, 1727, 1606.

2) To a solution of the compound obtained in the above step (1) (1.44 g) in ethanol (38 mL) is added 10% palladium-carbon (480 mg). The mixture is subjected to catalytic hydrogenation under atmospheric pressure. The reaction mixture is filtered to remove catalysts and the filtrate is concentrated to give (1α,4β)-2′-(2-hydroxyethyl)-3′,4′-dihydro-6′,7′-diethoxy-4-carboxy-spiro[cyclohexane-1,1′(2′H)-isoquinoline] (1.1 g) as an amorphous powder.

MS(APCI): 378(M+H), IR(neat)cm⁻¹: 3335, 1713, 1607.

### Reference Example 29

1) A mixture of the compound obtained in the Reference example 12(4) (824 mg), iodoethane (1.08 mL), and potassium carbonate (1.119 g) in dimethylformamide (8 mL) is stirred at room temperature for 1 hour and at 100° C. for 2 hours. After cooling, to the reaction mixture is added ethyl acetate, and then washed with water and saturated aqueous NaCl solution. The organic layer is dried over sodium sulfate and evaporated. The residue is purified by column chromatography on silica gel (chloroform:methanol:aqueous ammonia=300:10:1) to give (1α,4β)-2′-ethyl-3′,4′-dihydro-6′,7′-dimethoxy-4-ethoxycarbonyl-spiro[cyclohexane-1,1′(2′H)-isoquinoline] (1.08 g) as an oil.

MS(APCI)m/z: 362(M+H).

2) To a solution of the compound obtained in the above step (1) (1.08 g) in methanol (10 mL) is added 2M aqueous sodium hydroxide (4.1 mL). The mixture is stirred overnight at room temperature. After addition of 2M hydrochloric acid, the reaction mixture is concentrated. The residue is dissolved in ethanol (20 mL) and the insoluble materials are removed by filtration with Celite. The filtrate is concentrated and the residue is dissolved in chloroform. The solution is filtered through Celite and the filtrate is concentrated. To the residue is added conc.HCl-ethanol and the mixture is concentrated. The residue is crystallized from ethanol-isopropyl alcohol-ether to give (1α,4β)-2′-ethyl-3′,4′-dihydro-6′,7′-dimethoxy-4-carboxy-spiro[cyclohexane-1,1′(2′H)-isoquinoline] hydrochloride (539 mg) as crystals.

M.p. 254-259° C. (dec.).

### Reference Example 30

1) The compound obtained in Reference example 12(4) is treated in the same manner as described in Reference example 29(1) to give (1α,4β)-2′-propyl-3′,4′-dihydro-6′,7′-dimethoxy-4-propyloxy-carbonyl-spiro[cyclohexane-1,1′(2′H)-isoquinoline] hydrochloride as an oil.

MS(APCI): 390(M+H), IR(neat)cm⁻¹: 1727, 1607.

2) The compound obtained in the above step (1) is treated in the same manner as described in Reference example 29(2) to give (1α,4β)-2′-propyl-3′,4′-dihydro-6′,7′-dimethoxy-4-carboxy-spiro[cyclohexane-1,1′(2′H)-isoquinoline] hydrochloride as crystals.

M.p. 266-270° C.

### Reference Example 31

1) To a solution of the compound obtained in Reference example 16(1) (2.77 g) in tetrahydrofuran (30 mL) is added an aqueous formalin (30 mL) and sodium triacetoxyborohydride (4.45 g) under ice-cooling and the mixture is stirred at room temperature for 2 hours. To the reaction mixture is added saturated aqueous sodium hydrogencarbonate solution. The mixture is extracted with ethyl acetate and the extract is washed with water, dried over sodium sulfate, and concentrated. The residue is purified by column chromatography on silica gel (chloroform:methanol=20:1) to give (1α,4β)-2′-methyl-3′,4′-dihydro-6′,7′-dimethoxy-4-benzyloxycarbonyl-spiro[cyclohexane-1,1′(2′H)-isoquinoline] (2.76 g, 96.4%) as an amorphous powder.

MS(APCI): 410(M+H), IR(neat)cm⁻¹: 1727, 1608.

2) A solution of the compound obtained in the above step (1) (2.76 g) in ethanol (30 mL) is added palladium hydroxide (400 mg). The mixture is subjected to catalytic hydrogenation under at 3 atm pressure. To the reaction mixture is added water and the mixture is filtered to remove catalysts. The filtrate is concentrated to give (1α, 4β)-2′-methyl-3′,4′-dihydro-6′,7′-dimethoxy-4-carboxy-spiro[cyclohexane-1,1′(2′H)-isoquinoline] (1.87 g) as crystals.

M.p. 199° C., MS(APCI): 320(M+H), IR(neat)cm⁻¹: 3385, 1722, 1611.

### Reference Example 32

1) A mixture of the compound obtained in Reference example 16(1) (5.587 g), N-(2-bromoethyl)phthalimide (35.89 g) and diisopropylethylamine (18.26 g) in dimethylacetamide (10 mL) is stirred at 130° C. for 10 hours. To the reaction mixture is added additional N-(2-bromoethyl)phthalimide (13.50 g) and the mixture is stirred at 130° C. for 10 hours. After cooling, to the reaction mixture is added ethyl acetate and the mixture is washed with water and saturated aqueous NaCl solution. The organic layer is dried over sodium sulfate and concentrated. The residue is purified by column chromatography on silica gel (n-hexane:ethyl acetate=3:1 to 1:1) to give (1α,4β)-2′-(2-phthalimidoethyl)-3′,4′-dihydro-6′,7′-dimethoxy-4-benzyloxycarbonyl-spiro[cyclohexane-1,1′(2′H)-isoquinoline] (7.14 g) as a pale yellow amorphous powder.

MS(APCI)m/z: 569(M+H), IR(neat+chloroform)cm⁻¹1712, 1772.

2) A solution of the compound obtained in the above step (1) (7.09 g) in ethanol (30 mL) and tetrahydrofuran (30 mL) is added hydrazine hydrate (6.24 g). The mixture is stirred at room temperature for 7 hours. To the reaction mixture is added saturated aqueous sodium hydrogencarbonate solution. The mixture is extracted with chloroform. The extract is washed with saturated aqueous NaCl solution, dried over sodium sulfate, and concentrated. The residue is purified by column chromatography on silica gel (chloroform/methanol/aqueous ammonia=200:10:1) to give (1α,4β)-2′-(2-aminoethyl)-3′,4′-dihydro-6′,7′-dimethoxy-4-benzyloxy-carbonyl-spiro[cyclohexane-1,1′(2′H)-isoquinoline] (4.91 g) as pale yellow crystals.

M.p. 84-85° C., MS(APCI)m/z: 639(M+H), IR(neat+chloroform)cm⁻¹: 3373, 1726.

3) To a solution of the compound obtained in the above step (2) (4.88 g) in tetrahydrofuran (100 mL) and acetic acid (20 mL) is added 35% aqueous formalin (10 mL) under ice-cooling. Then to the mixture is added portionwise sodium borohydride (2.10 g) over a period of 20 minutes. The mixture is stirred at the same temperature for 30 minutes and at room temperature for 4 hours. The reaction mixture is concentrated and the residue is subjected to added saturated aqueous sodium hydrogencarbonate solution and the mixture is extracted with chloroform. The extract is washed with a saturated aqueous NaCl solution, dried over sodium sulfate, and concentrated. The residue is purified by column chromatography on silica gel (chloroform/methanol/aqueous ammonia=200:10:1) to give (1α,4β)-2′-(2-dimethylaminoethyl)-3′,4′-dihydro-6′,7′-dimethoxy-4-benzyloxycarbonyl-spiro[cyclohexane-1,1′(2′H)-isoquinoline] (4.81 g) as a pale yellow oil.

MS(APCI)m/z: 467(M+H), IR(neat+chloroform)cm⁻¹: 1728.

4) A mixture of the compound obtained in the above step (3) (4.78 g) and 10% palladium-carbon (500 mg) in ethanol (80 mL) is subjected to catalytic hydrogenation under atmospheric pressure. The reaction mixture is filtered and the catalyst on the filter is washed with methanol and water. The combined filtrate is concentrated. The residue is crystallized from ethyl acetate to give (1α,4β)-2′-(2-dimethylaminoethyl)-3′,4′-dihydro-6′,7′-dimethoxy-4-carboxy-spiro[cyclohexane-1,1′(2′H)-isoquinoline] (3.37 g) as colorless crystals.

M.p. 231-233° C.(dec.), MS(APCI)m/z: 377(M+H), IR(neat+chloroform)cm⁻¹: 1683.

### Reference Example 33

1) A mixture of m-methoxyphenthylamine (25.98 g) and 47% hydrobromic acid (150 mL) is stirred at 120-130° C. for 5 hours. The reaction mixture is concentrated. The residue is subjected to azeotropic distillation with ethanol and crystallized from isopropanol-ether to give 3-hydroxyphenethylamine hydrobromide (34.1 g, 90.9%). M.p. 118-120° C.

2) A mixture of the compound obtained in the above step (1) (34.08 g),4,4-bisethoxycarbonylcyclohexanone (90.7 g), triethylamine (43.5 g) and ethanol (300 mL) is stirred at 50-60° C. for 64 hours. The precipitates are collect and washed with ethanol and ether to give 3′,4′-dihydro-6′-hydroxy-4,4-bisethoxycarbonyl-spiro[cyclohexane-1,1′(2′H)-isoquinoline] (34.95 g, 62%) as crystals.

M.p. 188.5-190° C.

3) A solution of the compound obtained in the above step (2) (43.73 g) in ethylene chloride (500 mL) is added triethylamine (67.5 mL) and then cooled in an ice-bath. To the mixture is added dropwise chlorotrimethylsilane (39.43 g) and the mixture is stirred at room temperature for 26 hours. To the reaction mixture is added 10% aqueous citric acid. The organic layer is separated, washed with water and an aqueous NaCl solution, dried over sodium sulfate, and concentrated. The residue is triturated with isopropylether-n-hexane to give 3′,4′-dihydro-6′-trimethyl-silyloxy-4,4-bisethoxycarbonyl-spiro[cyclohexane-1,1′(2′H)-isoquinoline] (34.88 g) as a crude product.

4) To a solution of the compound obtained in the above step (3) and diisopropylethylamine (28 ml) in methylene chloride (400 mL) is added dropwise benzyl chloroformate (27.4 g) under ice-cooling and the mixture is stirred at room temperature for 8 hours. The reaction mixture is concentrated and to the residue is added ethyl acetate. The mixture is washed with water, 5% aqueous citric acid, saturated aqueous sodium hydrogencarbonate solution, and saturated aqueous NaCl solution, successively. The organic layer is dried over sodium sulfate and concentrated to give 2′-benzyloxycarbonyl-3′,4′-dihydro-6′-trimethylsilyloxy-4,4-bisethoxycarbonyl-spiro[cyclohexane-1,1′(2′H)-isoquinoline] (54.44 g) as a crude product.

5) To a solution of the compound obtained in the above step (4) in acetonitrile (250 mL) is added an aqueous 47% hydrofluoric acid under ice-cooling and the mixture is stirred at the same temperature for 30 minutes. To the reaction mixture is added sodium hydrogencarbonate to adjust its pH to 6 and then the mixture is concentrated. To the residue is added ethyl acetate and water and the mixture is adjusted its pH to 4 by addition of citric acid. The organic layer is separated, washed with water and saturated aqueous NaCl solution, dried over sodium sulfate, and concentrated. The residue is recrystallized from ethyl acetate-n-hexane to give 2′-benzyloxycarbonyl-3′,4′-dihydro-6′-hydroxy-4,4-bisethoxycarbonyl-spiro[cyclohexane-1,1′(2′H)-isoquinoline] (28.4 g, 71.2%) as crystals. M.p. 123-124° C.

The mother liquor obtained as above is purified by column chromatography on silica gel (ethyl acetate:n-hexane=1:4) to give the same final product as above (5.78 g, 14.5%).

6) To a solution of the compound obtained in the above step (5) (26.18 g) in dimethylformamide (60 mL) are added potassium carbonate (14.7 g) and iodoethane (8.5 mL). The mixture is stirred at 50-60° C. for 5 hours. The reaction mixture is poured into ice-cooled water and the mixture is extracted with ethyl acetate. The extract is washed with water and an aqueous NaCl solution, dried over sodium sulfate, and concentrated. The residue (27.6 g) is added to a mixture of ethanol (100 mL) and water (100 mL) and then. to the mixture is added an aqueous sodium hydroxide (21.1 g). The mixture is stirred at room temperature for 14 hours and refluxed for 7 hours. The reaction mixture is concentrated and the residual mixture is acidified with 10% HCl under ice-cooling, washed with ethyl acetate, the extract is washed with saturated aqueous NaCl, dried and concentrated. The residue is crystallized from isopropyl ether to give 2′-benzyloxycarbonyl-3′,4′-dihydro-6′-ethoxy-4,4-biscarboxy-spiro[cyclohexane-1,1′(2′H)-isoquinoline] (23.91 g) as crystals. M.p. 184-185° C.

7) A mixture of the compound obtained in the above step (6) (21.4 g) and pyridine (230 mL) is refluxed under heating for 5.5 hours. The reaction mixture is concentrated and the residue is dissolved in ethyl acetate. The solution is washed with 10% HCl and saturated aqueous NaCl solution and dried. The solution is concentrated and to the residue is added methanol (400 mL) and 5% palladium-carbon (2 g). The mixture is subjected to hydrogenation reaction at a normal pressure at room temperature. The reaction mixture is filtered to remove catalysts and the filtrate is concentrated. To the residue is added pyridine (200 mL) and the mixture is refluxed under heating for 24 hours. The reaction mixture is concentrated and the residue is subjected to azeotropic distillation with toluene. The residue is dissolved in methanol and the solution is treated with carbon powder. The treated solution is concentrated and the residue is recrystallized from ethyl acetate to give (1α,4β)-3′,4′-dihydro-6′-etoxy-4-carboxy-spiro[cyclohexane-1,1′(2′H)-isoquinoline] (6.04 g) as crystals. M.p. 209-211.5° C.

### Reference Example 34

1) To a mixture of 3-methoxyphenethylamine (25.5 g) and 4,4-bisethoxycarbonylcyclohexanone (68.5 g) is added polyphosphoric acid (250 g) and the mixture is stirred at 120° C. (bath temp.) for 40 minutes. To the reaction mixture is added water (500 mL) and saturated aqueous NaCl solution (500 mL) and the mixture is extracted with chloroform. The extract is washed with saturated aqueous sodium hydrogencarbonate solution and saturated aqueous NaCl solution. The organic layer is dried over sodium sulfate and concentrated. The residue is purified by chromatography on neutral silica gel (solvent; n-hexane/ethyl acetate (4:1) to ethyl acetate) to give 3′,4′-dihydro-6′-methoxy-4,4-bisethoxycarbonyl-spiro[cyclohexane-1,1′(2′H)-isoquinoline] (23.32 g, 36.8%) as crystals. M.p. 81-83° C.

2) To a solution of the compound obtained in the above step (1) (21.76 g) and diisopropylethylamine (11.24 g) in methylene chloride is added dropwise benzyl chloroformate (12.4 mL) and the mixture is stirred at room temperature for 1.5 hour and then refluxed for 2.5 hours. After cooling, the reaction mixture is concentrated and the residue is diluted with ethyl acetate. The mixture is washed with water and saturated aqueous NaCl solution. The organic layer is dried over sodium sulfate and concentrated. To a mixture of the residue, water (100 mL), and ethanol (100 mL) is added sodium hydroxide (23.1 g) under ice-cooling. The mixture is stirred for 16 hours at room temperature and then refluxed for 7 hours. The reaction mixture is concentrated and acidified with 10% aqueous HCl and then extracted with ethyl acetate. The extract is washed with saturated aqueous NaCl solution, dried over sodium sulfate, concentrated. The residue is crystallized from isopropylether to give 3′,4′-dihydro-6′-methoxy-2′-benzyloxycarbonyl-4,4-biscarboxy-spiro[cyclohexane-1,1′(2′H)-isoquinoline] (24.5 g, 93.3%) as crystals. M.p. 193-194° C.

3) The compound obtained in the above step (2) is treated in the same manner as described in Reference example 33(7) to give (1α,4β)-3′,4′-dihydro-6′-methoxy-4-carboxy-spiro[cyclohexane-1,1′(2′H)-isoquinoline] (Yield: 51.5%) as crystals. M.p. 201-205° C.

### Reference Example 35

1) To a solution of (1α,4β)-3′,4′-dihydro-6′-methoxy-4-carboxy-spiro[cyclohexane-1,1′(2′H)-isoquinoline] (3.0 g; compound obtained in Reference example 34(3)) in acetic acid (30 mL) is added dropwise sulfuryl chloride (1.75 mL) under ice-cooling and the mixture is stirred at room temperature for 2 hours. After addition of acetic acid (20 mL) and sulfuryl chloride (0.88 mL), the mixture is further stirred at room temperature for 4 hours. The reaction mixture is concentrated and the residue is subjected to azeotropic distillation with toluene. After addition of methanol, the residue is concentrated. To the residue is added saturated aqueous sodium hydrogencarbonate solution and the mixture is extracted with ethyl acetate. The extract is washed with saturated aqueous sodium hydrogencarbonate solution and saturated aqueous NaCl solution, and then dried over sodium sulfate The dried solution is concentrated and the residue is purified by column chromatography on neutral silica gel (chloroform:methanol:aqueous 28% ammonia=50:1:0.1) to give (1α,4β)-3′,4′-dihydro-5′-chloro-6′-methoxy-4-methoxycarbonyl-spiro[cyclohexane-1,1′(2′H)-isoquinoline] (2.09 g) as crystals.

M.p. 113-115° C.

2) A mixture of the compound obtained in the above step (1) (2.05 g), ethanol (20 mL), water (20 mL), and sodium hydroxide (12.7 g) is stirred at room temperature for 7 hours. The reaction mixture is acidified with 10% HCl under ice-cooling and concentrated. The residue is purified by reverse-phase column chromatography (column: ODS-S-5OB, Solvents: water to water/acetonitrile (7:1)) to give crystals. The crystals are collected and dissolved in dioxane and to the solution is added HCl-dioxane. The mixture is concentrated and the residue is triturated with a mixture of dioxane and ethyl acetate to give (1α,4β)-3′,4′-dihydro-5′-chloro-6′-methoxy-4-carboxy-spiro[cyclohexane-1,1′(2′H)-isoquinoline] (2.88 g) as a colorless powder. MS(APCI)m/z: 310/312(M+H), IR(Nujol)cm⁻¹: 1743, 1736, 1703

### Reference Example 36

1) A mixture of 3′,4′-dihydro-6′,7′-dihydhoxy-4,4-bisethoxycarbonyl-spiro[cyclohexane-1,1′(2′H)-isoquinoline] (14.767 g; compound obtained in Reference example 13(1)), potassium carbonate (27.03 g), iodoethane(15.64 mL) and dimethylacetamide (60 mL) is stirred for 30 minutes under ice-cooling, at room temperature for 1 hour and at 100° C. for 3 hours. The mixture is further stirred at room temperature overniyht. To the reaction mixture is added water and extracted with ethyl acetate. The extract is washed with water, saturated aqueous sodium hydrogencarbonate solution, and an aqueous NaCl solution, successively. The organic layer is dried over sodium sulfate and concentrated. The residue is column chromatography on silica gel (ethyl acetate:n-hexane=1:1) to give 2′-ethyl-3′,4′-dihydro-6′,7′-diethoxy-4,4-bisethoxymcarbonyl-spiro[cyclohexane-1,1′(2′H)-isoquinoline] (17.81 g) as an oil. MS(APCI)m/z: 462(M+H), IR(neat)cm⁻¹: 1730

2) A mixture of the compound obtained in the above step (1) (17.79 g), sodium hydroxide (7.75 g), ethanol (30 mL), and water (30 mL) is stirred at room temperature for 5 hours and then refluxed for 13 hours. After cooling, the reaction mixture is acidified with 10% HCl to adjust its pH to 1 to 2, and then concentrated. The residue is subjected to azeotropic distillation with toluene and the residue is dissolved in water. The solution is neutralized (pH 7) with 10% aqueous sodium hydroxide and treated with NaCl and then extracted with chloroform. The extract is washed with a little portion of aqueous NaCl solution, dried over sodium sulfate, concentrated. The residue is crystallized from a mixture of isopropanol and ethyl acetate to give (1α,4β) -2′-ethyl-3′,4′-dihydro-6′,7′-diethoxy-4-carboxy-spiro[cyclohexane-1,1′(2′H)-isoquinoline] (3.26 g) as crystals. M.p. 203-213° C. (dec.), MS(APCI)m/z: 362(M+H), IR(Nujol)cm⁻¹: 1715, 1735

### Reference Example 37

1) To a solution of 2′-benzyloxycarbonyl-3′,4′-dihydro-6′-hydhoxy-4,4-bisethoxycarbonyl-spiro[cyclohexane-1,1′(2′H)-isoquinoline] (2.03 g; compound obtained in Reference example 33(5)) in methylene chloride (40 mL) is added triethylamine (1.14 mL) and then added dropwise a solution of trifluoromethanesulfonic anhydride (1.74 g) in methylene chloride (10 mL) at −10 to −20° C. The mixture is stirred for 1 hour and washed with water, 5% HCL, saturated aqueous sodium hydrogencarbonate solution, and saturated aqueous NaCl solution, successively. The mixture is dried over sodium sulfate and concentrated. The residue is purified by column chromatography on silica gel (ethyl acetate:n-hexane=1:7) to quantitatively give 2′-benzyloxycarbonyl-3′,4′-dihydro-6′-trifluoromethane-sulfonyloxy-4,4-bisethoxycarbonyl-spiro[cyclohexane-1,1′(2′H)-isoquinoline] (2.57 g). MS(APCI)m/z: 628(M+H), IR(neat)cm⁻¹: 1730, 1490, 1420

2) To a solution of the compound obtained in the above step (1) (2.55 g) in dimethylformamide (20 mL) is added triethylamine (1.7 mL), palladium acetate (27 mg), triphenylphosphine (63 mg) and formic acid (313 μL) and the mixture is stirred at 60° C. for 3 hours. The reaction mixture is poured into ice-cooled water and extracted with ethyl acetate. The extract is washed with 5% HCL, water, saturated aqueous sodium hydrogencarbonate solution and saturated aqueous NaCl solution, successively. The organic layer is dried over sodium sulfate and concentrated. The residue is purified by column chromatography on silica gel (ethyl acetate:n-hexane 1:7) to give 2′-benzyloxycarbonyl-3′,4′-dihydro-4,4-bisethoxycarbonyl-spiro[cyclohexane-1,1′(2′H)-isoquinoline] (1.69 g, 86.8%).

MS(APCI)m/z: 480(M+H), IR(neat)cm⁻¹: 1730, 1715, 1450.

3) A mixture of the compound obtained in the above step (2) (19.31 g), ethanol (100 mL), water (100 mL) and sodium hydroxide (16.1 g) is stirred at room temperature for 13 hours and then refluxed for 7 hours. After cooling, the reaction mixture is concentrated. The residue is acidified with 10% HCL and extracted with ethyl acetate. The extract is washed with saturated aqueous NaCl solution, dried over sodium sulfate, concentrated. The residue is crystallized from isopropylether to give 2′-benzyloxycarbonyl-3′,4′-dihydro-4,4-biscarboxy-spiro[cyclohexane-1,1′(2′H)-isoquinoline] (16.16 g, 97.7%) as crystals. M.p. 199-200° C.

4) The compound obtained in the above step (3) is treated in the same manner as described in Reference example 33(7) to give (1α,4β)-3′,4′-dihydro-4-carboxy-spiro[cyclohexane-1,1′(2′H)-isoquinoline] as crystals. M.p. 221.5-223.5° C.

### Reference Examples 38 to 42

The corresponding materials are treated in the same manner as described in Reference example 17(3) to give the compounds as shown in the following table (Table.49).

TABLE 49

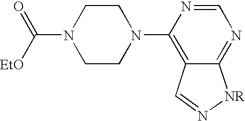

| Ref. Ex. No. | R | Physicochemical Properties etc. |
|---|---|---|
| 38 | 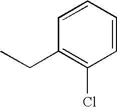 | M.p. 118-119° C. MS(APCI) 401(M + H) |
| 39 | 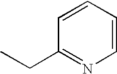 | Amorphous powder MS(APCI) 368(M + H) |
| 40 | 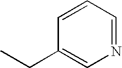 | M.p. 110-111° C. MS(APCI) 368(M + H) |
| 41 | 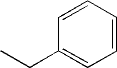 | M.p. 127-129° C. MS(APCI) 367(M + H) |
| 42 | -n-Bu | M.p. 56-57° C. |
| | | MS(APCI) 333(M + H) |

### Reference Examples 43 to 47

The corresponding materials are treated in the same manner as described in Reference example 17(4) to give the compounds as shown in the following table (Table 50).

TABLE 50

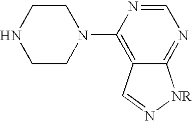

| Ref. Ex. No. | R | Physicochemical Properties etc. |
|---|---|---|
| 43 | 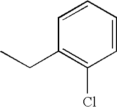 | Amorphous powder MS(APCI) 329(M + H) |
| 44 | 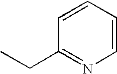 | Oil MS(APCI) 296(M + H) |
| 45 | 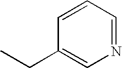 | M.p. 116-117° C. MS(APCI) 296(M + H) |
| 46 | 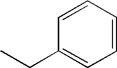 | Amorphous powder MS(APCI) 295(M + H) |
| 47 | -n-Bu | M.p. 205-207° C. |
| | | MS(APCI) 261(M + H) |

### Reference Example 48

Amixture of 4-(4-ethoxycarbonyl-1-piperazinyl)-1H-pyrazolo [3,4-d]pyrimidine (compound obtained in Reference example 17(1) or (2); 84.2 g), potassium hydroxide (170.9 g), water (600 mL) and ethanol (600 mL) is refluxed for 5 hours. After cooling, 600 mL of the solvent is removed in vacuo and the residue is acidified with conc.HCl. The mixture is concentrated. A mixture of the residue in water (100 mL) and dioxane (1000 mL) are added sodium hydrogencarbonate (102.4 g) and di-tert-butyl dicarbonate (99.7 g), and the mixture is stirred at room temperature for 12 hours. To the reaction mixture is added sodium hydroxide (80 g) and the mixture is stirred for at room temperature for 3 hours. The reaction mixture is neutralized with conc. HCl, and then the precipitates are collected and washed with water. Recrystallization from ethanol gave 4-(4-tert-butoxycarbonyl-1-piperazinyl)-1H-pyrazolo[3,4-d]pyrimidine (75.35 g, 81.2%) as crystals.

M.p. 230-231° C.

MS(APCI)m/z: 305(M+H), IR(Nujol)cm⁻¹: 1690.

2) To a suspension of the compound obtained in the above step (1) (15 g) in dimethylformamide (75 mL) and tetrahydrofuran (75 mL) is added sodium hydride (2.37 g) and the mixture is stirred at room temperature for 1.5 hours. To the reaction mixture is added dropwise 3-methylbenzylchloride (7.8 mL) and the mixture is stirred at room temperature for 3 hours. The reaction mixture is poured into water and extracted with ethyl acetate. The organic layer is separated, washed with saturated aqueous NaCl solution, dried over sodium sulfate, and concentrated. The residue is recrystallized from isopropanol to give 4-(4-tert-butoxycarbonyl-1-piperazinyl)-1-(3-methylbenzyl)-1H-pyrazolo[3,4-d]pyrimidine (9.6 g) as crystals.

M.p. 130-131° C., MS(APCI)m/z: 409(M+H), IR(Nujol)cm⁻¹: 1691, 1681.

3) To a suspension of the compound obtained in the above step (2) (9.58 g) in methanol (58 mL) is added 4N HCl-dioxane (58 mL) and the mixture is stirred at room temperature for 16 hours. After addition of ethyl acetate (100 mL), the reaction mixture is further stirred for 1 hour at room temperature. The precipitates are collected and washed with ethyl acetate to give 1-(3-methylbenzyl)-4-(1-piperazinyl)-1H-pyrazolo[3,4-d]pyrimidine 2HCL (9.06 g, quantitatively) as crystals.

M.p. 254-256° C., MS(APCI)m/z: 309(M+H).

### Reference Examples 49 to 60

The corresponding materials are treated in the same manner as described in Reference example 48(2) to give the compounds as shown in the following table (Table.51).

TABLE 51

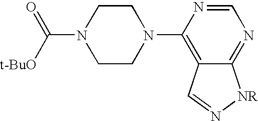

| Ref. Ex. No. | R | Physicochemical Properties etc. |
|---|---|---|
| 49 | 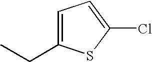 | M.p. 140-142° C. MS(APCI) 435(M + H) |
| 50 | 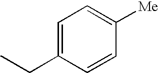 | M.p. 129-131° C. MS(APCI) 409(M + H) |
| 51 | 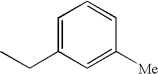 | M.p. 130-131° C. MS(APCI) 409(M + H) |
| 52 | 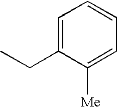 | M.p. 125-126° C. MS(APCI) 409(M + H) |
| 53 | 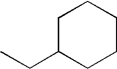 | M.p. 98-100° C. MS(APCI) 401(M + H) |
| 54 | 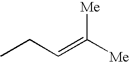 | M.p. 61° C. MS(APCI) 373(M + H) |
| 55 | 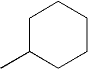 | M.p. 129-132° C. MS(APCI) 387(M + H) |
| 56 | Me | M.p. 91-93° C. |
| | | MS(APCI) 319(M + H) |
| 57 | Et | M.p. 92-95° C. |
| | | MS(APCI) 333(M + H) |
| 58 | 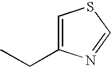 | Amorphous powder MS(APCI) 402(M + H) |
| 59 | 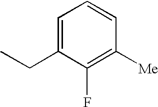 | Oil MS(APCI) 427(M + H) |
| 60 | 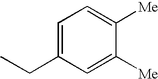 | Oil MS(APCI) 423(M + H) |

### Reference Example 61

The corresponding materials are treated in the same manner as described in Reference example 48 (3) to give the compounds as shown in the following table (Table.52).

TABLE 52

| Ref. Ex. No. | R | Physicochemical Properties etc. |
|---|---|---|
| 61 | 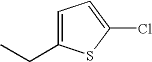 | M.p. 230-231° C. MS(APCI) 335(M + H) |
| 62 | 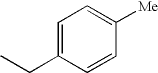 | M.p. 237-239° C. MS(APCI) 309(M + H) |
| 63 | 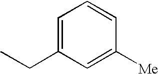 | M.p. 231-232° C. MS(APCI) 309(M + H) |
| 64 | 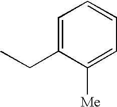 | M.p. 222-223° C. MS(APCI) 309(M + H) |
| 65 | 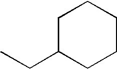 | M.p. 310-313° C. MS(APCI) 401(M + H) |
| 66 | 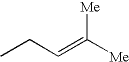 | M.p. 223-225° C. MS(APCI) 237(M + H) |
| 67 | 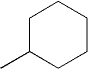 | M.p. 293-296° C. MS(APCI) 287(M + H) |
| 68 | Me | M.p. 350-360° C. |
| | | MS(APCI) 219(M + H) |
| 69 | Et | M.p. 193-196° C. |
| | | MS(APCI) 233(M + H) |
| 70 | 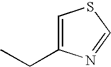 | M.p. 197-202° C. MS(APCI) 302(M + H) |
| 71 | 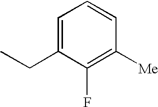 | M.p. 218-220° C. MS(APCI) 327(M + H) |
| 72 | 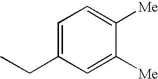 | M.p. 248-250° C. MS(APCI) 323(M + H) |

### Reference Example 73

1) To a solution of ethylpyridine (10.0 g) in acetic acid (50 mL) 30% added aqueous hydrogen peroxide (10.6 mL) and the mixture is refluxed for 24 hours. After cooling, to the reaction mixture is added dimethylsulfide (3.4 mL) at room temperature, and then stirred for 1 hour. The reaction mixture is concentrated under reduced pressure. To a solution of the residue and cyanotrimethylsilane (11.6 g) in dichloromethane (90 mL) is added dropwise dimethylaminocarbamoyl chloride (10.7 mL) in dichloromethane (25 mL) at room temperature over a period of 10 minutes. The mixture is stirred for 24 hours. To the reaction mixture is added 1i aquleoui potassium carbonate and the mixture is stirred for 10 minutes. The organic layer is separated and extracted twice with dichloromethane (100 mL). The combined extract is washed with saturated aqueous NaCl solution, dried over sodium sulfate, and concentrated. The residue is purified by flush-column chromatography on silica gel (chloroform) to give 2-cyano-6-ethylpyridine (9.45 g, 81.1%) as a yellow oil.

MS(APCI)m/z: 133(M+H), IR(neat)cm⁻¹: 2236.

2) A solution of the compound obtained in the above step (1) (9.44 g) in 6N HCl (50 mL) is refluxed for 24 hours. The reaction mixture is concentrated under reduced pressure. The residue is triturated with acetonitrile and collected to give 6-ethyl-picolinic acid hydrochloride as a crude product.

3) To a suspension of the compound obtained in the above step (2) (13.4 g) in tetrahydrofuran (200 mL) is added dropwise a solution of 10 N borane-dimethylsulfide complex in dimethylsulfide (19.3 mL) over a period of 10 minutes andthe mixture is refluxed for 6 hours. After cooling, to the reaction mixture is added 1N sodium hydroxide (100 mL) and the mixture is stirred for 15 minutes at room temperature. The reaction mixture is extracted twice with ethyl acetate (300 mL) and the extract is washed with water (200 mL) and saturated aqueous NaCl solution (200 mL). The organic solution is dried over sodium sulfate and concentrated under reduced pressure. The residue is purified by flush-column chromatography on silica gel (n-hexane:ethyl acetate=1:1) to give 6-ethyl-2-hydroxymethylpyridine (7.07 g, 72.2%) as an oil.

MS(APCI)m/z:138(M+H), IR(neat)cm−1: 3244, 1736.

4) To a solution of triphenylphosphine (7.56 g) in tetrahydrofuran (200 mL) is added dropwise diethylazodicarboxylate (4.54 mL) at −78° C. and the mixture is stirred for 5 minutes. To the reaction mixture is added the compound obtained in the above step (3) (4.35 g), neopentylamine (1.40 g) and the compound obtained in Reference example 48(1) (8.77 g) at −78° C. The mixture is stirred at −78° C. for 15 minutes and at room temperature for 17 hours. The reaction mixture is concentrated under reduced pressure and the residue is purified by flush-column chromatography on silica gel (chloroform chromatography on silica gel (chloroform-chloroform/methanol (20:1)) and concentrated. To a solution (40 mL) at room temperature and the mixture is stirred at room temperature for 17 hours. To the reaction mixture is added ethyl acetate (150 mL) and the precipitates are collected and washed with ethyl acetate to give 1-(6-ethyl-2-pyridylmethyl)-4-(1-piperazinyl)-1H-pyrazolo[3,4-d]pyrimidine 3HCl (6.15 g, 49.3%) as crystals.

M.p. 228° C., MS(APCI)m/z: 324(M+H), IR(Nujol)cm−1: 3281, 1985, 1733, 1635.

### Reference Examples 74 to 91

The corresponding materials are treated in the same manner as described in Reference example 73(4) to give the compounds as shown in the following table (Table.53).

TABLE 53

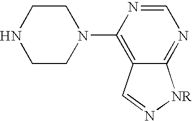

| Ref. Ex No. | R | salt | Physicochemical Properties etc. |
|---|---|---|---|
| 74 | 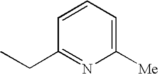 | 3HCl | M.p. 240-243° C. MS(APCI) 310(M + H) |
| 75 | 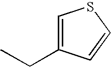 | 2HCl | M.p. 224-226° C. MS(APCI) 301(M + H) |
| 76 | 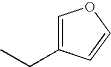 | 2HCl | M.p. 217-219° C. MS(APCI) 285(M + H) |
| 77 | 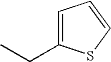 | 2HCl | M.p. 212-215° C. MS(APCI) 301(M + H) |
| 78 | 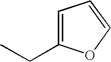 | 2HCl | M.p. 169-172° C. MS(APCI) 285(M + H) |
| 79 | 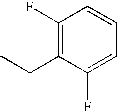 | 2HCl | M.p. 307-309° C. MS(APCI) 331(M + H) |
| 80 | 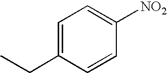 | 2HCl | M.p. >300° C. MS(APCI) 340(M + H) |
| 81 | 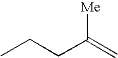 | 2HCl | M.p. 188-191° C. MS(APCI) 273(M + H) |
| 82 | 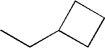 | 2HCl | M.p. 300-305° C. MS(APCI) 273(M + H) |
| 83 | 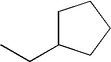 | 2HCl | M.p. 291-294° C. MS(APCI) 287(M + H) |
| 84 | 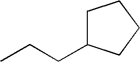 | 2HCl | M.p. 296-299° C. MS(APCI) 301(M + H) |
| 85 | n-Hex | 2HCl | M.p. 203-206° C. MS(APCI) 289(M + H) |
| 86 | 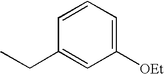 | 2HCl | M.p. 210-212° C. MS(APCI) 339(M + H) |
| 87 | 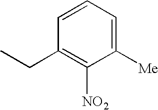 | 2HCl | M.p. 242-245° C. MS(APCI) 354(M + H) |
| 88 | 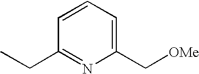 | 3HCl | M.p. 203-205° C. MS(APCI) 340(M + H) |
| 89 | 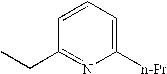 | 3HCl | Amorphous powder MS(APCI) 338(M + H) |
| 90 | 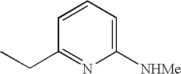 | 3HCl | Amorphous powder MS(APCI) 325(M + H) |
| 91 | 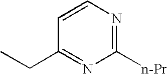 | 3HCl | Amorphous powder MS(APCI) 339(M + H) |

### Reference Example 92

1) A solution of thiopropionamide (500 mg) and 1,3-dichloro-2-propanone (748 mg) in dimethylformamide (10 mL) is refluxed for 3hours. The reaction mixture is concentrated under reduced pressure and the residue is dissolved in ethyl acetate. The solution is washed with water, dried over sodium sulfate, and then concentrated. The residue is purified by column chromatography on silica gel (n-hexane:ethyl acetate=7:3) to give 4-chloromethyl-2-ethylthiazol (458 mg, 50.5%) as an oil.

MS(APCI)162/163(M+H), IR(neat)cm⁻¹: 3109.

2) To a suspension of the compound obtained in the Reference example 48(1) (3.0 g) and lithium hydroxide (1.65 g) in dimethylacetamide (60 mL) is added the compound obtained in the above step (1) (2.07 g) and the mixture is stirred for 16 hours. To the reaction mixture is added water and the mixture is extracted with ethyl acetate. The extract is washed twice with an aqueous saturate NaCl solution, dried over sodium sulfate, concentrated under reduced pressure and the residue is purified by column chromatography on silica gel (chloroform:methanol=80:1) to give 4-(4-tert-butoxycarbonyl-1-piperazinyl)-1-(2-ethyl-4-thiazolylmethyl)-1H-pyrazolo[3,4-d]pyrimidine (2.33 g, 55%) as an oil.

MS(APCI)m/z: 430(M+H), IR(neat)cm⁻¹: 1682.

3) The compound obtained in the above step (1) (2.31 g) is treated in the same manner as described in Reference example 48(3) to give 1-(2-ethyl-4-thiazolylmethyl)-1H-pyrazolo[3,4-d]pyrimidine 3HCl (2.21 g, 94%) as crystals.

M.p. 210-211° C., MS(APCI)m/z: 330(M+H).

IR(neat+chloroform)cm⁻¹: 1633, 1613.

### Reference Examples 93 to 102

The corresponding materials are treated in the same manner as described in Reference example 92(2) to give the compounds as shown in the following table (Table.54).

TABLE 54

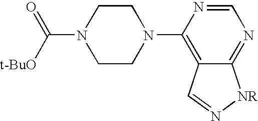

| Ref. Ex. | | |
|---|---|---|
| No. | R | Physicochemical Properties etc. |
| 93 | 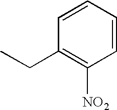 | M.p. 180-183° C. MS(APCI) 440(M + H) |
| 94 | 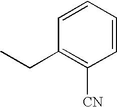 | M.p. 183-184° C. MS(APCI) 420(M + H) |
| 95 | 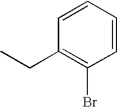 | M.p. 146-147° C. MS(APCI) 473, 475(M + H) |
| 96 | 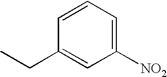 | M.p. 117-120° C. MS(APCI) 440(M + H) |
| 97 | 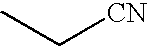 | M.p. 137-139° C. MS(APCI) 344(M + H) |
| 98 | 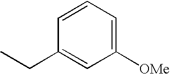 | M.p. 113-114.5° C. MS(APCI) 425(M + H) |
| 99 | 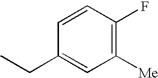 | M.p. 118-120° C. MS(APCI) 427(M + H) |
| 100 | 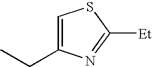 | Oil MS(APCI) 430(M + H) |
| 101 | 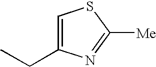 | Oil MS(APCI) 416(M + H) |
| 102 | 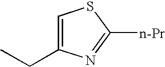 | Oil MS(APCI) 444(M + H) |

### Reference Examples 103 to 112

The corresponding materials are treated in the same manner as described in Reference example 48(3) to give the compounds as shown in the following table (Table.55).

TABLE 55

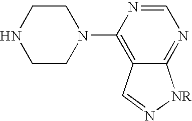

| Ref. Ex. | | | Physicochemical |
|---|---|---|---|
| No. | R | Salt | Properties etc. |
| 103 | 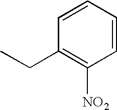 | 2HCl | M.p. 287-290° C. MS(APCI) 340(M + H) |
| 104 | 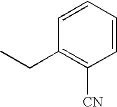 | Free | M.p. 148-149° C. MS(APCI) 320(M + H) |
| 105 | 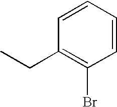 | Free | M.p. 156-157° C. MS(APCI) 373, 375(M + H) |
| 106 | 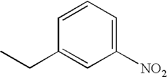 | 2HCl | M.p. 269-271° C. MS(APCI) 340(M + H) |
| 107 | 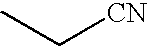 | 2HCl | M.p. 144-145° C. MS(APCI) 244(M + H) |
| 108 | 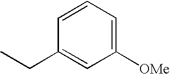 | 2HCl | M.p. 200-204° C. MS(APCI) 325(M + H) |
| 109 | 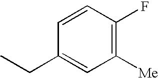 | 2HCl | M.p. 259-262° C. MS(APCI) 327(M + H) |
| 110 | 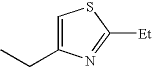 | 3HCl | M.p. 210-211° C. MS(APCI) 330(M + H) |
| 111 | 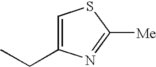 | 3HCl | M.p. 211-212° C. MS(APCI) 416(M + H) |
| 112 | 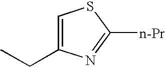 | 3HCl | M.p. 147-149° C. MS(APCI) 344(M + H) |

### Reference example 113

1) A solution of 6-bromopicolinic scid (8.85 g) and conc. H₂SO₄ (2 mL) in methanol (270 mL) is refluxed for 12 hours. The reaction mixture is concentrated under reduced pressure. The residue is dissolved in chloroform, dried over sodium sulfate, and then concentrated. To a solution of the resultant residue in tetrahydrofuran (240 mL) is slowly added portionwise lithium borohydride (1.9 g) under ice-cooling and the mixture is stirred under ice-cooling for 5 minutes and at room temperature for 3 hours. The reaction mixture is poured into ice-water. The mixture is acidified with iN HCl and stirred for 30 minutes. The mixture is basified with saturated aqueous sodium hydrogencarbonate and extracted with ethyl acetate. The organic layer is dried over sodium sulfate and concentrated. The residue is purified by column chromatography on silica gel (chloroform:methanol=10:1) to give 6-bromo-2-hydroxymethylpyridine (7.5 g, 91%) as a colorless oil.

2) The compound obtained in the above step (1) (5.96 g) and the compound obtained in Reference example 48(1) (8.77 g) are treated in the same manner as described in Reference example 49(4) to give 1-(6-bromo-2-pyridylmethyl)-4-(4-tert-butoxycarbonyl-1-piperazinyl)-1H-pyrazolo[3,4-d]pyrimidine (9.0 g, 66%) as crystals.

M.p. 126-128° C., MS(APCI)m/z: 475(M+H), IR(Nujol)cm⁻¹: 1683.

3) Sodium (1.80 g) is slowly dissolved in ethanol (210 mL) and to the solution is added the compound obtained in the above step (2) (7.0 g). The mixture is refluxed for 3 days. The reaction mixture is concentrated under reduced pressure and to the residue is added water. The mixture is extracted with chloroform and the extract is washed with water and saturated aqueous NaCl solution. The organic layer is dried over sodium sulfate and concentrated. The resultant residue purified by column chromatography on silica gel and recrystallized from chloroform-diethylether to give 1-(6-ethoxy-2-pyridylmethyl)-4-(4-tert-butoxycarbonyl-1-piperazinyl)-1H-pyrazolo[3,4-d]pyrimidine (5.0 g, 77%) as crystals.

M.p. 130-132° C., MS(APCI)m/z: 440(M+H), IR(Nujol)cm⁻¹: 1688.

4) The compound obtained in the above step (3) (5.0 g) is treated in the same manner as described in Reference example 48(3) to quantitatively give 1-(6-ethoxy-2-pyridylmethyl)-4-(1-piperazinyl)-1H-pyrazolo[3,4-d]pyrimidine 3HCl(5.1 g) as crystals.

M.p. 200° C., MS(APCI)m/z: 340(M+H), IR(Nujol)cm⁻¹: 1643, 1633.

### Reference Example 114

1) To a solution of the compound obtained in Reference example 96 (20.58 g) in methanol (80 mL) and tetrahydrofuran (80 mL) is added palladium-carbon (3.0 g) and the mixture is stirred under hydrogen gas atmosphere for 8 hours at room temperature. The reaction mixture is filtered to remove catalysts and the catalysts are washed with methanol. The combined filtrate is concentrated. The residue is purified by column chromatography on NH-silica gel (chloroform:hexane=1:1) and triturated with diisopropylether to give 1-(3-aminobenzyl)-4-(4-tert-butoxycarbonyl-1-piperazinyl)-1H-pyrazolo[3,4-d]pyrimidine (18.49 g, 96%) as an amorphous powder.

MS(APCI)m/z: 410(M+H).

2) Formic acid (1.5 mL) is added dropwise to acetic anhydride (3.0 mL) under ice-cooling and the mixture is stirred at 60° C. for 1 hour. The reaction mixture is added to tetrahydrofuran (10 mL) and to the solution is added dropwise a solution of the compound obtained in the above step (1) (5.0 g) in tetrahydrofuran (60 mL) under ice-cooling. The mixture is stirred for 1 hour and the reaction mixture is basified with saturated aqueous sodium hydrogencarbonate. The organic solvent is removed and the residue is extracted with ethyl acetate. The extract is washed with water and saturated aqueous NaCl solution, dried over sodium sulfate, concentrated. The residue is triturated with diisopropylether to give 1-(3-formylaminobenzyl)-4-(4-tert-butoxycarbonyl-1-piperazinyl)-1H-pyrazolo[3,4-d]pyrimidine (5.15 g) as an amorphous (5.15 g) as an amorphous

MS(APCI)m/z: 438(M+H).

3) To a suspension of 60% sodium hydride (16 mg) in tetrahydrifuran (0.5 mL) are added the compound obtained in the above step (2) (150 mg) and iodoethane (0.137 mL) and the mixture is stirred for 30 minutes. To the reaction mixture are added tetrahydrofuran 80.5 mL) and sodium hydride (33 mg), and the mixture is stirred at room temperature for 2 hours. To the reaction mixture is added ice-cooled water and the mixture is extracted with ethyl acetate. The extract is washed with water and saturated aqueous NaCl solution, dried over sodium sulfate, and concentrated. The residue is triturated with diisopropylether to give 1-(3-(N-ethyl-N-formylamino) diisopropylether to give 1-(3-(N-ethyl-N-formylamino)benzyl)-4-(4-tert-butoxycarbonyl-1-piperazinyl)-1H-pyrazolo[3,4-d]pyrimidine powder.

MS(APCI)m/z: 466(M+H).

4) A solution of the compound obtained in the above step

(3) (132 mg) in HCl (2 mL) is refluxed for 1 hour. The reaction mixture is diluted with ice-cooled water and basified with potassium carbonate. The mixture is extracted with chloroform. The extract is dried over sodium sulfate and concentrated to give 1-(3-ethylaminobenzyl)-4-(1-piperazinyl)-1H-pyrazolo[3,4-d]pyrimidine (98 mg, quantitatively) as an oil.

MS(APCI)m/z: 338(M+H).

1. A spiroisoquinoline derivative of the formula [I]: 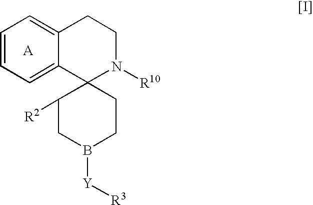
wherein ring A is an optionally substituted benzene ring,
R¹⁰ is a hydrogen atom or a group of the formula: —Z—R¹,
wherein R¹ is a hydrogen atom, an optionally substituted lower alkyl group, or an optionally substituted lower alkenyl group,
Z is a group of the formula: —CH₂— or —CO—,
R² is a hydrogen atom or an optionally substituted heterocyclic group,
B is a group of the formula: 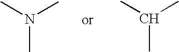
R³ is an optionally substituted amino group or an optionally substituted nitrogen-containing aliphatic heterocyclic group, and
Y is a group of the formula: —CH₂— or —CO—,
or a pharmaceutically acceptable salt thereof.

2. The compound according to claim 1, wherein ring A is a benzene ring which may be substituted by a group(s) selected from a lower alkyl group, a lower alkoxy group, an optionally protected hydroxyl group, a halogen atom, an amino group and a lower alkylenedioxy group,
R¹⁰ is a hydrogen atom or a group of the formula: —Z—R¹,
wherein R¹ is
(1) a hydrogen atom,
(2) a lower alkyl group which may be substituted by a group(s) selected from the group consisting of:
(i) a halogen atom,
(ii) an optionally protected hydroxyl group,
(iii) an amino group which may be substituted by a group(s) selected from a lower alkyl group; a lower cycloalkyl group; an aryl-lower alkyl group; a lower alkoxycarbonyl group; an acyl group; 1-amino-2-nitrovinyl group; 1-(mono- or di-)lower alkyl, amino-2-nitrovinyl group; 1-amino-2,2-dicyanovinyl group; 1-(mono- or di-)lower alkylamino-2,2-dicyanovinyl group; 3-aminocyclobut-3-en-1,2-dion-4-yl group; 3-(mono- or di-)lower alkylaminocyclobut-3-en-1,2-dion-4-yl group; and a group which can be removed by enzymatic or chemical metabolic process in vivo,
(iv) a guanidino group which may be substituted by a group(s) selected from a lower alkyl group, a lower cycloalkyl group and a cyano group,
(v) an ureido group which may be substituted by a group(s) selected from a lower alkyl group and a lower cycloalkyl group, and
(vi) a thioureido group which may be substituted by a group(s) selected from a lower alkyl group and a lower cycloalkyl group, or
(3) a lower alkenyl group,
Z is a group of the formula: —CH₂— or —CO—,
R² is
(1) a hydrogen atom, or
(2) a heterocyclic group which may be substituted by a group(s) selected from the group consisting of:
(i) a lower alkyl group,
(ii) a lower alkoxy group,
(iii) an optionally protected hydroxyl group,
(iv) a halogen atom,
(v) a lower alkylenedioxy group, and
(vi) an acyl group,
R³ is
(1) an amino group which may be substituted by a group(s) selected from the group consisting of:
(i) a lower alkyl group which may be substituted by a group(s) selected from an oxo group, an optionally protected amino group, a (mono- or di-)lower alkylamino group, an aryl-lower alkylimidazolylthio group, and a pyridylamino group (the pyridyl moiety of said pyridylamino group being optionally substituted by a lower alkyl group(s)),
(ii) an acyl group,
(iii) an amino group which may be substituted by a group(s) selected from a nitrogen-containing heterocyclic group which may be substituted by a lower alkyl group(s), and a lower alkyl group, and
(iv) a nitrogen-containing heterocyclic group which may be substituted by a group(s) selected from a lower alkyl group, a lower alkoxy group, an aryl-lower alkyl group, an optionally protected hydroxyl group, and an amino group, or
(2) a nitrogen-containing aliphatic heterocyclic group which may be substituted by a group(s) selected from the group consisting of:
(i) a nitroso group,
(ii) an optionally protected amino group,
(iii) a nitrogen-containing heterocyclic group or its onium salt on nitrogen atom which may be substituted by a group(s) selected from an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, mono- or di(lower alkyl)amino group, a lower alkoxy-lower alkyl group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom and a lower alkoxy group), a furyl-lower alkyl group (the furyl moiety of said furyl-lower alkyl group being optionally substituted by a group selected from a lower alkyl group and a mono- or di-lower alkylamino-lower alkyl group), an imidazolyl-lower alkyl group, a thiazolyl-lower alkyl group (the thiazolyl moiety of said thiazolyl-lower alkyl group being optionally substituted by a group(s) selected from a lower alkyl group, a hydroxy group, mono- or di-(lower alkyl)amino group and a lower alkoxy-lower alkyl group), a pyrazolyl-lower alkyl group, a pyrimidinyl-lower alkyl group (the pyrimidinyl moiety of said pyrimidinyl-lower alkyl group being optionally substituted by a lower alkyl group), a pyridazinyl-lower alkyl group, a pyridyl-lower group), a pyridazinyl-lower alkyl group, a pyridyl-lower group being optionally substituted by a group selected from a lower alkyl group, a halogen atom, a lower alkoxy group, a lower alkoxy-lower alkyl group, mono- or di-lower alkylamino group, a lower alkoxycarbonyl group, a mono- or di-lower alkyl-carbamoyl group, mono- or di-lower alkylamino-lower alkyl group, a hydroxy-lower alkyl group, an oxo group and an oxide group), a carboxyl group, a lower alkoxycarbonyl group, a halogen atom, a hydroxy-lower alkyl group, a carboxy-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group), and
(iv) a lower alkyl group which may be substituted by a group(s) selected from an oxo group, a pyridyl group, an imino group, a pyrazolyl group (said pyrazolyl group being optionally substituted by a group(s) selected from a lower alkyl group and a benzyl group), a carbamoyl group (said carbamoyl group being optionally substituted by a group(s) selected from a pyridyl group and a lower alkyl group), a thiocarbamoyl group (said thiocarbamoyl group being optionally substituted by a group(s) selected from a pyridyl group and a lower alkyl group), an amino group (said amino group being optionally substituted by a group(s) selected from an N-lower alkyl-N-pyridylcarbamoyl group, a lower alkylcarbamoyl group, a pyridylcarbamoyl group, a lower alkyl group, an aminio-protecting group, a pyridylcarbonyl group, a pyridylthiocarbonyl group, a pyridyl group, and a 1-cyanoimino-1-pyridylmethyl group).

3. The compound according to claim 2, wherein the group which can be removed by enzymatic or chemical metabolic process in vivo is a group of the formula: 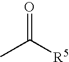
wherein R⁵ is a group of the formula: 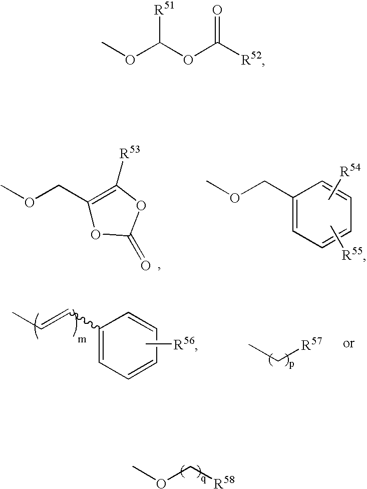
wherein R⁵¹ is a hydrogen atom or a lower alkyl group,
R⁵² is a lower alkyl group (said lower alkyl group being optionally substituted by a carboxyl group), a lower cycloalkyl group, a lower alkoxy group, a lower cycloalkoxy group or an aryl group,
R⁵³ is a lower alkyl group or an aryl group,
R⁵⁴ and R⁵⁵ are the same or different and each a hydrogen atom, a lower alkanoyloxy group, an arylcarbonyloxy group, a lower alkoxycarbonyloxy group, a lower alkanoyloxymethyloxy group, a halogen atom or a lower alkyl group,
R⁵⁶ is a hydrogen atom, a lower alkanoyloxy-lower alkyl group or an arylcarbonyloxy-lower alkyl group,
m is an integer of 0 or 1,
R⁵⁷ is an optionally protected amino group, a lower alkoxy group, a carbamoyloxy group, a (mono- or di-)lower alkylcarbamoyloxy group, or an acyl group,
P is an integer of 1 or 2,
R⁵⁸ is a lower alkoxy group, an acyl group, a carbamoyloxy group, or a (mono- or di-)lower alkylcarbamoyloxy group, and
q is an integer of 1 or 2.

4. The compound according to claim 3, wherein R² is a group of the formula: 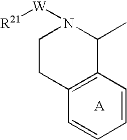
wherein ring A is a benzene ring which may be substituted by a group(s) selected from a lower alkyl group, a lower alkoxy group, an optionally protected hydroxyl group, a halogen atom, an amino group, and a lower alkylenedioxy group,
R²¹ is a hydrogen atom or a lower alkyl group,
W is a group of the formula: —CH₂— or —CO—.

5. The compound according to claim 3, wherein R² is a hydrogen atom.

6. The compound accordiLng to claim 3, wherein the heterocyclic group in R² is a nitrogen-containing hetero(mono- or bi-)cyclic group,
the nitrogen-containing aliphatic heterocyclic group in R³ is a nitrogen-containing aliphatic 4 to 8 membered heteromonocyclic group, the nitrogen-containing heterocyclic group in R³ is a nitrogen-containing hetero(mono-, bi- or tri-)cyclic group.

7. The compound according to claim 3, wherein the heterocyclic group in R² is a 1,2,3,4-tetrahydroisoquinolyl group, a 3,4-dihydroisoquinolyl group or an isoquinolyl group,
the nitrogen-containing aliphatic heterocyclic group in R³ is an azetidinyl group, a pyrrolidinyl group, an imidazolidinyl group, a pyrazolidinyl group, a piperidyl group, a piperazinyl group, an azepinyl group, a diazepinyl group, an azeocinyl group, or a diazeocinyl group,
the aryl group in R¹ or R³ is a phenyl group, a naphthyl group, an anthryl group or a phenanthryl group,
the nitrogen-containing heterocyclic group in R³ is a pyrrolyl group, an oxazolyl group, an isoxazolyl group, a thiazolyl group, an imidazolyl group, an imidazolinyl group, a pyrazolyl group, a pyridyl group, a dihydropyridyl group, a pyridazinyl group, a pyrimidinyl group, a tetrahydropyrimidinyl group, a pyrazinyl group, a pyrrolidinyl group, an imidazolidinyl group, a pyrazolidinyl group, a piperidyl group, a piperazinyl group, a triazinyl group, a morpholinyl group, an indolyl group, a quinolyl group, an isoquinolyl group, a purinyl group, an 1H-indazolyl group, a quinazolinyl group, a cinnolinyl group, a quinoxalinyl group, a phthalazinyl group, a pteridinyl group, a pyrazolopyrimidinyl group, a triazolopyrimidinyl group, an imidazopyrimidinyl group, a pyrazolopyridyl group, a triazolopyridyl group or an imidazopyridyl group.

8. The compound according to any one of claims 2, 3, 4, 5, 6 and 7, wherein R¹ is a group of the formula: 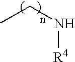
wherein R⁴ is a hydrogen atom or a lower alkyl group,
n is an integer from 1 to 6.

9. A prodrug of the compound according to claim 8.

10. The compound wherein, in the structure of the compound as claimed in claim 8, the nitrogen atom bonding to R⁴ is further substituted by a group which can be removed by enzymatic or chemical metabolic process in vivo.

11. The compound wherein, in the structure of the compound as claimed in claim 8, the nitrogen atom bonding to R⁴ is further substituted by a group of the formula: 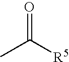
wherein R⁵ is a group of the formula: 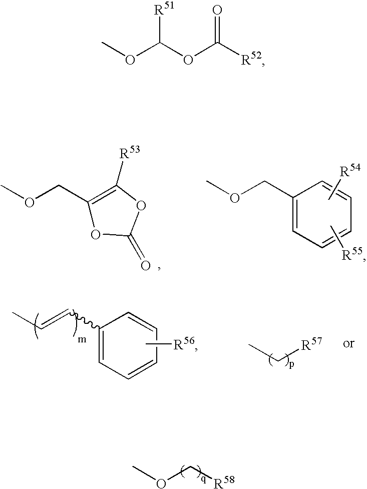
wherein R⁵¹ is a hydrogen atom or a lower alkyl group,
R⁵² is a lower alkyl group (said lower alkyl group being optionally substituted by a carboxyl group), a lower cycloalkyl group, a lower alkoxy group, a lower cycloalkoxy group or an aryl group,
R⁵³ is a lower alkyl group or an aryl group,
R⁵⁴ and R⁵⁵ are the same or different and each a hydrogen atom, a lower alkanoyloxy group, an arylcarbonyloxy group, a lower alkoxycarbonyloxy group, a lower alkanoyloxymethyloxy group, a halogen atom or a lower alkyl group,
R⁵⁶ is a hydrogen atom, a lower alkanoyloxy-lower alkyl group or an arylcarbonyloxy-lower alkyl group,
m is an integer of 0 or 1,
R⁵⁷ is an optionally protected amino group, a lower alkoxy group, a carbamoyloxy group, a (mono- or di-)lower alkylcarbamoyloxy group, or an acyl group,
P is an integer of 1 or 2,
R⁵⁸ is a lower alkoxy group, an acyl group, a carbamoyloxy group, or a (mono- or di-)lower alkylcarbamoyloxy group, and
q is an integer of 1 or 2.

12. The compound according to claim 11, wherein R⁵ is a group of the formula: 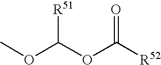
wherein R⁵¹ is a hydrogen atom or a lower alkyl group, and
R⁵² is a lower alkyl group (said lower alkyl group being optionally substituted by a carboxyl group), a lower cycloalkyl group, a lower alkoxy group, a lower cycloalkoxy group or an aryl group.

13. The compound according to claim 2, wherein ring A is a benzene ring which may be substituted by the same or different two groups selected from a lower alkoxy group and an optionally protected hydroxyl group,
R¹⁰ is a group of the formula: —Z—R¹,
wherein R¹ is a hydrogen atom or a lower alkyl group, and
Z is a group of the formula: —CH₂—,
R² is a 1,2,3,4-tetrahydroisoquinolyl group which may be substituted by a group(s) selected from a lower alkyl group, an acyl group, a lower alkoxy group and an optionally protected hydroxyl group,
R¹ is
R³ is alkylamino group, or
(2) a piperazinyl group which may be substituted by a group(s) selected from the group consisting of:
(i) a nitrogen-containing heteromonocyclic group or its onium salt on nitrogen atom which may be substituted by a group selected from a lower alkyl group, a carboxyl group, a lower alkoxycarbonyl group, a halogen atom, an oxo group, an oxide group and a hydroxy-lower alkyl group, and
(ii) a lower alkyl group which may be substituted by a group selected from an N-pyridyl-N-lower alkylcarbamoyl group, an oxo group, an imino group, an amino group and a pyridyl group,
Y is a group of the formula: —CO—.

14. The compound according to claim 2, wherein ring A is a benzene ring which may be substituted by the same or different two groups selected from a lower alkoxy group and an optionally protected hydroxyl group,
R¹⁰ is a group of the formula: —Z—R¹,
wherein R¹ is a lower alkyl group substituted by a (mono- or di-)lower alkylamino group,
Z is a group of the formula: —CH₂— or —CO—,
R² is
(1) a hydrogen atom, or
(2) a 1,2,3,4-tetrahydroisoquinolyl group which may be substituted by a group(s) selected from a lower alkyl group, an acyl group, a lower alkoxy group and an optionally protected hydroxyl group,
R³ is a piperazinyl group substituted by a nitrogen-containing hetero(mono- or bi-)cyclic group which may be substituted by a group(s) selected from the group consisting of an amino group, a lower alkyl group, a carboxyl-lower alkyl group, a lower alkoxycarbonyl-lower alkyl group, a hydroxy-lower alkyl group, an N-pyridyl-N-lower alkylcarbamoyl group, an aryl-lower alkyl group (the aryl moiety of said aryl-lower alkyl group being optionally substituted by a halogen atom or a lower alkyl group), a pyridyl-lower alkyl group (the pyridyl moiety of said pyridyl-lower alkyl group being optionally substituted by an oxide group), a thienyl-lower alkyl group, a lower alkylamino group, a halogenobenzyloxy-lower alkyl group, a lower alkenyl group, a lower cycloalkyl group, and an aryl group (said aryl group being optionally substituted by a group(s) selected from a trifluoromethyl group, a lower alkoxy group, and a nitro group), and
Y is a group of the formula: —CO—.

15. The compound according to claim 2, wherein ring A is a ring of the formula: 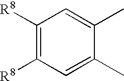
wherein R⁸ is a lower alkoxy group,
R¹⁰ is a group of the formula: —Z—R¹,
wherein R¹ is a lower alkyl group substituted by a (mono- or di-)lower alkylamino group, and
Z is a group of the formula: —CO—,
R² is a group of the formula: 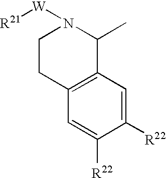
wherein R²¹ is a hydrogen atom or a lower alkyl group,
W is a group of the formula: —CH₂— or —CO—, and
R²² is a lower alkoxy group,
R³ is a piperazinyl group substituted by a group(s) selected from the group consisting of:
(1) a pyrazolopyrimidinyl group substituted by a group(s) selected from a lower alkyl group, a pyridyl-lower alkyl group and an aryl-lower alkyl group (the aryl moiety of said aryl-lower alkyl group being optionally substituted by a halogen atom or a lower alkyl group),
(2) an imidazopyridyl group substituted by a group(s) selected from a lower alkyl group or an aryl-lower alkyl group (the aryl moiety of said aryl-lower alkyl group being optionally substituted by a halogen atom or a lower alkyl group), and
(3) a triazolopyrimidinyl group substituted by a lower alkyl group or an aryl-lower alkyl group (the aryl moiety of said aryl-lower alkyl group being optionally substituted by a halogen atom or a lower alkyl group),
Y is a group of the formula: —CO—.

16. The compound according to claim 2, wherein ring A is a ring of the formula: 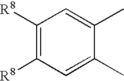
wherein R⁸ is a lower alkoxy group,
R¹⁰ is a group of the formula: —Z—R¹,
wherein R¹ is a lower alkyl group substituted by an amino group which may be substituted by a 1-(mono- or di-)lower alkylamino-2-nitrovinyl group),
Z is a group of the formula: —CH₂—,
R² is a hydrogen atom,
R³ is a piperazinyl group substituted by a pyrazolopyrimidinyl group substituted by a pyridyl-lower alkyl group or an aryl-lower alkyl group (the aryl moiety of said aryl-lower alkyl group being optionally substituted by a nitro group, a halogen atom or a lower alkyl group),
Y is a group of the formula: —CO—.

17. The compound according to claim 2, wherein ring A is a benzene ring which may be substituted by the same or different two groups selected from a lower alkoxy group and an optionally protected hydroxyl group,
R¹⁰ is a group of the formula: —Z—R¹,
wherein R¹ is an amino-substituted lower alkyl group (the amino group of said amino-substituted lower alkyl group being optionally substituted by a lower alkyl group and a group which can be removed by enzymatic or chemical metabolic process in vivo), and
Z is a group of the formula: —CH₂— or —CO—,
R² is
(1) a hydrogen atom, or
(2) a 1,2,3,4-tetrahydroisoquinolyl group which may be substituted by a group(s) selected from a lower alkyl group, an acyl group, a lower alkoxy group and an optionally protected hydroxyl group,
R³ is a piperazinyl groiup cih.tituted by a nitroqen-containing hetero(mono- or bi-)cyclic group which may be substituted by a group(s) selected from an amino group, a lower alkyl group, a carboxyl-lower alkyl group, a lower alkoxycarbonyl-lower alkyl group, a hydroxy-lower alkyl group, an N-pyridyl-N-lower alkylcarbamoyl group, an aryl-lower alkyl group (the aryl moiety of said aryl-lower alkyl group being optionally substituted by a halogen atom or a lower alkyl group), a pyridyl-lower alkyl group (the pyridyl moiety of said pyridyl-lower alkyl group being optionally substituted by an oxide group), a thienyl-lower alkyl group, a lower alkylamino group, a halogenobenzyloxy-lower alkyl group, a lower alkenyl group, a lower cycloalkyl group, and an aryl group (said aryl group being optionally substituted by a group(s) selected from a trifluoromethyl group, a lower alkoxy group, and a nitro group), and
Y is a group of the formula: —CO—.

18. The compound according to claim 2, wherein ring A is a ring of the formula: 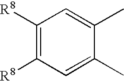
wherein R⁸ is a lower alkoxy group,
R¹⁰ is a group of the formula: —Z—R¹,
wherein R¹ is an amino-substituted lower alkyl group (the amino group of said amino-substituted lower alkyl group being optionally substituted by a lower alkyl group and a group which can be removed by enzymatic or chemical metabolic process in vivo), and
Z is a group of the formula: —CO—,
R² is a group of the formula: 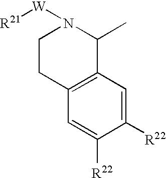
wherein R²¹ is a hydrogen atom or a lower alkyl group,
W is a group of the formula: —CH₂— or —CO—, and
R²² is a lower alkoxy group,
R³ is a piperazinyl group substituted by a group selected from the group consisting of:
(1) a pyrazolopyrimidinyl group substituted by a lower alkyl group, a pyridyl-lower alkyl group or an aryl-lower alkyl group (the aryl moiety of said aryl-lower alkyl group being optionally substituted by a halogen atom or a lower alkyl group),
(2) an imidazopyridyl group substituted by a lower alkyl group or an aryl-lower alkyl group (the aryl moiety of said aryl-lower alkyl group being optionally substituted by a halogen atom or a lower alkyl group), and
(3) a triazolopyrimidinyl group substituted by a lower alkyl group or an aryl-lower alkyl group (the aryl moiety of said aryl-lower alkyl group being optionally substituted by a halogen atom or a lower alkyl group),
Y is a group of the formula: —CO—.

19. A spiroisoquinoline comnpound of the formula [I-h]: 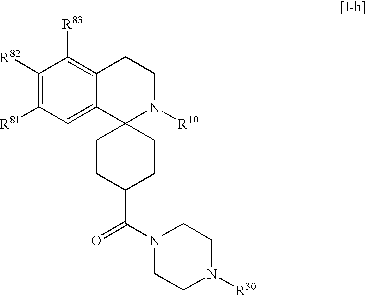
wherein R⁸¹, R⁸² and R⁸³ are the same or different groups selected from the group of a hydrogen atom, a lower alkoxy group, an optionally protected hydroxyl group and a halogen atom,
R¹⁰ is a hydrogen atom or a group of the formula: —Z—R¹,
wherein Z is a group of the formula: —CH₂— or —CO—, and
R¹ is
(1) a hydrogen atom,
(2) a lower alkyl group which may be substituted by a group(s) selected from the group consisting of:
(i) a halogen atom,
(ii) an optionally protected hydroxyl group,
(iii) an amino group which may be substituted by a group(s) selected from a lower alkyl group; a lower cycloalkyl group; an aryl-lower alkyl group; a lower group; 1-(mono- or di-)lower alkyl amino-2-nitrovinyl group; 1-amino-2,2-dicyanovinyl group; 1-(mono- or di-)lower alkylamino-2,2-dicyanovinyl group; 3-aminocyclobut-3-en-1,2-dion-4-yl group; 3-(mono- or di-)lower alkylaminocyclobut-3-en-1,2-dion-4-yl group; and a group which can be removed by enzymatic or chemical metabolic process in vivo,
(iv) a guanidino group which may be substituted by a group(s) selected from a lower alkyl group, a lower cycloalkyl group and a cyano group,
(v) an ureido group which may be substituted by a group(s) selected from a lower alkyl group and a lower cycloalkyl group, and
(vi) a thioureido group which may be substituted by a group(s) selected from a lower alkyl group and a lower cycloalkyl group, or
(3) a lower alkenyl group,
R³⁰ is a nitrogen-containing heterocyclic group or its onium salt on nitrogen atom which may be substituted by a group(s) selected from an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom), a furyl-lower alkyl group, an imidazolyllower alkyl group, a thiazolyl-lower alkyl group (the thiazolyl moiety of said thiazolyl-lower alkyl group being optionally substituted by a group(s) selected from a lower alkyl group), a pyrazolyl-lower alkyl group, a pyrimidinyl-lower alkyl group, a pyridazinyl-lower alkyl group, a pyridyl-lower alkyl group (the pyridyl moiety of said pyridyl-lower alkyl group being optionally substituted by a lower alkyl group or an oxide group), a carboxyl group, a lower alkoxycarbonyl group, a halogen atom, a hydroxy-lower alkyl group, a carboxyl-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group, or a pharmaceutically acceptable salt thereof.

20. The compound according to claim 19, wherein R³⁰ is a group of the formula: 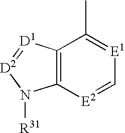
wherein D¹ and D² are the same or different and each a group of the formula: —N═ or —CH═,
one of E¹ and E² is a group of the formula: —N═, and the other is a group of the formula: —N═ or —CH═, and
R³¹ is a group selected from the group consisting of a hydrogen atom, an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidinylcarbonyl-lower alkyl group, a halogeno-lower alkyl group, a lower alkylthio-lower alkyl group, an aryl-lower alkyl group (the aryl moiety of said aryl-lower alkyl group being optionally substituted by a group(s) selected from a lower alkyl group, a lower alkoxy group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom), a furyl-lower alkyl group, an imidazolyl-lower alkyl group, a thiazolyl-lower alkyl group (the thiazolyl moiety of said thiazolyl-lower alkyl group being optionally substituted by a group(s) selected from a lower alkyl group), a pyrazolyl-lower alkyl group, a pyrimidinyl-lower alkyl group, a pyridazinyl-lower alkyl group, a pyridyl-lower alkyl group (the pyridyl moiety of said pyridyl-lower alkyl group being optionally substituted by a lower alkyl group or an oxide group), a carboxyl group, a lower alkoxycarbonyl group, a halogen atom, a hydroxy-lower alkyl group, a carboxyl-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group.

21. The compound according to claim 20, wherein R³⁰ is a group of the formula: 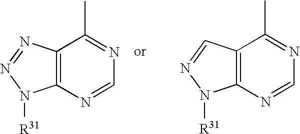
wherein R³¹ is a group selected from the group consisting of a lower alkyl group, a pyridyl-lower alkyl group (the pyridyl moiety of the pyridyl-lower alkyl group being optionally substituted by a lower alkyl group), a thiazolyl-lower alkyl group (the thiazolyl moiety of said thiazolyl-lower alkyl group being optionally substituted by a group(s) selected from a lower alkyl group), or a phenyl-lower alkyl group (the phenyl moiety of said phenyl-lower alkyl group being optionally substituted by a group(s) selected from a lower alkyl group,).

22. A compound selected from
(1α,4β)-2′-methyl-3′,4′-dihydro-6′,7′-dimethoxy-4-[4-(3-methyl-3H-1,2,3-triazolo[4,5-d]pyrimidin-7-yl)piperazin-1-yl]carbonyl-spiro[cyclohexane-1,1′(2′H)-isoquinoline],
(1α,4β)-3′,4′-dihydro-6′,7′-diethoxy-4-[4-(1-(3-ethoxyphenylmethyl)-1H-pyrazolo[3,4-d]pyrimidin-4-yl)piperazin-1-yl]carbonyl-spiro[cyclohexane-1,1′(2′H)-isoquinoline],
(1α,4β)-2′-methyl-3′,4′-dihydro-6′,7′-diethoxy-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-2′-dimethylaminoacetyl-3′,4′-dihydro-6′-methoxy-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-diethoxy-4-(4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-ishyl)-1H-pyrazolo[3,4-d]pyrimidin-4-yl)piperazin-1-yl]carbonyl-spiro[cyclohexane-1,1′(2′H)- isoquinoline], pyridin-2-ylmethyl)-1H-pyrazolo[3,4-d]pyrimidin-4-yl)piperazin-1-yl]carbonyl-spiro[cyclohexane-1,1′(2′H)-isoquinoline], ′(2′H)-isoquinoline], yridin-2-ylmethyl)-1H-pyrazolo[3,4-d]pyrimidin-4-yl)piperazin-1-yl]carbonyl-spiro[cycolhexane-1,1′(2′H)-isoquinoline],
(1α,4β)-2′-ethyl-3′,4′-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-2′-dimethylaminoacetyl-3′,4′-dihydro-6′,7′-diethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-4-[4-(1-(2-ethylthiazol-4-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-2′-dimethylaminoacetyl-3′,4′-dihydro-6′-ethoxy-4-[4-(1-(6-methylpyridin-2-ylmethyl)-1H-pyrazolo[3,4-d]-pyrimidin-4-yl)piperazin-1-yl]carbonyl-spiro[cyclohexane-1,1′(2′H)-isoquinoline],
(1α,4β)-2′-dimethylaminoacetyl-3′,4′-dihydro-6′,7′-dimethoxy-4-[4-(1-(2-methylthiazol-4-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-2′-[3-(N,N-dimethylamino)propionyl]-4-[4-(1-(3-methylbenzyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-2′-[3-(N,N-dimethylamino)propionyl]-4-[4-(1-(3-methoxybenzyl)-1H-pyrazolo[3,4-d]pyrimidin-4-yl)piperazin-1-yl]carbonyl-spiro [cyclohexane-1,1′(2′H)-isoquinoline],
(1α,4β)-3′,4′-dihydro-6′,7′-dimethoxy-2′-[2-(N,N-dimethylamino)ethyl]-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-diethoxy-4-[4-(1-(6-methylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-diethoxy-2′-[2-(N,N-dimethylamino)acetyl]-4-[4-(1-(3-methylbenzyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-4-[4-(1-(3-ethoxybenzyl)-1H-pyrazolo[3,4-d]pyrimidin-4-yl)piperazin-1-yl]carbonyl-spiro[cyclohexane-1,1′(2′H)-isoquinoline],
(1α,40β)-3′,4′-dihydro-6′-methoxy-4-[4-(1-(3-ethoxybenzyl)-1H-pyrazolo[3,4-d]pyrimidin-4-yl)piperazin-1-yl]carbonyl-spiro[cyclohexane-1,1′(2′H)-isoquinoline],
(1α,4β)-2′-[2-(N-methylamino)acetyl]-3′,4′-dihydro-6′,7′-dimethoxy-4-[4-(1-(3-ethoxybenzyl)-1H-pyrazolo[3,4-d]pyrimidin-4-yl)piperazin-1-yl]carbonyl-spiro[cyclohexane-1,1′(2′H)-isoquinoline],
(1α,4β)-2′-[2-(N-methylamino)acetyl]-3′,4′-dihydro-6′,7′-dimethoxy-4-[4-(1-(3-trifluoromethoxybenzyl)-1H-pyrazolo[3,4-d]pyrimidin-4-yl)piperazin-1-yl]carbonyl-spiro[cyclohexane-1,1′(2′H)-isoquinoline],
(1α,4β)-2′-[2-(N-methylamino)acetyl]-3′,4′-dihydro-6′,7′-dimethoxy-4-[4-(1-(6-ethylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-4-[4-(1-(6-n-propylpyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-2′-[2-(N,N-dimethylamino)acetyl]-3′,4′-dihydro-6′-ethoxy-4-[4-(1-(6-ethoxypyridin-2-ylmethyl)-1H-pyrazolo[3,4-d]pyrimidin-4-yl)piperazin-1-yl]carbonyl-spiro[cyclohexane-1,1′(2′H)-isoquinoline],
(1α,4β)-3′,4′-dihydro-6′,7′-dimethoxy-2′-[3-(1-methyl-amino-2-nitrovinylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(1-amino-2-nitrovinylamino)propyl]-4-[4-[1-(2-nitrobenzyl)-1H-pyrazolo[3,4-d]pyrimidin-4-yl]piperadin-
(1α,4β)-3′,4′-dihydro-6′,7′-dimethoxy-2′-[3-(N-(n-butyl)ureido)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(N-ethylureido)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(3-dimethylamino-3-cyclobuten-1,2-dion-4-yl)aminopropyl]-4-[4-[1-(2-nitrobenzyl)-1H-pyrazolo[3,4-d]pyrimidin-4-yl]-piperadin-1-yl]carbonyl-spiro[cyclohexane-1,1′(2′H)-isoquinoline],
(1α,4β)-3′,4′-dihydro-6′,7′-dimethoxy-2′-[3-(3-methylamino-3-cyclobuten-1,2-dion-4-yl)aminopropy]4-[4-[1-(2-nitrobenzyl)-1H-pyrazolo[3,4-d]pyrimidin-4-yl]-piperadin-1-yl]carbonyl-spiro[cyclohexane-1,1′(2′H)-isoquinoline],
(1α,4β)-3′,4′-dihydro-6′,7′-dimethoxy-2′-[3-(3-amino-3-cyclobuten-1,2-dion-4-yl)aminopropyl]-4-[4-[1-(2-nitro-benzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(1-amino-2,2-dicyanovinylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(1,3-dimethyl-2-cyanoguanidino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(N-isopropylamino)propyl]-4-[4-[1-(2-nitrobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-(N,N-dimethylaminoacetyl)-4-[4-(1-(2-nitrobenzyl)-1H-pyrazolo[3,4-d]pyrimidin-4-yl]piperadin-1-yl]carbonyl-spiro-[cyclohexane-1,1′(2′H)-isoquinoline],
(1α,4β)-3′,4′-dihydro-6′,7′-dimethoxy-2′-ethyl-4-[4-[1-(3-methylbenzyl)-1H-pyrazolo[3,4-d]pyrimidin-4-yl]-piperadin-1-yl)carbonyl-spiro[cyclohexane-1,1′(2′H)-isoquinoline],
(1α,4β)-3′,4′-dihydro-6′,7′-dimethoxy-2′-methyl-4-[4-[1-(2-fluorobenzyl)-1H-pyrazolo[3,4-d]pyrimidin-4-yl]-piperadin-1-yl]carbonyl-spiro[cyciohexane-1,1′(2′H)-isoquinoline],
(1α,4β)-3′,4′-dihydro-6′,7′-dimethoxy-2′-[3-(N-ethoxy-carbonylamino)propyl]-4-[4-[1-(2-bromobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(N-ethoxy-carbonylamino)propyl]-4-[4-[1-(2-chlorobenzyl)-1H-pyrazolo[3,4-d]pyrimidin-4-yl]piperadin-1-yl]carbonyl-spiro[cyclohexane-1,1′(2′H)-isoquinoline],
(1α,4β)-3′,4′-dihydro-6′,7′-dimethoxy-2′-[3-(N-ethoxy-carbonylamino)propyl]-4-[4-[1-(2-cyanobenzyl)-1H-pyrazolo[3,4-d]pyrimidin-4-yl)piperadin-1-yl]carbonyl-4-[4-[1-(2-cyanobenzyl)-1H-pyrazolo[3,4-d]pyrimidin-4-yl]piperadin-1-yl]carbonyl-
(1α,4β)-3′,4′-dihydro-6′,7′-diethoxy-2′-methyl-4-[4-[1-(3-nitrobenzyl)-1H-pyrazolo[3,4-d]pyrimidin-4-yl]piperadin-1-yl]carbonyl-spiro[cyclohexane-1,1′(2′H)-isoquinoline], and
(1α,4β)-3′,4′-dihydro-6′,7′-diethoxy-2′-methyl-4-[4-(1-(3-chlorobenzyl)-1H-pyrazolo[3,4-d]pyrimidin-4-yl]piperadin-1-yl]carbonyl-spiro[cyclohexane-1,1′(2′H)-isoquinoline],
or a pharmaceutically acceptable salt thereof.

23. A compound selected from
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinylmethyl]carbonyl-spiro[cyclohexane-1,1′(2-′H)-isoquinoline],
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline],
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(3-pyridylmethyl)-1H-pyrazolo[3,4-d]pyrimidin-4-yl]-1-piperazinyl]carbonyl-spiro[cyclohexane-1,1′(2′H)-isoquinoline), pyrimidin-4-yl]-1-piperazinyl]carbonyl-spiro[cyclohexane-1,1′(2′H)-isoquinoline], -tetrahydro-6,7-dimethoxy-1-isoquinolyl)-4-[4-(4-n-butyl-4H-imidazo[4,5-b]pyridin-7-yl)-1-piperazinyljcarbonyl-spiro[cyclohexane-1,1′(2′H)-isoquinoline), -yl)-1-piperazinyl]carbonyl-spiro[cyclohexane-1,1′(2′H)-isoquinoline], -tetrahydro-6,7-dimethoxy-1-isoquinolyl)-4-[4-(3-methyl-3H-1,2,3-triazolo[4,5-d]pyrimidin-7-yl)-1-piperazinyl]carbonyl-spiro[cyclohexane-1,1′(2′H)-isoquinoline],
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-(1-n-propyl-1H-pyrazolo[3,4-d]pyrimidin-4-yl)-1-piperazinyl]carbonyl-spiro[cyclohexane-1,1′(2′H)-isoquinoline],
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(2-chrolophenylmethyl)-1H-pyrazolo[3,4-d]pyrimidin-4-yl]-1-piperazinyl]carbonyl-spiro[cyclohexane-1,1′(2′H)-isoquinoline],
2′-[3-(methylamino)propionyl]-3′,4′-dihydro-6′,7′-dimethoxy-2-(2-ethyl-1,2,3,4-tetrahydro-6,7-dimethoxy-1-isoquinolyl)-4-[4-[1-(3-methylphenylmethyl)-1H-pyrazolo[3,4-d]pyrimidin-4-yl]-1-piperazinyl]carbonyl-spiro[cyclohexane-1,1′(2′H)-isoquinoline],
2′-(3-aminopropyl)-3′,4′-dihydro-6′,7′-dimethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline],
2′-(3-aminopropyl)-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline],
2′-[3-(2-cyano-3,3-dimethylguanidino)propyl]-3′,4′-dihydro-6′,7′-dimethoxy-4-4-[1-(2-nitrophenylmethyl)-1H-pyrazolo[3,4-d]pyrimidin-4-yl]-1-piperazinyl]carbonyl4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline], 4-[4-[1-(2-chlorophenylmethyl)-1H-pyrazolo[3,4-d]pyrimidin-4-yl]-1-piperazinyl]carbonyl-spiro[cyclohexane-1,1′(2′H)-isoquinoline],
2′-(3-aminopropyl)-3′,4′-dihydro-6′,7′-dimethoxy-4-[4-[1-(2-bromophenylmethyl)-1H-pyrazolo[3,4-d]pyrimidin-4-yl]-1-piperazinyl]carbonyl-spiro[cyclohexane-1,1′(2′H)-isoquinoline],
2′-[3-[N-(propionyloxymethyloxycarbonyl)-N-methylamino]-propionyl]-3′,4′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],
2′-[3-[N-(pivaloyloxymethyloxycarbonyl)-N-methylamino]-propionyl]-3′,4′-dihydro-6′,7′
2′-[3-[N-(pivaloyloxymethyloxycarbonyl)-N-methylamino-propionyl]-3′,4′-dihydro-6′,7′4-pyridylmethyl)-1H-pyrazolo[3,4-d]pyrimidin-4-yl]-1-piperazinyl]carbonyl-spiro[cyclohexane-1,1′(2′H)-isoquinoline],
2′-[3-[N-(pivaloyloxymethyloxycarbonyl)-N-methyl-amino]propionyl]-3′,4′-dihydro-6′,7′-dimethoxy-2-(2-ethyl-1,2,3,4-tetrahydro-6,7-dimethoxy-1-isoquinolyl)-4-[4-(3-methyl-3H-1,2,3-triazolo[4,5-d]pyrimidin-7-yl)-1-piperazinyl]carbonyl-spiro[cyclohexane-1,1′(2′H)-isoquinoline],
2′-[3-[N-(cyclopropylcarbonyloxymethyloxycarbonyl)-N-methylamino]propionyl]-3′,41′-dihydro-6′,7′-dimethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline],
2′-[3-(pivaloyloxymethyloxycarbonylamino)propyl]-3′,4′-dihydro-6′,7′-dimethoxy-4-[4-[1-(2-nitrophenylmethyl)-1H-pyrazolo[3,4-d]pyrimidin-4-yl]-1-piperazinyl]carbonyl-spiro[cyclohexane-1,1′(2′H)-isoquinoline],
(1α,4β)-3′,4′-dihydro-6′,7′-dimethoxy-4-[4-[1-(3-nitrobenzyl)-1H-pyrazolo[3,4-d]pyrimidin-4-yl]-1-piperazinyl]carbonyl-spiro[syclohexane-1,1′(2′H)-isoquinoline],
(1α,4β)-2′-dimethylaminoacetyl-3′,4′-dihydro-6′,7′-dimethoxy-4-[4-[1-(3-methylbenzyl)-1H-pyrazolo(3,4-d]pyrimidin-4-yl]-1-piperazinyl]carbonyl-spiro[cyclohexane--(3-methylbenzyl)-1H-pyrazolo[3,4-d]pyrimidin-4-yl]-1-piperazinyl]carbonyl-spiro[cyclohexane-
(1α,4β)-2′-methyl-3′,4′-dihydro-6′,7′-dimethoxy-4-[4-(3-methyl-3H-1,2,3-triazolo[4,5-d]pyrimidin-7-yl)-1-piperazinyl]carbonyl-spiro[cyclohexane-1,1′(2′H)-isoquinoline], and
(1α,4β)-2′-ethyl-3′,4′-dihydro-6′,7′-diethoxy-4-[4-[1-(2-pyridylmethyl)-1H-pyrazolo[3,4-d]pyrimidin-4-yl]-1-piperazinyl]carbonyl-spiro[cyclohexane-1,1′(2′H)-isoquinoline],
or a pharmaceutically acceptable salt thereof.

24. A compound selected from
(1R*,2R*(S*),4R*)-2′-[3-(methylamino)propionyl]-3′,4′-dihydro-6′,7′-dimethoxl-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′(2′H)isoquinoline],
(1R*,2R*(S*),4R*)-2′-[3-[N-(propionyloxymethyloxycarbonyl)-N-methylamino]propionyl]-3′,4′-dihydro-6′,7′-dirmethoxy-2-(2-ethyl-1,2,3,4-tetrahydro-6,7-dimethoxy-1-isoquinolyl)-4-[4-[1-(4-pyridylmethyl)-1H-pyrazolo[3,4-d]pyrimidin-4-yl]-1-piperazinyl]carbonyl-spiro[cyclohexane-1,1′(2′H)-isoquinoline], and
(1α,4β)-2′-methyl-3′,4′-dihydro-6′,7′-dimethoxy-4-[4-(3-methyl -3H-1,2,3-triazolo[4,5-d]pyrimidin-7-yl]-1-piperazinyl]carbonyl-spiro[cyclohexane-1,1′(2′H)-isoquinoline],
or a pharmaceutically acceptable salt thereof.

25. A process for preparing a compound of the formula [I-a]: 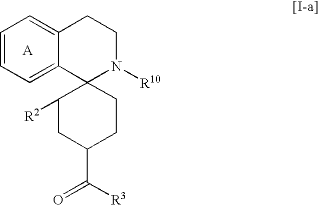
wherein ring A is an optionally substituted benzene ring,
R¹⁰ is a hydrogen atom or a group of the formula: —Z—R¹,
wherein R¹ is a hydrogen atom, an optionally substituted lower alkyl group, or an optionally substituted lower alkenyl group, and
Z is a group of the formula: —CH₂— or —CO—,
R² is a hydrogen atom or an optionally substituted heterocyclic group,
R³ is an optionally substituted amino group or an optionally substituted nitrogen-containing aliphatic heterocyclic group, or a pharmaceutically acceptable salt thereof,
which comprises reacting a compound of the formula[II-A]: 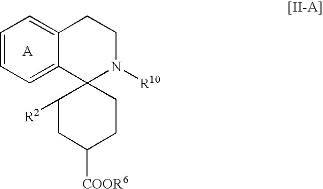
wherein R⁶ is a hydrogen atom, a lower alkyl group or a benzyl group and the other symbols are the same as defined above, or a salt thereof,
with a compound of the formula[16]:
R³—H [16]
wherein R³ is the same as defined above, or a salt thereof, and if necessary, followed by converting the product into a pharmaceutically acceptable salt thereof.

26. A process for preparing a compound of the formula[I-e]: 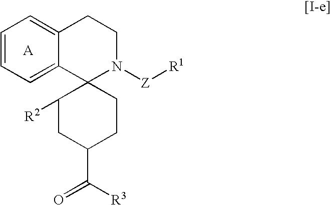
wherein ring A is an optionally substituted benzene ring,
R¹ is a hydrogen atom, an optionally substituted lower alkyl group, or an optionally substituted lower alkenyl group,
Z is a group of the formula: —CH₂— or —CO—,
R² is a hydrogen atom or an optionally substituted heterocyclic group, and
R³ is an optionally substituted amino group or an optionally substituted nitrogen-containing aliphatic heterocyclic group, or a pharmaceutically acceptable salt thereof,
which comprises reacting the compound of the formula[II-c]: 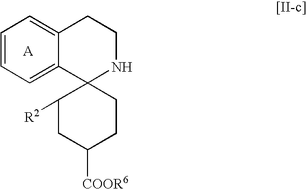
wherein R⁶ is a hydrogen atom, a lower alkyl group or a benzyl group and the other symbols are the same as defined above, or a salt thereof,
with a compound of the formula[9]:
X—Z—R¹ [9]
wherein X is a leaving group and the other symbols are the same as defined above, or a salt thereof,
to obtain a compound of the formula[II-b]: 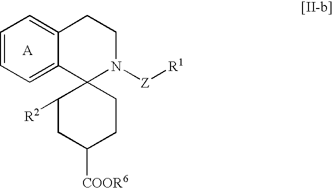
wherein the symbols are the same as defined above,
and then reacting thus obtained compound[II-b] with a compound of the formula[16]:
R³—H [16]
wherein R³ is the same as defined above, or a salt thereof,
and if necessary, followed by converting the product into a pharmaceutically acceptable salt thereof.

27. A process for preparing a compound of the formula[I-e]: 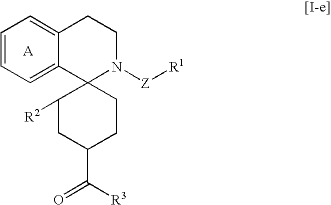
wherein ring A is an optionally substituted benzene ring,
R¹ is a hydrogen atom, an optionally substituted lower alkyl group, or an optionally substituted lower alkenyl group,
Z is a group of the formula: —CH₂— or —CO—,
R² is a hydrogen atom or an optionally substituted heterocyclic group, and
R³ is an optionally substituted amino group or an optionally substituted nitrogen-containing aliphatic heterocyclic group, or a pharmaceutically acceptable salt thereof,
which comprises reacting a compound of the formula[I-f]: 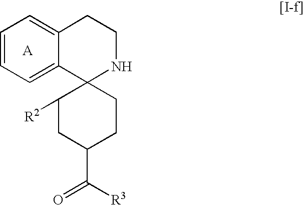
wherein the symbols are the same as defined above, or a salt thereof,
with a compound of the formula[9]:
X—Z—R¹ [9]
wherein X is a leaving group and the other symbols are the same as defined above, or a salt thereof,
and if necessary, followed by converting the product into a pharmaceutically acceptable salt thereof.

28. A process for preparing a compound of the formula[I-e]: 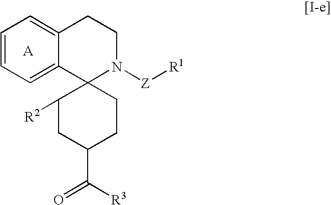
wherein ring A is an optionally substituted benzene ring,
R¹ is a hydrogen atom, an optionally substituted lower alkyl group, or an optionally substituted lower alkenyl group,
Z is a group of the formula: —CH₂— or —CO—,
R² is a hydrogen atom or an optionally substituted heterocyclic group, and R³ is an optionally substituted amino group or an optionally substituted nitrogen-containing aliphatic heterocyclic group, or a pharmaceutically acceptable salt thereof,
which comprises reacting a compound of the formula[II-c]: 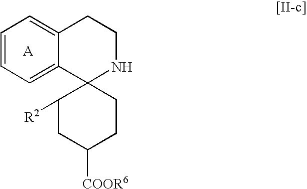
wherein R⁶ is a hydrogen atom, a lower alkyl group or benzyl croup and the other symbols are the same as defined above, or a salt thereof,
with a compound of the formula[16]:
R³—H [16]
wherein R³ is the same as defined above, or a salt thereof,
to obtain a compound of the formula[I-f]: 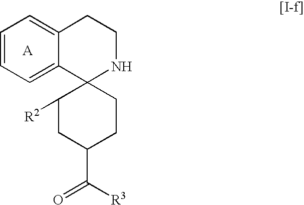
wherein the symbols are the same as defined above, or a salt thereof,
and then reacting thus obtained compound[I-f] with a compound of the formula[9]:
X—Z—R¹ [9]
wherein X is a leaving group and the other symbols are the same as defined above, or a salt thereof,
and if necessary, followed by converting the product into a
and if necessary, followed by converting the product into a

29. A process for preparing a compound of the formula[I-b]: 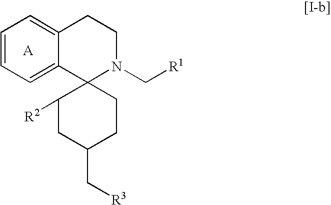
wherein ring A is an optionally substituted benzene ring,
R¹ is a hydrogen atom, an optionally substituted lower alkyl group, or an optionally substituted lower alkenyl group,
R² is a hydrogen atom or an optionally substituted heterocyclic group,
R³ is an optionally substituted amino group or an optionally substituted nitrogen-containing aliphatic heterocyclic group, or a pharmaceutically acceptable salt thereof,
which comprises reducing a compound of the formula[I-e]: 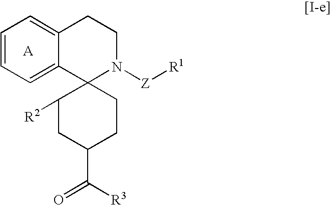
wherein Z is a group of the formula: —CH₂— or —CO— and the other symbols are the same as defined above, or a salt thereof,
and if necessary, followed by converting the product into a pharmaceutically acceptable salt thereof.

30. A process for preparing a compound of the formula[I-d₁]: 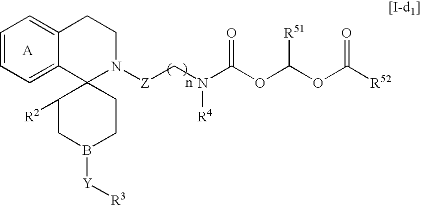
wherein ring A is an optionally substituted benzene ring,
Z is a group of the formula: —CH₂— or —CO—,
R² is a hydrogen atom or an optionally substituted heterocyclic group,
R³ is an optionally substituted amino group or an optionally substituted nitrogen-containing aliphatic heterocyclic group,
R⁴ is a hydrogen atom or a lower alkyl group,
n is an integer from 1 to 6,
R⁵¹ is a hydrogen atom or a lower alkyl group,
R⁵² is a lower alkyl group (said lower alkyl group being optionally substituted by a carboxyl group), a lower cycloalkyl group, a lower alkoxy group, a lower cycloalkoxy group or an aryl group,
B is a group of the formula: 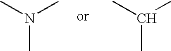
and Y is a group of the formula: —CH₂— or —CO—,
or a pharmaceutically acceptable salt thereof,
which comprises reacting the compound of the formula[I-c]: 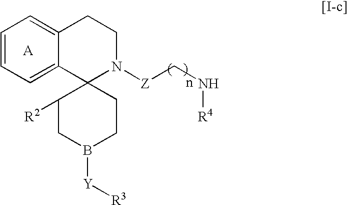
wherein the symbols are the same as defined above, or a salt thereof,
with a compound of the formula[17]: 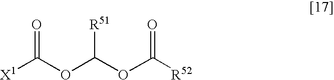
wherein X¹ is a leaving group and the other symbols are the same as defined above, or a salt thereof,
and if necessary, followed by converting the product into a pharmaceutically acceptable salt thereof.

31. A process for preparing a compound of the formula[I-d₁]: 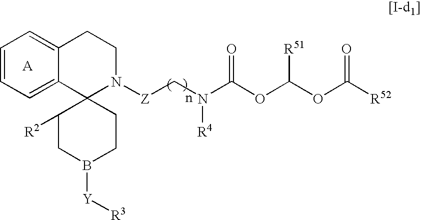
wherein ring A is an optionally substituted benzene ring,
Z is a group of the formula: —CH₂— or —CO—,
R² is a hydrogen atom or an optionally substituted heterocyclic group,
R³ is an optionally substituted amino group or an optionally substituted nitrogen-containing aliphatic heterocyclic group,
R⁴ is a hydrogen atom or a lower alkyl group,
n is an integer from 1 to 6,
n is an integer from 1 to 6,
R⁵² is a lower alkyl group (said lower alkyl group being optionally substituted by a carboxyl group), a lower cycloalkyl group, a lower alkoxy group, a lower cycloalkoxy group or an aryl group,
B is a group of the formula: 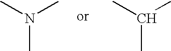
and Y is a group of the formula: —CH₂— or —CO—,
or a pharmaceutically acceptable salt thereof,
which comprises reacting a compound of the formula[I-c]: 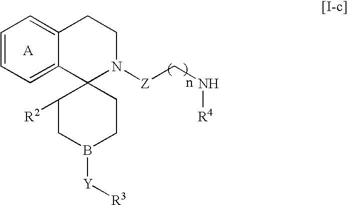
wherein the symbols are the same as defined above, or a salt thereof,
with a compound of the formula[18]: 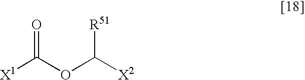
wherein X¹ is a leaving group, R⁵¹ is the same as defined
above and X² is a halogen atom, or a salt thereof,
to obtain a compound of the formula[I-d₇]: 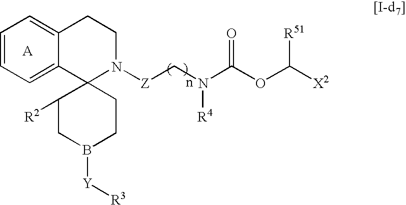
wherein the symbols are the same as defined above,
and then reacting thus obtained compound[I-d₇] with a compound of the formula[19]:
R⁵²—COOH [19]
wherein R⁵² is the same as defined above, or a salt thereof,
and if necessary, followed by converting the product into a pharmaceutically acceptable salt thereof.

32. A process for preparing a compound of the formula[I-g]: 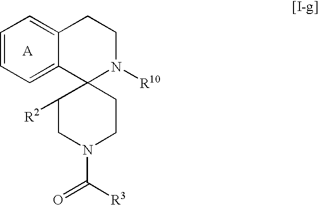
wherein ring A is an optionally substituted benzene ring,
R¹⁰ is a hydrogen atom or a group of the formula: —Z—R¹,
wherein R¹ is a hydrogen atom, an optionally substituted lower alkyl group or an optionally substituted lower alkenyl group,
Z is a group of the formula: —CH₂— or —CO—,
R² is a hydrogen atom or an optionally substituted
R² is a hydrogen atom or an optionally substituted amino group or an optionally substituted nitrogen-containing aliphatic heterocyclic group,
or a salt thereof,
which comprises reacting a compound of the formula[II-B]: 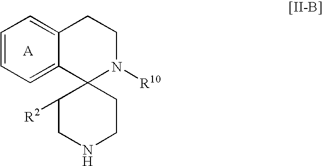
wherein the symbols are the same as defined above, or a salt thereof,
with a compound of the formula[16]:
R³—H [16]
wherein R³ is the same as defined above, or a salt thereof,
in the presence of a phosgene-equivalent,
and if necessary, followed by converting the product into a pharmaceutically acceptable salt thereof.

33. A spiroisoquinoline derivative of the formula[II-A]: 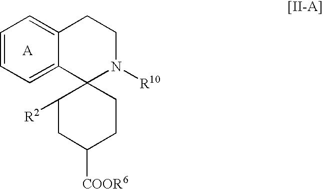
wherein ring A is an optionally substituted benzene ring,
R¹⁰ is a hydrogen atom or a group of the formula: —Z—R¹,
wherein R¹ is a hydrogen atom, an optionally substituted lower alkyl group or an optionally substituted lower alkenyl group, and
Z is a group of the formula: —CH₂— or —CO—,
R² is a hydrogen atom or an optionally substituted heterocyclic group, and
R⁶ is a hydrogen atom, a lower alkyl group or a benzyl group,
or a salt thereof.

34. A spiroisoquinoline derivative of the formula[II-B]: 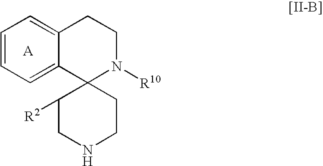
wherein ring A is an optionally substituted benzene ring,
R¹⁰ is a hydrogen atom or a group of the formula: —Z—R¹,
wherein R¹ is a hydrogen atom, an optionally substituted lower alkyl group or an optionally substituted lower aikenyl group,
Z is a group of the formula: —CH₂— or —CO—, and
R² is a hydrogen atom or an optionally substituted heterocyclic group,
or a salt thereof.

35. A process for preparing a compound of the formula[III]: 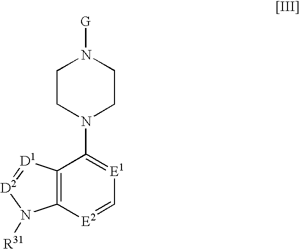
wherein D¹ and D² are the same or different and each a group of the formula: —N═ or —CH═,
one of E¹ and E² is a group of the formula: —N═, and the other is a group of the formula: —N═ or —CH═, other is a group of the formula: —N═ or —CH═, hydrogen atom, an oxo group, an oxide group, a lower alkyl group, a cyano lower alkyl group, a lower cycloalkyl-lower alkyl group (a carbon atom(s) on said lower cycloalkyl group being optionally substituted by a sulfur atom(s)), a pyrrolidonocarbonyl-lower alkyl group, a halogeno-lower alkyl group, a lower alkylthio-lower alkyl group, an aryl-lower alkyl group (the aryl moiety of said aryl-lower alkyl group being optionally substituted by a groupfs) selected from a lower alkyl group, a lower alkoxy group, a halogen atom, a tri-halogenomethyl group, a tri-halogenomethoxy group, a nitro group, and a cyano group), a thienyl-lower alkyl group (the thienyl moiety of said thienyl-lower alkyl group being optionally substituted by a group(s) selected from a halogen atom), a furyl-lower alkyl group, an imidazolyl-lower alkyl group, athiazolyl-lower alkyl group (the thiazolyl moiety of said thiazolyl-lower alkyl group being optionally substituted by a group(s) selected from a lower alkyl group), a pyrazolyl-lower alkyl group, a pyrimidinyl-lower alkyl group, a pyridazinyl-lower alkyl group, a pyridyl-lower alkyl group (the pyridyl moiety of said pyridyl-lower alkyl group being optionally substituted by a lower alkyl group or an oxide group), a carboxyl group, a lower alkoxycarbonyl group, a halogen atom, a hydroxy-lower alkyl group, a carboxyl-lower alkyl group, a lower alkoxycarbonyl-lower alkyl group, an aryl-lower alkoxy-lower alkyl group (the aryl moiety of said aryl-lower alkoxy-lower alkyl group being optionally substituted by a halogen atom(s)), an amino-protecting group, an amino group (said amino group being optionally substituted by a lower alkyl group(s)), a lower cycloalkyl group, an N-pyridyl-N-lower alkylcarbamoyl group, a lower alkenyl group, a halogeno-lower alkenyl group, and an aryl group (said aryl group being optionally substituted by a group(s) selected from a trifluoromethyl group, a lower alkoxy group, and a nitro group, and G is an amino protecting group, or a salt thereof,
which comprises reacting a compound of the formula [28]: 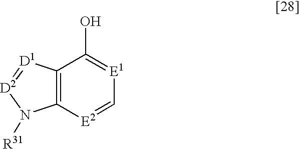
wherein the symbols are the same as defined above, or a salt thereof,
with a compound of the formula[29]: 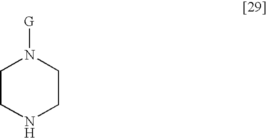
wherein G is the same as defined above, or a salt thereof, and if necessary, followed by converting the product into a salt thereof.

36. A medicament for prophylaxis or treatment of constipation, irritable bowel syndrome, gastroesophageal reflux disease or post-operative ileus, which comprises as an active ingredient a compound having SK channel blocking activity.

37. A medicament for prophylaxis or treatment of gastrointestinal motility disorders, central nervous system disorders, memory and learning disorders including Alzheimer's disease, emotional disorders, myotonic muscular dystrophy or sleep apnea, which comprises as an active ingredient a compound having Doth SK channel blocking activity and acetylcholinesterase inhibiting activity.

38. A pharmaceutical composition, which comprises as an active ingredient a compound according to any one of claims 1 to 24 or a pharmaceutically acceptable salt thereof and a pharmaceutically acceptable carrier therefor.

39. The pharmaceutical composition according to claim 38, which is used for treatment or prophylaxis of gastrointestinal motility disorders, central nervous system disorders, memory and learning disorders including Alzheimer's disease, emotional disorders, myotonic muscular dystrophy or sleep apnea.

40. Use of a compound according to any one of claims 1 to 24 or a pharmaceutically acceptable salt thereof in the preparation of a medicament for treatment or prophylaxis of SK channel-related diseases.

41. The use according to claim 40 wherein the SK channel-related diseases is gastrointestinal motility disorders, central nervous system disorders, myotonic muscular dystrophy or sleep apnea.

42. The use according to claim 40 wherein the SK channel-related diseases is constipation, irritable bowel syndrome, gastroesophageal reflux disease, post-operative ileus, memory and learning disorders including Alzheimer's disease, emotional disorders, myotonic muscular dystrophy or sleep apnea.

43. A method for treatment or prophylaxis of SK channel-related diseases, which comprises administering an effective amount of a compound according to any one of claims 1 to 24 or a pharmaceutically acceptable salt thereof, to a patient in need thereof.

44. The method according to claim 43 wherein the SK channel-related diseases is gastrointestinal motility disorders, central nervous system disorders, myotonic muscular dystrophy or sleep apnea.

45. The method according to claim 43 wherein the SK channel-related diseases is constipation, irritable bowel syndrome, gastroesophageal reflux disease, post-operative ileus, memory and learning disorders including Alzheimer's disease, emotional disorders, myotonic muscular dystrophy or sleep apnea.
